(19) United States
(12) Patent Application Publication
(10) Pub. No.: US 2025/0368722 A1
(43) Pub. Date: Dec. 4, 2025

(54) MULTISPECIFIC ANTIBODIES TARGETING MULTIPLE EPITOPES ON THE HIV-1 ENVELOPE

(71) Applicants: University of Maryland, College Park, College Park, MD (US); University of Maryland, Baltimore, Baltimore, MD (US); The U.S.A, as Represented by the Secretary, Department of Health and Human Services, Bethesda, MD (US); International AIDS Vaccine Initiative, New York, NY (US)

(72) Inventors: Yuxing Li, Boyds, MD (US); James Steinhardt, Westminster, MD (US); Javier Guenaga, San Diego, CA (US); John R. Mascola, Rockville, MD (US); Tae-Wook Chun, North Bethesda, MD (US); Susan Moir, Washington, DC (US); Chi-I Chiang, Rockville, MD (US)

(21) Appl. No.: 19/231,954

(22) Filed: Jun. 9, 2025

Related U.S. Application Data
(62) Division of application No. 17/285,956, filed on Apr. 16, 2021, now Pat. No. 12,365,722.
(60) Provisional application No. 62/748,228, filed on Oct. 19, 2018.
(60) Provisional application No. 62/749,510, filed on Oct. 23, 2018.

(51) Int. Cl. C07K 16/10; A61K 39/00; A61K 39/42; A61K 45/06; A61P 31/18

(52) U.S. Cl. CPC C07K 16/1063; A61K 39/42; A61K 45/06; A61P 31/18; C07K 16/1045; A61K 2039/505; C07K 2317/31; C07K 2317/52; C07K 2317/565; C07K 2317/622; C07K 2317/76; C07K 2317/92

(57) ABSTRACT

The present invention provides a multispecific anti-HIV antibody that binds to multiple epitopes on HIV envelope protein, wherein the antibody comprises:
i. an amino acid sequence that binds to a V1/V2 apex glycan epitope;
ii. an amino acid sequence that binds to a V3-base glycan region epitope;
iii. an amino acid sequence that binds to a CD4 binding site (CD4bs) epitope;
iv. an amino acid sequence that binds to a gp120/gp41 interface epitope; and
v. an amino acid sequence that binds to a membrane proximal external region (MPER) epitope.

This application claims priority to U.S. Ser. No. 17/285,956, filed on Apr. 16, 2021, which is a 371 of International Application No. PCT/US2019/057089, filed on Oct. 18, 2019, which claims priority to U.S. Provisional Application No. 62/749,510, filed Oct. 23, 2018 and U.S. Provisional Application No. 62/748,228 filed Oct. 19, 2018. The entire disclosures of all of the foregoing applications are incorporated by reference herein.

This invention was made with government support under AI136756 awarded by the National Institutes of Health. The government has certain rights in the invention.

### FIELD OF THE INVENTION

The field of the invention relates to medicine, infectious disease and in particular antibodies which can neutralize HIV-1 virus strains.

### SEQUENCE LISTING

The application contains a Sequence Listing which has been submitted electronically in .XML format and is hereby incorporated by reference in its entirety. Said. XML copy, created on Aug. 25, 2025, is named “1475-59 PCT.xml” and is 220,353 bytes in size. The sequence listing contained in this .XML file is part of the specification and is hereby incorporated by reference herein in its entirety.

### BACKGROUND OF THE INVENTION

The acquired immunodeficiency syndrome (AIDS) is caused by the human immunodeficiency virus type 1 (HIV-1) (Gallo et al., Science 224, 500-503. (1984); Barre-Sinoussi et al., Science 220, 868-871. (1983)), and results from infection and depletion of human CD4+ lymphocytes. HIV-1 infection of CD4+ host cells is mediated by the viral envelope glycoproteins (Env), which are displayed as trimeric spikes that sparsely coat the surface of the HIV virion. Each trimeric Env complex is composed of three of each exterior envelope glycoprotein, gp120, and the gp41 transmembrane envelope glycoprotein (Kowalski et al., Science 237, 1351-1355. (1987); Lu et al., Nat Struct Biol 2, 1075-1082 (1995). This trimeric Env complex is the main target of neutralizing antibody responses [reviewed by (Wyatt et al., Science 280, 1884-1888. (1998)].

Over the past two decades, advances in the treatment of human immunodeficiency virus (HIV) infection have led to dramatic improvements in health outcomes of infected individuals receiving antiretroviral therapy (ART). Nonetheless, the vast majority of HIV-infected individuals must remain on continuous, life-long ART in order to maintain suppression of HIV replication and prevent progression to AIDS. While effective at suppressing HIV replication, current ART requires life-long adherence to daily medication regimens and is associated with significant costs, cumulative toxicities, and the potential for emergence of drug-resistant virus.

Consequently, there has been considerable interest in strategies that would allow for discontinuation of ART while maintaining suppression of plasma viremia for prolonged periods. In this regard, recent advances in immunogen and antibody cloning technologies have led to the isolation of several highly potent and broadly neutralizing HIV-specific antibodies (bNAb) from B cells of infected individuals. Some bNAbs have exhibited strong activities against HIV and SIV in vitro, in infected animals, and in infected individuals who were not receiving ART.

Recently, broadly neutralizing antibodies (bNAbs) have been explored as prevention and therapy agents for the treatment and management of HIV-1, as they can i) inhibit virus entry by neutralizing the free infectious virus particles, ii) prevent virus cell-to-cell spread, and iii) eliminate virus-infected cells by binding the Env molecules expressed on the surface of the infected cells and triggering antibody-dependent cell-mediated cytotoxicity (ADCC) through interactions between the IgG Fc region and the Fc receptors on effector cells (primarily NK cells) [reviewed in Burton et al., Nat Immunol 16, 571-576 (2015)]. Furthermore, the repertoire of isolated bNAbs has drastically increased recently as the process for bNAb isolation and characterization has accelerated with the integration of emerging functional and structural information and new technologies of single B cell sorting and cloning (Burton et al., Proc Natl Acad Sci USA 88, 10134-10137 (1991); Buchacher et al., AIDS Res Hum Retroviruses 10, 359-369 (1994); Huang et al., Nature 491, 406-412 (2012); Scheid et al., Science 333, 1633-1637 (2011); Wu et al., Science 329, 856-861 (2010); Walker et al., Nature 477, 466-470 (2011)). The characterization of HIV-1 bNAbs and their cognate epitopes on the Env spikes has identified five conserved Env sites of vulnerability including, in the order from the apex to the stem of Env: the V1/V2-glycan region, the V3-glycan region, the CD4-binding site (CD4bs), the gp120-gp41 interface (Burton et al., Nat Immunol 16, 571-576 (2015)), and the gp41 membrane proximal external region (MPER) (Burton et al., Nat Immunol 16, 571-576 (2015); Kwong et al., Nat Rev Immunol 13, 693-701 (2013)) (FIG. 1).

Administration of a single bNAb as a therapeutic agent has successfully cleared phase I safety clinical trials, demonstrating temporary HIV-1 viremia suppression in the majority of patients (Caskey et al., Nature 522, 487-491 (2015); Ledgerwood et al., Clin Exp Immunol 182, 289-301 (2015)). Unfortunately, the HIV virus rapidly develops resistance mutations under the selective pressure of single bNAb, suggesting that passive treatment with a single bNAb is unlikely to result in long-term viremia suppression (Caskey et al., Nature 522, 487-491 (2015); Lynch et al., Sci Transl Med 7, 319ra206 (2015); Bar et al., N Engl J Med 375, 2037-2050 (2016); Caskey et al., Nat Med 23, 185-191 (2017)). Fortunately, some Env mutations associated with bNAb resistance can reduce viral fitness, suggesting that simultaneously targeting different Env epitopes may compromise viral replication (Tebit et al., AIDS Rev 9, 75-87 (2007); Sather et al., J Virol 86, 12676-12685 (2012); Lynch et al., J Virol 89, 4201-4213 (2015); Pietzsch et al., J Exp Med 207, 1995-2002 (2010)). The quick onset of escaping virus quasi species in these clinical trials with single bNAb agent strongly highlights the need to develop combinational therapy regimens to control virus rebound by preventing emergence of resistant virus. Additionally, in vitro data from previous studies have demonstrated that combinatorial bNAb therapies display a substantial gain of neutralization potency and breadth when two or more bNAbs targeting independent epitopes are combined (Kong et al., J Virol 89, 2659-2671 (2015); Doria-Rose et al., J Virol 86, 3393-3397 (2012)). This in vitro data is further supported by a number of in vivo animal studies in which dual-, triple-, and penta-combinations of bNAbs resulted in improved protective efficacy compared to mono bNAb therapy (Shingai et al., Nature 503, 277-280 (2013); Klein et al., Nature 492, 118-122 (2012)).

Collectively, these findings suggest that passive immunotherapy with a bNAb(s) could potentially prevent plasma viral rebound in HIV-infected individuals following cessation of ART. For this reason, we recently conducted a clinical trial to investigate whether VRC01 could prevent plasma viral rebound upon discontinuation of ART. While multiple infusions of VRC01 were safe and well-tolerated, the majority of patients experienced plasma viral rebound due to pre-existing and emergent VRC01-resistant HIV despite adequate levels of antibody in plasma (Lynch, Sci Transl Med 7, 319ra206 (2015); Bar et al., N Engl J Med 375, 2037-2050 (2016)). Therefore, therapeutic strategies involving passive transfer of bNAbs may require a combination of bNAbs that targets multiple regions on HIV Env protein in order to achieve sustained virologic control upon withdrawal of ART.

Of note, the potential significance of the above concept was recently highlighted in an animal experiment in which a trimeric antibody consisting of VRC01, PGDM1400, and 10E8 showed profound potency and breadth against a mixture of SHIV (Xu et al., Science 358, 85-90 (2017)). Building upon these findings, it is of great importance to explore the effect of multimeric bNAbs (or bNAb-like molecules) that possess broad and potent neutralization capacity against highly heterogeneous infectious viral isolates.

While antibody cocktails demonstrated improved efficacy in preclinical studies, multispecific “single agents” are desirable for manufacturing purposes (Hu et al., Adv Drug Deliv Rev 98, 19-34 (2016) as well as for improved avidity that may result in enhanced neutralization breadth and potency (Galimidi et al., Cell 160, 433-446 (2015)). Previous bispecific bNAb designs utilizing CrossMab technology to combine two bNAb Fabs first proved the concept that empirical combinations of bNAb functional moieties in bi-valence format could achieve breadth and coverage (94-97%) superior to individual parental bNAbs (70-90%) (Asokan et al., J Virol 89, 12501-12512 (2015)). These empirical combinations were recently improved upon with cross-over dual variable (CODV) technology to develop Tri-NAbs with improved potency and breadth (Xu et al., Science 358, 85-90 (2017)). In addition, using tandem ScFvs format, we engineered a trispecific HIV-1 neutralizing antibody and two tetra-specific antibodies, consisting of functional moieties of three and four HIV bNAbs, respectively (Steinhardt et al., Nat Commun 9, 877 (2018)) (PCT/US2017/057053), which displays elevated neutralization breadth and potency compared to the parental bNAbs.

This background information is provided for informational purposes only. No admission is necessarily intended, nor should it be construed, that any of the preceding information constitutes prior art against the present invention.

### SUMMARY OF THE INVENTION

It is to be understood that both the foregoing general description of the embodiments and the following detailed description are exemplary, and thus do not restrict the scope of the embodiments.

Provided herein are multispecific neutralizing antibodies against HIV which in some embodiments utilize a tandem ScFv format to combine five HIV Env bNAb moieties via structure-based rational design. In some embodiments, these antibodies can achieve simultaneous engagement of five separate epitopes by each respective individual bNAb functional moiety to achieve superior binding avidity, profoundly enhanced viral inhibition breadth and potency, and ADCC functions.

By targeting >3 Env epitopes, these multi-NAbs display near pan-isolate neutralization breadth (99.6% coverage), high potency (GMT IC50=0.006 μg/mL) as assessed by a 208 virus panel and effectively neutralize viral quasi-species isolated from VRC01 clinical trials that are frequently resistant to VRC01. Furthermore, the data herein suggest that these multi-NAbs possess substantially higher antibody-dependent cell-mediated cytotoxicity (ADCC) capacity than their parental bNAbs. Taken together, the data suggest that multi-NAbs may be used as a novel candidate format for the treatment and management of persistent HIV-1 infection.

In some embodiments, the present disclosure describes synergistically combined epitope-binding moieties from five bNAbs that target the CD4 binding site, V2 and V3 conserved glycans, gp120/gp41 interface, as well as the membrane exterior proximal region into a ‘single’ penta-specific antibody (penta-NAb). The penta-Nabs exhibited superior degrees of inhibition against pseudo-typed viruses, exceptional neutralization capacity over individual bNAbs, and the capacity to neutralize replication-competent HIV isolates from infected individuals.

In one aspect, the invention provides a multispecific anti-HIV antibody that binds to multiple epitopes on HIV envelope protein, wherein the antibody comprises:
i. an amino acid sequence that binds to a V1/V2 apex glycan epitope;
ii. an amino acid sequence that binds to a V3-base glycan region epitope;
iii. an amino acid sequence that binds to a CD4 binding site (CD4bs) epitope;
iv. an amino acid sequence that binds to a gp120/gp41 interface epitope; and
v. an amino acid sequence that binds to a membrane proximal external region (MPER) epitope.

In some embodiments, the amino acid sequences of parts i-v) comprise amino acid sequences of single chain fragment variable (ScFv) moieties, wherein each ScFv moiety comprises an amino acid sequence from a light chain variable region (VL) and an amino acid sequence from a heavy chain variable region (VH) of an antibody. In some embodiments, the VL and VH sequences are separated by one or more linking amino acids. In some embodiments, the linking amino acids comprise one or more tetra-glycine serine (G₄S) linkers. In some embodiments, the antibody further comprises an Fc region of an immunoglobulin or a variant thereof. In some embodiments, the antibody comprises a first and second polypeptide chain, wherein the first and second polypeptide chains each comprise five ScFv moieties, wherein each ScFv moiety on a single chain recognizes an individual epitope, wherein each ScFv moiety comprises an amino acid sequence from a light chain variable region (VL) and an amino acid sequence from a heavy chain variable region (VH) of an antibody and an Fc region of an immunoglobulin or a variant thereof.

In some embodiments, the amino acid sequence that binds to the epitope of the V1/V2-glycan region comprises an amino acid sequence from an antibody selected from the group consisting of VRC26.25 and PGDM1400; the amino acid sequence that binds to the epitope of the V3-glycan region comprises an amino acid sequence from antibody PGT121; the amino acid sequence that binds to the epitope of the CD4-binding site (CD4bs) comprises an amino acid sequence from antibody N6; the amino acid sequence that binds to the epitope of the gp120/gp41 interface comprises an amino acid sequence from antibody 35O22; and the amino acid sequence that binds to the epitope of the membrane proximal external region (MPER) comprises an amino acid sequence from an antibody selected from the group consisting of 10E8v4, 10E8v4_S100cF, and 10E8v4_V5R_S100cF.

In some embodiments, the amino acid sequence from the antibody VRC26.25 comprises an amino acid sequence from the VH region comprising CDR H1, CDR H2 and CDR H3, wherein CDR H1 comprises QFRFDGYG, CDR H2 comprises ISHDGIKK and CDR H3 comprises AKDLREDECEEWWSDDFGKQLPCAKSRGGLVGIADN; and an amino acid sequence from the VL region comprising CDR L1, CDR L2 and CDR L3, wherein CDR L1 comprises TSNIGNNF, CDR L2 comprises ETD and CDR L3 comprises ATWAASLSSARV;
the amino acid sequence from the antibody PGDM1400 comprises an amino acid sequence from the VH region comprising CDR H1, CDR H2 and CDR H3, wherein CDR H1 comprises GNTLKTYD, CDR H2 comprises ISHEGDKK and CDR H3 comprises AKGSKHRLRDYALDDDGALNWAVDVDYLSNLEF; and an amino acid sequence from the VL region comprising CDR L1, CDR L2 and CDR L3, wherein CDR L1 comprises HSLIHGDRNNY, CDR L2 comprises LAS and CDR L3 comprises MQGRESPWT;
the amino acid sequence from the antibody PGT121 comprises an amino acid sequence from the VH region comprising CDR H1, CDR H2 and CDR H3, wherein CDR H1 comprises GASISDSY, CDR H2 comprises VHKSGDT and CDR H3 comprises ARTLHGRRIYGIVAFNEWFTYFYMDV; and an amino acid sequence from the VL region comprising CDR L1, CDR L2 and CDR L3, wherein CDR L1 comprises SLGSRA, CDR L2 comprises NNQ and CDR L3 comprises HIWDSRVPTKWV;
the amino acid sequence from the antibody N6 comprises an amino acid sequence from the VH region comprising CDR H1, CDR H2 and CDR H3, wherein CDR H1 comprises GYTFTAHI, CDR H2 comprises IKPQYGAV and CDR H3 comprises AR; and an amino acid sequence from the VL region comprising CDR L1, CDR L2 and CDR L3, wherein CDR L1 comprises QGVGSD, CDR L2 comprises HTS and CDR L3 comprises QVLQF;
the amino acid sequence from the antibody 35O22 comprises an amino acid sequence from the VH region comprising CDR H1, CDR H2 and CDR H3, wherein CDR H1 comprises GYRFNFYH, CDR H2 comprises ISPYSGDK and CDR H3 comprises DDTGTYFCAKGLLRDGSSTWLPYL; and an amino acid sequence from the VL region comprising CDR L1, CDR L2 and CDR L3, wherein CDR L1 comprises NSVCCSHKS, CDR L2 comprises EDN and CDR L3 comprises CSYTHNSGCV;
the amino acid sequence from the antibody 10E8v4 comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWSGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV;
the amino acid sequence from the antibody 10E8v4_S100cF comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWFGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV;
the amino acid sequence from the antibody 10E8v4_V5R_S100cF comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWFGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV.

In another aspect, the invention provides one or more vectors comprising a nucleic acid encoding the antibody or polypeptide of the invention.

In another aspect, the invention provides a cell comprising the one or more vectors of the invention.

In another aspect, the invention provides an engineered cell that expresses an antibody of the invention. In some embodiments, the cell is an immune cell. In some embodiments, the cell is a B cell.

In another aspect, the invention provides a pharmaceutical composition comprising an antibody of the invention and/or cells of the invention and a pharmaceutically acceptable carrier.

In another aspect, the invention provides a method for treating or preventing HIV infection in a subject, comprising administering to the subject an effective amount of a composition comprising an effective amount of a multispecific antibody as described herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The skilled artisan will understand that the drawings, described below, are for illustration purposes only. The drawings are not intended to limit the scope of the present teachings in any way.

FIG. 1. HIV-1 Env major epitope clusters and their spatial relationships.

FIG. 2A-C. Schematic of bNAb entities linked together in tandem to form PentaNAb 2.0. (A) Topology of PentaNAb design: from the Env MPER to the Env apex, with the shortest distance between bNAb entities marked. Modeling of Env trimer BG505 SOSIP.664 binding to five bNAbs targeting the five conserved epitopes including the V1V2 glycan, V3 base glycan, CD4 binding site (CD4bs), gp120/gp41 interface, and membrane proximate exterior region (MPER) simultaneously by superimposing individual bNAb Fab-Env complex structure, only VH-VL regions of Fab are shown. Env trimer in grey, and the bNAb moieties in color; (B) Scheme of the PentaNAb design, with MPER bNAb at the N terminus and V1V2 bNAb connected with CH2-CH3 domains of IgG Fc. Strategies of engineering penta-ScFv by joining the termini of VHs and VLs by linkers in dash lines optimized to accommodate individual VHs and VLs to achieve simultaneous epitope engagement; (C) Linear presentation of PentaNAb 2.0 design.

FIG. 3A-B. Summary of neutralization breadth and potency of PentaNAb2.0 in a comprehensive panel of viruses, in comparison with other multi-specific antibody designs. (A) Comparison of potency-breadth curves and epitope specificities of Tri- and Penta-NAb against a panel of 208 circulating primary HIV-1 strains; (B) Heat maps of IC50, breadth and potency are shown as in (A) and FIG. 4. GMT, geometric mean of titer (IC50). The IC50 was adjusted to account for the molarity difference between the antibodies.

FIG. 4A-B. (A) IC50 values (μg/ml) for PentaNAb2.0 and other antibodies tested in a 208 virus panel. The IC50 was adjusted to account for the molarity difference between the antibodies. (B) Summary of IC50 values for PentaNAb2.0 and other antibodies.

FIG. 5A-C. Neutralization capacity of PentaNAbs tested against primary HIV-1 viruses isolated from patients involved in VRC01 therapy treatment. Neutralization of virus from patient V07-RD (A), and V09-RD (B), tested with antibodies at 2.5 μg/ml (left) and 10 μg/ml (right), respectively. (C) Summary of IC50/IC80 (μg/ml) for PentaNAbs tested against primary VRC01 resistant isolates. Neutralization IC50 and IC80 titers (μg/ml) of Penta-scFV and Penta-NAb IgG against HIV isolated from VRC01 clinical trial. The IC50 and IC80 values were adjusted to account for the molarity difference between the antibodies.

FIG. 6A-B. Schemes of various iterations in which bNAb entities can be linked together in tandem to form PentaNAbs in the “Reverse” or “Forward/Reverse” orientation. (A) The “reverse” orientation of the PentaNAb design, in contrast to that in FIG. 2; (B) The PentaNAb “Forward/Reverse” orientation configuration conferred by IgG Fc bearing “knob-into-hole” mutations to form heterodimer.

FIG. 7A-C. Comparison of PentaNAbs in both the Forward, Reverse and Forward/Reverse orders. (A) SDS PAGE gel analysis for PentaNAbs; (B) Summary of neutralization breadth and potency of PentaNAbs against a 26-virus panel of high stringency; (C) Neutralization IC50 values of PentaNAbs against the 26-virus panel. The IC50 was adjusted to account for the molarity difference between the antibodies.

FIG. 8A-C. Spatial distance of bNAb functional moieties.

FIG. 9A-B. Bi-ScFv and bi-NAb of VRC01-PGT121 design and EM analysis. (A) Schematic presentation of VRC01-PGT121 ScFv and full length IgG. (B) EM analysis of Env trimer BG505 SOSIP.664 and VRC01-PGT121 ScFv complex suggests bi-specific binding mode. Left, representative EM image of Env trimer BG505 SOSIP.664 and VRC01-PGT121 bi-ScFv complex. Arrows indicate ScFv molecules. Right, EM image of PGV04 (green) and PGT122 (blue) Fab complex with BG505 SOSIP.664 simultaneously.

FIG. 10. Levels of HIV replication in NOD-scid IL2Rg^{null} (NSG) mice. PBMCs from an HIV-infected, ART-naïve individuals were injected to 8 mice and plasma viremia was monitored over time. The limit of detection was 20 copies/ml as measured by Roche Ampliprep/COBAS HIV-1 Test Version 2.

FIG. 11A-B. (A) Design of PentaNAb4.0 Forward ScFV (FIG. 11A) and PentaNAb 4.0 Reverse ScFv (FIG. 11B).

FIG. 12A-B. Design of a multi-specific antibody HexaNAb 1.0 ScFv which contains PentaNAb4.0 Reverse ScFv with variable domains of dB4C7/UB-421, an anti-CD4 antibody.

FIG. 13A-C. Design of a Penta-specific antibody in full-length IgG form, namely PentaNAb4.0 Stem HC LS v2 IgG (FIG. 13A), expressed as an IgG assembled from a heavy chain (FIG. 13B) and a light chain (FIG. 13C). The antibody contains the M428L/N434S (LS) mutation in the Fc domain to elongate antibody in vivo half-life. The heavy chain consists of V2 bNAb PGDM1400, V3 bNAb PGT121, and the VH domain of CD4bs bNAb N6 (FIG. 13B). The light chain contains the VL domain of N6 and 35O22 and 10E8v4 S100cF (FIG. 13C).

FIG. 14A-B depicts the neutralization profiles of PentaNAb2.0 Reverse ScFv, PentaNAb4.0 Forward ScFv, PentaNAb4.0 Reverse ScFv, HexaNAb 1.0 ScFv and PentaNAb4.0 Stem HC LS v2 IgG tested with a 208-virus panel (FIGS. 14A & B). The PentaNAb2.0 Reverse ScFv has incremental improvement in potency (IC 50 geometric mean) compared to the initial design, PentaNAb2.0 ScFv. The HexaNAb 1.0 ScFv (FIG. 12) shows 100% virus neutralization breadth and promising potency (IC50 geometric mean ˜0.005 ug/mL) (FIGS. 14A & B). The PentaNAb4.0 Stem HC LS v2 IgG demonstrates 99.5% virus coverage (207/208 viruses tested) and high potency (IC50 geometric mean ˜0.02 ug/mL) (FIGS. 14A & B).

### DETAILED DESCRIPTION OF THE INVENTION

The present disclosure is based on the discovery of highly neutralizing and potent HIV antibodies that are capable of engaging at least five epitopes on HIV envelope protein. In some embodiments, the antibody comprises single-chain variable fragment (ScFv) domains of five bNAbs, specific for the HIV-1 envelope epitopes that have been joined to form penta-specific ScFvs (penta-ScFvs). The penta-ScFv crosslinks adjacent HIV-1 envelope protomers and demonstrates superior neutralization breadth over its parental bNAbs. The epitopes recognized are the V1/V2 glycan region, the V3-glycan region, the CD4-binding site (CD4bs), the gp120-gp41 interface, and the gp41 membrane proximal external region (MPER). Furthermore, the present disclosure shows that using an Fc moiety to combine two penta-ScFv molecules that recognize the same series of HIV-1 epitopes in either the forward or reverse orientation resulted in a penta-specific bNAb, which displays near-pan neutralization breadth potently. Thus, penta-specific antibodies combining functional moieties of Env bNAbs could achieve exceptional neutralization capacity with profoundly augmented avidity. The penta-specific antibodies described herein can be used in studies aimed at preventing HIV disease progression or mother to child transmission, and curing HIV. Furthermore, the approach described herein, that combines multi-functional moieties of individual bNAbs with profoundly elevated avidity and cooperative effect of multivalence interactions, may be applied to generate superior antibody-based anti-viral therapeutics against other infectious agents.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. As used in the specification and claims, the singular form “a,” “an” and “the” include plural references unless the context clearly dictates otherwise. For example, the term “a cell” includes a plurality of cells, including mixtures thereof. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

Abbreviations for amino acids are used throughout this disclosure and follow the standard nomenclature known in the art. For example, as would be understood by those of ordinary skill in the art, Alanine is Ala or A; Arginine is Arg or R; Asparagine is Asn or N; Aspartic Acid is Asp or D; Cysteine is Cys or C; Glutamic acid is Glu or E; Glutamine is Gln or Q; Glycine is Gly or G; Histidine is His or H; Isoleucine is Ile or I; Leucine is Leu or L; Lysine is Lys or K; Methionine is Met or M; Phenylalanine is Phe or F; Proline is Pro or P; Serine is Ser or S; Threonine is Thr or T; Tryptophan is Trp or W; Tyrosine is Tyr or Y; and Valine is Val or V.

As used herein, the term “about” means plus or minus 10% of the numerical value of the number with which it is being used.

The term “broad neutralizing antibody” refers to an antibody which inhibits HIV-1 infection, as defined by at least about 50% inhibition of infection in vitro, in more than 50%, 60%, 70%, 80%, 90%, 95%, 99% or greater, of a large panel of (greater than 100) HIV-1 envelope pseudotyped viruses and/or viral isolates. In some embodiments, the broad neutralizing antibody is an antibody that inhibits HIV-1 infection as defined by at least about 70%, 80%, 90%, 91%, 92%, 93%, 94%, 95%, 96%, 97%, 98%, 99% or 100% inhibition of infection in vitro in more than about 50%, 60%, 70%, 80%, 90%, 95%, 99% or greater, of a large panel of (greater than 100) HIV-1 envelope pseudotyped viruses and/or viral isolates. In some embodiments, the disclosure relates to a composition comprising one or a plurality of broad neutralizing antibodies.

As used herein, the term “in combination with,” is not intended to imply that the therapy or the therapeutic agents must be administered at the same time and/or formulated for delivery together, although these methods of delivery are within the scope described herein.

The therapeutic agents can be administered concurrently with, prior to, or subsequent to, one or more other additional therapies or therapeutic agents.

The term “antibody” means a molecule that recognizes and specifically binds to a target, such as a protein, polypeptide, peptide, carbohydrate, polynucleotide, lipid, or combinations of the foregoing through at least one antigen recognition site within a variable region of the molecule, or any functional fragment, mutant, variant, or derivative thereof which retains the epitope binding features of an immunoglobulin molecule. As used herein, the term “antibody” encompasses intact polyclonal antibodies, intact monoclonal antibodies, antibody fragments (such as Fab, Fab′, F(ab′)2, and Fv fragments, dual affinity retargeting antibodies (DART)), single chain Fv (scFv) mutants, multispecific antibodies such as pentaspecific antibodies generated from at least five intact immunoglobulins, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

In some embodiments, the antibody can comprise a sequence from any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is generally composed of three CDRs and four FRs, arranged from amino-terminus to carboxy-terminus in the following order: FR1, CDR1, FR2, CDR2, FR3, CDR3, FR4.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the invention do not abrogate the binding of the polypeptide or antibody containing the amino acid sequence, to the antigen(s), i.e., the envelope protein to which the polypeptide or antibody binds. Methods of identifying nucleotide and amino acid conservative substitutions which do not eliminate antigen binding are well-known in the art (see, e.g., Brummell et al., Biochem. 32:1180-1187 (1993); Kobayashi et al., Protein Eng. 12 (10): 879-884 (1999); and Burks et al., Proc. Natl. Acad. Sci. USA 94:412-417 (1997)).

As used herein, the term “CDR” refers to the complementarity determining region within antibody variable sequences. In some embodiments, there are three CDRs in each of the variable regions of the heavy chain and the light chain of an antibody, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia et al., J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “fragment” is defined as a physically contiguous portion of the primary structure of a biomolecule. In the case of polypeptides, a fragment may be defined by a contiguous portion of the amino acid sequence of a protein and may be at least 3-5 amino acids, at least 6-10 amino acids, at least 11-15 amino acids, at least 16-24 amino acids, at least 25-30 amino acids, at least 30-45 amino acids and up to the full length of the protein minus a few amino acids. In the case of polynucleotides, a fragment is defined by a contiguous portion of the nucleic acid sequence of a polynucleotide and may be at least 9-15 nucleotides, at least 15-30 nucleotides, at least 31-45 nucleotides, at least 46-74 nucleotides, at least 75-90 nucleotides, and at least 90-130 nucleotides. In some embodiments, fragments of bio molecules are immunogenic fragments.

In some embodiments, the term “functional fragment” means any portion of a polypeptide or amino acid sequence that is of a sufficient length to retain at least partial biological function that is similar to or substantially similar to the parental polypeptide or amino acid sequence upon which the fragment is based. If the fragment is a functional fragment of an antibody or antibody-like molecule, the fragment can possess a binding avidity for one or a plurality of antigens. In some embodiments, a functional fragment is a polypeptide that comprises 80, 85, 90, 95, 96, 97, 98, or 99% sequence identity of any variable region of a polypeptide antibody disclosed herein and has sufficient length to retain at least partial binding affinity to one or a plurality of antigens that bind to the amino acid sequence. In some embodiments, the fragment has a length of at least about 10, about 20, about 30, about 40, about 50, about 60, about 70, about 80, about 90, or about 100 contiguous amino acids. In some embodiments, the fragment is a fragment of any amino acid sequence disclosed herein and has a length of at least about 50 amino acids. In some embodiments, the fragment has a length of at least about 100, 150, 200, 250, 300, 350, 400, 450, 500, or 550 amino acids.

The term “antigen binding portion” or “antigen binding fragment” of an antibody (or simply “antibody portion” or “antibody fragment”), as used herein, refers to one or more fragments of an antibody that retain the ability to specifically bind to an antigen (e.g., HIV gp120). It has been shown that the antigen-binding function of an antibody can be performed by fragments of a full-length antibody. Such antibody embodiments can be multi-specific formats; specifically binding to two or more different antigens. Examples of binding fragments encompassed within the term “antigen-binding portion” or “antigen binding fragment” of an antibody include (i) a Fab fragment, a monovalent fragment consisting of the VL, VH, CL and CHI domains; (ii) a F(ab′) 2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment (Ward et al, (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” or “antigen binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “multispecific antibody” refers to an antibody or antibody-like molecule, or fragment thereof, capable of binding two or more related or unrelated targets, or antigens. Antibody specificity refers to selective recognition of the antibody for a particular epitope, or amino acid sequence, of an antigen. Natural antibodies, for example, are monospecific. Pentaspecific antibodies are antibodies which have five different antigen-binding specificities.

The term “epitope” includes any polypeptide determinant capable of specific binding to an antibody. In certain embodiments, epitope determinants include chemically active surface groupings of molecules such as amino acids, sugar side chains, phosphoryl, or sulfonyl, and, in certain embodiments, may have specific three-dimensional structural characteristics, and or specific charge characteristics. An epitope is a region of an antigen that is bound by an antibody.

The term “antigen” refers to a polypeptide that can stimulate the production of antibodies or a T cell response in an animal, including polypeptides that are injected or absorbed into an animal. An antigen reacts with the products of specific humoral or cellular immunity.

The term “HIV Envelope protein (Env)” refers to the glycoprotein that is found on the surface of HIV. The HIV envelope protein is initially synthesized as a longer precursor protein of 845-870 amino acids in size, designated gp160. gp160 forms a homotrimer and undergoes glycosylation within the Golgi apparatus. In vivo, it is then cleaved by a cellular protease into gp120 and gp41. gp120 contains most of the external, surface-exposed, domains of the HIV envelope glycoprotein complex, and it is gp120 which binds both to cellular CD4 receptors and to cellular chemokine receptors (such as CCR5). gp41 contains a transmembrane domain and remains in a trimeric configuration within the membrane of the virus or the membrane of a host cell; it interacts with gp120 in a noncovalent manner. The mature gp120 wildtype polypeptides have about 500 amino acids in the primary sequence. gp120 is heavily N-glycosylated giving rise to an apparent molecular weight of 120 kD. The polypeptide is comprised of five conserved regions (C1-05) and five regions of high variability (V1-V5). Exemplary sequence of wt gp120 polypeptides are shown on GENBANK, for example accession numbers AAB05604 and AAD12142 (as available on Oct. 16, 2009), incorporated by reference herein. It is understood that there are numerous variation in the sequence of gp120 from what is given in GENBANK, for example accession numbers AAB05604 and AAD12142, and that these variants are skill recognized in the art as gp120. The gp120 core has a molecular structure, which includes two domains: an “inner” domain (which faces gp41) and an “outer” domain (which is mostly exposed on the surface of the oligomeric envelope glycoprotein complex). The two gp120 domains are separated by a “bridging sheet” that is not part of either domain. The gp120 core includes 25 beta strands, 5 alpha helices, and 10 defined loop segments.

The term “CD4 binding site (CD4BS) antibodies” refers to antibodies that bind to the CD4 binding surface of a gp120 polypeptide. The antibodies interfere with or prevent CD4 from binding to a gp120 polypeptide.

The term “V3 loops” refers to a loop of about 35 amino acids critical for the binding of the co-receptor and determination of which of the co-receptors will bind. In certain examples the V3 loop includes residues 296-331.

The term “membrane-proximal external region or MPER” refers to a highly conserved region of the gp41 envelope protein. The MPER comprises the last 24 C-terminal amino acids of the gp41 ectodomain, LLELDKWASLWNWF (N/D) ITNWLWYIK (aa 660 to 683) (Zwick et al. J Virol. 2005 January; 79 (2): 1252-61).

The term “V1V2 glycan antibody” refers to antibodies that bind to the V1V2 apex and associated conserved glycans N160 and/or N156.

The term “gp120/gp41 interface antibody” refers to antibodies that bind to a conserved epitope that stretches across gp120 and gp41.

The term “vector,” as used herein, is intended to refer to a nucleic acid molecule. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome.

Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), (0)NR2 (“amidate”), P(0)R, P(0)OR, CO, or CH2 (“formacetal”), in which each R or R′ is independently H or substituted or unsubstituted alkyl (1-20C) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “host cell” as used herein is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. The host cells can include prokaryotic and eukaryotic cells selected from any of the Kingdoms of life. In some embodiments, eukaryotic cells include protist, fungal, plant and animal cells. In some embodiments, host cells include but are not limited to the prokaryotic cell line E. coli; mammalian cell lines CHO, HEK 293 and COS; the insect cell line Sf9; and the fungal cell Saccharomyces cerevisiae. In some embodiments, the host cell is an immune cell.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

The term “inhibit” and its various grammatical forms is used to refer to a restraining, blocking, or limiting of the range or extent of a certain biological event or effect.

The term “effective amount,” is used herein to include the amount of an agent (e.g. a multispecific antibody) that, when administered to a patient for treating an infection, is sufficient to effect treatment of the disease (e.g., by diminishing, ameliorating or maintaining the existing disease or one or more symptoms of disease or its related comorbidities). The “effective amount” may vary depending on the agent, how it is administered, the disease and its severity and the history, age, weight, family history, genetic makeup, stage of pathological processes, the types of preceding or concomitant treatments, if any, and other individual characteristics of the patient to be treated. An effective amount includes an amount that results in a clinically relevant change or stabilization, as appropriate, of an indicator of a disease or condition. “Effective amount” refers to an amount of a compound, material, or composition, as described herein effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. The effective amount of the composition may be dependent on any number of variables, including without limitation, the species, breed, size, height, weight, age, overall health of the subject, the type of formulation, the mode or manner or administration, the type and/or severity of the particular condition being treated, or the need to modulate the activity of the molecular pathway induced by association of the analog to its receptor. The appropriate effective amount can be routinely determined by those of skill in the art using routine optimization techniques and the skilled and informed judgment of the practitioner and other factors evident to those skilled in the art. An effective dose of the antibodies or mutants or variants described herein may provide partial or complete biological activity as compared to the biological activity induced by the wild-type or naturally occurring polypeptides upon which the antibodies or mutants or variants are derived. A therapeutically effective dose of the antibodies or mutants or variants described herein may provide a sustained biochemical or biological effect and/or an increased resistance to degradation when placed in solution as compared with the normal affect observed when the naturally occurring and fully processed translated protein is administered to the same subject.

An “immunoconjugate” is an antibody or multispecific antibody conjugated to one or more heterologous molecule(s), including but not limited to a cytotoxic agent.

The term “cytotoxic agent” as used herein refers to a substance that inhibits or prevents a cellular function and/or causes cell death or destruction. Cytotoxic agents include, but are not limited to, radioactive isotopes; growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof.

The term “administer” as used herein means to give or to apply. The term “administering” as used herein includes in vivo administration.

The term “linker” refers to a chemical moiety that connects one peptide sequence to another, e.g., one antibody or antigen binding fragment to another. Linkers can also be used to attach antibodies to labels or solid substrates. A linker can include amino acids. Linkers can be straight or branched, saturated or unsaturated carbon chains. They can also include one or more heteroatoms within the chain.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present disclosure can be administered by a variety of methods known in the art. As will be appreciated by the skilled artisan, the route and/or mode of administration will vary depending upon the desired results. To administer an antibody according to the disclosure by certain routes of administration, it may be necessary to coat the antibody with, or coadminister the antibody with, a material to prevent its inactivation. For example, the antibody may be administered to a subject in an appropriate carrier, for example, liposomes, or a diluent. Pharmaceutically acceptable diluents include saline and aqueous buffer solutions.

A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In one embodiment, the carrier is suitable for intravenous, intramuscular, subcutaneous, parenteral, spinal or epidermal administration (e.g. by injection or infusion).

The pharmaceutical compositions according to the disclosure can also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The term “subject” is used throughout the specification to describe an animal to which one or more compositions comprising the antibody or antibodies disclosed herein. In some embodiment, the subject is a human. The term “subject” and “patient” may be interchangeably used. In some instances in the description of the present disclosure, the term “patient” will refer to human patients suffering from a particular disease or disorder. In some embodiments, the subject may be a human suspected of having or being identified as at risk to develop HIV infection. In some embodiments, the subject is suspected of having or has been diagnosed with HIV or HIV-1 infection or AIDS. In some embodiments, the subject may be a human suspected of having or being identified as at risk to develop AIDS or an AIDS-associated disorder. In some embodiments, the subject may be a mammal. In some embodiments, the subject may be a non-human animal. In one embodiment, the subject is a human, such as a human being treated or assessed for an HIV infection; or a human having an HIV infection that would benefit from a multispecific antibody as described herein. In some embodiments, the subject is in need of the treatment being administered.

In some embodiments, the compositions or pharmaceutical compositions comprise crystalline forms or lyophilized forms of the antibodies, antibody-like molecules or salts thereof.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “potency” as used herein refers to the neutralization capacity, i.e. the IC₅₀ or IC₈₀ of the antibody, or fragment thereof. The term “IC50” as used herein refers to the concentration of an inhibitor, such as a multispecific antibody, where the response or biological activity is reduced by half. The term “IC₈₀” as used herein refers to the concentration of an inhibitor (e.g. a multispecific antibody) where the response or biological activity is reduced by eighty percent.

Humanization and primatization refer to in cases where the multi-specific antibodies or the five antibodies forming the multi-specific fusion antibody are non-human antibodies, the antibody can be “humanized” to reduce immunogenicity to a human recipient. Methods for humanizing non-human antibodies have been described in the art. See, e.g., Jones et al., Nature 321:522-525 (1986); Riechmann et al, Nature 332:323-327 (1988); Verhoeyen et al., Science 239:1534-1536 (1988), and U.S. Pat. No. 4,816,567. Generally, residues from the variable domain of a non-human antibody are “imported” into a human immunoglobulin molecule, resulting in antibodies in which some hypervariable region residues and possibly some FR residues of a human antibody are substituted by residues from analogous sites of non-human antibodies. It is important to humanize a non-human antibody while retaining high affinity for the antigen. To this end, three dimensional immunoglobulin models are commonly available and suitable for use in analyzing proposed humanized sequences in comparison to the parental non-human antibodies. Such analysis permits identification of residues likely involved in recognition and binding of the antigen, and therefore rational design of humanized sequences that retain the specificity and affinity for the antigen.

In some embodiments, multispecific antibodies are formed from anti-HIV human or humanized antibodies. Similarly, a penta-specific fusion antibody or the five antibodies forming the fusion can be “primatized” to reduce immunogenicity to another primate, non-human recipient, e.g., a rhesus recipient. Residues from the variable domain of a donor antibody (such as a non-primate antibody or an antibody of a primate species different from the recipient primate) are “imported” into a nonhuman primate recipient immunoglobulin molecule, resulting in antibodies in which some hypervariable region residues and possibly some FR residues of a nonhuman primate antibody are substituted by residues from analogous sites of donor antibodies. Alternatively, primatized antibodies can be made for use in a desirable primate species by using a recipient immunoglobulin having non-primate sequences or sequences from a different primate species by introducing the Fc fragment, and/or residues, including particularly framework region residues, from the desirable primate, into the recipient immunoglobulin.

By “affinity maturation” is meant when one or more hypervariable region residues of an antibody can be substituted to select for variants that have improved biological properties relative to the parent antibody by employing, e.g., affinity maturation using phage or yeast display. For example, the Fab region of an anti-HIV antibody can be mutated at several sites selected based on available structural information to generate all possible amino substitutions at each site. The antibody variants thus generated are displayed in a monovalent fashion from phage particles or on the surface of yeast cells. The displayed variants are then screened for their biological activity (e.g. binding affinity).

### Anti-HIV Antibodies

In some embodiments, the invention provides multispecific antibodies that are broadly neutralizing antibodies against HIV.

HIV-1 is among the most genetically diverse viral pathogens. Of the three main branches of the HIV-1 phylogenetic tree, the M (main), N (new), and O (outlier) groups, group M viruses are the most widespread, accounting for over 99% of global infections. This group is presently divided into nine distinct genetic subtypes, or clades (A through K), based on full-length sequences. Env is the most variable HIV-1 gene, with up to 35% sequence diversity between clades, 20% sequence diversity within clades, and up to 10% sequence diversity in a single infected person (Shankarappa, R. et al. 1999. J. Virol. 73:10489-10502). Clade B is dominant in Europe, the Americas, and Australia. Clade C is common in southern Africa, China, and India and presently infects more people worldwide than any other clade (McCutchan, F E. 2000. Understanding the genetic diversity of HIV-1. AIDS 14 (Suppl. 3): S31-S44). Clades A and D are prominent in central and eastern Africa.

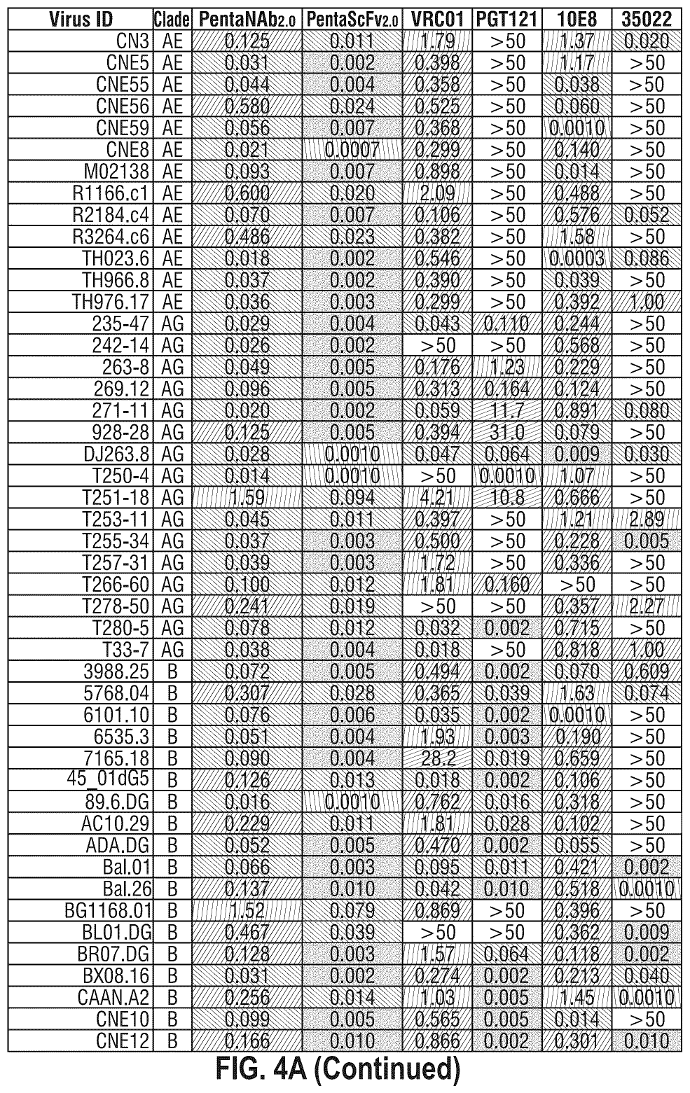

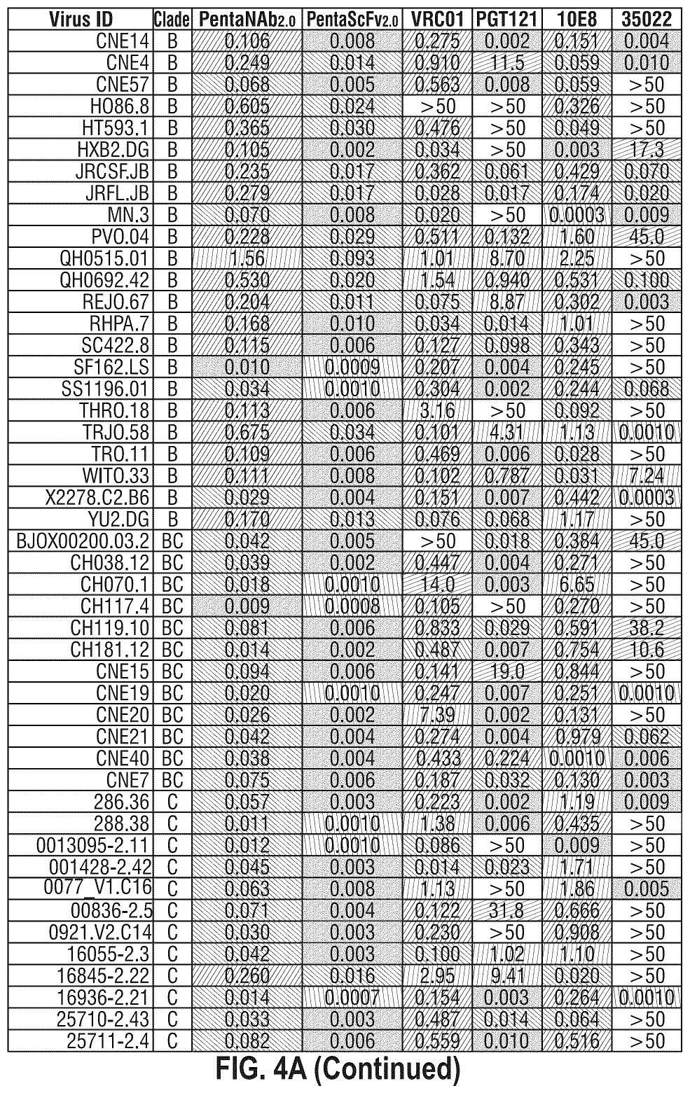

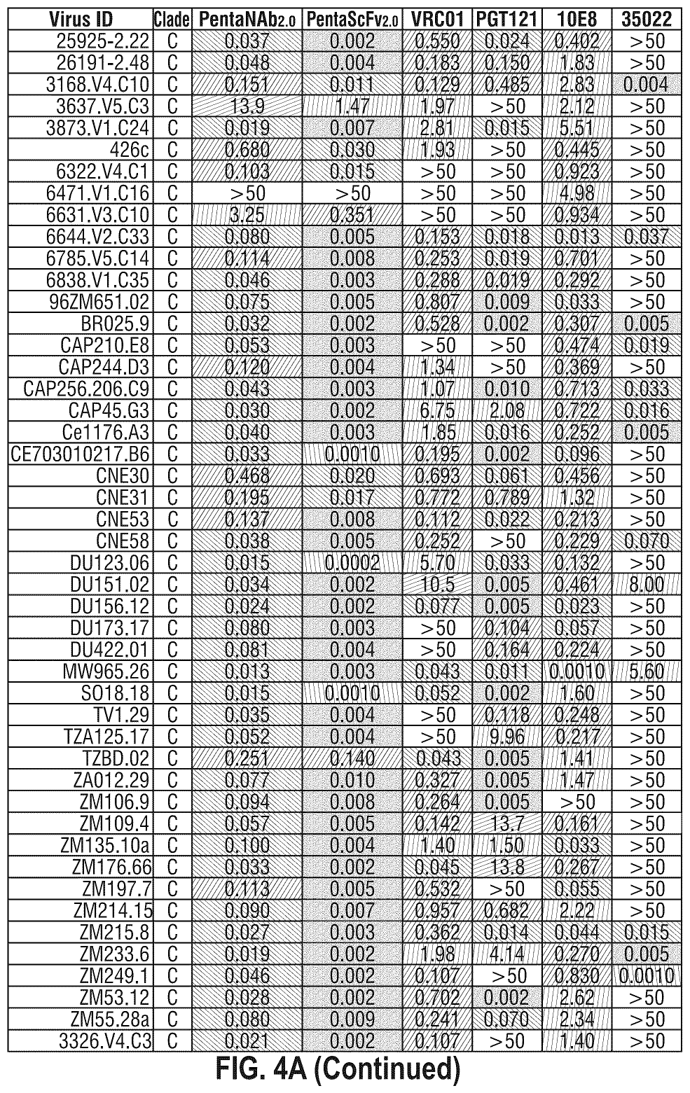

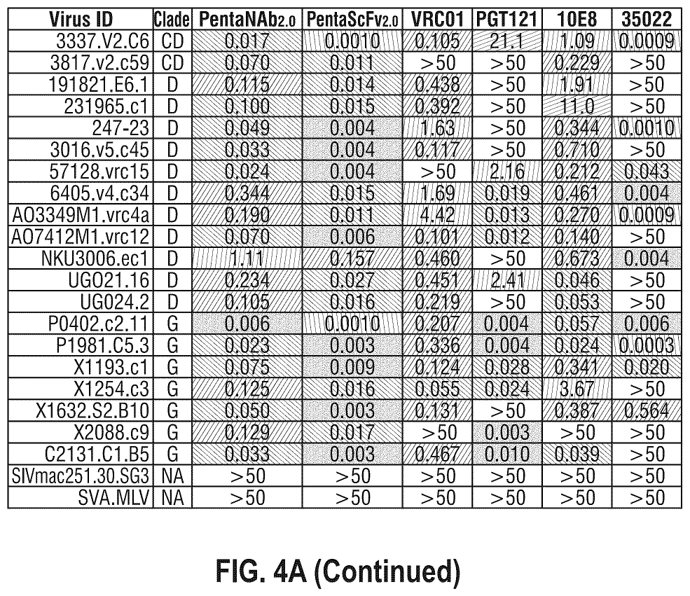

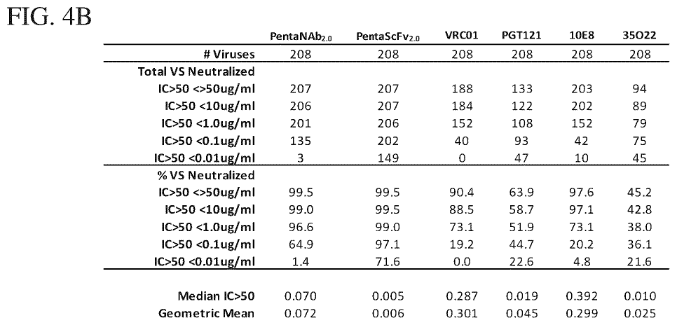

In some embodiments, the invention provides multispecific antibodies that are broadly neutralizing against HIV. In some embodiments, the antibody has a particularly high potency in neutralizing HIV infection in vitro across multiple clades as shown in the Table 1 and FIG. 4A herein. In some embodiments, only low concentrations are required in order to neutralize a given amount of virus. This facilitates higher levels of protection while administering lower amounts of antibody.

In some embodiments, the invention provides a multispecific anti-HIV antibody that binds to multiple epitopes on HIV envelope protein, wherein the antibody comprises:
i. an amino acid sequence that binds to a V1/V2 apex glycan epitope;
ii. an amino acid sequence that binds to a V3-base glycan region epitope;
iii. an amino acid sequence that binds to a CD4 binding site (CD4bs) epitope;
iv. an amino acid sequence that binds to a gp120/gp41 interface epitope; and
v. an amino acid sequence that binds to a membrane proximal external region (MPER) epitope.

The amino acid sequences of parts i-v) of the multispecific anti-HIV antibody can be present on one or more polypeptide chains. In some embodiments, the amino acid sequences of parts i-v) are present on a single polypeptide chain. In some embodiments, the multispecific anti-HIV antibody comprises two polypeptide chains, with each chain comprising amino acid sequences of parts i-v). In some embodiments, the antibody simultaneously binds the multiple epitopes.

In some embodiments, the multispecific anti-HIV antibody is capable of neutralizing at least 99% of the HIV viruses listed in Table 1 with an IC50 value of less than 50 μg/mL. In some embodiments, the multispecific anti-HIV antibody is capable of neutralizing at least 99.5% of the HIV pseudoviruses listed in Table 1 with an IC50 value of less than 50 μg/mL. In some embodiments, the multispecific anti-HIV antibody has an IC50 geomean of less than or equal to 0.10 μg/ml. In some embodiments, the multispecific anti-HIV antibody has an IC50 geomean of less than or equal to 0.072 μg/ml. In some embodiments, the multispecific anti-HIV antibody has an IC50 geomean of less than or equal to 0.006 μg/ml.

TABLE 1
List of HIV-1 Pseudovirus Panel:
208 Total Pseudoviruses Included.

| Virus ID | Clade |
|---|---|
| 0260.v5.c36 | A |
| 0330.v4.c3 | A |
| 0439.v5.c1 | A |
| 3365.v2.c20 | A |
| 3415.v1.c1 | A |
| 3718.v3.c11 | A |
| 398-F1_F6_20 | A |
| BB201.B42 | A |
| BB539.2B13 | A |
| BG505.W6M.C2 | A |
| BI369.9A | A |
| BS208.B1 | A |
| KER2008.12 | A |
| KER2018.11 | A |
| KNH1209.18 | A |
| MB201.A1 | A |
| MB539.2B7 | A |
| MI369.A5 | A |
| MS208.A1 | A |
| Q23.17 | A |
| Q259.17 | A |
| Q769.d22 | A |
| Q769.h5 | A |
| Q842.d12 | A |
| QH209.14M.A2 | A |
| RW020.2 | A |
| UG037.8 | A |
| 246-F3.C10.2 | AC |
| 3301.V1.C24 | AC |
| 3589.V1.C4 | AC |
| 6540.v4.c1 | AC |
| 6545.V4.C1 | AC |
| 0815.V3.C3 | ACD |
| 6095.V1.C10 | ACD |
| 3468.V1.C12 | AD |
| Q168.a2 | AD |
| Q461.e2 | AD |
| 620345.c1 | AE |
| BJOX009000.02.4 | AE |
| BJOX010000.06.2 | AE |
| BJOX025000.01.1 | AE |
| BJOX028000.10.3 | AE |
| C1080.c3 | AE |
| C2101.c1 | AE |
| C3347.c11 | AE |
| C4118.09 | AE |
| CM244.ec1 | AE |
| CNE3 | AE |
| CNE5 | AE |
| CNE55 | AE |
| CNE56 | AE |
| CNE59 | AE |
| CNE8 | AE |
| M02138 | AE |
| R1166.c1 | AE |
| R2184.c4 | AE |
| R3265.c6 | AE |
| TH023.6 | AE |
| TH966.8 | AE |
| TH976.17 | AE |
| 235-47 | AG |
| 242-14 | AG |
| 263-8 | AG |
| 269-12 | AG |
| 271-11 | AG |
| 928-28 | AG |
| DJ263.8 | AG |
| T250-4 | AG |
| T251-18 | AG |
| T253-11 | AG |
| T255-34 | AG |
| T257-31 | AG |
| T266-60 | AG |
| T278-50 | AG |
| T280-5 | AG |
| T33-7 | AG |
| 3988.25 | B |
| 5768.04 | B |
| 6101.10 | B |
| 6535.3 | B |
| 7165.18 | B |
| 45_01dG5 | B |
| 89.6.DG | B |
| AC10.29 | B |
| ADA.DG | B |
| Bal.01 | B |
| BaL.26 | B |
| BG1168.01 | B |
| BL01.DG | B |
| BR07.DG | B |
| BX08.16 | B |
| CAAN.A2 | B |
| CNE10 | B |
| CNE12 | B |
| CNE14 | B |
| CNE4 | B |
| CNE57 | B |
| HO86.8 | B |
| HT593.1 | B |
| HXB2.DG | B |
| JRCSF.JB | B |
| JRFL.JB | B |
| MN.3 | B |
| PVO.04 | B |
| QH0515.01 | B |
| QH0692.42 | B |
| REJO.67 | B |
| RHPA.7 | B |
| SC422.8 | B |
| SF162.LS | B |
| SS1196.01 | B |
| THRO.18 | B |
| TRJO.58 | B |
| TRO.11 | B |
| WITO.33 | B |
| X2278.C2.B6 | B |
| YU2.DG | B |
| BJOX002000.03.2 | BC |
| CH038.12 | BC |
| CH070.1 | BC |
| CH117.4 | BC |
| CH119.10 | BC |
| CH181.12 | BC |
| CNE15 | BC |
| CNE19 | BC |
| CNE20 | BC |
| CNE21 | BC |
| CNE40 | BC |
| CNE7 | BC |
| 286.36 | C |
| 288.38 | C |
| 0013095-2.11 | C |
| 001428-2.42 | C |
| 0077_V1.C16 | C |
| 00836-2.5 | C |
| 0921.V2.C14 | C |
| 16055-2.3 | C |
| 16845-2.22 | C |
| 16936-2.21 | C |
| 25710-2.43 | C |
| 25711-2.4 | C |
| 25925-2.22 | C |
| 26191-2.48 | C |
| 3168.V4.C10 | C |
| 3637.V5.C3 | C |
| 3873.V1.C24 | C |
| 426c | C |
| 6322.V4.C1 | C |
| 6471.V1.C16 | C |
| 6631.V3.C10 | C |
| 6644.V2.C33 | C |
| 6785.V5.C14 | C |
| 6838.V1.C35 | C |
| 96ZM651.02 | C |
| BR025.9 | C |
| CAP210.E8 | C |
| CAP244.D3 | C |
| CAP256.206.C9 | C |
| CAP45.G3 | C |
| Ce1176.A3 | C |
| CE703010217.B6 | C |
| CNE30 | C |
| CNE31 | C |
| CNE53 | C |
| CNE58 | C |
| DU123.06 | C |
| DU151.02 | C |
| DU156.12 | C |
| DU172.17 | C |
| DU422.01 | C |
| MW965.26 | C |
| SO18.18 | C |
| TV1.29 | C |
| TZA125.17 | C |
| TZBD.02 | C |
| ZA012.29 | C |
| ZM106.9 | C |
| ZM109.4 | C |
| ZM135.10a | C |
| ZM176.66 | C |
| ZM197.7 | C |
| ZM214.15 | C |
| ZM215.8 | C |
| ZM233.6 | C |
| ZM249.1 | C |
| ZM53.12 | C |
| ZM55.28a | C |
| 3326.V4.C3 | CD |
| 3337.V2.C6 | CD |
| 3817.v2.c59 | CD |
| 191821.E6.1 | D |
| 231965.c1 | D |
| 247-23 | D |
| 3016.v5.c45 | D |
| 57128.vrc15 | D |
| 6405.v4.c34 | D |
| A03349M1.vrc4a | D |
| A07412M1.vrc12 | D |
| NKU3006.ec1 | D |
| UG021.16 | D |
| UG024.2 | D |
| P0402.c2.11 | G |
| P1981.C5.3 | G |
| X1193.c1 | G |
| X1254.c3 | G |
| X1632.S2.B10 | G |
| X2088.c9 | G |
| X2131.C1.B5 | G |

In some embodiments, the amino acid sequences of parts i-v) comprise amino acid sequences of single chain fragment variable (ScFv) moieties, wherein each ScFv moiety comprises an amino acid sequence from a light chain variable region (VL) and an amino acid sequence from a heavy chain variable region (VH) of an antibody. In some embodiments, one or more of the ScFv moieties is organized such that the VL is at the amino terminal end of the ScFv moiety and the VH is at the carboxy terminal end of the ScFv moiety. In some embodiments, one or more of the ScFv moieties is organized such that the VH is at the amino terminal end of the ScFv moiety and the VL is at the carboxy terminal end of the ScFv moiety. In some embodiments, each ScFv moiety is organized such that the VL is at the amino terminal end of the ScFv moiety and the VH is at the carboxy terminal end of the ScFv moiety.

In some embodiments, the amino acid sequences that bind to the various epitope are separated from each other by one more linking amino acids. In some embodiments, the VL and VH sequences are separated by one or more linking amino acids. The linking amino acid sequences are not limiting. In some embodiments, the linking amino acids comprise one or more tetra-glycine serine (G₄S) linkers. In some embodiments, the VL and VH sequences are separated by 1-10 tetra-glycine serine (G₄S) linkers. In some embodiments, the VL and VH sequences are separated by 3 tetra-glycine serine (G₄S) linkers.

In some embodiments, one or more of the polypeptide chains of the multispecific anti-HIV antibody can further comprises an Fc region of an immunoglobulin or a variant thereof. In some embodiments, the immunoglobulin is IgG. In some embodiments, the immunoglobulin is IgG1. In some embodiments, the antibody comprises an IgG1 Fc region variant comprising mutations corresponding to M428L and N434S. In some embodiments, the antibody comprises an immunoglobulin Fc region that has been modified to facilitate heterodimerization.

In some embodiments, the antibody further comprises a sequence tag that facilitates purification of the antibody. In some embodiments, the sequence tag comprises a polyhistidine tag.

In some embodiments, the multispecific anti-HIV antibody comprises a first and second polypeptide chain, wherein the first and second polypeptide chains each comprise
a. five ScFv moieties, wherein each ScFv moiety on a single chain recognizes an individual epitope (V1/V2 apex glycan, V3-base glycan region, CD4 binding site (CD4bs), gp120/gp41 interface, membrane proximal external region (MPER), wherein each ScFv moiety comprises an amino acid sequence from a light chain variable region (VL) and an amino acid sequence from a heavy chain variable region (VH) of an antibody; and
b. an Fc region of an immunoglobulin or a variant thereof.

In some embodiments, the ScFv moieties are separated on the polypeptide chain by one or more linking amino acids. In some embodiments, the linking amino acids comprise one or more tetra-glycine serine (G₄S) linkers. In some embodiments, each ScFv moiety is separated by 5 tetra-glycine serine (G₄S) linkers.

In some embodiments, the amino acid sequences of parts i-v) are organized on a single polypeptide chain in the following order, from its N-terminus to its C-terminus:
i. an amino acid sequence that binds to the V1/V2 apex glycan epitope;
ii. an amino acid sequence that binds to the V3-base glycan region epitope;
iii. an amino acid sequence that binds to the CD4 binding site (CD4bs) epitope;
iv. an amino acid sequence that binds to the gp120/gp41 interface epitope; and
v. an amino acid sequence that binds to the membrane proximal external region (MPER) epitope.

In some embodiments, the amino acid sequences of parts i-v) are organized on a single polypeptide chain in the following order, from its N-terminus to its C-terminus:
i. an amino acid sequence that binds to the membrane proximal external region (MPER) epitope;
ii. an amino acid sequence that binds to the gp120/gp41 interface epitope;
iii. an amino acid sequence that binds to the CD4 binding site (CD4bs) epitope;
iv. an amino acid sequence that binds to the V3-base glycan region epitope; and
v. an amino acid sequence that binds to the V1/V2 apex glycan epitope.

In some embodiments, the multispecific anti-HIV antibody comprises a first and second polypeptide chain, wherein the amino acid sequences of parts i-v) are organized in the following order, from its N-terminus to its C-terminus:
i. an amino acid sequence that binds to the membrane proximal external region (MPER) epitope;
ii. an amino acid sequence that binds to the gp120/gp41 interface epitope;
iii. an amino acid sequence that binds to the CD4 binding site (CD4bs) epitope;
iv. an amino acid sequence that binds to the V3-base glycan region epitope;
v. an amino acid sequence that binds to the V1/V2 apex glycan epitope; and
vi. an Fc region of an immunoglobulin or a variant thereof.

In some embodiments, the multispecific anti-HIV antibody comprises a first and second polypeptide chain, wherein the amino acid sequences of parts i-v) are organized in the following order, from its N-terminus to its C-terminus:
i. an amino acid sequence that binds to the V1/V2 apex glycan epitope;
ii. an amino acid sequence that binds to the V3-base glycan region epitope;
iii. an amino acid sequence that binds to the CD4 binding site (CD4bs) epitope;
iv. an amino acid sequence that binds to the gp120/gp41 interface epitope;
v. an amino acid sequence that binds to the membrane proximal external region (MPER) epitope; and
vi. an Fc region of an immunoglobulin or a variant thereof.

In some embodiments, the multispecific anti-HIV antibody comprises
1) a first polypeptide chain, wherein the amino acid sequences of parts i-v) are organized in the following order, from its N-terminus to its C-terminus:
   i. an amino acid sequence that binds to the membrane proximal external region (MPER) epitope;
   ii. an amino acid sequence that binds to the gp120/gp41 interface epitope;
   iii. an amino acid sequence that binds to the CD4 binding site (CD4bs) epitope;
   iv. an amino acid sequence that binds to the V3-base glycan region epitope;
   v. an amino acid sequence that binds to the V1/V2 apex glycan epitope; and
   vi. an Fc region of an immunoglobulin or a variant thereof; and
2) a second polypeptide chain, wherein the amino acid sequences of parts i-v) are organized in the following order, from its N-terminus to its C-terminus:
   i. an amino acid sequence that binds to the V1/V2 apex glycan epitope;
   ii. an amino acid sequence that binds to the V3-base glycan region epitope;
   iii. an amino acid sequence that binds to the CD4 binding site (CD4bs) epitope;
   iv. an amino acid sequence that binds to the gp120/gp41 interface epitope;
   v. an amino acid sequence that binds to the membrane proximal external region (MPER) epitope; and
   vi. an Fc region of an immunoglobulin or a variant thereof.

In some embodiments, the amino acid sequence that binds to the epitope of the V1/V2-glycan region comprises an amino acid sequence from an antibody selected from the group consisting of VRC26.25 and PGDM1400; the amino acid sequence that binds to the epitope of the V3-glycan region comprises an amino acid sequence from antibody PGT121; the amino acid sequence that binds to the epitope of the CD4-binding site (CD4bs) comprises an amino acid sequence from antibody N6; the amino acid sequence that binds to the epitope of the gp120/gp41 interface comprises an amino acid sequence from antibody 35O22; and the amino acid sequence that binds to the epitope of the membrane proximal external region (MPER) comprises an amino acid sequence from an antibody selected from the group consisting of 10E8v4, 10E8v4_S100cF, and 10E8v4_V5R_S100cF.

In some embodiments, the amino acid sequence from the antibody VRC26.25 comprises an amino acid sequence from the VH region comprising CDR H1, CDR H2 and CDR H3, wherein CDR H1 comprises QFRFDGYG, CDR H2 comprises ISHDGIKK and CDR H3 comprises AKDLREDECEEWWSDDFGKQLPCAKSRGGLVGIADN; and an amino acid sequence from the VL region comprising CDR L1, CDR L2 and CDR L3, wherein CDR L1 comprises TSNIGNNF, CDR L2 comprises ETD and CDR L3 comprises ATWAASLSSARV;

In some embodiments, the amino acid sequence from the antibody PGDM1400 comprises an amino acid sequence from the VH region comprising CDR H1, CDR H2 and CDR H3, wherein CDR H1 comprises GNTLKTYD, CDR H2 comprises ISHEGDKK and CDR H3 comprises AKGSKHRLRDYALDDDGALNWAVDVDYLSNLEF; and an amino acid sequence from the VL region comprising CDR L1, CDR L2 and CDR L3, wherein CDR L1 comprises HSLIHGDRNNY, CDR L2 comprises LAS and CDR L3 comprises MQGRESPWT.

In some embodiments, the amino acid sequence from the antibody PGT121 comprises an amino acid sequence from the VH region comprising CDR H1, CDR H2 and CDR H3, wherein CDR H1 comprises GASISDSY, CDR H2 comprises VHKSGDT and CDR H3 comprises ARTLHGRRIYGIVAFNEWFTYFYMDV; and an amino acid sequence from the VL region comprising CDR L1, CDR L2 and CDR L3, wherein CDR L1 comprises SLGSRA, CDR L2 comprises NNQ and CDR L3 comprises HIWDSRVPTKWV.

In some embodiments, the amino acid sequence from the antibody N6 comprises an amino acid sequence from the VH region comprising CDR H1, CDR H2 and CDR H3, wherein CDR H1 comprises GYTFTAHI, CDR H2 comprises IKPQYGAV and CDR H3 comprises AR; and an amino acid sequence from the VL region comprising CDR L1, CDR L2 and CDR L3, wherein CDR L1 comprises QGVGSD, CDR L2 comprises HTS and CDR L3 comprises QVLQF.

In some embodiments, the amino acid sequence from the antibody 35O22 comprises an amino acid sequence from the VH region comprising CDR H1, CDR H2 and CDR H3, wherein CDR H1 comprises GYRFNFYH, CDR H2 comprises ISPYSGDK and CDR H3 comprises DDTGTYFCAKGLLRDGSSTWLPYL; and an amino acid sequence from the VL region comprising CDR L1, CDR L2 and CDR L3, wherein CDR L1 comprises NSVCCSHKS, CDR L2 comprises EDN and CDR L3 comprises CSYTHNSGCV.

In some embodiments, the amino acid sequence from the antibody 10E8v4 comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWSGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV.

In some embodiments, the amino acid sequence from the antibody 10E8v4_S100cF comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWFGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV.

In some embodiments, the amino acid sequence from the antibody 10E8v4_V5R_S100cF comprises an amino acid sequence from the VH region comprising CDR H1, CDR H2 and CDR H3, wherein CDR H1 comprises GFDFDNAW, CDR H2 comprises ITGPGEGWSV and CDR H3 comprises TGYYFCARTGKYYDFWFGYPPGEEYFQD; and an amino acid sequence from the VL region comprising CDR L1, CDR L2 and CDR L3, wherein CDR L1 comprises RGDSLRSHYAS, CDR L2 comprises GKNNRPS and CDR L3 comprises SSRDKSGSRLSV.

In some embodiments, the amino acid sequence from the antibody VRC26.25 comprises the VH region or a variant thereof comprising 1, 2, 3, or 4 conservative amino acid substitutions, wherein the VH region comprises SEQ ID NO:1; and the VL region or a variant thereof comprising 1, 2, 3, or 4 conservative amino acid substitutions, wherein the VL region comprises amino acids SEQ ID NO:5.

In some embodiments, the amino acid sequence from the antibody PGDM1400 comprises the VH region or a variant thereof comprising 1, 2, 3, or 4 conservative amino acid substitutions, wherein the VH region comprises SEQ ID NO:9; and the VL region or a variant thereof comprising 1, 2, 3, or 4 conservative amino acid substitutions, wherein the VL region comprises amino acids SEQ ID NO:13.

In some embodiments, the amino acid sequence from the antibody PGT121 comprises the VH region or a variant thereof comprising 1, 2, 3, or 4 conservative amino acid substitutions, wherein the VH region comprises SEQ ID NO:17; and the VL region or a variant thereof comprising 1, 2, 3, or 4 conservative amino acid substitutions, wherein the VL region comprises amino acids SEQ ID NO:21.

In some embodiments, the amino acid sequence from the antibody N6 comprises the VH region or a variant thereof comprising 1, 2, 3, or 4 conservative amino acid substitutions, wherein the VH region comprises SEQ ID NO:25; and the VL region or a variant thereof comprising 1, 2, 3, or 4 conservative amino acid substitutions, wherein the VL region comprises amino acids SEQ ID NO:29.

In some embodiments, the amino acid sequence from the antibody 35O22 comprises the VH region or a variant thereof comprising 1, 2, 3, or 4 conservative amino acid substitutions, wherein the VH region comprises SEQ ID NO:33; and the VL region or a variant thereof comprising 1, 2, 3, or 4 conservative amino acid substitutions, wherein the VL region comprises amino acids SEQ ID NO:37.

In some embodiments, the amino acid sequence from the antibody 10E8v4 comprises the VH region or a variant thereof comprising 1, 2, 3, or 4 conservative amino acid substitutions, wherein the VH region comprises SEQ ID NO:41; and VL region or a variant thereof comprising 1, 2, 3, or 4 conservative amino acid substitutions, wherein the VL region comprises amino acids SEQ ID NO:45.

In some embodiments, the amino acid sequence from the antibody 10E8v4_S100cF comprises the VH region or a variant thereof comprising 1, 2, 3, or 4 conservative amino acid substitutions, wherein the VH region comprises SEQ ID NO:49; and the VL region or a variant thereof comprising 1, 2, 3, or 4 conservative amino acid substitutions, wherein the VL region comprises amino acids SEQ ID NO:45.

In some embodiments, the amino acid sequence from the antibody 10E8v4_V5R_S100cF comprises the VH region or a variant thereof comprising 1, 2, 3, or 4 conservative amino acid substitutions, wherein the VH region comprises SEQ ID NO:51; and the VL region or a variant thereof comprising 1, 2, 3, or 4 conservative amino acid substitutions, wherein the VL region comprises amino acids SEQ ID NO:45.

Variable regions and CDR sequences of the antibodies described herein are shown in Table 2, below.

TABLE 2
Amino Acid Sequences of variable domains and CDR sequences.

| SEQ ID NO | DESCRIPTION | AMINO ACID SEQUENCE |
|---|---|---|
| 1 | VRC26.25 VH | QVQLVESGGGVVQPGTSLRLSCAASQFRFDGYGMHWVRQA |
| | | PGKGLEWVASISHDGIKKYHAEKVWGRFTISRDNSKNTLY |
| | | LQMNSLRPEDTALYYCAKDLREDECEEWWSDDFGKQLPCA |
| | | KSRGGLVGIADNWGQGTMVTVSS |
| 2 | VRC26.25 VH CDR1 | QFRFDGYG |
| 3 | VRC26.25 VH CDR2 | ISHDGIKK |
| 4 | VRC26.25 VH CDR3 | AKDLREDECEEWWSDDFGKQLPCAKSRGGLVGIADN |
| 5 | VRC26.25 VL | QSVLTQPPSVSAAPGQKVTISCSGNTSNIGNNFVSWYQQR |
| | | PGRAPQLLIYETDKRPSGIPDRFSASKSGTSGTLAITGLQ |
| | | TGDEADYYCATWAASLSSARVFGTGTKVIVLV |
| 6 | VRC26.25 VL CDR1 | TSNIGNNF |
| 7 | VRC26.25 VL CDR2 | ETD |
| 8 | VRC26.25 VL CDR3 | ATWAASLSSARV |
| 9 | PGDM1400 VH | QAQLVQSGPEVRKPGTSVKVSCKAPGNTLKTYDLHWVRSV |
| | | PGQGLQWMGWISHEGDKKVIVERFKAKVTIDWDRSTNTAY |
| | | LQLSGLTSGDTAVYYCAKGSKHRLRDYALDDDGALNWAVD |
| | | VDYLSNLEFWGQGTAVTVSS |
| 10 | PGDM1400 VH CDR1 | GNTLKTYD |
| 11 | PGDM1400 VH CDR2 | ISHEGDKK |
| 12 | PGDM1400 VH CDR3 | AKGSKHRLRDYALDDDGALNWAVDVDYLSNLEF |
| 13 | PGDM1400 VL | DFVLTQSPHSLSVTPGESASISCKSSHSLIHGDRNNYLAW |
| | | YVQKPGRSPQLLIYLASSRASGVPDRFSGSGSDKDFTLKI |
| | | SRVETEDVGTYYCMQGRESPWTFGQGTKVDIK |
| 14 | PGDM1400 VL CDR1 | HSLIHGDRNNY |
| 15 | PGDM1400 VL CDR2 | LAS |
| 16 | PGDM1400 VL CDR3 | MQGRESPWT |
| 17 | PGT121 VH | QMQLQESGPGLVKPSETLSLTCSVSGASISDSYWSWIRRS |
| | | PGKGLEWIGYVHKSGDTNYSPSLKSRVNLSLDTSKNQVSL |
| | | SLVAATAADSGKYYCARTLHGRRIYGIVAFNEWFTYFYMD |
| | | VWGNGTQVTVSS |
| 18 | PGT121 VH CDR1 | GASISDSY |
| 19 | PGT121 VH CDR2 | VHKSGDT |
| 20 | PGT121 VH CDR3 | ARTLHGRRIYGIVAFNEWFTYFYMDV |
| 21 | PGT121 VL | SDISVAPGETARISCGEKSLGSRAVQWYQHRAGQAPSLII |
| | | YNNQDRPSGIPERFSGSPDSPFGTTATLTITSVEAGDEAD |
| | | YYCHIWDSRVPTKWVFGGGTTLTVL |
| 22 | PGT121 VL CDR1 | SLGSRA |
| 23 | PGT121 VL CDR2 | NNQ |
| 24 | PGT121 VL CDR3 | HIWDSRVPTKWV |
| 25 | N6 VH | RAHLVQSGTAMKKPGASVRVSCQTSGYTFTAHILFWFRQA |
| | | PGRGLEWVGWIKPQYGAVNFGGGFRDRVTLTRDVYREIAY |
| | | MDIRGLKPDDTAVYYCARDRSYGDSSWALDAWGQGTTVVV |
| | | SA |
| 26 | N6 VH CDR1 | GYTFTAHI |
| 27 | N6 VH CDR2 | IKPQYGAV |
| 28 | N6 VH CDR3 | AR |
| 29 | N6 VL | YIHVTQSPSSLSVSIGDRVTINCQTSQGVGSDLHWYQHKP |
| | | GRAPKLLIHHTSSVEDGVPSRFSGSGFHTSFNLTISDLQA |
| | | DDIATYYCQVLQFFGRGSRLHIK |
| 30 | N6 VL CDR1 | QGVGSD |
| 31 | N6 VL CDR2 | HTS |
| 32 | N6 VL CDR3 | QVLQF |
| 33 | 35O22 VH | QGQLVQSGAELKKPGASVKISCKTSGYRFNFYHINWIRQT |
| | | AGRGPEWMGWISPYSGDKNLAPAFQDRVIMTTDTEVPVTS |
| | | FTSTGAAYMEIRNLKFDDTGTYFCAKGLLRDGSSTWLPYL |
| | | WGQGTLLTVSS |
| 34 | 35O22 VH CDR1 | GYRFNFYH |
| 35 | 35O22 VH CDR2 | ISPYSGDK |
| 36 | 35O22 VH CDR3 | DDTGTYFCAKGLLRDGSSTWLPYL |
| 37 | 35O22 VL | QSVLTQSASVSGSLGQSVTISCTGPNSVCCSHKSISWYQW |
| | | PPGRAPTLIIYEDNERAPGISPRFSGYKSYWSAYLTISDL |
| | | RPEDETTYYCCSYTHNSGCVFGTGTKVSVL |
| 38 | 35O22 VL CDR1 | NSVCCSHKS |
| 39 | 35O22 VL CDR2 | EDN |
| 40 | 35O22 VL CDR3 | CSYTHNSGCV |
| 41 | 10E8v4 VH | EVRLVESGGGLVKPGGSLRLSCSASGFDFDNAWMTWVRQP |
| | | PGKGLEWVGRITGPGEGWSVDYAESVKGRFTISRDNTKNT |
| | | LYLEMNNVRTEDTGYYFCARTGKYYDFWSGYPPGEEYFQD |
| | | WGQGTLVIVSS |
| 42 | 10E8v4 VH CDR1 | GFDFDNAW |
| 43 | 10E8v4 VH CDR2 | ITGPGEGWSV |
| 44 | 10E8v4 VH CDR3 | TGYYFCARTGKYYDFWSGYPPGEEYFQD |
| 45 | 10E8v4 VL | SELTQDPAVSVALKQTVTITCRGDSLRSHYASWYQKKPGQ |
| | | APVLLFYGKNNRPSGIPDRFSGSASGNRASLTITGAQAED |
| | | EADYYCSSRDKSGSRLSVFGGGTKLTVL |
| 46 | 10E8v4 VL CDR1 | RGDSLRSHYAS |
| 47 | 10E8v4 VL CDR2 | GKNNRPS |
| 48 | 10E8v4 VL CDR3 | SSRDKSGSRLSV |
| 49 | 10E8v4_S100cF VH | EVRLVESGGGLVKPGGSLRLSCSASGFDFDNAWMTWVRQP |
| | | PGKGLEWVGRITGPGEGWSVDYAESVKGRFTISRDNTKNT |
| | | LYLEMNNVRTEDTGYYFCARTGKYYDFWFGYPPGEEYFQD |
| | | WGQGTLVIVSS |
| 10E8v4 VH CDR1 | 10E8v4_S100cF VH | GFDFDNAW |
| | CDR1 | |
| 10E8v4 VH CDR2 | 10E8v4_S100cF VH | ITGPGEGWSV |
| | CDR2 | |
| 50 | 10E8v4_S100cF VH | TGYYFCARTGKYYDFWFGYPPGEEYFQD |
| | CDR3 | |
| 51 | 10E8v4_V5R_S100cF | EVRLRESGGGLVKPGGSLRLSCSASGFDFDNAWMTWVRQP |
| | VH | PGKGLEWVGRITGPGEGWSVDYAESVKGRFTISRDNTKNT |
| | | LYLEMNNVRTEDTGYYFCARTGKYYDFWFGYPPGEEYFQD |
| | | WGQGTLVIVSS |
| 10E8v4 VH CDR1 | 10E8v4_V5R_S100cF | GFDFDNAW |
| | VH CDR1 | |
| 10E8v4 VH CDR2 | 10E8v4_V5R_S100cF | ITGPGEGWSV |
| | VH CDR2 | |
| 10E8v4_S100cF | 10E8v4_V5R_S100cF | TGYYFCARTGKYYDFWFGYPPGEEYFQD |
| VHBCDR3 | VH CDR3 | |

In some embodiments, the multispecific anti-HIV antibody is capable of binding five different antigen targets. The disclosure features a multispecific antibody that binds five non-overlapping epitopes. In some embodiments, the multispecific anti-HIV antibody comprises an amino acid sequence selected from any of SEQ ID NOS: 72-80, as shown below in Table 3.

TABLE 3
Penta-Nab names, orientations of variable light and variable heavy chains,
and amino acid sequences.

| SEQ | | | |
|---|---|---|---|
| ID | | | |
| NO: | Name | Orientation | Amino Acid Sequence |
| 72 | PentaNA | 10E8v4-5X-35O22- | MGWSCIILFLVATATGVHSSELTQDPAVSVALKQTVTITCRGD |
| | b_{1.0} | 5X-N6-5X-PGT121- | SLRSHYASWYQKKPGQAPVLLFYGKNNRPSGIPDRFSGSASGN |
| | | 5X-VRC26.25-His | RASLTITGAQAEDEADYYCSSRDKSGSRLSVFGGGTKLTVLGG |
| | | | GGSGGGGSGGGGSEVRLVESGGGLVKPGGSLRLSCSASGFDFD |
| | | | NAWMTWVRQPPGKGLEWVGRITGPGEGWSVDYAESVKGRFTIS |
| | | | RDNTKNTLYLEMNNVRTEDTGYYFCARTGKYYDFWSGYPPGEE |
| | | | YFQDWGQGTLVIVSSGGGGSGGGGSGGGGSGGGGSGGGGSQSV |
| | | | LTQSASVSGSLGQSVTISCTGPNSVCCSKKSISWYQWPPGRAP |
| | | | TLIIYEDNERAPGISPRFSGYKSYWSAYLTISDLRPEDETTYY |
| | | | CCSYTHNSGCVFGTGTKVSVLGGGGSGGGGSGGGGSQGQLVQS |
| | | | GAELKKPGASVKISCKTSGYRFNFYHINWIRQTAGRGPEWMGW |
| | | | ISPYSGDKNLAPAFQDRVIMTTDTEVPVTSFTSTGAAYMEIRN |
| | | | LKFDDTGTYFCAKGLLRDGSSTWLPYLWGQGTLLTVSSGGGGS |
| | | | GGGGSGGGGSGGGGSGGGGSYIHVTQSPSSLSVSIGDRVTINC |
| | | | QTSQGVGSDLHWYQHKPGRAPKLLIHHTSSVEDGVPSRFSGSG |
| | | | FHTSFNLTISDLQADDIATYYCQVLQFFGRGSRLHIKGGGGSG |
| | | | GGGSGGGGSRAHLVQSGTAMKKPGASVRVSCQTSGYTFTAHIL |
| | | | FWFRQAPGRGLEWVGWIKPQYGAVNFGGGFRDRVTLTRDVYRE |
| | | | IAYMDIRGLKPDDTAVYYCARDRSYGDSSWALDAWGQGTTVVV |
| | | | SAGGGGSGGGGSGGGGSGGGGSGGGGSSDISVAPGETARISCG |
| | | | EKSLGSRAVQWYQHPAGQAPSLIIYNNQDRPSGIPERFSGSPD |
| | | | SPFGTTATLTITSVEAGDEADYYCHIWDSRVPTKWVFGGGTTL |
| | | | TVLGGGGSGGGGSGGGGSQMQLQESGPGLVKPSETLSLTCSVS |
| | | | GASISDSYWSWIRRSPGKGLEWIGYVHKSGDTNYSPSLKSRVN |
| | | | LSLDTSKNQVSLSLVAATAADSGKYYCARTLHGRRIYGIVAFN |
| | | | EWFTYFYMDVWGNGTQVTVSSGGGGSGGGGSGGGGSGGGGSGG |
| | | | GGSQSVLTQPPSVSAAPGQKVTISCSGNTSNIGNNFVSWYQQR |
| | | | PGPAPQLLIYETDKRPSGIPDRFSASKSGTSGTLAITGLQTGD |
| | | | EADYYCATWAASLSSARVFGTGTKVIVLVGGGGSGGGGSGGGG |
| | | | SQVQLVESGGGVVQPGTSLRLSCAASQFRFDGYGMHWVRQAPG |
| | | | KGLEWVASISHDGIKKYHAEKVWGRFTISRDNSKMTLYLQMNS |
| | | | LRPEDTALYYCAKDLREDECEEWWSDYYDFGKQLPCAKSRGGL |
| | | | VGIADNWGQGTMVTVSSGSGHHHHHH |
| 73 | PentaNA | 10E8v4_S100cF- | MGWSCIILFLVATATGVHSSELTQDPAYSVALKQTVTITCRGD |
| | b_{2.0} | 5X-35O22-5X-N6- | SLRSHYASWYQKKPGQAPVLLFYGKNNRPSGIPDRFSGSASGN |
| | | 5X-PGT121-5X- | RASLTITGAQAEDEADYYCSSRDKSGSRLSVFGGGTKLTVLGG |
| | | VRC26.25-His | GGSGGGGSGGGGSEVRLVESGGGLVKPGGSLRLSCSASGFDFD |
| | | | NAWMTWVRQPPGKGLEWVGRITGPGEGWSVDYAESVKGRFTIS |
| | | | RDNTKNTLYLEMNNVRTEDTGYYFCARTGKYYDFWFGYPPGEE |
| | | | YFQDWGQGTLVIVSSGGGGSGGGGSGGGGSGGGGSGGGGSQSV |
| | | | LTQSASVSGSLGQSVTISCTGPNSVCCSHKSISWYQWPPGRAP |
| | | | TLIIYEDNERAPGISPRFSGYKSYWSAYLTISDLRPEDETTYY |
| | | | CCSYTHNSGCVFGTGTKVSVLGGGGSGGGGSGGGGSQGQLVQS |
| | | | GAELKKPGASVKISCKTSGYRFNFYHINWIRQTAGRGPEWMGW |
| | | | ISPYSGDKNLAPAFQBRVIMTTDTEVPVTSFTSTGAAYMEIRN |
| | | | LKFDDTGTYFCAKGLLRDGSSTWLPYLWGQGTLLTVSSGGGGS |
| | | | GGGGSGGGGSGGGGSGGGGSYIHVTQSPSSLSVSIGDRVTINC |
| | | | QTSQGVGSDLHWYQHKPGRAPKLLIHHTSSVEDGVPSRFSGSG |
| | | | FHTSFNLTISDLQADDIATYYCQVLQFFGRGSRLHIKGGGGSG |
| | | | GGGSGGGGSRAHLVQSGTAMKKPGASVRVSCQTSGYTFTAHIL |
| | | | FWFRQAPGRGLEWVGWIKPQYGAVNFGGGFRDRVTLTRDVYRE |
| | | | IAYMDIRGLKPDDTAVYYCARDRSYGDSSWALDAWGQGTTVVV |
| | | | SAGGGGSGGGGSGGGGSGGGGSGGGGSSDISVAPGETARISCG |
| | | | EKSLGSRAVQWYQHRAGQAPSLIIYNNQDRPSGIPERFSGSPD |
| | | | SPFGTTATLTITSVEAGDEADYYCHIWDSRVPTKWVFGGGTTL |
| | | | TVLGGGGSGGGGSGGGGSQMQLQESGPGLVKPSETLSLTCSVS |
| | | | GASISDSYWSWIRRSPGKGLEWIGYVHKSGDTNYSPSLKSRVN |
| | | | LSLDTSKNQVSLSLVAATAADSGKYYCARTLHGRRIYGIVAFN |
| | | | EWFTYFYMDVWGNGTQVTVSSGGGGSGGGGSGGGGSGGGGSGG |
| | | | GGSQSVLTQPPSVSAAPGQKVTISCSGNTSNIGNNFVSWYQQR |
| | | | PGRAPQLLIYETDKRPSGIPDRFSASKSGTSGTLAITGLQTGD |
| | | | EADYYCATWAASLSSARVFGTGTKVIVTVGGGGSGGGGSGGGG |
| | | | SQVQLVESGGGVVQPGTSLRLSCAASQFRFDGYGMHWVRQAPG |
| | | | KGLEWVASISRDGIKKYHAEKVWGRFTISRDNSKNTLYLQMNS |
| | | | LRPEDTALYYCAKDLREDECEEWWSDYYDFGKQLPCAKSRGGL |
| | | | VGIADNWGQGTMVTVSSGSGHHHKHH |
| 74 | PentaNA | VRC26.25-5X- | MGWSCIILFLVATATGVHSQVQLVESGGGVVQPGTSLRLSCAA |
| | b_{2.0} | PGT121-5X-N6-5X- | SQFRFDGYGMHWVRQAPGKGLEWVASISHDGIKKYHAEKVWGR |
| | Reverse | 35O22-5X- | FTISRDNSKNTLYLQMNSLRPEDTALYYCAKDLREDECEEWWS |
| | | 10E8v4_S100cF-His | DYYDFGKQLPCAKSRGGLVGIADNWGQGTMVTVSSGGGGSGGG |
| | | | GSGGGGSQSVLTQPPSVSAAPGQKVTISCSGNTSNIGNNFVSW |
| | | | YQQRPGRAPQLLIYETDKRPSGIPDRFSASKSGTSGTLAITGL |
| | | | QTGDEADYYCATWAASLSSARVFGTGTKVIVLVGGGGSGGGGS |
| | | | GGGGSGGGGSGGGGSQMQLQESGPGLVKPSETLSLTCSVSGAS |
| | | | ISDSYWSWIRRSPGKGLEWIGYVHKSGDTNYSPSLKSRVNLSL |
| | | | DTSKNQVSLSLVAATAADSGKYYCARTLHGRRIYGIVAFNEWF |
| | | | TYFYMDVWGNGTQVTVSSGGGGSGGGGSGGGGSSDISVAPGET |
| | | | ARISCGEKSLGSRAVQWYQHRAGQAPSLIIYNNQDRPSGIPER |
| | | | FSGSPDSPFGTTATLTITSVEAGDEADYYCHIWDSRVPTKWVF |
| | | | GGGTTLTVLGGGGSGGGGSGGGGSGGGGSGGGGSRAHLVQSGT |
| | | | AMKKPGASVRVSCQTSGYTFTAHILFWFRQAPGRGLEWVGWIK |
| | | | PQYGAVNFGGGFRDRVTLTRDVYREIAYMDIRGLKPDDTAVYY |
| | | | CARDRSYGDSSWALDAWGQGTTVVVSAGGGGSGGGGSGGGGSY |
| | | | IHVTQSPSSLSVSIGDRVTINCQTSQGVGSDLHWYQHKPGRAP |
| | | | KLLIHHTSSVEDGVPSRFSGSGFHTSFNLTISDLQADDIATYY |
| | | | CQVLQFFGRGSRLHIKGGGGSGGGGSGGGGSGGGGSGGGGSQG |
| | | | QLVQSGAELKKPGASVKISCKTSGYRFNFYHINWIRQTAGRGP |
| | | | EWMGWISPYSGDKNLAPAFQDRVIMTTDTEVPVTSFTSTGAAY |
| | | | MEIRNLKFDDTGTYFCAKGLLRDGSSTWLPYLWGQGTLLTVSS |
| | | | GGGGSGGGGSGGGGSQSVLTQSASVSGSLGQSVTISCTGPNSV |
| | | | CCSHKSISWYQWPPGRAFTLIIYEDNERAPGISPRFSGYKSYW |
| | | | SAYLTISDLRPEDETTYYCCSYTHNSGCVFGTGTKVSVLGGGG |
| | | | SGGGGSGGGGSGGGGSGGGGSEVRLVESGGGLVKPGGSLRLSC |
| | | | SASGFDFDNAWMTWVRQPPGKGLEWVGRITGPGEGWSVDYAES |
| | | | VKGRFTISRDNTKNTLYLEMNNVRTEDTGYYFCARTGKYYDFW |
| | | | FGYPPGEEYFQDWGQGTLVIVSSGGGGSGGGGSGGGGSSELTQ |
| | | | DPAYSVALKQTVTITCRGDSLRSHYASWYQKKPGQAPVLLFYG |
| | | | KNNRPSGIFDRFSGSASGNRASLTITGAQAEDEADYYCSSRDK |
| | | | SGSRLSVFGGGTKLTVLGSGHHHHHH |
| 75 | PentaNA | 10E8v4_S100cF- | MGWSCIILFLVATATGVHSSELTQDPAVSVALKQTVTITCRGD |
| | b_{3.0} | 5X-35O22-5X-N6- | SLRSHYASWYQKKPGQAPVLLFYGKNNRPSGIPDRFSGSASGN |
| | | 5X-PGT121-5X- | RASLTITGAQAEDEADYYCSSRDKSGSRLSVFGGGTKLTVLGG |
| | | PGDM1400-His | GGSGGGGSGGGGSEVRLVESGGGLVKPGGSLRLSCSASGFDFD |
| | | | NAWMTWVRQPPGKGLSWVGRITGPGEGWSVDYAESVKGRFTIS |
| | | | RDNTKNTLYLEMNNVRTEDTGYYFCARTGKYYDFWFGYPPGEE |
| | | | YFQDWGQGTLVIVSSGGGGSGGGGSGGGGSGGGGSGGGGSQSV |
| | | | LTQSASVSGSLGQSVTISCTGPNSVCCSHKSISWYQWPPGRAP |
| | | | TLIIYEDNERAPGISPRFSGYKSYWSAYLTISDLRPEDETTYY |
| | | | CCSYTHNSGCVFGTGTKVSVLGGGGSGGGGSGGGGSQGQLVQS |
| | | | GAELKKFGASVKISCKTSGYRFNFYHINWIRQTAGRGPEWMGW |
| | | | ISPYSGDKNLAPAFQDRVIMTTDTEVPVTSFTSTGAAYMEIRN |
| | | | LKFDDTGTYFCAKGLLRDGSSTWLPYLWGQGTLLTVSSGGGGS |
| | | | GGGGSGGGGSGGGGSGGGGSYIHVTQSPSSLSVSIGDRVTINC |
| | | | QTSQGVGSDLHWYQHKPGRAPKLLIHHTSSVEDGVPSRFSGSG |
| | | | FHTSFNLTISDLQADDIATYYCQVLQFFGRGSRLHIKGGGGSG |
| | | | GGGSGGGGSRAHLVQSGTAMKKPGASVRVSCQTSGYTFTAHIL |
| | | | FWFRQAPGRGLEWVGWIKPQYGAVNFGGGFRDRVTLTRDVYRE |
| | | | IAYMDIRGLKPDDTAVYYCARDRSYGDSSWALDAWGQGTTVVV |
| | | | SAGGGGSGGGGSGGGGSGGGGSGGGGSSDISVAPGETARISCG |
| | | | EKSLGSPAVQWYQHRAGQAPSLIIYNNQDRPSGIPERFSGSPD |
| | | | SPFGTTATLTITSVEAGDEADYYCMIWDSRVPTKWVFGGGTTL |
| | | | TVLGGGGSGGGGSGGGGSQMQLQESGPGLVKPSETLSLTCSVS |
| | | | GASISDSYWSWIRRSPGKGLEWIGYVHKSGDTNYSPSLKSRVN |
| | | | LSLDTSKNQVSLSLVAATAADSGKYYCARTLHGRRIYGIVAFN |
| | | | EWFTYFYMDVWGNGTQVTVSSGGGGSGGGGSGGGGSGGGGSGG |
| | | | GGSDFVLTQSPHSLSVTPGESASISCKSSHSLIHGDRNNYLAW |
| | | | YVQKPGRSPQLLIYLASSRASGVPDRFSGSGSDKDFTLKISRV |
| | | | ETEDVGTYYCMQGRESPWTFGQGTKVDIKGGGGSGGGGSGGGG |
| | | | SQAQLVQSGPEVRKPGTSVKVSCKAPGNTLKTYDLHWVRSVPG |
| | | | QGLQWMGWISHEGDKKVIVERFKAKVTIDWDRSTNTAYLQLSG |
| | | | LTSGDTAVYYCAKGSKHRLRDYALYDDDGALNWAVDVDYLSNL |
| | | | EFWGQGTAVTVSSGSGHHHHHH |
| 76 | PentaNA | PGDM1400-5X- | MGWSCIILFLVATATGVHSQAQLVQSGPEVRKPGTSVKVSCKA |
| | b_{3.0} | PGT121-5X-N6-5X- | PGNTLKTYDLHWVRSVPGQGLQWMGWISHEGDKKVIVERFKAK |
| | Reverse | 35O22-5X- | VTIDWDRSTNTAYLQLSGLTSGDTAVYYCAKGSKHRLRDYALY |
| | | 10E8v4S100cF-His | DDDGALNWAVDVDYLSNLEFWGQGTAVTVSSGGGGSGGGGSGG |
| | | | GGSDFVLTQSPHSLSVTPGESASISCKSSHSLIHGDRNNYLAW |
| | | | YVQKPGRSPQLLIYLASSRASGVPDRFSGSGSDKDFTLKISRV |
| | | | ETEDVGTYYCMQGRESPWTFGQGTKVDIKGGGGSGGGGSGGGG |
| | | | SGGGGSGGGGSQMQLQESGPGLVKPSETLSLTCSVSGASISDS |
| | | | YWSWIRRSPGKGLEWIGYVHKSGDTNYSPSLKSRVNLSLDTSK |
| | | | NQVSLSLVAATAADSGKYYCARTLHGRRIYGIVAFNEWFTYFY |
| | | | MDVWGNGTQVTVSSGGGGSGGGGSGGGGSSDISVAPGETARIS |
| | | | CGEKSLGSRAVQWYQHRAGQAPSLIIYNNQDRPSGIPERFSGS |
| | | | PDSPFGTTATLTITSVEAGDEADYYCHIWDSRVPTKWVFGGGT |
| | | | TLTVLGGGGSGGGGSGGGGSGGGGSGGGGSRAHLVQSGTAMKK |
| | | | PGASVRVSCQTSGYTFTAHILFWFRQAPGRGLEWVGWIKPQYG |
| | | | AVNFGGGFRDRVTLTRDVYREIAYMDIRGLKPDDTAVYYCARD |
| | | | RSYGDSSWALDAWCQGTTVVVSAGGGGSGGGGSGGGGSYIHVT |
| | | | QSPSSLSVSIGDRVTINCQTSQGVGSDLKWYQHKPGRAPKLLI |
| | | | HHTSSVEDGVPSRFSGSGFKTSFNLTISDLQADDIATYYCQVL |
| | | | QFFGRGSRLHIKGGGGSGGGGSGGGGSGGGGSGGGGSQGQLVQ |
| | | | SGAELKKPGASVKISCKTSGYRFNFYHINWIRQTAGRGPEWMG |
| | | | WISPYSGDKNLAPAFQDRVIMTTDTEVPVTSFTSTGAAYMEIR |
| | | | NLKFDDTGTYFCAKGLLRDGSSTWLPYLWGQGTLLTVSSGGGG |
| | | | SGGGGSGGGGSQSVLTQSASVSGSLGQSVTISCTGPNSVCCSH |
| | | | KSISWYQWPPGRAFTLIIYEDNERAPGISPRFSGYKSYWSAYL |
| | | | TISDLRPEDETTYYCCSYTHNSGCVFGTGTKVSVLGGGGSGGG |
| | | | GSGGGGSGGGGSGGGGSEVRLVESGGGLVKPGGSLRLSCSASG |
| | | | FDFDNAWMTWVRQPPGKGLEWVGPITGPGEGWSVDYAESVKGR |
| | | | FTISRDNTKNTLYLEMNNVRTEDTGYYFCARTGKYYDFWFGYP |
| | | | PGEEYFQDWGQGTLVTVSSGGGGSGGGGSGGGGSSELTQDPAV |
| | | | SVALKQTVTITCRGDSLRSHYASWYQKKPGQAPVLLFYGKNNR |
| | | | PSGIPDRFSGSASGNRASLTITGAQAEDSADYYCSSRPKSGSR |
| | | | LSVFGGGTKLTVLGSGHHHHHH |
| 77 | PentaNA | 10E8v4 | MGWSCIILFINATATGVHSSELTQDPAVSVALKQTVTITCRGD |
| | b_{4.0} | V5R_S100cF-5X- | SLRSHYASWYQKKPGQAPVLLFYGKNNRPSGIPDRFSGSASGN |
| | | 35O22-5X-N6-SX- | RASLTITGAQAEDEADYYCSSRDKSGSPISVFGGGTKLTVLGG |
| | | PGT121-5X- | GGSGGGGSGGGGSEVRLRESGGGLVKPGGSLRLSCSASGFDFD |
| | | PGTDM1400-His | NAWMTWVRQPPGKGLEWVGRITGPGEGWSVDYAESVKGRFTIS |
| | | | RDNTKNTLYLEMNNVRTEDTGYYFCARTGKYYDFWFGYPPGEE |
| | | | YFQDWGQGTLVIVSSGGGGSGGGGSGGGGSGGGGSGGGGSQSV |
| | | | LTQSASVSGSLGQSVTISCTGPNSVCCSHKSISWYQWPPGRAP |
| | | | TLIIYEDNERAPGISPRFSGYKSYWSAYLTISDLRPEDETTYY |
| | | | CCSYTHNSGCVFGTGTKVSVLGGGGSGGGGSGGGGSQGQLVQS |
| | | | GAELKKPGASVKISCKTSGYRFNFYHINWIRQTAGRGPEWMGW |
| | | | ISPYSGDKNLAPAFQDRVIMTTDTEVPVTSFTSTGAAYMEIRN |
| | | | LKFDDTGTYFCAKGLLRDGSSTWLPYLWGQGTLLTVSSGGGGS |
| | | | GGGGSGGGGSGGGGSGGGGSYIHVTQSPSSLSVSIGDRVTINC |
| | | | QTSQGVGSDLHVYQHKPGRAPKLLIHHTSSVEDGVPSRFSGSG |
| | | | FHTSFNLTISDLQADDIATYYCQVLQFFGRGSRLHIKGGGGSG |
| | | | GGGSGGGGSRAHLVQSGTAMKKPGASVRVSCQTSGYTFTAHIL |
| | | | FWFRQAPGRGLEWVGWIKPQYGAVNFGGGFRDRVTLTRDVYRE |
| | | | IAYMDIRGLKPDDTAVYYCARDRSYGDSSWALDAWGQGTTVVV |
| | | | SAGGGGSGGGGSGGGGSGGGGSGGGGSSDISVAPGETARISCG |
| | | | EKSLGSRAVQWYQHRAGQAPSLIIYNNQDRPSGIPERFSGSPD |
| | | | SPFGTTATLTITSVEAGDEADYYCHIWDSRVPTKWVFGGGTTL |
| | | | TVLGGGGSGGGGSGGGGSQMQLQESGPGLVKPSETLSLTCSVS |
| | | | GASISDSYWSWIRRSPGKGLEWIGYVHKSGDTNYSPSLKSRVN |
| | | | LSLDTSKNQVSLSLVAATAADSGKYYCARTLHGRRIYGIVAFN |
| | | | EWFTYFYMDVWGNGTQVTVSSGGGGSGGGGSGGGGSGGGGSGG |
| | | | GGSDFVLTQSPHSLSVTPGSSASISCKSSHSLIHGDRNNYLAW |
| | | | YVQKPGRSPQLLIYLASSRASGVPDRFSGSGSDKDFTLKISRV |
| | | | ETEDVGTYYCMQGRESPWTFGQGTKVDIKGGGGSGGGGSGGGG |
| | | | SQAQLVQSGPEVRKPGTSVKVSCKAPGNTLKTYDLHWVRSVPG |
| | | | QGLQWMGWISHEGDKKVTVERFKAKVTIDWDRSTNTAYLQLSG |
| | | | LTSGDTAVYYCAKGSKHRLRDYALYDDDGALNWAVDVDYLSNL |
| | | | EFWGQGTAVTVSSGSGHHHHHH |
| 78 | PentaNA | PGDM1400-5X- | MGWSCIILFLVATATGVHSQAQLVQSGPEVRKPGTSVKVSCKA |
| | b_{4.0} | PGT121-5X-N6-5X- | PGNTLKTYDLHWVRSVPGQGLQWMGWISHEGDKKVIVERFKAK |
| | Reverse | 35O22-5X-10E8v4 | VTIDWDRSTNTAYLQLSGLTSGDTAVYYCAKGSKHRLRDYALY |
| | | V5R S100cF-His | DDDGALNWAVDVDYLSNLEFWGQGTAVTVSSGGGGSGEGGSGG |
| | | | GGSDFVLTQSPHSLSVTPGESASISCKSSHSLIHGDRNNYLAW |
| | | | YVQKPGRSPQLLIYLASSRASGVPDRFSGSGSDKDFTLKISRV |
| | | | ETEDVGTYYCMQGRESPWTFGQGTKVDIKGGGGSGGGGSGGGG |
| | | | SGGGGSGGGGSQMQLQESGPGLVKPSETLSLTCSVSGASISDS |
| | | | YWSWIRRSPGKGLEWIGYVHKSGDTNYSPSLKSRVNLSLDTSK |
| | | | NQVSLSLVAATAADSGKYYCARTLHGRRIYGIVAFNEWFTYFY |
| | | | MDVWGNGTQVTVSSGGGGSGGGGSGGGGSSDISVAPGETARIS |
| | | | CGEKSLGSRAVQWYQHRAGQAPSLIIYNNQDRPSGIPERFSGS |
| | | | PDSPFGTTATLTITSVEAGDEADYYCHIWDSRVPTKWVFGGGT |
| | | | TLTVLGGGGSGGEGSGGGGSEGGGSGGGGSRAHLVQSGTAMKK |
| | | | PGASVRVSCQTSGYTFTAHILFWFRQAPGRGLEWVGWIKPQWG |
| | | | AVNFGGGFRDPVTITRDVYRETAYMDIRGLKPDDTAVYYCARD |
| | | | RSYGDSSWALDAWGQGTTVVVSAGGGGSGGGGSGGGGSYIHVT |
| | | | QSPSSLSVSIGDRVTINCQTSQGVGSDLHWYQHKPGRAPKLLI |
| | | | HHTSSVEDEVPSRFSGSGFHTSFNLTISDLQADDIATYYCQVL |
| | | | QFFGRGSRLHIKGGGGSGGGGSGGGGSGGGGSGEGGSQGQLVQ |
| | | | SGAELKKPGASVKISCKTSGYRFNFYHINWIRQTAGRGPEWMG |
| | | | WISPYSGDKNLAPAFQDRVIMTTDTEVPVTSFTSTGAAYMEIR |
| | | | NLKFDDTGTYFCAKGLLRDGSSTWLPYLWGQGYLLTVSSGGGG |
| | | | SGGGGSGGGGSQSVITQSASVSGSLGQSVTISCTGPNSVCCSH |
| | | | KSISWYQWPPGRAPTLIIYEDNERAPGISPRFSGYKSYWSAYL |
| | | | TISDLRPEDETTYYCCSYTHNSGCVFGTGTKVSVLGGGGSGGG |
| | | | GSGGGGSGGGGSGGGGSEVRLRESGGGLVKPGGSLRLSCSASG |
| | | | FDFDNAWMTWVRQPPGKGLEWVGRITGPGEGWSVDYAESVKGR |
| | | | FTISRDNTKNTLYLEMNNVRTEDTGYYFCARTGKYYDFWFGYP |
| | | | PGEEYFQDWGQGTLVIVSSGGGGSGGGGSGGGGSSELTQDPAV |
| | | | SVALKQTVTITCRGDSLRSHYASWYQKKPGQAPVLLFYGKNNR |
| | | | PSGIPDRFSGSASGNRASLTITGAQAEDEADYYCSSRDESGSR |
| | | | LSVFGGGTKLTVLGSGHHHHHH |
| 79 | PentaNA | PGDM1400-5X- | MGWSCIILFLVATATGVHSQAQLVQSGPEVRKPGTSVKVSCKA |
| | b_{4.0} | PGT121-5X-N6-5X- | PGNTLKTYDLHWVRSVPGQGLQWMGWISHEGDKKVIVERFKAK |
| | Reverse | 35O22-5X-10E8v4 | VTIDWDRSTNTAYLQLSGLTSGDTAVYYCAKGSKHRLRDYALY |
| | | V5R S100cF VH | DDDGALNWAVDVDYLSNLEFWGQGTAVTVSSGGGGSGEGGSGG |
| | | fused to IgG1 LS | GGSDFVLTQSPHSLSVTPGESASISCKSSHSLIHGDPNNYLAW |
| | | HC with His Tag | YVQKPGRSPQLLIYLASSRASGVPDRFSGSGSDKDFTLKISRV |
| | | | ETEDVGTYYCMQGRESPWTFGQGTKVDIKGGGGSGGGGSGGGG |
| | | | SGGGGSGGGGSQMQLQESGPGLVKPSETLSLTCSVSGASISDS |
| | | | YWSWIRRSPGKGLEWIGYVHKSGDTNYSPSLKSRVNLSLDTSK |
| | | | NQVSLSLVAATAADSGKYYCARTLHGRRIYGIVAFNEWFTYFY |
| | | | MDVWGNGTQVTVSSGGGGSGGGGSGGGGSSDISVAPGETARIS |
| | | | CGEKSLGSRAVQWYQHRAGQAPSLIIYNNQDRPSGIPERFSGS |
| | | | PDSPFGTTATLTITSVEAGDEADYYCHIWDSRVPTKWVFGGGT |
| | | | TLTVLGGGGSGGGGSGGGGSGGGGSGGGGSRAHLVQSGTAMKK |
| | | | PGASVRVSCQTSGYTFTAHILFWFRQAPGRGLEWVGWIKPQYG |
| | | | AVNFGGGFRDRVTLTRDVYREIAYMDIRGLKPDDTAVYYCARD |
| | | | RSYGDSSWALDAWGQGTTVVVSAGGGGSGGGGSGGGGSYIHVT |
| | | | QSPSSLSVSIGDRVTINCQTSQGVGSDLHWYQHKPGPAPKLLI |
| | | | HHTSSVEDGVPSRFSGSGFHTSFNLTISDLQADDIATYYCQVL |
| | | | QFFGRGSRLHIKGGGGSGGGGSGGGGSGGGGSGGGGSQGQLVQ |
| | | | SGAELKKPGASVKISCKTSGYRFNFYHINWIRQTAGRGPEWMG |
| | | | WISPYSGDKNLAPAFQDRVIMTTDTEVPVTSFTSTGAAYMEIR |
| | | | NLKFDDTGTYFCAKGLLRDGSSTWLPYLWGQGTLLTVSSGGGG |
| | | | SGGGGSGGGGSQSVLTQSASVSGSLGQSVTISCTGPNSVCCSH |
| | | | KSISWYQWPPGRAPTLIIYEDNERAPGISPRFSGYKSYWSAYL |
| | | | TISDLRPEDETTYYCCSYTHNSGCVFGTGTKVSVLGGGGSGGG |
| | | | GSGGGGSGGGGSGGGGSEVRLRESGGGLVKPGGSLRLSCSASG |
| | | | FDFDNAWMTWVRQPPGKGLEWVGRITGPGEGWSVDYAESVKGR |
| | | | FTISRDNTKNTLYLEMNNVRTEDTGYYFCARTGKYYDFWFGYP |
| | | | PGEEYFQDWGQGTLVIVSSASTKGPSVFPLAPSSKSTSGGTAA |
| | | | LGCLVKDYFPEPVTVSWNSGALTSGVHTFPAVLQSSGLYSLSS |
| | | | VVTVPSSSLGTQTYICNVNHKPSNTKVDKKVEPKSCDKTHTCP |
| | | | PCPAPELLGGPSVFLFPPKPKDTLMISRTPEVTCVVVDVSHED |
| | | | PEVKFNWYVDGVEVHNAKTKPREEQYNSTYRVVSVLTVLHQDW |
| | | | LNGKEYKCKVSNKALPAPIEKTISKAKGQPREPQVYTLPPSRE |
| | | | EMTKNQVSLTCLVKGFYPSDIAVEWESNGQPENNYKTTPPVLD |
| | | | SDGSFFLYSKLTVDKSRWQQGNVFSCSVLHEALHSHYTQKSLS |
| | | | LSPGKGSGHHHHHH |
| 80 | PentaNA | PGDM1400-5X- | MGWSCIILFLVATATGVHSQAQLVQSGPEVRKPGTSVKVSCKA |
| | b_{4.0} | PGT121-5X-N6-5X- | PGNTLKTYDLHWVRSVPGQGLQWMGWISHEGDKKVIVERFKAK |
| | Reverse | 35O22-5X-10E8v4 | VTIDWDRSTNTAYLQLSGLTSGDTAVYYCAKGSKHRLPDYALY |
| | | V5R S100cF VH- | DDDGAINWAVDVDYLSNLEFWGQGTAVTVSSGGGGSGGGGSGG |
| | | VL-fused to IgG1 LS | GGSDFVLTQSPHSLSVTPGESASISCKSSHSLIHGDRNNYLAW |
| | | FC with His Tag | YVQKPGRSPQLLIYLASSRASGVPDRFSGSGSDKDFTLKISRV |
| | | | ETEDVGTYYCMQGRESPWTFGQGYKVDIKGGGGSGGGGSGGGG |
| | | | SGGGGSGGGGSQMQLQESGPGLVKPSETLSLTCSVSGASISDS |
| | | | YWSWIRRSPGKGLEWIGYVHKSGDTNYSPSLKSRVNLSLDTSK |
| | | | NQVSLSLVAATAADSGKYYCARTLHGRRIYGIVAFNEWFTYFY |
| | | | MDVWGNGTQVTVSSGGGGSGGGGSGGGGSSDISVAPGETARIS |
| | | | CGEKSLGSRAVQWYQHRAGQAPSLIIYNNQDRPSGIPERFSGS |
| | | | PDSPFGTTATLTITSVEAGDEADYYCHIWDSRVPTKWVFGGGT |
| | | | TLTVLGGGGSGGGGSGGGGSGGGGSGGGGSRAHLVQSGTAMKK |
| | | | PGASVRVSCQTSGYTFTAHILFWFRQAPGRGLEWVGWIKPQYG |
| | | | AVNFGGGFRDRVTLTRDVYREIAYMDIRGLKPDDTAVYYCARD |
| | | | RSYGDSSWALDAWGQGTTVVVSAGGGGSGGGGSGGGGSYIHVT |
| | | | QSPSSLSVSIGDRVTINCQTSQGVGSDLHWYQHKPGRAPKLLI |
| | | | HHTSSVEDGVPSRFSGSGFHTSFNLTISDLQADDIATYYCQVL |
| | | | QFFGRGSRLHIKGGGGSGGGGSGGGGSGGGGSGGGGSQGQLVQ |
| | | | SGAELKKPGASVKISCKTSGYRFNFYHINWIRQTAGRGPEWMG |
| | | | WISPYSGDKNLAPAFQDRVIMTTDTEVPVTSFTSTGAAYMEIR |
| | | | NLKFDDTGTYFCAKGLLRDGSSTWLPYLWGQGTLLTVSSGGGG |
| | | | SGGGGSGGGGSQSVLTQSASVSGSLGQSVTISCTGPNSVCCSH |
| | | | KSISWYQWPPGRAPTLIIYEDNERAPGISPRFSGYKSYWSAYL |
| | | | TISDLRPEDETTYYCCSYTHNSGCVFGTGTKVSVLGGGGSGGG |
| | | | GSGGGGSGGGGSGGGGSEVRLRESGGGLVKPGGSLRLSCSASG |
| | | | FDFDNAWMTWVRQPPGKGLEWVGRITGPGEGWSVDYAESVKGR |
| | | | FTISRDNTKNTLYLEMNNVRTEDTGYYFCARTGKYYDFWFGYP |
| | | | PGEEYFQDWGQGTLVIVSSGGGGSGGGGSGGGGSSELTQDPAV |
| | | | SVALKQTVTITCRGDSLRSHYASWYQKKPGQAPVLLFYGKNNR |
| | | | PSGIPDRFSGSASGNRASLTITGAQAEDEADYYCSSRDKSGSR |
| | | | LSVFGGGTKLTVLgsgPKSCDKTHTCPPCPAPELLGGPSVFLF |
| | | | PPKPKDTLMISRTPEVTCVVVDVSHEDPEVKFNWYVDGVEVHN |
| | | | AKTKPREEQYNSTYRVVSVLTVLHQDWLNGKEYKCKVSNKALP |
| | | | APIEKTISKAKGQPREPQVYTLPPSREEMTKNQVSLTCLVKGF |
| | | | YPSDIAVEWESNGQPENNYKTTPPVLDSDGSFFLYSKLTVDKS |
| | | | RWQQGNVFSCSVLHEALHSHYTQKSLSLSPGKGSGHHHHHH |

In some embodiments, 3D structure information of broadly neutralizing antibodies (bNAbs) in complex with HIV-1 envelope glycoproteins (Env) was used to perform rational design of penta-specific antibodies (PentaNAbs) capable of targeting five major Env neutralizing epitopes. In some embodiments, “PentaNAbs” as referred to herein comprise five functional single chain fragment variable (ScFv) moieties connected in tandem via (G4S) n linkers, such that penta-valence engagement of five individual epitopes on the HIV-1 Env by each respective moiety is possible. The bNAb functional moieties incorporated in the design target five major HIV neutralization epitopes including the i) V1/V2 apex glycan, ii) V3-base glycan, iii) CD4 binding site, iv) gp120/gp41 interface, and v) the membrane proximal external region (MPER). In addition, these PentaScFv entities can be fused to the fragment crystallizable region (Fc) region of IgG1 to accommodate effector functions in a Penta-IgG format.

The term “protomer” is the structural unit of an oligomeric protein unit. A protomer may span a single segment of a protein's primary structure, or may be composed of one or more different protein sequences that spans across a protein's three-dimensional folded or unfolded protein structure. A protomer may be a repeating structural unit.

In some embodiments, to design the Penta-specific ScFv and bNAbs, the physical distance between the compatible N/C termini of parental bNAb variable heavy (VH) and light chain (VL) domains was estimated to identify the shortest linker combinations allowing optimal VH/VL connection within each bNAb ScFv entity. In some embodiments multiple tandem (G4S)n linkers were used. In some embodiments, at least three G4S linkers were used between the VH and VL domains of a bNAb in the PentaScFv or Penta-bNAb molecule. The number of G4S linkers between VH and VL regions is not limiting and may not be required. In some embodiments there is no linker between the VH and VL regions. The number of G4S linkers between VH and VL domains of a bNab may include at least one, at least two, at least three, at least four, at least five, at least six, at least seven, at least eight, at least nine, at least ten, at least eleven, at least twelve, at least thirteen, at least fourteen, or at least fifteen linkers. In some embodiments multiple tandem (G4S)n linkers can be used between individual bNab moieties in the PentaScFv and Penta-bNAb molecules. In some embodiments at least five G4S linkers were used between individual bNAbs. The number of G4S linkers between individual bNabs may include at least one, at least two, at least three, at least four, at least five, at least six, at least seven, at least eight, at least nine, at least ten, at least eleven, at least twelve, at least thirteen, at least fourteen, or at least fifteen linkers.

In some embodiments, five G4S linkers were used to connect moieties of bNAbs targeting the V1V2 (e.g. VRC26.25) and V3 (e.g. PGT121) glycans. A linker of sufficient length (e.g. a linker with five G4S units) can be utilized to connect VRC26.25 or PGDM1400 with PGT121. In some embodiments, epitopes located on adjacent protomers of the HIV-1 trimer are often more proximate to each other than those on the same protomer. Thus, in some embodiments, two functional bNAb moieties can be arranged binding to the same protomer (intra-protomer mode) and in some cases to separate protomers (inter-protomers) of the HIV-1 Env trimer. In some embodiments, the shortest distance between the CD4bs targeting bNAb moiety, N6 (with VRC01 as surrogate), and the gp120/gp41 interface targeting 35O22 entity was determined to be an intra-protomer distance. Using the model in PDB 5FYK, the distance between the 35O22 and N6 functional variable domains were determined to be 57 Å between the C-terminus of 35O22 VH domain and the N-terminus of N6 VL domain with the topology of 35O22 (VL-VH)-N6 (VL-VH), and 47 Å between the C-terminus of N6 VL and the N-terminus of 35O22 VH with the topology of N6 (VH-VL)-35O22 (VH-VL), respectively. Thus, five G4S linkers can be used to connect N6 and 35O22. The length of the linkers is not limiting and may be varied.

In some embodiments, five G4S linkers can be used to connect the V3-glycan targeting PGT121 and the CD4bs targeting N6 ScFv entities. The shortest distance between PGT122 (a surrogate for PGT121) and VRC01 (a surrogate for N6) was previously determined between adjacent protomers within an Env trimer (inter-protomer). Using the PDB model 5FYK, the distances between the VRC01 and PGT121 functional variable domains were determined as the follows: 37 Å between the C-terminus of VRC01 VH domain and the N-terminus of PGT121 VL domain with VRC01 (VL-VH)-PGT121 (VL-VH) topology, and 53 Å between the C-terminus of PGT121 VL and the N-terminus of VRC01 VH with PGT121 (VH-VL)-VRC01 (VH-VL) topology. Furthermore, negative stain electron microscopy data demonstrated that one of the Bi-ScFvs previously generated, dVRC01 (VL-VH)-5X-PGT121 (VL-VH), was able to simultaneously engage both the PGT121 and VRC01 epitopes in an inter-protomer manner as predicted (31), which supports rationale for this linker design.

In some embodiments, the shortest distances between the gp120/gp41 interface targeting 35O22 entity and the MPER targeting 10E8v4 entity was determined to be inter-protomer manner. Using PDB 5IQ7 superimposed with PDB 5FYK, a model for Env engaged with both 35O22 and 10E8 was generated. In this model, the distance between the 10E8v4 and 35O22 functional variable domains were determined to be 48 Å between the C-terminus of 10E8v4 VH domain and the N-terminus of 35O22 VL domain with the topology of 10E8v4 (VL-VH)-35O22 (VL-VH), and 25 Å between the C-terminus of 35O22 VL and the N-terminus of 10E8v4 VH with the topology of 35O22 (VH-VL)-10E8v4 (VH-VL) respectively. Therefore, five G4S linkers can be used to connect 35O22 and 10E8v4. The linkers used are not limiting and can be optimized.

The type of linker between VH and VL domains of a bNAb is not limited to the use of G4S linkers. The type of linker between individual bNAbs is not limited to the use of G4S linkers. The length, sequence, and/or identity between the linkers separating the VH and VL regions and the linkers separating the individual bNAb moieties may be the same or may be different. The relationship between the types and characteristics of linkers used is not limiting. Such linkers are typically polypeptide sequences of between about 1 and 200 amino acids. The linkers can be any combination of natural or unnatural amino acids, and the sequence identity of the linkers is not limiting. Linkers can be flexible or rigid amino acid subsequences which are synthesized as part of a recombinant fusion protein.

The linkers may be flexible, the linkers may be rigid, or the linkers may be cleavable. Linkers may offer many other advantages for the production of fusion proteins, such as improving biological activity, increasing expression yield, and achieving desirable pharmacokinetic profiles. As such, the linkers in certain embodiments are not limiting and may be optimized for length, flexibility, rigidity, identity, cleavability, chemical composition, or other physical or chemical properties, for reasons that may or may not affect yield, half-life, biological activity, neutralization activity, pharmacokinetic properties, bioavailability, binding affinity, protein size, or stoichiometric binding ratios.

In some embodiments, the linker can be non-peptide based. A chemical linker may be used to connect protein sequences. Such linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages. In addition to covalent linkage of bNab sequences and linkers, non-covalent methods can be used to produce molecules with bNabs and linkers. Disulfide bonds may be used to form linkers. Chemical conjugation methods are known to persons of skill in the art and may be used to link VH and VL regions, or individual bNab moieties.

In some embodiments, the disclosure relates to an antibody comprising a variable portion and a constant portion, the variable portion comprising any one or plurality of variable heavy and/or light antibody fragments (such as a scFv fragment) capable of binding to the epitopes disclosed herein, and the constant portion comprising an IgG-like domain.

The antibody can be modified to improve certain biological properties of the antibody, e.g., to improve stability, to enhance or reduce effector functions such as antigen-dependent cell-mediated cytoxicity (ADCC) and/or complement dependent cytotoxicity (CDC) of the antibody, improved or decreased internalization and/or recycling, among others.

In certain embodiments, the multispecific antibody as described herein further includes a modification in the Fc region. In certain embodiments, the multispecific antibodies of the disclosure include mutations that increase binding to the neonatal Fc receptor (FcRn), which recycles IgG in intestinal epithelial cells and increases levels in the serum, extended half-life, enhanced mucosal localization, and conferred more efficient protection against lentivirus infection relative to the wild-type antibody. Enhanced neonatal Fc receptor function improves protection against primate SHIV infection. Such mutations are described, for example in Ko et al. (Nature 514, 642-645 (2014)).

For example, the Fc fragment of some antibodies (derived from human Ig4) can be replaced with human IgG1 that increases effector function mediated through FcRs (except FcRn). Such modification may improve the stability of the resulting antibody by about 5 fold. In another example, the IgG1 Fc fragment can be modified to improve the recycling of the antibody via the antibody salvage pathway.

Still another type of modification involves alteration of the glycosylation pattern of a parent antibody, including deletions of one or more carbohydrate moieties found in the parent antibody, or addition of one or more carbohydrates (via addition of one or more glycosylation sites) that are not present in the parent antibody.

In some embodiments, “knob into hole” technology (J. B. Ridgway, L. G. Presta, P. Carter, ‘Knobs-into-holes’ engineering of antibody CH3 domains for heavy chain heterodimerization. Protein Eng 9, 617-621 (1996)) can be utilized to generate a heterodimer of the “Forward” and “Reverse” PentaNAb arms, with LS mutations (M428L/N434S) in the Fc fragment for elongated antibody in vivo half-lives as well as improved biodistribution into the mucosal compartment. The use of this “knob into hole” technology is not limiting any method of generating PentaNAb dimers known to persons of skill in the art may be used.

PentaNAbs fused to a C-terminal fragment crystallizable region (Fc) region of IgG1, which may or may not contain a Hinge, CH2, and CH3 domains, can accommodate effector functions in a Penta-IgG format. The Fc region may or may not be fused to the C-terminus of each pentaNAb. Such recombinant formats are known to persons of skill in the art. The formats may include, but are not limited to the use of diabodies, minibodies, scFab, or scFv-zippers. For example, the types of recombinant formats may be found in the literature (See Frenzel A, Hust M, and Schirrmann T., Frontiers in Immunology. 2013; 4:217).

In some embodiments, two Forwards PentaNAbs can be combined through the use of the Fc regions of IgG1. In some embodiments, one Forward PentaNAb can be combined with one Reverse PentaNAb through the use of Fc regions of IgG1. The combinations of Forward and Reverse PentaNAbs are advantageous but not limiting.

### Polynucleotides

In some embodiments, the invention encompasses polynucleotides comprising polynucleotides that encode a polypeptide as described herein, such as a sequence of an HIV antibody or a fragment of such a polypeptide. For example, in some embodiments, the invention provides a polynucleotide comprising a nucleic acid sequence that encodes a multispecific antibody to HIV Env protein or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotides are isolated. In certain embodiments, the polynucleotides are substantially pure.

In some embodiments, the invention provides isolated nucleic acid molecules that encode multispecific anti-HIV antibodies as described herein. In some embodiments, the invention provides isolated nucleic acid molecules that encode isolated variable domains of the antibodies described herein. In some embodiments, the nucleotide sequence is selected from any one of SEQ ID NOS: 52-71 and SEQ ID NOS: 81-89, as shown below in Table 4 and below.

In some embodiments, the invention provides a polynucleotide comprising a polynucleotide encoding a polypeptide comprising a sequence selected from the group consisting of SEQ ID NOS: 1-51 and 72-80.

In some embodiments, the invention provides a polynucleotide comprising a polynucleotide encoding a polypeptide comprising the heavy or light chain variable region selected from the group consisting of SEQ ID NOS: 1, 5, 9, 13, 17 21, 25, 29, 33, 37, 41, 45, 49 and 51.

Also provided is a polynucleotide encoding a polypeptide comprising the heavy or light chain variable region having at least about 95%, at least about 96%, at least about 97%, at least about 98%, or at least about 99% sequence identity to any of SEQ ID NOS: 1, 5, 9, 13, 17 21, 25, 29, 33, 37, 41, 45, 49 and 51.

In some embodiments, the invention provides a polynucleotide comprising a polynucleotide encoding a polypeptide comprising an amino acid sequence selected from the group consisting of SEQ ID NOS: 72-80 and SEQ ID NOS: 90, 92, 94, 96, 98, 99 and 107.

Also provided is a polynucleotide encoding a polypeptide having at least about 95%, at least about 96%, at least about 97%, at least about 98%, or at least about 99% sequence identity to any of SEQ ID NOS: 72-80 and SEQ ID NOS: 90, 92, 94, 96, 98, 99 and 107.

The invention further provides a polynucleotide comprising a sequence selected from the group consisting of SEQ ID NOS: 52-71 and SEQ ID NOS: 81-89.

Also provided is a polynucleotide having at least 70%, 75%, 80%, 85%, 86%, 87%, 88%, 89%, 90%, 91%, 92%, 93%, 94%, 95%, 96%, 97%, 98%, or 99% sequence identity to any of SEQ ID NOS: 52-71, SEQ ID NOS: 81-89 and SEQ ID NOS 91, 93, 95, 97 and 106.

In some embodiments the polynucleotides comprise the coding sequence for the mature polypeptide fused in the same reading frame to a polynucleotide which aids, for example, in expression and secretion of a polypeptide from a host cell (e.g. a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g. COS-7 cells) is used.

The present invention further relates to variants of the hereinabove described polynucleotides encoding, for example, fragments, analogs, and derivatives.

The polynucleotide variants can contain alterations in the coding regions, non-coding regions, or both. In some embodiments the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

Vectors and cells comprising the polynucleotides described herein are also provided. The term “vector” means a construct, which is capable of delivering, and expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. “Vector” also includes shuttle and expression vectors. In some embodiments, the vector is a plasmid construct and also includes an origin of replication (e.g., the ColE1 origin of replication) and a selectable marker (e.g., ampicillin or tetracycline resistance), for replication and selection, respectively. An “expression vector” refers to a vector that contains the necessary control sequences or regulatory elements for expression of the antibodies including antibody fragments of the invention, in bacterial or eukaryotic cells.

TABLE 4
Nucleotide Sequences of variable domain anti-HIV antibodies.

| SEQ ID | | |
|---|---|---|
| NO | DESCRIPTION | NUCLEOTIDE SEQUENCE |
| 52 | VRC26.25 VH | caggtgcagttggtggagtctgggggaggcgtggtccagcctgggacgtcc |
| | (Original Seq) | ctgagactctcctgtgcagcctctcaattcaggtttgatggttatggcatg |
| | | cactgggtccgccaggccccaggcaaggggctggagtgggtggcatctata |
| | | tcacatgatggaattaaaaagtatcacgcagaaaaagtgtggggccgcttc |
| | | accatctccagagacaattccaagaacacactgtatctacaaatgaacagc |
| | | ctgcgacctgaggacacggctctctactactgtgcgaaagatttgcgagaa |
| | | gacgaatgtgaagagtggtggtcggatgattttgggaaacaactcccttgc |
| | | gcaaagtcacgcggcggcttggttggaattgctgataactggggccaaggg |
| | | acaatggtcaccgtctcttca |
| 53 | VRC26.25 VL | cagtctgtgctgacgcagccgccctcagtgtctgcggccccaggacagaag |
| | (Original Seq) | gtcaccatctcctgctctggaaacacctccaacattggcaataattttgtg |
| | | tcctggtaccaacagcgccccggcagagccccccaactcctcatttatgaa |
| | | actgacaagcgaccctcagggattcctgaccgattctctgcttccaagtct |
| | | ggtacgtcaggcaccctggccatcaccgggctgcagactggggacgaggcc |
| | | gattattactgcgccacatgggctgccagcctgagttccgcgcgtgtcttc |
| | | ggaactgggaccaaggtcatcgtccta |
| 54 | 35O22 VH | cagggtcaactagtccagtctggagctgaattgaaaaagcctggcgcctcg |
| | (Original Seq) | gtgaagatttcctgtaagacttcgggttataggtttaatttctatcatatt |
| | | aattggattcgacaaactgcaggacgtggacctgagtggatgggatggatc |
| | | agcccttacagtggtgacaaaaacctcgcacctgcctttcaagacagagtc |
| | | attatgacgacagacacagaagtccctgtgacctcattcacgtccacgggc |
| | | gcagcctacatggaaataaggaacctgaaatttgacgacacaggcacctat |
| | | ttctgtgcaaaaggcctcctgcgtgacggttcgtcgacgtggcttccttat |
| | | ttgtggggccagggtaccctactcaccgtctcgtca |
| 55 | 35O22 VL | cagtctgtgctgacgcagtctgcctccgtgtctgggtctcttggacagtcg |
| | (Original Seq) | gtcaccatctcctgcactggacccaatagtgtttgttgcagtcacaaatct |
| | | atctcctggtatcagtggcccccaggcagagcccccacactcatcatttat |
| | | gaggacaatgaaagggccccgggaatctctcctcgcttctctggctacaag |
| | | tcgtattggtcggcctacttgacaatctctgatctccggcctgaagacgag |
| | | accacttactactgttgctcatacactcacaatagcggctgtgtcttcggg |
| | | actgggaccaaggtctccgtcttg |
| 56 | 10E8v4 VH | gaggttagactggtggagtcaggaggggggcttgtgaagcccggtgggtct |
| | (Original Seq) | ctccgcctgagctgttctgcctccggctttgatttcgataacgcctggatg |
| | | acctgggtcaggcagcctccaggtaagggactggagtgggtgggaagaatc |
| | | acaggtccaggcgagggctggtccgtggactacgcggaatctgttaaaggg |
| | | cggtttacaatctcaagggacaataccaagaataccttgtatttggagatg |
| | | aacaacgtgagaactgaagacaccggatattacttctgtgccagaacaggc |
| | | aaatactacgacttctggtccggctatccccctggcgaggaatattttcaa |
| | | gactggggtcagggaacccttgttatcgtgtcctcc |
| 57 | 10E8v4 VL | tccgaactgactcaggaccctgccgtctctgtggcactgaagcagactgtg |
| | (Original Seq) | actattacttgccgaggcgactcactgcggagccactacgcttcctggtat |
| | | cagaagaaacccggccaggcacctgtgctgctgttctacggaaagaacaat |
| | | aggccatctggcatccccgaccgcttttctggcagtgcatcagggaaccga |
| | | gccagtctgaccattaccggcgcccaggctgaggacgaagccgattactat |
| | | tgcagctcccgggataagagcggctccagactgagcgtgttcggaggagga |
| | | actaaactgaccgtcctc |
| 58 | VRC26.25 VH | caggtgcagctggtggagagcggcggcggcgtggtgcagccaggcacctcc |
| | (Optimized Seq 1) | ctgaggctgtcttgtgcagcaagccagttcagatttgatggctacggcatg |
| | | cactgggtgcgccaggcaccaggcaagggcctggagtgggtagcctctatc |
| | | agccacgacggcatcaagaagtaccacgccgagaaagtgtggggcaggttc |
| | | accatctcccgcgataactctaaaaacacactgtatctgcagatgaactcc |
| | | ctgaggcccgaggacaccgccctgtactattgcgccaaggacctgcgcgag |
| | | gatgagtgtgaggagtggtggtccgacgattttggcaagcagctgccttgc |
| | | gcaaagagcaggggaggcctggtgggaatcgccgataattggggccagggc |
| | | accatggtgacagtgtctagc |
| 59 | VRC26.25 VH | caggtgcagctggtggagtccggaggaggagtggtgcagccaggcacctct |
| | (Optimized Seq 2) | ctgaggctgagctgtgcagcatcccagttcagatttgatggctacggaatg |
| | | cactgggtgaggcaggcaccaggcaagggactggagtgggtggccagcatc |
| | | tcccacgacggcatcaagaagtaccacgccgagaaagtgtggggcaggttc |
| | | accatctctcgcgataacagcaagaatacactgtatctgcagatgaacagc |
| | | ctgaggcccgaggacaccgccctgtactattgcgccaaggacctgcgcgag |
| | | gatgagtgtgaggagtggtggtccgacgattttggcaagcagctgccttgc |
| | | gcaaagagcaggggaggactggtgggaatcgccgacaattggggccagggc |
| | | accatggtgacagtgagcagc |
| 60 | VRC26.25 VL | cagagcgtgctgacccagccaccttccgtgtctgccgcaccaggacagaag |
| | (Optimized Seq 1) | gtgaccatcagctgttccggcaacacatccaatatcggcaacaatttcgtg |
| | | tcttggtaccagcagaggccaggaagggcaccacagctgctgatctatgag |
| | | acagacaagcggccttccggcatcccagatagattttctgccagcaagtcc |
| | | ggcaccagcggcacactggcaatcaccggcctgcagacaggcgacgaagct |
| | | gattactattgcgcaacctgggcagcctccctgagctccgccagggtgttc |
| | | ggaaccggaacaaaagtgatcgtgctggtg |
| 61 | VRC26.25 VL | cagagcgtgctgacccagccaccttccgtgtctgccgcaccaggacagaag |
| | (Optimized Seq 2) | gtgaccatcagctgttccggcaacacatccaatatcggcaacaatttcgtg |
| | | tcttggtaccagcagaggcctggaagagcaccacagctgctgatctatgag |
| | | acagacaagaggccctccggcatccctgatcgcttttctgccagcaagtcc |
| | | ggcaccagcggcacactggcaatcaccggactgcagacaggcgacgaggca |
| | | gattactattgcgcaacctgggcagcctccctgtctagcgccagggtgttc |
| | | ggcaccggcacaaaagtgatcgtgctggtg |
| 62 | PGDM1400 VH | caggcacagctggtgcagagcggacccgaagtgagaaaacctgggactagc |
| | (Optimized Seq 1) | gtcaaagtgtcatgtaaagcccctggaaataccctgaagacctacgatctg |
| | | cactgggtgcggtccgtgcctggacagggcctgcagtggatgggatggatc |
| | | tctcacgagggcgacaagaaagtgatcgtggagcggttcaaggccaaggtg |
| | | acaatcgattgggacagatccaccaacacagcctacctgcagctgtctggc |
| | | ctgaccagcggcgatacagccgtgtactactgtgccaagggctctaagcac |
| | | cggctgagagactacgccctggacgatgacggcgccctgaactgggccgtg |
| | | gatgtggactatctgtccaatctggagttctggggacagggaaccgcagtg |
| | | acagtgagctcc |
| 63 | PGDM1400 VH | caggcacagctggtgcagagcggaccagaggtgaggaagccaggcacctct |
| | (Optimized Seq 2) | gtgaaggtgagctgtaaggcccctggcaacaccctgaagacatacgatctg |
| | | cactgggtgcggtctgtgccaggacagggcctgcagtggatgggatggatc |
| | | agccacgagggcgacaagaaagtgatcgtggagcggtttaaggccaaggtg |
| | | acaatcgattgggacagaagcaccaatacagcctatctgcagctgtccggc |
| | | ctgacctctggcgatacagccgtgtactattgcgccaagggctccaagcac |
| | | cggctgagagactacgccctggacgatgacggcgccctgaattgggcagtg |
| | | gacgtggactatctgagtaatctggagttttgggggcagggcaccgcagtg |
| | | acagtgtctagc |
| 64 | PGDM1400 VL | gattttgtgctgacccagtctccacacagcctgtccgtgacacccggcgag |
| | (Optimized Seq 1) | tctgccagcatctcctgcaagtctagccacagcctgatccacggcgacagg |
| | | aacaattacctggcctggtacgtgcagaagccaggccgcagccctcagctg |
| | | ctgatctatctggcatcctctagggcctccggagtgccagatcgcttctct |
| | | ggcagcggctccgataaggactttaccctgaagatcagccgggtggagaca |
| | | gaggacgtgggcacatactattgtatgcagggccgagaatcaccttggaca |
| | | tttgggcagggaactaaagtcgacatcaaa |
| 65 | PGDM1400 VL | gatttcgtgctgacccagtctccacatagtctgagcgtgacacccggcgaa |
| | (Optimized Seq 2) | agcgcatcaatttcttgtaaatcatctcatagtctgatccacggcgatagg |
| | | aacaattacctggcctggtacgtgcagaagccaggccgcagccctcagctg |
| | | ctgatctacctggcaagctccagggcatccggagtgccagatcgcttctct |
| | | ggcagcggctccgataaggactttaccctgaagatctcccgggtggagaca |
| | | gaggacgtgggcacatactattgcatgcagggcagagagtctccttggacc |
| | | ttcggccagggcacaaaggtggacatcaag |
| 66 | 35O22 VH | cagggccagctggtgcagagcggagcagagctgaagaagcctggagccagc |
| | (Optimized Seq 1) | gtgaagatctcctgtaagacatctggctaccggttcaacttttatcacatc |
| | | aattggatcaggcagaccgcaggaaggggaccagagtggatgggctggatc |
| | | tccccctactctggcgataagaacctggccccagccttccaggacagagtg |
| | | atcatgaccacagataccgaggtgccagtgaccagcttcacctccaccgga |
| | | gccgcctacatggagatcaggaatctgaagttcgacgatacaggcacctat |
| | | ttttgcgcaaagggcctgctgagggacggctcctctacctggctgccttac |
| | | ctgtggggacagggcaccctgctgacagtgagctcc |
| 67 | 35O22 VH | cagggccagctggtgcagagcggagcagagctgaagaagccaggagcctct |
| | (Optimized Seq 2) | gtgaagatcagctgtaagacatccggctaccggttcaacttttatcacatc |
| | | aattggatcaggcagaccgcaggaaggggaccagagtggatgggctggatc |
| | | tccccctactctggcgataagaacctggccccagccttccaggacagagtg |
| | | atcatgaccacagataccgaggtgccagtgaccagcttcacctccaccgga |
| | | gccgcctacatggagatccggaatctgaagttcgacgatacaggcacctat |
| | | ttttgcgccaagggcctgctgagagacggctctagcacatggctgccatac |
| | | ctgtggggacagggcaccctgctgacagtgtcctct |
| 68 | 35O22 VL | cagtccgtgctgacccagtctgccagcgtgtccggctctctgggacagagc |
| | (Optimized Seq 1) | gtgaccatctcctgtacaggccccaacagcgtgtgctgtagccacaagagc |
| | | atctcctggtaccagtggcctccaggaagggcacctaccctgatcatctat |
| | | gaggacaatgagcgggccccaggcatctcccccagattctctggctacaag |
| | | tcttattggagcgcctacctgacaatcagcgacctgcgccccgaggatgag |
| | | acaacatactattgctgttcctatacccacaactctggctgcgtgtttggc |
| | | acaggcaccaaggtgtccgtgctg |
| 69 | 35O22 VL | caaagcgtgctgacccagtccgcctctgtgagcggctccctgggccagtct |
| | (Optimized Seq 2) | gtgaccatcagctgtacaggccccaactccgtgtgctgttctcacaagtct |
| | | atcagctggtaccagtggccaccaggaagggcacctaccctgatcatctat |
| | | gaggacaatgagagggcaccaggaatcagccctcgcttctccggctacaag |
| | | tcttattggagcgcctacctgaccatttccgacctgcgccccgaggatgag |
| | | accacatactattgctgtagctatacccacaactccggctgcgtgtttggc |
| | | acaggcaccaaggtgagcgtgctg |
| 70 | 10E8v4_V5R_S1 | gaggtgcggctgcgggagagcggcggcggcctggtgaagccaggcggctct |
| | 00cF VH | ctgagactgtcctgttctgccagcggcttcgactttgataatgcctggatg |
| | (Optimized Seq) | acatgggtgcggcagcctcctggcaaggggctggagtgggtgggaagaatc |
| | | accggaccaggagagggatggtctgtggactacgccgagagcgtgaagggc |
| | | cggttcaccatcagcagagataacactaaaaatacactgtatctggagatg |
| | | aacaatgtgcggaccgaggacacaggctactatttctgcgccagaaccggc |
| | | aagtactatgatttctggtttggctacccccctggcgaggagtattttcag |
| | | gactggggccagggcaccctggtcatcgtgagcagc |
| 71 | 10E8v4 VL | agcgagctgacccaggaccccgccgtgtccgtggccctgaagcagacagtg |
| | | accatcacatgcaggggcgactccctgcgctctcactacgccagctggtat |
| | | cagaagaagccaggacaggcacccgtgctgctgttctacggcaagaacaat |
| | | cggccttccggcatcccagatagattttccggctctgccagcggaaacagg |
| | | gccagcctgaccatcacaggagcacaggcagaggatgaagcagattactat |
| | | tgttcctctcgggacaagtccggctctagactgagcgtgttcggcggcgga |
| | | accaagctgacagtgctg |

Nucleotide sequences of the Penta-Nabs shown in Table 3 are provided below.
1. PentaNAb_{1.0} 10E8v4-5X-35O22-5X-N6-5X-PGT121-5X-VRC26.25-His
   SEQ ID NO:81

GAATTCGCCGCCACCATGGGATGGAGCTGTATTATTCTGTTTCTGGTCGCTAC
CGCTACCGGAGTGCATTCTTCTGAACTGACCCAGGACCCCGCCGTGAGCGTG
GCCCTGAAGCAGACCGTGACAATCACCTGCAGGGGCGACAGCCTGCGCTCCC
ACTACGCCAGCTGGTATCAGAAGAAGCCTGGCCAGGCCCCAGTGCTGCTGTT
CTACGGCAAGAACAATAGGCCCTCCGGCATCCCTGATCGCTTTTCCGGCTCTG
CCAGCGGAAACAGGGCCAGCCTGACAATCACCGGAGCACAGGCAGAGGACG
AGGCAGATTACTATTGCAGCTCCCGGGACAAGTCCGGCTCTAGACTGAGCGT
GTTCGGCGGCGGCACCAAGCTGACAGTGCTGGGAGGAGGAGGCAGCGGCGG
AGGAGGCTCCGGAGGCGGCGGCTCTGAGGTGCGGCTGGTGGAGTCTGGAGG
AGGCCTGGTGAAGCCAGGAGGCAGCCTGAGACTGAGCTGTTCCGCCTCTGGC
TTCGACTTTGATAATGCCTGGATGACATGGGTGCGGCAGCCACCTGGCAAGG
GCCTGGAGTGGGTGGGAAGAATCACCGGACCAGGAGAGGGATGGTCTGTGG
ACTACGCCGAGAGCGTGAAGGGCCGGTTCACCATCTCCAGAGATAACACCAA
GAATACACTGTATCTGGAGATGAACAATGTGCGGACCGAGGACACAGGCTAC
TATTTCTGCGCCAGAACCGGCAAGTACTATGATTTTTGGAGCGGCTACCCACC
CGGCGAGGAGTATTTTCAGGACTGGGGCCAGGGCACCCTGGTCATCGTGAGC
AGCGGCGGCGGCGGCAGCGGCGGCGGCGGCTCCGGAGGAGGCGGCTCTGGA
GGAGGAGGCAGCGGGGGAGGGGGCAGCCAGTCCGTGCTGACCCAGTCTGCC
AGCGTGTCCGGCTCTCTGGGACAGAGCGTGACCATCTCCTGTACAGGCCCCA
ACAGCGTGTGCTGTAGCCACAAGAGCATCTCCTGGTACCAGTGGCCTCCAGG
AAGGGCACCTACCCTGATCATCTATGAGGACAATGAGCGGGCCCCAGGCATC
TCCCCCAGATTCTCTGGCTACAAGTCTTATTGGAGCGCCTACCTGACAATCAG
CGACCTGCGCCCCGAGGATGAGACAACATACTATTGCTGTTCCTATACCCACA
ACTCTGGCTGCGTGTTTGGCACAGGCACCAAGGTGTCCGTGCTGGGCGGCGG
CGGCAGCGGGGGCGGGGGCTCCGGAGGGGGCGGCTCTCAGGGCCAGCTGGT
GCAGAGCGGAGCAGAGCTGAAGAAGCCTGGAGCCAGCGTGAAGATCTCCTG
TAAGACATCTGGCTACCGGTTCAACTTTTATCACATCAATTGGATCAGGCAGA
CCGCAGGAAGGGGACCAGAGTGGATGGGCTGGATCTCCCCCTACTCTGGCGA
TAAGAACCTGGCCCCAGCCTTCCAGGACAGAGTGATCATGACCACAGATACC
GAGGTGCCAGTGACCAGCTTCACCTCCACCGGAGCCGCCTACATGGAGATCA
GGAATCTGAAGTTCGACGATACAGGCACCTATTTTTGCGCAAAGGGCCTGCT
GAGGGACGGCTCCTCTACCTGGCTGCCTTACCTGTGGGGACAGGGCACCCTG
CTGACAGTGAGCTCCGGCGGCGGGGGCAGCGGCGGCGGGGGCTCCGGAGGA
GGAGGCTCTGGAGGAGGGGGCAGCGGAGGAGGCGGCTCCTACATCCACGTG
ACCCAGTCCCCATCTAGCCTGTCTGTGAGCATCGGCGATCGGGTGACCATCAA
CTGTCAGACATCTCAGGGCGTGGGCAGCGACCTGCACTGGTATCAGCACAAG
CCTGGCAGGGCCCCAAAGCTGCTGATCCACCACACATCCTCTGTGGAGGATG
GAGTGCCAAGCCGCTTCTCCGGCTCTGGATTCCACACCTCCTTTAATCTGACA
ATCTCTGACCTGCAGGCCGACGATATCGCCACCTACTATTGCCAGGTGCTGCA
GTTCTTTGGCCGGGGCTCCAGACTGCACATCAAGGGAGGAGGAGGCTCCGGG
GGCGGAGGCTCTGGCGGCGGCGGCAGCCGGGCCCACCTGGTGCAGAGCGGC
ACCGCCATGAAGAAGCCTGGCGCCAGCGTGAGAGTGTCCTGTCAGACATCTG
GCTACACCTTCACCGCCCACATCCTGTTCTGGTTTAGGCAGGCACCAGGAAGA
GGCCTGGAGTGGGTGGGCTGGATCAAGCCCCAGTATGGAGCAGTGAACTTCG
GAGGAGGCTTTCGGGACAGAGTGACACTGACCCGGGACGTGTACAGAGAGA
TCGCCTATATGGATATCAGGGGCCTGAAGCCAGACGATACCGCCGTGTACTA
TTGCGCCAGGGACCGCTCCTACGGCGATAGCTCCTGGGCACTGGACGCATGG
GGACAGGGCACCACAGTGGTGGTGAGCGCCGGCGGCGGAGGCTCCGGCGGC
GGGGGCTCTGGAGGAGGCGGCAGCGGAGGGGGAGGCTCCGGAGGGGGAGGC
TCTAGCGACATCTCCGTGGCCCCTGGCGAGACAGCCAGAATCTCTTGTGGCG
AGAAGTCTCTGGGCAGCAGGGCCGTGCAGTGGTACCAGCACAGGGCAGGAC
AGGCACCATCTCTGATCATCTATAACAATCAGGATAGGCCAAGCGGCATCCC
TGAGCGGTTCAGCGGCTCCCCCGACAGCCCTTTTGGCACCACAGCCACACTG
ACCATCACATCCGTGGAGGCAGGCGACGAAGCCGATTACTATTGCCACATCT
GGGATTCCAGAGTGCCAACCAAGTGGGTGTTCGGAGGAGGAACCACACTGAC
AGTGCTGGGAGGGGGGGGCTCTGGCGGCGGGGGCAGCGGGGGAGGAGGCTC
CCAGATGCAGCTGCAGGAGAGCGGACCAGGCCTGGTGAAGCCTAGCGAGAC
ACTGAGCCTGACATGTTCTGTGAGCGGCGCCTCCATCTCTGACAGCTACTGGT
CTTGGATCAGACGGAGCCCCGGCAAGGGCCTGGAATGGATCGGCTACGTGCA
CAAGTCCGGCGATACAAACTATTCCCCATCTCTGAAGTCTCGGGTGAACCTGT
CTCTGGACACCAGCAAGAATCAGGTGAGCCTGTCCCTGGTGGCAGCAACCGC
AGCAGATAGCGGCAAGTACTATTGCGCCAGAACACTGCACGGCAGGCGCATC
TACGGCATCGTGGCCTTTAACGAGTGGTTCACCTACTTTTATATGGACGTGTG
GGGCAATGGCACCCAGGTGACAGTGTCCTCTGGCGGGGGCGGCTCCGGAGGC
GGAGGCTCTGGCGGGGGCGGCAGCGGCGGGGGCGGCTCCGGGGGAGGCGGC
TCTCAGAGCGTGCTGACCCAGCCACCTTCCGTGTCTGCCGCACCAGGACAGA
AGGTGACCATCAGCTGTTCCGGCAACACATCCAATATCGGCAACAATTTCGT
GTCTTGGTACCAGCAGAGGCCAGGAAGGGCACCACAGCTGCTGATCTATGAG
ACAGACAAGCGGCCTTCCGGCATCCCAGATAGATTTTCTGCCAGCAAGTCCG
GCACCAGCGGCACACTGGCAATCACCGGCCTGCAGACAGGCGACGAAGCTG
ATTACTATTGCGCAACCTGGGCAGCCTCCCTGAGCTCCGCCAGGGTGTTCGGA
ACCGGAACAAAAGTGATCGTGCTGGTGGGCGGCGGAGGCTCTGGCGGAGGC
GGCAGCGGCGGGGGGGGCTCCCAGGTGCAGCTGGTGGAGAGCGGCGGCGGC
GTGGTGCAGCCAGGCACCTCCCTGAGGCTGTCTTGTGCAGCAAGCCAGTTCA
GATTTGATGGCTACGGCATGCACTGGGTGCGCCAGGCACCAGGCAAGGGCCT
GGAGTGGGTAGCCTCTATCAGCCACGACGGCATCAAGAAGTACCACGCCGAG
AAAGTGTGGGGCAGGTTCACCATCTCCCGCGATAACTCTAAAAACACACTGT
ATCTGCAGATGAACTCCCTGAGGCCCGAGGACACCGCCCTGTACTATTGCGC
CAAGGACCTGCGCGAGGATGAGTGTGAGGAGTGGTGGTCCGACTACTATGAT
TTTGGCAAGCAGCTGCCTTGCGCAAAGAGCAGGGGAGGCCTGGTGGGAATCG
CCGATAATTGGGGCCAGGGCACCATGGTGACAGTGTCTAGCGGATCCGGACA
CCACCATCACCATCATTAGTGAAAGCTT

2. PentaNAb_{2.0} 10E8v4_S100cF-5X-35O22-5X-N6-5X-PGT121-5X-VRC26.25-His
   SEQ ID NO:82

GAATTCGCCGCCACCATGGGATGGAGCTGTATTATTCTGTTTCTGGTCGCTAC
CGCTACCGGAGTGCATTCTTCTGAACTGACCCAGGACCCCGCCGTGAGCGTG
GCCCTGAAGCAGACCGTGACAATCACCTGCAGGGGCGACAGCCTGCGCTCCC
ACTACGCCAGCTGGTATCAGAAGAAGCCTGGCCAGGCCCCAGTGCTGCTGTT
CTACGGCAAGAACAATAGGCCCTCCGGCATCCCTGATCGCTTTTCCGGCTCTG
CCAGCGGAAACAGGGCCAGCCTGACAATCACCGGAGCACAGGCAGAGGACG
AGGCAGATTACTATTGCAGCTCCCGGGACAAGTCCGGCTCTAGACTGAGCGT
GTTCGGCGGCGGCACCAAGCTGACAGTGCTGGGAGGAGGAGGCAGCGGCGG
AGGAGGCTCCGGAGGCGGCGGCTCTGAGGTGCGGCTGGTGGAGTCTGGAGG
AGGCCTGGTGAAGCCAGGAGGCAGCCTGAGACTGAGCTGTTCCGCCTCTGGC
TTCGACTTTGATAATGCCTGGATGACATGGGTGCGGCAGCCACCTGGCAAGG
GCCTGGAGTGGGTGGGAAGAATCACCGGACCAGGAGAGGGATGGTCTGTGG
ACTACGCCGAGAGCGTGAAGGGCCGGTTCACCATCTCCAGAGATAACACCAA
GAATACACTGTATCTGGAGATGAACAATGTGCGGACCGAGGACACAGGCTAC
TATTTCTGCGCCAGAACCGGCAAGTACTATGATTTTTGGTTTGGCTACCCACC
CGGCGAGGAGTATTTTCAGGACTGGGGCCAGGGCACCCTGGTCATCGTGAGC
AGCGGCGGCGGCGGCAGCGGCGGCGGCGGCTCCGGAGGAGGCGGCTCTGGA
GGAGGAGGCAGCGGGGGAGGGGGCAGCCAGTCCGTGCTGACCCAGTCTGCC
AGCGTGTCCGGCTCTCTGGGACAGAGCGTGACCATCTCCTGTACAGGCCCCA
ACAGCGTGTGCTGTAGCCACAAGAGCATCTCCTGGTACCAGTGGCCTCCAGG
AAGGGCACCTACCCTGATCATCTATGAGGACAATGAGCGGGCCCCAGGCATC
TCCCCCAGATTCTCTGGCTACAAGTCTTATTGGAGCGCCTACCTGACAATCAG
CGACCTGCGCCCCGAGGATGAGACAACATACTATTGCTGTTCCTATACCCACA
ACTCTGGCTGCGTGTTTGGCACAGGCACCAAGGTGTCCGTGCTGGGCGGCGG
CGGCAGCGGGGGCGGGGGCTCCGGAGGGGGCGGCTCTCAGGGCCAGCTGGT
GCAGAGCGGAGCAGAGCTGAAGAAGCCTGGAGCCAGCGTGAAGATCTCCTG
TAAGACATCTGGCTACCGGTTCAACTTTTATCACATCAATTGGATCAGGCAGA
CCGCAGGAAGGGGACCAGAGTGGATGGGCTGGATCTCCCCCTACTCTGGCGA
TAAGAACCTGGCCCCAGCCTTCCAGGACAGAGTGATCATGACCACAGATACC
GAGGTGCCAGTGACCAGCTTCACCTCCACCGGAGCCGCCTACATGGAGATCA
GGAATCTGAAGTTCGACGATACAGGCACCTATTTTTGCGCAAAGGGCCTGCT
GAGGGACGGCTCCTCTACCTGGCTGCCTTACCTGTGGGGACAGGGCACCCTG
CTGACAGTGAGCTCCGGCGGCGGGGGCAGCGGCGGCGGGGGCTCCGGAGGA
GGAGGCTCTGGAGGAGGGGGCAGCGGAGGAGGCGGCTCCTACATCCACGTG
ACCCAGTCCCCATCTAGCCTGTCTGTGAGCATCGGCGATCGGGTGACCATCAA
CTGTCAGACATCTCAGGGCGTGGGCAGCGACCTGCACTGGTATCAGCACAAG
CCTGGCAGGGCCCCAAAGCTGCTGATCCACCACACATCCTCTGTGGAGGATG
GAGTGCCAAGCCGCTTCTCCGGCTCTGGATTCCACACCTCCTTTAATCTGACA
ATCTCTGACCTGCAGGCCGACGATATCGCCACCTACTATTGCCAGGTGCTGCA
GTTCTTTGGCCGGGGCTCCAGACTGCACATCAAGGGAGGAGGAGGCTCCGGG
GGCGGAGGCTCTGGCGGCGGCGGCAGCCGGGCCCACCTGGTGCAGAGCGGC
ACCGCCATGAAGAAGCCTGGCGCCAGCGTGAGAGTGTCCTGTCAGACATCTG
GCTACACCTTCACCGCCCACATCCTGTTCTGGTTTAGGCAGGCACCAGGAAGA
GGCCTGGAGTGGGTGGGCTGGATCAAGCCCCAGTATGGAGCAGTGAACTTCG
GAGGAGGCTTTCGGGACAGAGTGACACTGACCCGGGACGTGTACAGAGAGA
TCGCCTATATGGATATCAGGGGCCTGAAGCCAGACGATACCGCCGTGTACTA
TTGCGCCAGGGACCGCTCCTACGGCGATAGCTCCTGGGCACTGGACGCATGG
GGACAGGGCACCACAGTGGTGGTGAGCGCCGGCGGCGGAGGCTCCGGCGGC
GGGGGCTCTGGAGGAGGCGGCAGCGGAGGGGGAGGCTCCGGAGGGGGAGGC
TCTAGCGACATCTCCGTGGCCCCTGGCGAGACAGCCAGAATCTCTTGTGGCG
AGAAGTCTCTGGGCAGCAGGGCCGTGCAGTGGTACCAGCACAGGGCAGGAC
AGGCACCATCTCTGATCATCTATAACAATCAGGATAGGCCAAGCGGCATCCC
TGAGCGGTTCAGCGGCTCCCCCGACAGCCCTTTTGGCACCACAGCCACACTG
ACCATCACATCCGTGGAGGCAGGCGACGAAGCCGATTACTATTGCCACATCT
GGGATTCCAGAGTGCCAACCAAGTGGGTGTTCGGAGGAGGAACCACACTGAC
AGTGCTGGGAGGGGGGGGCTCTGGCGGCGGGGGCAGCGGGGGAGGAGGCTC
CCAGATGCAGCTGCAGGAGAGCGGACCAGGCCTGGTGAAGCCTAGCGAGAC
ACTGAGCCTGACATGTTCTGTGAGCGGCGCCTCCATCTCTGACAGCTACTGGT
CTTGGATCAGACGGAGCCCCGGCAAGGGCCTGGAATGGATCGGCTACGTGCA
CAAGTCCGGCGATACAAACTATTCCCCATCTCTGAAGTCTCGGGTGAACCTGT
CTCTGGACACCAGCAAGAATCAGGTGAGCCTGTCCCTGGTGGCAGCAACCGC
AGCAGATAGCGGCAAGTACTATTGCGCCAGAACACTGCACGGCAGGCGCATC
TACGGCATCGTGGCCTTTAACGAGTGGTTCACCTACTTTTATATGGACGTGTG
GGGCAATGGCACCCAGGTGACAGTGTCCTCTGGCGGGGGCGGCTCCGGAGGC
GGAGGCTCTGGCGGGGGCGGCAGCGGCGGGGGCGGCTCCGGGGGAGGCGGC
TCTCAGAGCGTGCTGACCCAGCCACCTTCCGTGTCTGCCGCACCAGGACAGA
AGGTGACCATCAGCTGTTCCGGCAACACATCCAATATCGGCAACAATTTCGT
GTCTTGGTACCAGCAGAGGCCAGGAAGGGCACCACAGCTGCTGATCTATGAG
ACAGACAAGCGGCCTTCCGGCATCCCAGATAGATTTTCTGCCAGCAAGTCCG
GCACCAGCGGCACACTGGCAATCACCGGCCTGCAGACAGGCGACGAAGCTG
ATTACTATTGCGCAACCTGGGCAGCCTCCCTGAGCTCCGCCAGGGTGTTCGGA
ACCGGAACAAAAGTGATCGTGCTGGTGGGCGGCGGAGGCTCTGGCGGAGGC
GGCAGCGGCGGGGGGGGCTCCCAGGTGCAGCTGGTGGAGAGCGGCGGCGGC
GTGGTGCAGCCAGGCACCTCCCTGAGGCTGTCTTGTGCAGCAAGCCAGTTCA
GATTTGATGGCTACGGCATGCACTGGGTGCGCCAGGCACCAGGCAAGGGCCT
GGAGTGGGTAGCCTCTATCAGCCACGACGGCATCAAGAAGTACCACGCCGAG
AAAGTGTGGGGCAGGTTCACCATCTCCCGCGATAACTCTAAAAACACACTGT
ATCTGCAGATGAACTCCCTGAGGCCCGAGGACACCGCCCTGTACTATTGCGC
CAAGGACCTGCGCGAGGATGAGTGTGAGGAGTGGTGGTCCGACTACTATGAT
TTTGGCAAGCAGCTGCCTTGCGCAAAGAGCAGGGGAGGCCTGGTGGGAATCG
CCGATAATTGGGGCCAGGGCACCATGGTGACAGTGTCTAGCGGATCCGGAGA
GCTGAAGACCCCTCTGGGCGATACCACACACACATCCCCACGGAGCCCCGAG
CCAAAGTCCTCTGACACCCCACCCCCTAGCCCTAGATCCCCTGAGCCAAAGA
GCTCCGATACACCACCCCCTTCTCCAAGGAGCCCCGAGCCTAAGTCTAGCGA
CACCCCACCCCCTTGCCCCCGCTGTCCAGCACCAGAGCTGCTGGGAGGACCA
AGCGTGTTCCTGTTTCCACCCAAGCCTAAGGATACACTGATGATCTCTCGCAC
CCCCGAGGTGACATGCGTGGTGGTGGACGTGAGCCACGAGGACCCCGAGGTG
AAGTTCAACTGGTACGTGGACGGCGTGGAGGTGCACAATGCCAAGACCAAGC
CCAGGGAGGAGCAGTACAACAGCACCTATCGCGTGGTGTCCGTGCTGACAGT
GCTGCACCAGGACTGGCTGAACGGCAAGGAGTACAAGTGCAAGGTGTCCAAT
AAGGCCCTGCCTGCCCCAATCGAGAAGACAATCAGCAAGGCAAAGGGACAG
CCAAGGGAGCCACAGGTGTACACCCTGCCTCCAAGCCGCGAGGAGATGACCA
AGAACCAGGTGTCCCTGACATGTCTGGTGAAGGGCTTCTATCCTAGCGATATC
GCCGTGGAGTGGGAGTCCAATGGCCAGCCAGAGAACAATTACAAGACCACA
CCCCCTGTGCTGGACTCCGATGGCTCTTTCTTTCTGTATTCTAAGCTGACCGTG
GACAAGAGCCGGTGGCAGCAGGGCAACGTGTTCAGCTGCTCTGTGCTGCACG
AAGCCCTGCACAGCCATTACACCCAGAAGAGCCTGAGCCTGAGCCCCGGAAA
ATAGTGAAAGCTT

3. PentaNAb_{2.0} Reverse VRC26.25-5X-PGT121-5X-N6-5X-35O22-5X-10E8v4_S100cF-His
   SEQ ID NO:83

GAATTCGCCGCCACCATGGGCTGGAGCTGCATCATCCTGTTCCTGGTG
GCAACCGCAACAGGAGTGCACAGCCAGGTGCAGCTGGTGGAGTCCGG
AGGAGGAGTGGTGCAGCCAGGCACCTCTCTGAGGCTGAGCTGTGCAG
CATCCCAGTTCAGATTTGATGGCTACGGAATGCACTGGGTGAGGCAGG
CACCAGGCAAGGGACTGGAGTGGGTGGCCAGCATCTCCCACGACGGC
ATCAAGAAGTACCACGCCGAGAAAGTGTGGGGCAGGTTCACCATCTCT
CGCGATAACAGCAAGAATACACTGTATCTGCAGATGAACAGCCTGAG
GCCCGAGGACACCGCCCTGTACTATTGCGCCAAGGACCTGCGCGAGGA
TGAGTGTGAGGAGTGGTGGTCCGACTACTATGATTTTGGCAAGCAGCT
GCCTTGCGCAAAGAGCAGGGGAGGACTGGTGGGAATCGCCGACAATT
GGGGCCAGGGCACCATGGTGACAGTGAGCAGCGGAGGAGGAGGCTCT
GGAGGAGGAGGCAGCGGA
GGCGGCGGCTCTCAGAGCGTGCTGACCCAGCCACCTTCCGTGTCTGCC
GCACCAGGACAGAAGGTGACCATCAGCTGTTCCGGCAACACATCCAAT
ATCGGCAACAATTTCGTGTCTTGGTACCAGCAGAGGCCTGGAAGAGCA
CCACAGCTGCTGATCTATGAGACAGACAAGAGGCCCTCCGGCATCCCT
GATCGCTTTTCTGCCAGCAAGTCCGGCACCAGCGGCACACTGGCAATC
ACCGGACTGCAGACAGGCGACGAGGCAGATTACTATTGCGCAACCTG
GGCAGCCTCCCTG
TCTAGCGCCAGGGTGTTCGGCACCGGCACAAAAGTGATCGTGCTGGTG
GGAGGAGGAGGCTCCGGCGGCGGGGGCTCTGGCGGCGGCGGCAGCGG
AGGAGGCGGCTCCGGAGGAGGCGGCTCTCAGATGCAGCTGCAGGAGA
GCGGACCAGGACTGGTGAAGCCTTCCGAGACCCTGTCTCTGACATGTT
CTGTGAGCGGCGCCTCCATCTCTGATAGCTACTGGAGCTGGATCAGAC
GGAGCCCTGGCAAGGGCCTGGAGTGGATCGGCTACGTGCACAAGTCT
GGCGATACAAACTATTCCCCATCTCTGAAGAGCCGGGTGAACCTGAGC
CTGGACACCTCCAAGAATCAGGTGAGCCTGTCCCTGGTGGCAGCAACC
GCAGCAGACAGCGGCAAGTACTATTGCGCCAGAACACTGCACGGCAG
GCGCATCTACGGCATCGTGGCCTTTAACGAGTGGTTCACCTACTTTTAT
ATGGACGTGTGGGGCAATGGCACCCAGGTGACAGTGTCCTCTGGCGGC
GGCGGCTCTGGCGGAGGAGGCAGCGGAGGAGGAGGCAGCTCCGACAT
CTCTGTGGCACCTGGAGAGACCGCAAGGATCAGCTGTGGAGAGAAGT
CTCTGGGCAGCAGGGCCGTGCAGTGGTACCAGCACAGGGCAGGACAG
GCACCATCCCTGATCATCTATAACAATCAGGACCGGCCATCTGGCATC
CCCGAGAGATTCTCTGGCAGCCCCGATAGCCCTTTTGGCACCACAGCC
ACCCTGACAATCACCTCCGTGGAGGCCGGCGACGAAGCAGATTACTAT
TGCCACATCTGGGACTCCAGAGTGCCAACCAAGTGGGTGTTCGGAGGA
GGAACCACACTGACAGTGCTGGGCGGCGGAGGCTCCGGCGGGGGCGG
CTCTGGAGGCGGCGGCAGCGGAGGGGGCGGCTCCGGCGGCGGCGGCT
CTAGGGCACACCTGGTGCAGAGCGGAACCGCAATGAAGAAGCCTGGC
GCCTCTGTGCGCGTGAGCTGTCAGACATCCGGCTACACCTTCACCGCC
CACATCCTGTTCTGGTTTAGGCAGGCACCAGGAAGAGGACTGGAGTGG
GTGGGCTGGATCAAGCCCCAGTATGGAGCAGTGAACTTCGGAGGA
GGCTTTCGGGACAGAGTGACACTGACCCGGGACGTGTACAGAGAGAT
CGCCTATATGGATATCAGGGGCCTGAAGCCCGACGATACCGCCGTGTA
CTATTGCGCCAGGGACCGCTCCTACGGCGATTCTAGCTGGGCACTGGA
CGCATGGGGACAGGGAACCACAGTGGTGGTGAGCGCCGGAGGCGGGG
GCAGCGGCGGCGGGGGCTCCGGAGGCGGAGGCTCTTACATCCACGTG
ACCCAGTCCCCTTCCTCTCTGTCCGTGTCTATCGGCGATCGCGTGACCA
TCAACTGTCAGACAAGCCAGGGAGTGGGCTCCGACCTGCACTGGTATC
AGCACAAGCCTGGCAGGGCCCCAAAGCTGCTGATCCACCACACAAGC
TCCGTGGAGGATGGAGTGCCAAGCCGCTTCAGCGGCTCCGGATTCCAC
ACCAGCTTTAATCTGACAATCTCCGACCTGCAGGCCGACGATATCGCC
ACCTACTATTGCCAGGTGCTGCAGTTCTTTGGCAGGGGCTCCCGCCTGC
ACATCAAGGGCGGCGGCGGCTCTGGGGGCGGGGGCAGCGGCGGGGGG
GGCTCCGGGGGAGGAGGCTCTGGCGGAGGGGGCAGCCAGGGCCAGCT
GGTGCAGAGCGGAGCAGAGCTGAAGAAGCCAGGAGCCTCTGTGAAGA
TCAGCTGTAAGACATCCGGCTACCGGTTCAACTTTTATCACATCAATTG
GATCAGGCAGACCGCAGGAAGGGGACCAGAGTGGATGGGCTGGATCT
CCCCCTACTCTGGCGATAAGAACCTGGCCCCAGCCTTCCAGGACAGAG
TGATCATGACCACAGATACCGAGGTGCCAGTGACCAGCTTCACCTCCA
CCGGAGCCGCCTACATGGAGATCCGGAATCTGAAGTTCGACGATACAG
GCACCTATTTTTGCGCCAAGGGCCTGCTGAGAGACGGCTCTAGCACAT
GGCTGCCATACCTGTGGGGACAGGGCACCCTGCTGACAGTGTCCTCTG
GAGGAGGAGGCTCCGGGGGCGGCGGCTCTGGAGGAGGAGGCTCTCAA
AGCGTGCTGACCCAGTCCGCCTCTGTGAGCGGCTCCCTGGGCCAGTCT
GTGACCATCAGCTGTACAGGCCCCAACTCCGTGTGCTGTTCTCACAAG
TCTATCAGCTGGTACCAGTGGCCACCAGGAAGGGCACCTACCCTGATC
ATCTATGAGGACAATGAGAGGGCACCAGGAATCAGCCCTCGCTTCTCC
GGCTACAAGTCTTATTGGAGCGCCTACCTGACCATTTCCGACCTGCGC
CCCGAGGATGAGACCACATACTATTGCTGTAGCTATACCCACAACTCC
GGCTGCGTGTTTGGCACAGGCACCAAGGTGAGCGTGCTGGGAGGAGG
GGGCTCTGGCGGCGGGGGCAGCGGCGGAGGCGGCTCCGGAGGGGGCG
GCTCTGGCGGAGGCGGCAGCGAGGTGCGGCTGGTGGAGAGCGGCGGC
GGCCTGGTGAAGCCAGGCGGCTCTCTGAGACTGTCCTGTTCTGCCAGC
GGCTTCGACTTTGATAATGCCTGGATGACATGGGTGCGGCAGCCTCCT
GGCAAGGGGCTGGAGTGGGTGGGAAGAATCACCGGACCAGGAGAGG
GATGGTCTGTGGACTACGCCGAGAGCGTGAAGGGCCGGTTCACCATCA
GCAGAGATAACACTAAAAATACACTGTATCTGGAGATGAACAATGTG
CGGACCGAGGACACAGGCTACTATTTCTGCGCCAGAACCGGCAAGTAC
TATGATTTCTGGTTTGGCTACCCCCCTGGCGAGGAGTATTTTCAGGACT
GGGGCCAGGGCACCCTGGTCATCGTGAGCAGCGGCGGGGGAGGCTCC
GGCGGGGGGGGCTCTGGAGGAGGGGGCTCTAGCGAGCTGACCCAGGA
CCCCGCCGTGTCCGTGGCCCTGAAGCAGACAGTGACCATCACATGCAG
GGGCGACTCCCTGCGCTCTCACTACGCCAGCTGGTATCAGAAGAAGCC
AGGACAGGCACCCGTGCTGCTGTTCTACGGCAAGAACAATCGGCCTTC
CGGCATCCCAGATAGATTTTCCGGCTCTGCCAGCGGAAACAGGGCCAG
CCTGACCATCACAGGAGCACAGGCAGAGGATGAAGCAGATTACTATT
GTTCCTCTCGGGACAAGTCCGGCTCTAGACTGAGCGTGTTCGGCGGCG
GAACCAAGCTGACAGTGCTGGGATCCGGCCACCACCATCACCATCATT
AGTGAAGCTT

4. PentaNAb_{3.0} 10E8v4_S100cF-5X-35O22-5X-N6-5X-PGT121-5X-PGDM1400-His
   SEQ ID NO:84

GAATTCGCCG CCACCATGGG ATGGAGCTGT ATTATTCTGT
TTCTGGTCGC TACCGCTACC GGAGTGCATT CTTCTGAACT
GACCCAGGAC CCCGCCGTGA GCGTGGCCCT GAAGCAGACC
GTGACAATCA CCTGCAGGGG CGACAGCCTG CGCTCCCACT
ACGCCAGCTG GTATCAGAAG AAGCCTGGCC AGGCCCCAGT
GCTGCTGTTC TACGGCAAGA ACAATAGGCC CTCCGGCATC
CCTGATCGCT TTTCCGGCTC TGCCAGCGGA AACAGGGCCA
GCCTGACAAT CACCGGAGCA CAGGCAGAGG ACGAGGCAGA
TTACTATTGC AGCTCCCGGG ACAAGTCCGG CTCTAGACTG
AGCGTGTTCG GCGGCGGCAC CAAGCTGACA GTGCTGGGAG
GAGGAGGCAG CGGCGGAGGA GGCTCCGGAG GCGGCGGCTC
TGAGGTGCGG CTGGTGGAGT CTGGAGGAGG CCTGGTGAAG
CCAGGAGGCA GCCTGAGACT GAGCTGTTCC GCCTCTGGCT
TCGACTTTGA TAATGCCTGG ATGACATGGG TGCGGCAGCC
ACCTGGCAAG GGCCTGGAGT GGGTGGGAAG AATCACCGGA
CCAGGAGAGG GATGGTCTGT GGACTACGCC GAGAGCGTGA
AGGGCCGGTT CACCATCTCC AGAGATAACA CCAAGAATAC
ACTGTATCTG GAGATGAACA ATGTGCGGAC CGAGGACACA
GGCTACTATT TCTGCGCCAG AACCGGCAAG TACTATGATT
TTTGGTTTGG CTACCCACCC GGCGAGGAGT ATTTTCAGGA
CTGGGGCCAG GGCACCCTGG TCATCGTGAG CAGCGGCGGC
GGCGGCAGCG GCGGCGGCGG CTCCGGAGGA GGCGGCTCTG
GAGGAGGAGG CAGCGGGGGA GGGGGCAGCC AGTCCGTGCT
GACCCAGTCT GCCAGCGTGT CCGGCTCTCT GGGACAGAGC
GTGACCATCT CCTGTACAGG CCCCAACAGC GTGTGCTGTA
GCCACAAGAG CATCTCCTGG TACCAGTGGC CTCCAGGAAG
GGCACCTACC CTGATCATCT ATGAGGACAA TGAGCGGGCC
CCAGGCATCT CCCCCAGATT CTCTGGCTAC AAGTCTTATT
GGAGCGCCTA CCTGACAATC AGCGACCTGC GCCCCGAGGA
TGAGACAACA TACTATTGCT GTTCCTATAC CCACAACTCT
GGCTGCGTGT TTGGCACAGG CACCAAGGTG TCCGTGCTGG
GCGGCGGCGG CAGCGGGGGC GGGGGCTCCG GAGGGGGCGG
CTCTCAGGGC CAGCTGGTGC AGAGCGGAGC AGAGCTGAAG
AAGCCTGGAG CCAGCGTGAA GATCTCCTGT AAGACATCTG
GCTACCGGTT CAACTTTTAT CACATCAATT GGATCAGGCA
GACCGCAGGA AGGGGACCAG AGTGGATGGG CTGGATCTCC
CCCTACTCTG GCGATAAGAA CCTGGCCCCA GCCTTCCAGG
ACAGAGTGAT CATGACCACA GATACCGAGG TGCCAGTGAC
CAGCTTCACC TCCACCGGAG CCGCCTACAT GGAGATCAGG
AATCTGAAGT TCGACGATAC AGGCACCTAT TTTTGCGCAA
AGGGCCTGCT GAGGGACGGC TCCTCTACCT GGCTGCCTTA
CCTGTGGGGA CAGGGCACCC TGCTGACAGT GAGCTCCGGC
GGCGGGGGCA GCGGCGGCGG GGGCTCCGGA GGAGGAGGCT
CTGGAGGAGG GGGCAGCGGA GGAGGCGGCT CCTACATCCA
CGTGACCCAG TCCCCATCTA GCCTGTCTGT GAGCATCGGC
GATCGGGTGA CCATCAACTG TCAGACATCT CAGGGCGTGG
GCAGCGACCT GCACTGGTAT CAGCACAAGC CTGGCAGGGC
CCCAAAGCTG CTGATCCACC ACACATCCTC TGTGGAGGAT
GGAGTGCCAA GCCGCTTCTC CGGCTCTGGA TTCCACACCT
CCTTTAATCT GACAATCTCT GACCTGCAGG CCGACGATAT
CGCCACCTAC TATTGCCAGG TGCTGCAGTT CTTTGGCCGG
GGCTCCAGAC TGCACATCAA GGGAGGAGGA GGCTCCGGGG
GCGGAGGCTC TGGCGGCGGC GGCAGCCGGG CCCACCTGGT
GCAGAGCGGC ACCGCCATGA AGAAGCCTGG CGCCAGCGTG
AGAGTGTCCT GTCAGACATC TGGCTACACC TTCACCGCCC
ACATCCTGTT CTGGTTTAGG CAGGCACCAG GAAGAGGCCT
GGAGTGGGTG GGCTGGATCA AGCCCCAGTA TGGAGCAGTG
AACTTCGGAG GAGGCTTTCG GGACAGAGTG ACACTGACCC
GGGACGTGTA CAGAGAGATC GCCTATATGG ATATCAGGGG
CCTGAAGCCA GACGATACCG CCGTGTACTA TTGCGCCAGG
GACCGCTCCT ACGGCGATAG CTCCTGGGCA CTGGACGCAT
GGGGACAGGG CACCACAGTG GTGGTGAGCG CCGGCGGCGG
AGGCTCCGGC GGCGGGGGCT CTGGAGGAGG CGGCAGCGGA
GGGGGAGGCT CCGGAGGGGG AGGCTCTAGC GACATCTCCG
TGGCCCCTGG CGAGACAGCC AGAATCTCTT GTGGCGAGAA
GTCTCTGGGC AGCAGGGCCG TGCAGTGGTA CCAGCACAGG
GCAGGACAGG CACCATCTCT GATCATCTAT AACAATCAGG
ATAGGCCAAG CGGCATCCCT GAGCGGTTCA GCGGCTCCCC
CGACAGCCCT TTTGGCACCA CAGCCACACT GACCATCACA
TCCGTGGAGG CAGGCGACGA AGCCGATTAC TATTGCCACA
TCTGGGATTC CAGAGTGCCA ACCAAGTGGG TGTTCGGAGG
AGGAACCACA CTGACAGTGC TGGGAGGGGG GGGCTCTGGC
GGCGGGGGCA GCGGGGGAGG AGGCTCCCAG ATGCAGCTGC
AGGAGAGCGG ACCAGGCCTG GTGAAGCCTA GCGAGACACT
GAGCCTGACA TGTTCTGTGA GCGGCGCCTC CATCTCTGAC
AGCTACTGGT CTTGGATCAG ACGGAGCCCC GGCAAGGGCC
TGGAATGGAT CGGCTACGTG CACAAGTCCG GCGATACAAA
CTATTCCCCA TCTCTGAAGT CTCGGGTGAA CCTGTCTCTG
GACACCAGCA AGAATCAGGT GAGCCTGTCC CTGGTGGCAG
CAACCGCAGC AGATAGCGGC AAGTACTATT GCGCCAGAAC
ACTGCACGGC AGGCGCATCT ACGGCATCGT GGCCTTTAAC
GAGTGGTTCA CCTACTTTTA TATGGACGTG TGGGGCAATG
GCACCCAGGT GACAGTGTCC TCTGGCGGGG GCGGCTCCGG
AGGCGGAGGC TCTGGCGGGG GCGGCAGCGG CGGGGGCGGC
TCCGGGGGAG GGGCTCTGA TTTCGTGCTG ACCCAGTCTC
CACATAGTCT GAGCGTGACA CCCGGCGAAA GCGCATCAAT
TTCTTGTAAA TCATCTCATA GTCTGATCCA CGGCGATAGG
AACAATTACC TGGCCTGGTA CGTGCAGAAG CCAGGCCGCA
GCCCTCAGCT GCTGATCTAC CTGGCAAGCT CCAGGGCATC
CGGAGTGCCA GATCGCTTCT CTGGCAGCGG CTCCGATAAG
GACTTTACCC TGAAGATCTC CCGGGTGGAG ACAGAGGACG
TGGGCACATA CTATTGCATG CAGGGCAGAG AGTCTCCTTG
GACCTTCGGC CAGGGCACAA AGGTGGACAT CAAGGGAGGA
GGAGGCAGCG GCGGAGGAGG CTCCGGCGGC GGCGGCTCTC
AGGCACAGCT GGTGCAGAGC GGACCAGAGG TGAGGAAGCC
AGGCACCTCT GTGAAGGTGA GCTGTAAGGC CCCTGGCAAC
ACCCTGAAGA CATACGATCT GCACTGGGTG CGGTCTGTGC
CAGGACAGGG CCTGCAGTGG ATGGGATGGA TCAGCCACGA
GGGCGACAAG AAAGTGATCG TGGAGCGGTT TAAGGCCAAG
GTGACAATCG ATTGGGACAG AAGCACCAAT ACAGCCTATC
TGCAGCTGTC CGGCCTGACC TCTGGCGATA CAGCCGTGTA
CTATTGCGCC AAGGGCTCCA AGCACCGGCT GAGAGACTAC
GCCCTGTATG ACGATGACGG CGCCCTGAAT TGGGCAGTGG
ACGTGGACTA TCTGAGTAAT CTGGAGTTTT GGGGGCAGGG
CACCGCAGTG ACAGTGTCTA
GCGGATCCGGACACCACCATCACCATCATTAGTGAAAGCTT

5. PentaNAb_{3.0} Reverse PGDM1400-5X-PGT121-5X-N6-5X-35O22-5X-10E8v4S100cF-His
   SEQ ID NO:85

GAATTCGCCG CCACCATGGG CTGGAGCTGC ATCATCCTGT
TCCTGGTGGC AACCGCAACA GGAGTGCACA GCCAGGCACA
GCTGGTGCAG AGCGGACCCG AAGTGAGAAA ACCTGGGACT
AGCGTCAAAG TGTCATGTAA AGCCCCTGGA AATACCCTGA
AGACCTACGA TCTGCACTGG GTGCGGTCCG TGCCTGGACA
GGGCCTGCAG TGGATGGGAT GGATCTCTCA CGAGGGCGAC
AAGAAAGTGA TCGTGGAGCG GTTCAAGGCC AAGGTGACAA
TCGATTGGGA CAGATCCACC AACACAGCCT ACCTGCAGCT
GTCTGGCCTG ACCAGCGGCG ATACAGCCGT GTACTACTGT
GCCAAGGGCT CTAAGCACCG GCTGAGAGAC TACGCCCTGT
ATGACGATGA CGGCGCCCTG AACTGGGCCG TGGATGTGGA
CTATCTGTCC AATCTGGAGT TCTGGGGACA GGGAACCGCA
GTGACAGTGA GCTCCGGAGG AGGAGGCTCC GGCGGCGGAG
GCTCTGGGGG AGGCGGCAGC GATTTTGTGC TGACCCAGTC
TCCACACAGC CTGTCCGTGA CACCCGGCGA GTCTGCCAGC
ATCTCCTGCA AGTCTAGCCA CAGCCTGATC CACGGCGACA
GGAACAATTA CCTGGCCTGG TACGTGCAGA AGCCAGGCCG
CAGCCCTCAG CTGCTGATCT ATCTGGCATC CTCTAGGGCC
TCCGGAGTGC CAGATCGCTT CTCTGGCAGC GGCTCCGATA
AGGACTTTAC CCTGAAGATC AGCCGGGTGG AGACAGAGGA
CGTGGGCACA TACTATTGTA TGCAGGGCCG AGAATCACCT
TGGACATTTG GGCAGGGAAC TAAAGTCGAC ATCAAAGGGG
GGGGGGGCTC CGGCGGCGGG GGCTCTGGCG GCGGCGGCAG
CGGAGGAGGC GGCTCCGGAG GAGGCGGCTC TCAGATGCAG
CTGCAGGAGA GCGGACCAGG ACTGGTGAAG CCTTCCGAGA
CCCTGTCTCT GACATGTTCT GTGAGCGGCG CCTCCATCTC
TGATAGCTAC TGGAGCTGGA TCAGACGGAG CCCTGGCAAG
GGCCTGGAGT GGATCGGCTA CGTGCACAAG TCTGGCGATA
CAAACTATTC CCCATCTCTG AAGAGCCGGG TGAACCTGAG
CCTGGACACC TCCAAGAATC AGGTGAGCCT GTCCCTGGTG
GCAGCAACCG CAGCAGACAG CGGCAAGTAC TATTGCGCCA
GAACACTGCA CGGCAGGCGC ATCTACGGCA TCGTGGCCTT
TAACGAGTGG TTCACCTACT TTTATATGGA CGTGTGGGGC
AATGGCACCC AGGTGACAGT GTCCTCTGGC GGCGGCGGCT
CTGGCGGAGG AGGCAGCGGA GGAGGAGGCA GCTCCGACAT
CTCTGTGGCA CCTGGAGAGA CCGCAAGGAT CAGCTGTGGA
GAGAAGTCTC TGGGCAGCAG GGCCGTGCAG TGGTACCAGC
ACAGGGCAGG ACAGGCACCA TCCCTGATCA TCTATAACAA
TCAGGACCGG CCATCTGGCA TCCCCGAGAG ATTCTCTGGC
AGCCCCGATA GCCCTTTTGG CACCACAGCC ACCCTGACAA
TCACCTCCGT GGAGGCCGGC GACGAAGCAG ATTACTATTG
CCACATCTGG GACTCCAGAG TGCCAACCAA GTGGGTGTTC
GGAGGAGGAA CCACACTGAC AGTGCTGGGC GGCGGAGGCT
CCGGCGGGGG CGGCTCTGGA GGCGGCGGCA GCGGAGGGGG
CGGCTCCGGC GGCGGCGGCT CTAGGGCACA CCTGGTGCAG
AGCGGAACCG CAATGAAGAA GCCTGGCGCC TCTGTGCGCG
TGAGCTGTCA GACATCCGGC TACACCTTCA CCGCCCACAT
CCTGTTCTGG TTTAGGCAGG CACCAGGAAG AGGACTGGAG
TGGGTGGGCT GGATCAAGCC CCAGTATGGA GCAGTGAACT
TCGGAGGAGG CTTTCGGGAC AGAGTGACAC TGACCCGGGA
CGTGTACAGA GAGATCGCCT ATATGGATAT CAGGGGCCTG
AAGCCCGACG ATACCGCCGT GTACTATTGC GCCAGGGACC
GCTCCTACGG CGATTCTAGC TGGGCACTGG ACGCATGGGG
ACAGGGAACC ACAGTGGTGG TGAGCGCCGG AGGCGGGGGC
AGCGGCGGCG GGGGCTCCGG AGGCGGAGGC TCTTACATCC
ACGTGACCCA GTCCCCTTCC TCTCTGTCCG TGTCTATCGG
CGATCGCGTG ACCATCAACT GTCAGACAAG CCAGGGAGTG
GGCTCCGACC TGCACTGGTA TCAGCACAAG CCTGGCAGGG
CCCCAAAGCT GCTGATCCAC CACACAAGCT CCGTGGAGGA
TGGAGTGCCA AGCCGCTTCA GCGGCTCCGG ATTCCACACC
AGCTTTAATC TGACAATCTC CGACCTGCAG GCCGACGATA
TCGCCACCTA CTATTGCCAG GTGCTGCAGT TCTTTGGCAG
GGGCTCCCGC CTGCACATCA AGGGCGGCGG CGGCTCTGGG
GGCGGGGGCA GCGGGGGGG GGGCTCCGGG GGAGGAGGCT
CTGGCGGAGG GGGCAGCCAG GGCCAGCTGG TGCAGAGCGG
AGCAGAGCTG AAGAAGCCAG GAGCCTCTGT GAAGATCAGC
TGTAAGACAT CCGGCTACCG GTTCAACTTT TATCACATCA
ATTGGATCAG GCAGACCGCA GGAAGGGGAC CAGAGTGGAT
GGGCTGGATC TCCCCCTACT CTGGCGATAA GAACCTGGCC
CCAGCCTTCC AGGACAGAGT GATCATGACC ACAGATACCG
AGGTGCCAGT GACCAGCTTC ACCTCCACCG GAGCCGCCTA
CATGGAGATC CGGAATCTGA AGTTCGACGA TACAGGCACC
TATTTTTGCG CCAAGGGCCT GCTGAGAGAC GGCTCTAGCA
CATGGCTGCC ATACCTGTGG GGACAGGGCA CCCTGCTGAC
AGTGTCCTCT GGAGGAGGAG GCTCCGGGGG CGGCGGCTCT
GGAGGAGGAG GCTCTCAAAG CGTGCTGACC CAGTCCGCCT
CTGTGAGCGG CTCCCTGGGC CAGTCTGTGA CCATCAGCTG
TACAGGCCCC AACTCCGTGT GCTGTTCTCA CAAGTCTATC
AGCTGGTACC AGTGGCCACC AGGAAGGGCA CCTACCCTGA
TCATCTATGA GGACAATGAG AGGGCACCAG GAATCAGCCC
TCGCTTCTCC GGCTACAAGT CTTATTGGAG CGCCTACCTG
ACCATTTCCG ACCTGCGCCC CGAGGATGAG ACCACATACT
ATTGCTGTAG CTATACCCAC AACTCCGGCT GCGTGTTTGG
CACAGGCACC AAGGTGAGCG TGCTGGGAGG AGGGGGCTCT
GGCGGCGGGG GCAGCGGCGG AGGCGGCTCC GGAGGGGGCG
GCTCTGGCGG AGGCGGCAGC GAGGTGCGGC TGGTGGAGAG
CGGCGGCGGC CTGGTGAAGC CAGGCGGCTC TCTGAGACTG
TCCTGTTCTG CCAGCGGCTT CGACTTTGAT AATGCCTGGA
TGACATGGGT GCGGCAGCCT CCTGGCAAGG GGCTGGAGTG
GGTGGGAAGA ATCACCGGAC CAGGAGAGGG ATGGTCTGTG
GACTACGCCG AGAGCGTGAA GGGCCGGTTC ACCATCAGCA
GAGATAACAC TAAAAATACA CTGTATCTGG AGATGAACAA
TGTGCGGACC GAGGACACAG GCTACTATTT CTGCGCCAGA
ACCGGCAAGT ACTATGATTT CTGGTTTGGC TACCCCCCTG
GCGAGGAGTA TTTTCAGGAC TGGGGCCAGG GCACCCTGGT
CATCGTGAGC AGCGGCGGGG GAGGCTCCGG CGGGGGGGGC
TCTGGAGGAG GGGGCTCTAG CGAGCTGACC CAGGACCCCG
CCGTGTCCGT GGCCCTGAAG CAGACAGTGA CCATCACATG
CAGGGGCGAC TCCCTGCGCT CTCACTACGC CAGCTGGTAT
CAGAAGAAGC CAGGACAGGC ACCCGTGCTG CTGTTCTACG
GCAAGAACAA TCGGCCTTCC GGCATCCCAG ATAGATTTTC
CGGCTCTGCC AGCGGAAACA GGGCCAGCCT GACCATCACA
GGAGCACAGG CAGAGGATGA AGCAGATTAC TATTGTTCCT
CTCGGGACAA GTCCGGCTCT AGACTGAGCG TGTTCGGCGG
CGGAACCAAG CTGACAGTGC
TGGGATCCGGACACCACCATCACCATCATTAGTGAAAGCTT

6. PentaNAb_{4.0} 10E8v4 V5R_S100cF-5X-35O22-5X-N6-5X-PGT121-5X-PGDM1400-His
   SEQ ID NO:86

gaattcgccgccaccatgggatggagctgtattattctgtttctggtcgctaccgctacc
ggagtgcattcttctgaactgacccaggaccccgccgtgagcgtggccctgaagcagacc
gtgacaatcacctgcaggggcgacagcctgcgctcccactacgccagctggtatcagaag
aagcctggccaggccccagtgctgctgttctacggcaagaacaataggccctccggcatc
cctgatcgcttttccggctctgccagcggaaacagggccagcctgacaatcaccggagca
caggcagaggacgaggcagattactattgcagctcccgggacaagtccggctctagactg
agcgtgttcggcggcggcaccaagctgacagtgctgggaggaggaggcagcggcggagga
ggctccggaggcggcggctctgaggtgcggctgCGGgagtctggaggaggcctggtgaag
ccaggaggcagcctgagactgagctgttccgcctctggcttcgactttgataatgcctgg
atgacatgggtgcggcagccacctggcaagggcctggagtgggtgggaagaatcaccgga
ccaggagagggatggtctgtggactacgccgagagcgtgaagggccggttcaccatctcc
agagataacaccaagaatacactgtatctggagatgaacaatgtgcggaccgaggacaca
ggctactatttctgcgccagaaccggcaagtactatgatttttggtttggctacccaccc
ggcgaggagtattttcaggactggggccagggcaccctggtcatcgtgagcagcggcggc
ggcggcagcggcggcggcggctccggaggaggcggctctggaggaggaggcagcggggga
gggggcagccagtccgtgctgacccagtctgccagcgtgtccggctctctgggacagagc
gtgaccatctcctgtacaggccccaacagcgtgtgctgtagccacaagagcatctcctgg
taccagtggcctccaggaagggcacctaccctgatcatctatgaggacaatgagcgggcc
ccaggcatctcccccagattctctggctacaagtcttattggagcgcctacctgacaatc
agcgacctgcgccccgaggatgagacaacatactattgctgttcctatacccacaactct
ggctgcgtgtttggcacaggcaccaaggtgtccgtgctgggcggcggcggcagcgggggc
gggggctccggagggggcggctctcagggccagctggtgcagagcggagcagagctgaag
aagcctggagccagcgtgaagatctcctgtaagacatctggcTATcggttcaacttttat
cacatcaattggatcaggcagaccgcaggaaggggaccagagtggatgggctggatctcc
ccctactctggcgataagaacctggccccagccttccaggacagagtgatcatgaccaca
gataccgaggtgccagtgaccagcttcacctccaccggagccgcctacatggagatcagg
aatctgaagttcgacgatacaggcacctatttttgcgcaaagggcctgctgagggacggc
tcctctacctggctgccttacctgtggggacagggcaccctgctgacagtgagctccggc
ggcgggggcagcggcggcgggggctccggaggaggaggctctggaggagggggcagcgga
ggaggcggctcctacatccacgtgacccagtccccatctagcctgtctgtgagcatcggc
gatcgggtgaccatcaactgtcagacatctcagggcgtgggcagcgacctgcactggtat
cagcacaagcctggcagggccccaaagctgctgatccaccacacatcctctgtggaggat
ggagtgccaagccgcttctccggctctggattccacacctcctttaatctgacaatctct
gacctgcaggccgacgatatcgccacctactattgccaggtgctgcagttctttggccgg
ggctccagactgcacatcaagggaggaggaggctccgggggcggaggctctggcggcggc
ggcagccgggcccacctggtgcagagcggcaccgccatgaagaagcctggcgccagcgtg
agagtgtcctgtcagacatctggctacaccttcaccgcccacatcctgttctggtttagg
caggcaccaggaagaggcctggagtgggtgggctggatcaagccccagtatggagcagtg
aacttcggaggaggctttcgggacagagtgacactgacccgggacgtgtacagagagatc
gcctatatggatatcaggggcctgaagccagacgataccgccgtgtactattgcgccagg
gaccgctcctacggcgatagctcctgggcactggacgcatggggacagggcaccacagtg
gtggtgagcgccggcggcggaggctccggcggcgggggctctggaggaggcggcagcgga
gggggaggctccggagggggaggctctagcgacatctccgtggcccctggcgagacagcc
agaatctcttgtggcgagaagtctctgggcagcagggccgtgcagtggtaccagcacagg
gcaggacaggcaccatctctgatcatctataacaatcaggataggccaagcggcatccct
gagcggttcagcggctcccccgacagcccttttggcaccacagccacactgaccatcaca
tccgtggaggcaggcgacgaagccgattactattgccacatctgggattccagagtgcca
accaagtgggtgttcggaggaggaaccacactgacagtgctgggaggggggggctctggc
ggcgggggcagcgggggaggaggctcccagatgcagctgcaggagagcggaccaggcctg
gtgaagcctagcgagacactgagcctgacatgttctgtgagcggcgcctccatctctgac
agctactggtcttggatcagacggagccccggcaagggcctggaatggatcggctacgtg
cacaagtccggcgatacaaactattccccatctctgaagtctcgggtgaacctgtctctg
gacaccagcaagaatcaggtgagcctgtccctggtggcagcaaccgcagcagatagcggc
aagtactattgcgccagaacactgcacggcaggcgcatctacggcatcgtggcctttaac
gagtggttcacctacttttatatggacgtgtggggcaatggcacccaggtgacagtgtcc
tctggcgggggcggctccggaggcggaggctctggcgggggcggcagcggcgggggcggc
tccgggggaggcggctctgatttcgtgctgacccagtctccacatagtctgagcgtgaca
cccggcgaaagcgcatcaatttcttgtaaatcatctcatagtctgatccacggcgatagg
aacaattacctggcctggtacgtgcagaagccaggccgcagccctcagctgctgatctac
ctggcaagctccagggcatccggagtgccagatcgcttctctggcagcggctccgataag
gactttaccctgaagatctcccgggtggagacagaggacgtgggcacatactattgcatg
cagggcagagagtctccttggaccttcggccagggcacaaaggtggacatcaagggagga
ggaggcagcggcggaggaggctccggcggcggcggctctcaggcacagctggtgcagagc
ggaccagaggtgaggaagccaggcacctctgtgaaggtgagctgtaaggcccctggcaac
accctgaagacatacgatctgcactgggtgcggtctgtgccaggacagggcctgcagtgg
atgggatggatcagccacgagggcgacaagaaagtgatcgtggagcggtttaaggccaag
gtgacaatcgattgggacagaagcaccaatacagcctatctgcagctgtccggcctgacc
tctggcgatacagccgtgtactattgcgccaagggctccaagcaccggctgagagactac
gccctgtatgacgatgacggcgccctgaattgggcagtggacgtggactatctgagtaat
ctggagttttgggggcagggcaccgcagtgacagtgtctagcggatccGGACACCACCAT
CACCATCATTAGTGAAAGCTT

7. PentaNAb_{4.0} Reverse PGDM1400-5X-PGT121-5X-N6-5X-35O22-5X-10E8v4 V5R S100cF-His
   SEQ ID NO:87

gaattcgccgccaccatgggctggagctgcatcatcctgttcctggtggcaaccgcaaca
ggagtgcacagccaggcacagctggtgcagagcggacccgaagtgagaaaacctgggact
agcgtcaaagtgtcatgtaaagcccctggaaataccctgaagacctacgatctgcactgg
gtgcggtccgtgcctggacagggcctgcagtggatgggatggatctctcacgagggcgac
aagaaagtgatcgtggagcggttcaaggccaaggtgacaatcgattgggacagatccacc
aacacagcctacctgcagctgtctggcctgaccagcggcgatacagccgtgtactactgt
gccaagggctctaagcaccggctgagagactacgccctgtatgacgatgacggcgccctg
aactgggccgtggatgtggactatctgtccaatctggagttctggggacagggaaccgca
gtgacagtgagctccggaggaggaggctccggcggcggaggctctgggggaggcggcagc
gattttgtgctgacccagtctccacacagcctgtccgtgacacccggcgagtctgccagc
atctcctgcaagtctagccacagcctgatccacggcgacaggaacaattacctggcctgg
tacgtgcagaagccaggccgcagccctcagctgctgatctatctggcatcctctagggcc
tccggagtgccagatcgcttctctggcagcggctccgataaggactttaccctgaagatc
agccgggtggagacagaggacgtgggcacatactattgtatgcagggccgagaatcacct
tggacatttgggcagggaactaaaGTTgacatcaaagggggggggggctccggcggcggg
ggctctggcggcggcggcagcggaggaggcggctccggaggaggcggctctcagatgcag
ctgcaggagagcggaccaggactggtgaagccttccgagaccctgtctctgacatgttct
gtgagcggcgcctccatctctgatagctactggagctggatcagacggagccctggcaag
ggcctggagtggatcggctacgtgcacaagtctggcgatacaaactattccccatctctg
aagagccgggtgaacctgagcctggacacctccaagaatcaggtgagcctgtccctggtg
gcagcaaccgcagcagacagcggcaagtactattgcgccagaacactgcacggcaggcgc
atctacggcatcgtggcctttaacgagtggttcacctacttttatatggacgtgtggggc
aatggcacccaggtgacagtgtcctctggcggcggcggctctggcggaggaggcagcgga
ggaggaggcagctccgacatctctgtggcacctggagagaccgcaaggatcagctgtgga
gagaagtctctgggcagcagggccgtgcagtggtaccagcacagggcaggacaggcacca
tccctgatcatctataacaatcaggaccggccatctggcatccccgagagattctctggc
agccccgatagcccttttggcaccacagccaccctgacaatcacctccgtggaggccggc
gacgaagcagattactattgccacatctgggactccagagtgccaaccaagtgggtgttc
ggaggaggaaccacactgacagtgctgggcggcggaggctccggcgggggcggctctgga
ggcggcggcagcggagggggcggctccggcggcggcggctctagggcacacctggtgcag
agcggaaccgcaatgaagaagcctggcgcctctgtgcgcgtgagctgtcagacatccggc
tacaccttcaccgcccacatcctgttctggtttaggcaggcaccaggaagaggactggag
tgggtgggctggatcaagccccagtatggagcagtgaacttcggaggaggctttcgggac
agagtgacactgacccgggacgtgtacagagagatcgcctatatggatatcaggggcctg
aagcccgacgataccgccgtgtactattgcgccagggaccgctcctacggcgattctagc
tgggcactggacgcatggggacagggaaccacagtggtggtgagcgccggaggcgggggc
agcggcggcgggggctccggaggcggaggctcttacatccacgtgacccagtccccttcc
tctctgtccgtgtctatcggcgatcgcgtgaccatcaactgtcagacaagccagggagtg
ggctccgacctgcactggtatcagcacaagcctggcagggccccaaagctgctgatccac
cacacaagctccgtggaggatggagtgccaagccgcttcagcggctccggattccacacc
agctttaatctgacaatctccgacctgcaggccgacgatatcgccacctactattgccag
gtgctgcagttctttggcaggggctcccgcctgcacatcaagggcggcggcggctctggg
ggcgggggcagcggcggggggggctccgggggaggaggctctggcggagggggcagccag
ggccagctggtgcagagcggagcagagctgaagaagccaggagcctctgtgaagatcagc
tgtaagacatccggcTATcggttcaacttttatcacatcaattggatcaggcagaccgca
ggaaggggaccagagtggatgggctggatctccccctactctggcgataagaacctggcc
ccagccttccaggacagagtgatcatgaccacagataccgaggtgccagtgaccagcttc
acctccaccggagccgcctacatggagatccggaatctgaagttcgacgatacaggcacc
tatttttgcgccaagggcctgctgagagacggctctagcacatggctgccatacctgtgg
ggacagggcaccctgctgacagtgtcctctggaggaggaggctccgggggcggcggctct
ggaggaggaggctctcaaagcgtgctgacccagtccgcctctgtgagcggctccctgggc
cagtctgtgaccatcagctgtacaggccccaactccgtgtgctgttctcacaagtctatc
agctggtaccagtggccaccaggaagggcacctaccctgatcatctatgaggacaatgag
agggcaccaggaatcagccctcgcttctccggctacaagtcttattggagcgcctacctg
accatttccgacctgcgccccgaggatgagaccacatactattgctgtagctatacccac
aactccggctgcgtgtttggcacaggcaccaaggtgagcgtgctgggaggagggggctct
ggcggcgggggcagcggcggaggcggctccggagggggcggctctggcggaggcggcagc
gaggtgcggctgCGGgagagcggcggcggcctggtgaagccaggcggctctctgagactg
tcctgttctgccagcggcttcgactttgataatgcctggatgacatgggtgcggcagcct
cctggcaaggggctggagtgggtgggaagaatcaccggaccaggagagggatggtctgtg
gactacgccgagagcgtgaagggccggttcaccatcagcagagataacactaaaaataca
ctgtatctggagatgaacaatgtgcggaccgaggacacaggctactatttctgcgccaga
accggcaagtactatgatttctggtttggctacccccctggcgaggagtattttcaggac
tggggccagggcaccctggtcatcgtgagcagcggcgggggaggctccggcggggggggc
tctggaggagggggctctagcgagctgacccaggaccccgccgtgtccgtggccctgaag
cagacagtgaccatcacatgcaggggcgactccctgcgctctcactacgccagctggtat
cagaagaagccaggacaggcacccgtgctgctgttctacggcaagaacaatcggccttcc
ggcatcccagatagattttccggctctgccagcggaaacagggccagcctgaccatcaca
ggagcacaggcagaggatgaagcagattactattgttcctctcgggacaagtccggctct
agactgagcgtgttcggcggcggaaccaagctgacagtgctgggatccGGACACCACCAT
CACCATCATTAGTGAAAGCTT

8. PentaNAb_{4.0} Reverse PGDM1400-5X-PGT121-5X-N6-5X-35O22-5X-10E8v4 V5R S100cFvVH-fused to IgG1 LS HC with His Tag
   SEQ ID NO:88
   SEQ ID NO:88

ATGGGCTGGAGCTGCATCATCCTGTTCCTGGTGGCAACCGCAACAGGAGT
GCACAGCCAGGCACAGCTGGTGCAGAGCGGACCCGAAGTGAGAAAACCTG
GGACTAGCGTCAAAGTGTCATGTAAAGCCCCTGGAAATACCCTGAAGACC
TACGATCTGCACTGGGTGCGGTCCGTGCCTGGACAGGGCCTGCAGTGGAT
GGGATGGATCTCTCACGAGGGCGACAAGAAAGTGATCGTGGAGCGGTTCA
AGGCCAAGGTGACAATCGATTGGGACAGATCCACCAACACAGCCTACCTG
CAGCTGTCTGGCCTGACCAGCGGCGATACAGCCGTGTACTACTGTGCCAA
GGGCTCTAAGCACCGGCTGAGAGACTACGCCCTGTATGACGATGACGGCG
CCCTGAACTGGGCCGTGGATGTGGACTATCTGTCCAATCTGGAGTTCTGG
GGACAGGGAACCGCAGTGACAGTGAGCTCCGGAGGAGGAGGCTCCGGCGG
CGGAGGCTCTGGGGGAGGCGGCAGCGATTTTGTGCTGACCCAGTCTCCAC
ACAGCCTGTCCGTGACACCCGGCGAGTCTGCCAGCATCTCCTGCAAGTCT
AGCCACAGCCTGATCCACGGCGACAGGAACAATTACCTGGCCTGGTACGT
GCAGAAGCCAGGCCGCAGCCCTCAGCTGCTGATCTATCTGGCATCCTCTA
GGGCCTCCGGAGTGCCAGATCGCTTCTCTGGCAGCGGCTCCGATAAGGAC
TTTACCCTGAAGATCAGCCGGGTGGAGACAGAGGACGTGGGCACATACTA
TTGTATGCAGGGCCGAGAATCACCTTGGACATTTGGGCAGGGAACTAAAG
TTGACATCAAAGGGGGGGGGGGCTCCGGCGGCGGGGGCTCTGGCGGCGGC
GGCAGCGGAGGAGGCGGCTCCGGAGGAGGCGGCTCTCAGATGCAGCTGCA
GGAGAGCGGACCAGGACTGGTGAAGCCTTCCGAGACCCTGTCTCTGACAT
GTTCTGTGAGCGGCGCCTCCATCTCTGATAGCTACTGGAGCTGGATCAGA
CGGAGCCCTGGCAAGGGCCTGGAGTGGATCGGCTACGTGCACAAGTCTGG
CGATACAAACTATTCCCCATCTCTGAAGAGCCGGGTGAACCTGAGCCTGG
ACACCTCCAAGAATCAGGTGAGCCTGTCCCTGGTGGCAGCAACCGCAGCA
GACAGCGGCAAGTACTATTGCGCCAGAACACTGCACGGCAGGCGCATCTA
CGGCATCGTGGCCTTTAACGAGTGGTTCACCTACTTTTATATGGACGTGT
GGGGCAATGGCACCCAGGTGACAGTGTCCTCTGGCGGCGGCGGCTCTGGC
GGAGGAGGCAGCGGAGGAGGAGGCAGCTCCGACATCTCTGTGGCACCTGG
AGAGACCGCAAGGATCAGCTGTGGAGAGAAGTCTCTGGGCAGCAGGGCCG
TGCAGTGGTACCAGCACAGGGCAGGACAGGCACCATCCCTGATCATCTAT
AACAATCAGGACCGGCCATCTGGCATCCCCGAGAGATTCTCTGGCAGCCC
CGATAGCCCTTTTGGCACCACAGCCACCCTGACAATCACCTCCGTGGAGG
CCGGCGACGAAGCAGATTACTATTGCCACATCTGGGACTCCAGAGTGCCA
ACCAAGTGGGTGTTCGGAGGAGGAACCACACTGACAGTGCTGGGCGGCGG
AGGCTCCGGCGGGGGCGGCTCTGGAGGCGGCGGCAGCGGAGGGGGCGGCT
CCGGCGGCGGCGGCTCTAGGGCACACCTGGTGCAGAGCGGAACCGCAATG
AAGAAGCCTGGCGCCTCTGTGCGCGTGAGCTGTCAGACATCCGGCTACAC
CTTCACCGCCCACATCCTGTTCTGGTTTAGGCAGGCACCAGGAAGAGGAC
TGGAGTGGGTGGGCTGGATCAAGCCCCAGTATGGAGCAGTGAACTTCGGA
GGAGGCTTTCGGGACAGAGTGACACTGACCCGGGACGTGTACAGAGAGAT
CGCCTATATGGATATCAGGGGCCTGAAGCCCGACGATACCGCCGTGTACT
ATTGCGCCAGGGACCGCTCCTACGGCGATTCTAGCTGGGCACTGGACGCA
TGGGGACAGGGAACCACAGTGGTGGTGAGCGCCGGAGGCGGGGGCAGCGG
CGGCGGGGGCTCCGGAGGCGGAGGCTCTTACATCCACGTGACCCAGTCCC
CTTCCTCTCTGTCCGTGTCTATCGGCGATCGCGTGACCATCAACTGTCAG
ACAAGCCAGGGAGTGGGCTCCGACCTGCACTGGTATCAGCACAAGCCTGG
CAGGGCCCCAAAGCTGCTGATCCACCACACAAGCTCCGTGGAGGATGGAG
TGCCAAGCCGCTTCAGCGGCTCCGGATTCCACACCAGCTTTAATCTGACA
ATCTCCGACCTGCAGGCCGACGATATCGCCACCTACTATTGCCAGGTGCT
GCAGTTCTTTGGCAGGGGCTCCCGCCTGCACATCAAGGGCGGCGGCGGCT
CTGGGGGCGGGGGCAGCGGCGGGGGGGGCTCCGGGGGAGGAGGCTCTGGC
GGAGGGGGCAGCCAGGGCCAGCTGGTGCAGAGCGGAGCAGAGCTGAAGAA
GCCAGGAGCCTCTGTGAAGATCAGCTGTAAGACATCCGGCTATCGGTTCA
ACTTTTATCACATCAATTGGATCAGGCAGACCGCAGGAAGGGGACCAGAG
TGGATGGGCTGGATCTCCCCCTACTCTGGCGATAAGAACCTGGCCCCAGC
CTTCCAGGACAGAGTGATCATGACCACAGATACCGAGGTGCCAGTGACCA
GCTTCACCTCCACCGGAGCCGCCTACATGGAGATCCGGAATCTGAAGTTC
GACGATACAGGCACCTATTTTTGCGCCAAGGGCCTGCTGAGAGACGGCTC
TAGCACATGGCTGCCATACCTGTGGGGACAGGGCACCCTGCTGACAGTGT
CCTCTGGAGGAGGAGGCTCCGGGGGCGGCGGCTCTGGAGGAGGAGGCTCT
CAAAGCGTGCTGACCCAGTCCGCCTCTGTGAGCGGCTCCCTGGGCCAGTC
TGTGACCATCAGCTGTACAGGCCCCAACTCCGTGTGCTGTTCTCACAAGT
CTATCAGCTGGTACCAGTGGCCACCAGGAAGGGCACCTACCCTGATCATC
TATGAGGACAATGAGAGGGCACCAGGAATCAGCCCTCGCTTCTCCGGCTA
CAAGTCTTATTGGAGCGCCTACCTGACCATTTCCGACCTGCGCCCCGAGG
ATGAGACCACATACTATTGCTGTAGCTATACCCACAACTCCGGCTGCGTG
TTTGGCACAGGCACCAAGGTGAGCGTGCTGGGAGGAGGGGGCTCTGGCGG
CGGGGGCAGCGGCGGAGGCGGCTCCGGAGGGGGCGGCTCTGGCGGAGGCG
GCAGCGAGGTGCGGCTGCGGGAGAGCGGCGGCGGCCTGGTGAAGCCAGGC
GGCTCTCTGAGACTGTCCTGTTCTGCCAGCGGCTTCGACTTTGATAATGC
CTGGATGACATGGGTGCGGCAGCCTCCTGGCAAGGGGCTGGAGTGGGTGG
GAAGAATCACCGGACCAGGAGAGGGATGGTCTGTGGACTACGCCGAGAGC
GTGAAGGGCCGGTTCACCATCAGCAGAGATAACACTAAAAATACACTGTA
TCTGGAGATGAACAATGTGCGGACCGAGGACACAGGCTACTATTTCTGCG
CCAGAACCGGCAAGTACTATGATTTCTGGTTTGGCTACCCCCCTGGCGAG
GAGTATTTTCAGGACTGGGGCCAGGGCACCCTGGTCATCGTGAGCAGCGC
GTCGACCAAGGGCCCATCGGTCTTCCCCCTGGCACCCTCCTCCAAGAGCA
CCTCTGGGGGCACAGCGGCCCTGGGCTGCCTGGTCAAGGACTACTTCCCC
GAACCTGTGACGGTGTCGTGGAACTCAGGCGCCCTGACCAGCGGCGTGCA
CACCTTCCCGGCTGTCCTACAGTCCTCAGGACTCTACTCCCTCAGCAGCG
TGGTGACCGTGCCCTCCAGCAGCTTGGGCACCCAGACCTACATCTGCAAC
GTGAATCACAAGCCCAGCAACACCAAGGTGGACAAGAAAGTTGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTACAGCAAGCTCACCGTGGACAAGAGCAGGTGGCA
GCAGGGGAACGTCTTCTCATGCTCCGTGCTGCATGAGGCTCTGCACAGCC
ACTACACGCAGAAGAGCCTCTCCCTGTCTCCGGGTAAATGA

9. PentaNAb_{4.0} Reverse PGDM1400-5X-PGT121-5X-N6-5X-35O22-5X-10E8v4 V5R S100cF VH-VL-fused to IgG1 LS FC with His Tag
   SEQ ID NO:89

gaattcgccgccaccatgggctggagctgcatcatcctgttcctggtggcaaccgcaaca
ggagtgcacagccaggcacagctggtgcagagcggacccgaagtgagaaaacctgggact
agcgtcaaagtgtcatgtaaagcccctggaaataccctgaagacctacgatctgcactgg
gtgcggtccgtgcctggacagggcctgcagtggatgggatggatctctcacgagggcgac
aagaaagtgatcgtggagcggttcaaggccaaggtgacaatcgattgggacagatccacc
aacacagcctacctgcagctgtctggcctgaccagcggcgatacagccgtgtactactgt
gccaagggctctaagcaccggctgagagactacgccctgtatgacgatgacggcgccctg
aactgggccgtggatgtggactatctgtccaatctggagttctggggacagggaaccgca
gtgacagtgagctccggaggaggaggctccggcggcggaggctctgggggaggcggcagc
gattttgtgctgacccagtctccacacagcctgtccgtgacacccggcgagtctgccagc
atctcctgcaagtctagccacagcctgatccacggcgacaggaacaattacctggcctgg
tacgtgcagaagccaggccgcagccctcagctgctgatctatctggcatcctctagggcc
tccggagtgccagatcgcttctctggcagcggctccgataaggactttaccctgaagatc
agccgggtggagacagaggacgtgggcacatactattgtatgcagggccgagaatcacct
tggacatttgggcagggaactaaaGTTgacatcaaagggggggggggctccggcggcggg
ggctctggcggcggcggcagcggaggaggcggctccggaggaggcggctctcagatgcag
ctgcaggagagcggaccaggactggtgaagccttccgagaccctgtctctgacatgttct
gtgagcggcgcctccatctctgatagctactggagctggatcagacggagccctggcaag
ggcctggagtggatcggctacgtgcacaagtctggcgatacaaactattccccatctctg
aagagccgggtgaacctgagcctggacacctccaagaatcaggtgagcctgtccctggtg
gcagcaaccgcagcagacagcggcaagtactattgcgccagaacactgcacggcaggcgc
atctacggcatcgtggcctttaacgagtggttcacctacttttatatggacgtgtggggc
aatggcacccaggtgacagtgtcctctggcggcggcggctctggcggaggaggcagcgga
ggaggaggcagctccgacatctctgtggcacctggagagaccgcaaggatcagctgtgga
gagaagtctctgggcagcagggccgtgcagtggtaccagcacagggcaggacaggcacca
tccctgatcatctataacaatcaggaccggccatctggcatccccgagagattctctggc
agccccgatagcccttttggcaccacagccaccctgacaatcacctccgtggaggccggc
gacgaagcagattactattgccacatctgggactccagagtgccaaccaagtgggtgttc
ggaggaggaaccacactgacagtgctgggcggcggaggctccggcgggggcggctctgga
ggcggcggcagcggagggggcggctccggcggcggcggctctagggcacacctggtgcag
agcggaaccgcaatgaagaagcctggcgcctctgtgcgcgtgagctgtcagacatccggc
tacaccttcaccgcccacatcctgttctggtttaggcaggcaccaggaagaggactggag
tgggtgggctggatcaagccccagtatggagcagtgaacttcggaggaggctttcgggac
agagtgacactgacccgggacgtgtacagagagatcgcctatatggatatcaggggcctg
aagcccgacgataccgccgtgtactattgcgccagggaccgctcctacggcgattctagc
tgggcactggacgcatggggacagggaaccacagtggtggtgagcgccggaggcgggggc
agcggcggcgggggctccggaggcggaggctcttacatccacgtgacccagtccccttcc
tctctgtccgtgtctatcggcgatcgcgtgaccatcaactgtcagacaagccagggagtg
ggctccgacctgcactggtatcagcacaagcctggcagggccccaaagctgctgatccac
cacacaagctccgtggaggatggagtgccaagccgcttcagcggctccggattccacacc
agctttaatctgacaatctccgacctgcaggccgacgatatcgccacctactattgccag
gtgctgcagttctttggcaggggctcccgcctgcacatcaagggcggcggcggctctggg
ggcgggggcagcggcggggggggctccgggggaggaggctctggcggagggggcagccag
ggccagctggtgcagagcggagcagagctgaagaagccaggagcctctgtgaagatcagc
tgtaagacatccggcTATcggttcaacttttatcacatcaattggatcaggcagaccgca
ggaaggggaccagagtggatgggctggatctccccctactctggcgataagaacctggcc
ccagccttccaggacagagtgatcatgaccacagataccgaggtgccagtgaccagcttc
acctccaccggagccgcctacatggagatccggaatctgaagttcgacgatacaggcacc
tatttttgcgccaagggcctgctgagagacggctctagcacatggctgccatacctgtgg
ggacagggcaccctgctgacagtgtcctctggaggaggaggctccgggggcggcggctct
ggaggaggaggctctcaaagcgtgctgacccagtccgcctctgtgagcggctccctgggc
cagtctgtgaccatcagctgtacaggccccaactccgtgtgctgttctcacaagtctatc
agctggtaccagtggccaccaggaagggcacctaccctgatcatctatgaggacaatgag
agggcaccaggaatcagccctcgcttctccggctacaagtcttattggagcgcctacctg
accatttccgacctgcgccccgaggatgagaccacatactattgctgtagctatacccac
aactccggctgcgtgtttggcacaggcaccaaggtgagcgtgctgggaggagggggctct
ggcggcgggggcagcggcggaggcggctccggagggggcggctctggcggaggcggcagc
gaggtgcggctgCGGgagagcggcggcggcctggtgaagccaggcggctctctgagactg
tcctgttctgccagcggcttcgactttgataatgcctggatgacatgggtgcggcagcct
cctggcaaggggctggagtgggtgggaagaatcaccggaccaggagagggatggtctgtg
gactacgccgagagcgtgaagggccggttcaccatcagcagagataacactaaaaataca
ctgtatctggagatgaacaatgtgcggaccgaggacacaggctactatttctgcgccaga
accggcaagtactatgatttctggtttggctacccccctggcgaggagtattttcaggac
tggggccagggcaccctggtcatcgtgagcagcggcgggggaggctccggcggggggggc
tctggaggagggggctctagcgagctgacccaggaccccgccgtgtccgtggccctgaag
cagacagtgaccatcacatgcaggggcgactccctgcgctctcactacgccagctggtat
cagaagaagccaggacaggcacccgtgctgctgttctacggcaagaacaatcggccttcc
ggcatcccagatagattttccggctctgccagcggaaacagggccagcctgaccatcaca
ggagcacaggcagaggatgaagcagattactattgttcctctcgggacaagtccggctct
agactgagcgtgttcggcggcggaaccaagctgacagtgctgggatccCCCAAATCTTGT
GACAAAACTCACACATGCCCACCGTGCCCAGCACCTGAACTCCTGGGGGGACCGTCAGTC
TTCCTCTTCCCCCCAAAACCCAAGGACACCCTCATGATCTCCCGGACCCCTGAGGTCACA
TGCGTGGTGGTGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAG
TGCAAGGTCTCCAACAAAGCCCTCCCAGCCCCCATCGAGAAAACCATCTCCAAAGCCAAA
GGGCAGCCCCGAGAACCACAGGTGTACACCCTGCCCCCATCCCGGGAGGAGATGACCAAG
AACCAGGTCAGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTGCTGGACTCC
GACGGCTCCTTCTTCCTCTACAGCAAGCTCACCGTGGACAAGAGCAGGTGGCAGCAGGGG
AACGTCTTCTCATGCTCCGTGCTGCATGAGGCTCTGCACAGCCACTACACGCAGAAGAGC
CTCTCCCTGTCTCCGGGTAAATGA

Methods for producing antibodies, such as those disclosed herein, are known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains.

Standard techniques of molecular biology may be used to prepare DNA sequences coding for the antibodies or fragments of the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention or fragments thereof. Bacterial, for example E. coli, and other microbial systems may be used, in part, for expression of antibody fragments such as Fab and F(ab′)₂ fragments, and especially Fv fragments and single chain antibody fragments, for example, single chain Fvs. Eukaryotic, e.g. mammalian, host cell expression systems may be used for production of larger antibody molecules, including complete antibody molecules. Suitable mammalian host cells include CHO, HEK293T, PER.C6, myeloma or hybridoma cells.

In some embodiments, antibodies according to the invention may be produced by i) expressing a nucleic acid sequence according to the invention in a cell, and ii) isolating the expressed antibody product. Additionally, the method may include iii) purifying the antibody.

For the antibodies of the present invention to be expressed, the protein coding sequence should be “operably linked” to regulatory or nucleic acid control sequences that direct transcription and translation of the protein. As used herein, a coding sequence and a nucleic acid control sequence or promoter are said to be “operably linked” when they are covalently linked in such a way as to place the expression or transcription and/or translation of the coding sequence under the influence or control of the nucleic acid control sequence. The “nucleic acid control sequence” can be any nucleic acid element, such as, but not limited to promoters, enhancers, IRES, introns, and other elements described herein that direct the expression of a nucleic acid sequence or coding sequence that is operably linked thereto. The term “promoter” will be used herein to refer to a group of transcriptional control modules that are clustered around the initiation site for RNA polymerase II and that when operationally linked to the protein coding sequences of the invention lead to the expression of the encoded protein. The expression of the antibodies of the present invention can be under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the antibodies of the invention. For example, suitable promoters and/or enhancers can be selected from the Eukaryotic Promoter Database (EPDB).

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions. Specific expression and purification conditions will vary depending upon the expression system employed.

Following expression, the antibodies and/or antigens of the invention can be isolated and/or purified or concentrated using any suitable technique known in the art. For example, anion or cation exchange chromatography, phosphocellulose chromatography, hydrophobic interaction chromatography, affinity chromatography, immuno-affinity chromatography, hydroxyapatite chromatography, lectin chromatography, molecular sieve chromatography, isoelectric focusing, gel electrophoresis, or any other suitable method or combination of methods can be used.

In some embodiments, the antibodies can be made using recombinant DNA methods as described in U.S. Pat. No. 4,816,567. The polynucleotides encoding a monoclonal antibody can be isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells.

The anti-HIV antibodies can also include insertions, deletions, substitutions, or other selected modifications of particular regions or specific amino acids residues. It should be understood that the antibodies of the invention may differ from the exact sequences illustrated and described herein. Thus, the invention contemplates deletions, additions and substitutions to the sequences shown, so long as the sequences function in accordance with the methods of the invention. In this regard, particularly preferred substitutions will generally be conservative in nature, i.e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. For example, leucine can be replaced with isoleucine or valine, or vice versa; an aspartate with a glutamate or vice versa; a threonine with a serine or vice versa; or a similar conservative replacement of an amino acid with a structurally related amino acid can be made.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

For the purposes of the present invention, it should be appreciated that modified antibodies can comprise any type of variable region that provides for the association of the antibody with the polypeptides of HIV such as the envelope protein.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and can also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, the anti-HIV antibodies provide for altered effector functions that, in turn, affect the biological profile of the administered antibody. For example, the deletion or inactivation (through point mutations or other means) of a constant region domain can reduce Fc receptor binding of the circulating modified antibody thereby increasing tumor localization. In other cases it may be that constant region modifications, consistent with this invention, moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this invention can easily be made using well known biochemical or molecular engineering techniques well within the purview of the skilled artisan.

### Methods

The multispecific anti-HIV antibodies of the invention are useful in a variety of applications including, but not limited to, therapeutic treatment methods, such as the treatment, cure, functional cure, or prevention of HIV infection. The methods of use may be in vitro, ex vivo, or in vivo methods.

In some embodiments, the multispecific antibodies disclosed herein may be used as neutralizing antibodies, passively administered or given via gene therapies.

In one aspect, the anti-HIV antibodies are useful for detecting the presence of HIV in a biological sample. The term “detecting” as used herein encompasses quantitative or qualitative detection. In certain embodiments, a biological sample comprises a cell or tissue.

Certain other methods can be used to detect binding of anti-HIV antibodies to antigens such as envelope protein. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC).

In certain embodiments, the antibodies are labeled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

In certain embodiments, the antibodies are immobilized on an insoluble matrix. Immobilization entails separating the antibody from any antigen that remains free in solution. This conventionally is accomplished by either insolubilizing the antibody before the assay procedure, as by adsorption to a water-insoluble matrix or surface (Bennich et al., U.S. Pat. No. 3,720,760), or by covalent coupling (for example, using glutaraldehyde cross-linking), or by insolubilizing the antibody after formation of a complex between the antibody and antigen, e.g., by immunoprecipitation.

The present invention provides for methods of treating or preventing HIV infection comprising administering a therapeutically effective amount of a multispecific antibody as described herein to a subject (e.g., a subject in need of treatment). In some embodiments, the subject is a human.

Subjects at risk for HIV-related diseases or disorders include patients who have come into contact with an infected person or who have been exposed to HIV-1 in some other way. Administration of a prophylactic agent can occur prior to the manifestation of symptoms characteristic of HIV-1-related disease or disorder, such that a disease or disorder is prevented or, alternatively, delayed in its progression.

Methods for preventing an increase in HIV-1 virus titer, virus replication, virus proliferation or an amount of an HIV-1 viral protein in a subject are further provided. In one embodiment, a method includes administering to the subject an amount of an anti-HIV antibody effective to prevent an increase in HIV-1 titer, virus replication or an amount of an HIV-1 protein of one or more HIV strains or isolates in the subject.

For in vivo treatment of human patients, the patient is usually administered or provided a pharmaceutical formulation including a multispecific anti-HIV antibody of the invention. When used for in vivo therapy, the antibodies of the invention are administered to the patient in therapeutically effective amounts (i.e., amounts that eliminate or reduce the patient's viral burden). The antibodies can be administered to a human patient, in accordance with known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. The antibodies may be administered parenterally, when possible, at the target cell site, or intravenously. Intravenous or subcutaneous administration of the antibody is preferred in certain embodiments. Therapeutic compositions of the invention are administered to a patient or subject systemically, parenterally, or locally.

For parenteral administration, the antibodies can be formulated in a unit dosage injectable form (solution, suspension, emulsion) in association with a pharmaceutically acceptable, parenteral vehicle. Examples of such vehicles are water, saline, Ringer's solution, dextrose solution, and 5% human serum albumin. Nonaqueous vehicles such as fixed oils and ethyl oleate are also used. Liposomes are used as carriers. The vehicle contains minor amounts of additives such as substances that enhance isotonicity and chemical stability, e.g., buffers and preservatives. In some embodiments, the antibodies are typically formulated in such vehicles at concentrations of about 1 mg/ml to 10 mg/ml.

The dose and dosage regimen depends upon a variety of factors readily determined by a physician, such as the nature of the infection and the characteristics of the particular cytotoxic agent or growth inhibitory agent conjugated to the antibody (when used), e.g., its therapeutic index, the patient, and the patient's history. Generally, a therapeutically effective amount of an antibody is administered to a patient. In particular embodiments, the amount of antibody administered is in the range of about 0.1 mg/kg to about 20 mg/kg of patient body weight. Depending on the type and severity of the infection, about 0.1 mg/kg to about 20 mg/kg body weight (e.g., about 0.1-15 mg/kg/dose) of antibody is an initial candidate dosage for administration to the patient, whether, for example, by one or more separate administrations, or by continuous infusion. The progress of this therapy is readily monitored by conventional methods and assays and based on criteria known to the physician or other persons of skill in the art.

Antibodies of the invention can be coupled to a drug for delivery to a treatment site or coupled to a detectable label to facilitate imaging of a site comprising cells of interest, such as cells infected with HIV. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody of the invention and an epitope of interest (an HIV epitope) can be facilitated by attaching a detectable substance to the antibody. Suitable detection means include the use of labels such as radionucleotides, enzymes, coenzymes, fluorescers, chemiluminescers, chromogens, enzyme substrates or co-factors, enzyme inhibitors, prosthetic group complexes, free radicals, particles, dyes, and the like. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin; and examples of suitable radioactive material include ¹²⁵I, ¹³¹I, ³⁵S, or .sup.3H. Such labeled reagents may be used in a variety of well-known assays, such as radioimmunoassays, enzyme immunoassays, e.g., ELISA, fluorescent immunoassays, and the like.

The antibodies can be tagged with such labels by known methods. For instance, coupling agents such as aldehydes, carbodiimides, dimaleimide, imidates, succinimides, bid-diazotized benzadine and the like are used to tag the antibodies with the above-described fluorescent, chemiluminescent, and enzyme labels. An enzyme is typically combined with an antibody using bridging molecules such as carbodiimides, periodate, diisocyanates, glutaraldehyde and the like. Various labeling techniques are described in Morrison, Methods in Enzymology 32b, 103 (1974), Syvanen et al., J. Biol. Chem. 284, 3762 (1973) and Bolton and Hunter, Biochem J. 133, 529 (1973).

In one embodiment, the antibodies can be administered as immunoconjugates, conjugated to a second molecule. For example, the second molecule can be a toxin, a label, a radioisotope, a drug, or a chemical compound.

An antibody according to the invention may be conjugated to a therapeutic moiety such as a cytotoxin, a therapeutic agent, or a radioactive metal ion or radioisotope. Examples of radioisotopes include, but are not limited to, I-131, I-123, I-125, Y-90, Re-188, Re-186, At-211, Cu-67, Bi-212, Bi-213, Pd-109, Tc-99, In-111, and the like. Such antibody conjugates can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin, TLR agonists (such as TLR7 agonist), or monomethylauristatin E.

Other therapeutic regimens can be combined with the administration of the anti-HIV antibody of the present invention. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

For any application, the antibody, antigen binding fragment, or nucleic acid encoding the antibody or antigen binding fragment can be combined with anti-retroviral therapy. Antiretroviral drugs are broadly classified by the phase of the retrovirus life-cycle that the drug inhibits. The disclosed antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some examples, a disclosed antibody or active fragment thereof or nucleic acids encoding such is administered in conjunction with IL-15, or conjugated to IL-15.

Single or multiple administrations of the compositions including the antibody, antigen binding fragment, or nucleic acid encoding the antibody or antigen binding fragment, that are disclosed herein, are administered depending on the dosage and frequency as required and tolerated by the patient. In any event, the composition should provide a sufficient quantity of at least one of the antibodies disclosed herein to effectively treat the patient. The dosage can be administered once, but may be applied periodically until either a therapeutic result is achieved or until side effects warrant discontinuation of therapy.

One approach to administration of nucleic acids is direct administration with plasmid DNA, such as with a mammalian expression plasmid. The nucleotide sequence encoding the disclosed antibody, or antibody binding fragments thereof, can be placed under the control of a promoter to increase expression. Another approach is to administer the nucleic acids in the form of mRNA.

In some embodiments, the subject is administered cells that are engineered to express the anti-HIV antibody. In some embodiments, the cells are engineered immune cells, such as B cells. In some embodiments, the cells are engineered, autologous cells.

In another approach to using nucleic acids, an anti-HIV antibody, or antibody binding fragment thereof can also be expressed by attenuated viral hosts or vectors or bacterial vectors. Recombinant vaccinia virus, adeno-associated virus (AAV), herpes virus, retrovirus, cytomegalovirus or other viral vectors can be used to express the antibody. For example, vaccinia vectors and methods useful protocols are described in U.S. Pat. No. 4,722,848. BCG (Bacillus Calmette Guerin) provides another vector for expression of the disclosed antibodies (see Stover, Nature 351:456-460, 1991).

### Compositions

The present invention also encompasses compositions comprising one or more antibodies of the invention. In certain embodiments, the compositions are pharmaceutical compositions. In some embodiments, formulations are prepared for storage and use by combining an antibody with a pharmaceutically acceptable vehicle (e.g. carrier, excipient) (Remington, The Science and Practice of Pharmacy 20th Edition Mack Publishing, 2000). Suitable pharmaceutically acceptable vehicles include, but are not limited to, nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (e.g. less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosacchandes, disaccharides, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and non-ionic surfactants such as TWEEN or polyethylene glycol (PEG).

For the treatment or prevention of HIV, the appropriate dosage of an antibody or combination of antibodies of the present invention can depend on a variety of factors, such as the severity and course of the disease, the responsiveness of the disease, whether the antibody or agent is administered for therapeutic or preventative purposes, previous therapy, patient's clinical history, and so on all at the discretion of the treating physician. The antibody or agent can be administered one time or over a series of treatments lasting from several days to several months, or until a cure is effected or a diminution of the disease state is achieved. The administering physician can easily determine optimum dosages, dosing methodologies and repetition rates. In certain embodiments, dosage is from 0.01 μg to 100 mg per kg of body weight, and can be given once or more daily, weekly, monthly or yearly. In certain embodiments, the antibody or combination of antibodies is given once every two weeks or once every three weeks. In certain embodiments, the dosage of the antibody is from about 0.1 mg to about 20 mg per kg of body weight. The treating physician can estimate repetition rates for dosing based on measured residence times and concentrations of the drug in bodily fluids or tissues.

Effective dosages and schedules for administering embodiments of the present invention can be determined empirically. In some embodiments, and effective amount of one or more antibodies are administered to neutralize, treat, prevent or eradicate HIV infection. In some embodiments, compositions comprising one or more nucleic acid molecules of the invention are administered to the subject. In some embodiments, genetic constructs capable of inducing production of antibodies of the present invention may be administered to a patient in need thereof.

Controlled-release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995). Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992).

Polymers can be used for ion-controlled release of the antibody compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44 (2): 58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

In some embodiments, the compositions of the invention may be injectable suspensions, solutions, sprays, lyophilized powders, syrups, elixirs and the like. Any suitable form of composition may be used. To prepare such a composition, a nucleic acid or vector of the invention, having the desired degree of purity, is mixed with one or more pharmaceutically acceptable carriers and/or excipients. The carriers and excipients must be “acceptable” in the sense of being compatible with the other ingredients of the composition. Acceptable carriers, excipients, or stabilizers are nontoxic to recipients at the dosages and concentrations employed, and include, but are not limited to, water, saline, phosphate buffered saline, dextrose, glycerol, ethanol, or combinations thereof, buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

The compositions can be designed to introduce the antibodies, nucleic acids or expression vectors to a desired site of action and release it at an appropriate and controllable rate. Methods of preparing controlled-release formulations are known in the art. For example, controlled release preparations can be produced by the use of polymers to complex or absorb the immunogen and/or immunogenic composition. A controlled-release formulations can be prepared using appropriate macromolecules (for example, polyesters, polyamino acids, polyvinyl, pyrrolidone, ethylenevinylacetate, methylcellulose, carboxymethylcellulose, or protamine sulfate) known to provide the desired controlled release characteristics or release profile. Another possible method to control the duration of action by a controlled-release preparation is to incorporate the active ingredients into particles of a polymeric material such as, for example, polyesters, polyamino acids, hydrogels, polylactic acid, polyglycolic acid, copolymers of these acids, or ethylene vinylacetate copolymers. Alternatively, instead of incorporating these active ingredients into polymeric particles, it is possible to entrap these materials into microcapsules prepared, for example, by coacervation techniques or by interfacial polymerization, for example, hydroxymethylcellulose or gelatin-microcapsule and poly-(methylmethacrylate) microcapsule, respectively, in colloidal drug delivery systems (for example, liposomes, albumin microspheres, microemulsions, nano-particles and nanocapsules) or in macroemulsions. Such techniques are disclosed in New Trends and Developments in Vaccines, Voller et al. (eds.), University Park Press, Baltimore, Md., 1978 and Remington's Pharmaceutical Sciences, 16th edition.

The compositions can be administered using any suitable delivery method including, but not limited to, intramuscular, intravenous, intradermal, mucosal, and topical delivery. Such techniques are well known to those of skill in the art. More specific examples of delivery methods are intramuscular injection, intradermal injection, and subcutaneous injection. However, delivery need not be limited to injection methods. Further, delivery of DNA to animal tissue has been achieved by cationic liposomes (Watanabe et al., (1994) Mol. Reprod. Dev. 38:268-274; and WO 96/20013), direct injection of naked DNA into animal muscle tissue (Robinson et al., (1993) Vaccine 11:957-960; Hoffman et al., (1994) Vaccine 12:1529-1533; Xiang et al., (1994) Virology 199:132-140; Webster et al., (1994) Vaccine 12:1495-1498; Davis et al., (1994) Vaccine 12:1503-1509; and Davis et al., (1993) Hum. Mol. Gen. 2:1847-1851), or intradermal injection of DNA using “gene gun” technology (Johnston et al., (1994) Meth. Cell Biol. 43:353-365). Alternatively, delivery routes can be oral, intranasal or by any other suitable route. Delivery also be accomplished via a mucosal surface such as the anal, vaginal or oral mucosa.

Dosing schedules (or regimens) can be readily determined for the particular subject and composition. Hence, the composition can be administered one or more times to the subject. Preferably, there is a set time interval between separate administrations of the composition. While this interval varies for every subject, typically it can range from 10 days to several weeks, and is often 2, 4, 6 or 8 weeks. In some embodiments, the interval can be typically from 2 to 6 weeks.

The compositions of the invention can be administered alone, or can be co-administered, or sequentially administered, with other HIV immunogens and/or HIV immunogenic compositions, e.g., with “other” immunological, antigenic or vaccine or therapeutic compositions thereby providing multivalent or “cocktail” or combination compositions of the invention and methods of employing them. Again, the ingredients and manner (sequential or co-administration) of administration, as well as dosages can be determined taking into consideration such factors as the age, sex, weight, species and condition of the particular subject, and the route of administration.

### Kits

The present invention also includes kits useful in performing diagnostic and prognostic assays using the antibodies of the present invention. Kits of the invention include a suitable container comprising an HIV-1 antibody of the invention in either labeled or unlabeled form. In addition, when the antibody is supplied in a labeled form suitable for an indirect binding assay, the kit further includes reagents for performing the appropriate indirect assay. For example, the kit includes one or more suitable containers including enzyme substrates or derivatizing agents, depending on the nature of the label. Control samples and/or instructions are also included.

Application of the teachings of the present invention to a specific problem is within the capabilities of one having ordinary skill in the art in light of the teaching contained herein. Examples of the compositions and methods of the invention appear in the following non-limiting Examples.

### EXAMPLES

### Example 1. Multi-Specific Antibodies for HIV-1 and their Utility

Here, 3D structure information of broadly neutralizing antibodies (bNAbs) in complex with HIV-1 envelope glycoproteins (Env) was used to perform rational design of penta-specific antibodies (PentaNAbs) capable of targeting five major Env neutralizing epitopes. These inventions, herein referred to as “PentaNAbs”, consist of five functional single chain fragment variable (ScFv) moieties connected in tandem via (G4S) n linkers, such that penta-valence engagement of five individual epitopes on the HIV-1 Env by each respective moiety is possible. The bNAb functional moieties incorporated in the design target five major HIV neutralization epitopes including the i) V1/V2 apex glycan, ii) V3-base glycan, iii) CD4 binding site, iv) gp120/gp41 interface, and v) the membrane proximal external region (MPER). The sequence information of the penta-specific antibodies is listed in Table 4. In addition, these PentaScFv entities were fused to the fragment crystallizable region (Fc) region of IgG1 to accommodate effector functions in a Penta-IgG format.

To design the PentaScFvs, the physical distance between the compatible N/C termini of parental bNAb variable heavy (VH) and light chain (VL) domains was estimated to identify the shortest linker combinations allowing optimal VH/VL connection within each bNAb ScFv entity. Furthermore, to generate molecules capable of simultaneously engaging (“draping” the HIV-1 Env) contiguous epitopes on the HIV-1 Env surface, the spatial relationship between each neighboring bNAb moiety in the state of cognate epitope binding was assessed. It was found that epitopes located on adjacent protomers of the HIV-1 trimer are often more proximate to each other than those on the same protomer. Thus, in some designs, two functional bNAb moieties binding to the same protomer (intra-protomer mode) were arranged and in some cases to separate protomers (inter-protomers) of the HIV-1 Env trimer. Finally, PentaNAbs with bNAb moieties connected from N to C terminus in two orientations were designed: from the MPER to V1V2 apex (forward order) or from the V1V2 apex to MPER (reverse order) followed by the Fc fragment to form PentaNAb IgG1 molecules. The purpose of these designs is to minimize potential steric hindrance imposed by the addition of Fc fragment to the C terminus of the connected ScFv entities. PentaNAb2.0 was initially constructed, which consists of functional moieties of five bNAbs including 10E8v4 S100cF, 35O22, N6, PGT121, and VRC26.25 to test the rationale. In this design, an molecular orientation was used, namely “Forward” orientation with which the MPER bNAb 10E8v4 S100cF moiety is at the N terminus of the PentaNAb and the V1V2 glycan bNAb VRC26.25 moiety on the distal side followed by Fc fragment.

Using a 208-virus panel representing the worldwide diverse circulating HIV-1 virus isolates to assess neutralization breath, PentaNAb2.0, in both ScFv and IgG form, displayed improved virus isolate coverage of 99.6%, as compared to individual bNAbs 10E8, VRC01, PGT121 and 35O22, which had 97.6%, 90.4%, 63.9%, and 45.2% coverage, respectively. Most significantly, the PentaNAb2.0, in both ScFv and IgG form, displayed significantly improved IC50 geomeans of 0.006 and 0.072 μg/mL, respectively, as compared to 10E8, which has an IC50 mean of 0.299 μg/mL with the best neutralization breadth among all known bNAbs. In addition, PentaNAb2.0 ScFv displays neutralization potency and breadth superior to previously engineered tri- and tetra-specific antibodies to HIV-1 Env. Furthermore, PentaNAb2.0, in both ScFv and IgG form effectively neutralizes VRC01-resistant replication-competent viral isolates from patients participated in the clinical trial involving passive transfer of VRC01. Finally, PentaNAbs were engineered with a “Reverse” orientation with which the V1V2 glycan bNAb VRC26.25 or PGDM1400 moiety is at the N terminus of the PentaNAb and the MPER bNAb 10E8v4 S100cF moiety on the distal side followed by Fc fragment. The neutralization capacities of these PentaNAb iterations were assessed with a panel of 26 HIV-1 Env-pseudoviruses in TZM-bl target cells. It was found that all of the PentaNAbs in the ScFv format displayed a greater breadth and potency than their respective IgG1 counterparts with Fc fragment. Additionally, it was observed that the PentaNAbs in the “reverse” order displayed a greater potency than the “forward” order while maintaining the same breadth. Taken together, penta-specific antibodies were engineered that are capable of engaging five major neutralization epitopes of HIV-1 envelope glycoproteins possess superior antiviral activities to conventional HIV-1 bNAbs and previously engineered tri- and tetra-specific HIV-1 bNAbs, which may serve as effective agents for HIV-1 prevention, treatment and diagnosis.

### Results

Spatial Relationships Between HIV-1 Env-Specific bNAb Epitopes and the Design of Connecting Linker Length

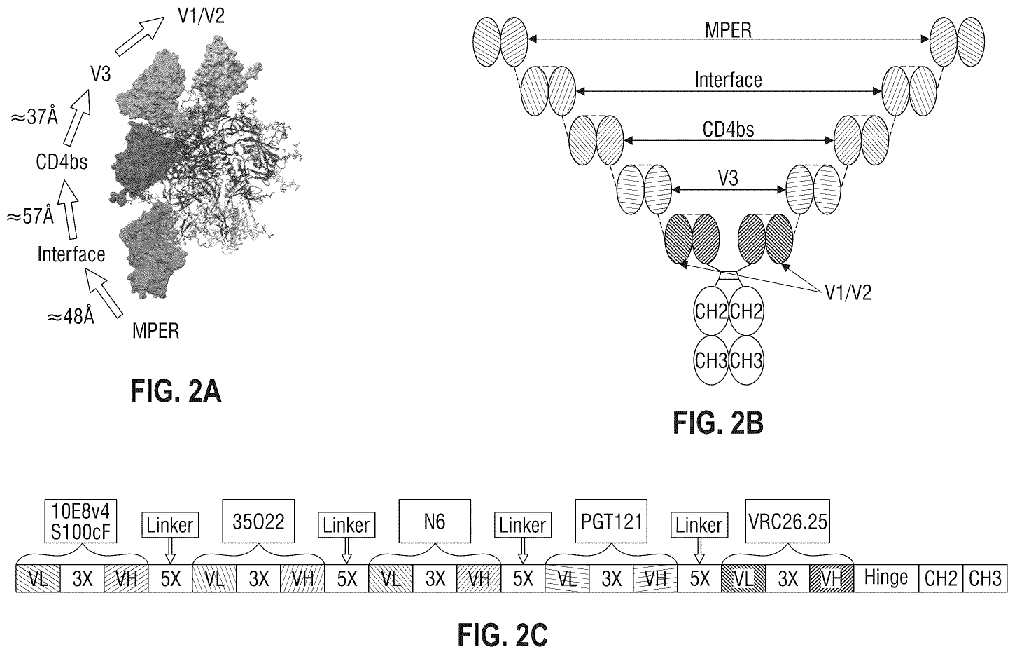

To constitute PentaNAb molecules, five top lead antibodies displaying the best neutralization potency and breadth among bNAbs within each individual epitope cluster were first selected. For example, in one combination VRC26.25 or PGDM1400, PGT121, N6, 35022, and 10E8v4 V5R S100cF from the V1/V2, V3-glycan, CD4bs, gp120/gp41 interface, and MPER epitope cluster, respectively were picked. In order to develop a PentaNAb capable of simultaneously engaging five separate epitopes, the physical distance between the compatible variable heavy (VH) and light (VL) chain region termini (e.g. VH C-terminus followed by N-terminus of VL) of the selected bNAbs was measured to identify the shortest combinations (FIG. 2). The PentaNAb was designed such that the VH and VL of each individual bNAb entity was connected between termini with flexible linkers such that the flexible linker would traverse the shortest unimpeded distance possible. It is believed that joining the individual entities of the PentaNAb between the termini with the shortest unimpeded distance in between would avoid excess linker length, which would i) improve protein folding, ii) improve the expression level/yield of the molecule, and iii) improve the coordination of simultaneous engagement of all five epitopes.

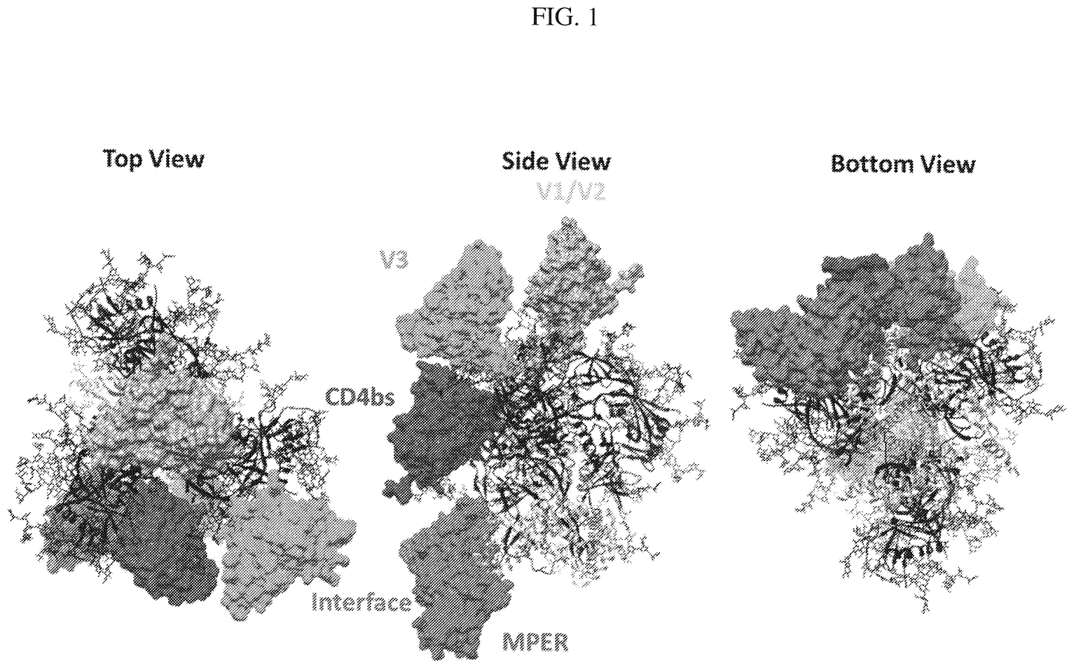

Five G4S linkers were used to connect moieties of bNAbs targeting the V1V2 (e.g. VRC26.25) and V3 (e.g. PGT121) glycans. The distance between V1/V2 and the V3-glycan targeting antibodies VRC26.25 or PGDM1400 and PGT121, respectively, was not determined as crystallography data of gp120 bound VRC26.25 or PGDM1400 is currently unavailable. Using the EMD data for VRC26.25 bound to BG505 SOSIP.664, EMD-5856 (Doria-Rose et al., Nature 509, 55-62 (2014)), the crystal structure of the VRC26.25 Fab (PDB: 5DT1) (Doria-Rose et al., J Virol 90, 76-91 (2015)), as well as the crystal structure of PGT122 bound BG505 SOSIP.664 (PDB: 5FYK) (Stewart-Jones et al., Cell 165, 813-826 (2016)), a model of VRC26.25 & PGT121 bound Env trimer were generated by manual superposition of BG505 trimer elements. This model was subsequently used to roughly estimate the spatial relationship between VRC26.25 and PGT121 with results similar to other modeling attempts using a combination of molecular dynamics (MD) and molecular dynamics flexible-fitting (MDFF) (Gorman et al., Nat Struct Mol Biol 23, 81-90 (2016)). Similarly this EM model, EMD-5856 (Doria-Rose et al., Nature 509, 55-62 (2014)), was used to manually fit the crystal structure of the PGDM1400 Fab (PDB: 4RQQ) (Sok et al., Proc Natl Acad Sci USA 111, 17624-17629 (2014)), with the crystal structure of PGT122 bound BG505 SOSIP.664 (PDB: 5FYK) (Stewart-Jones et al., Cell 165, 813-826 (2016)), to briefly estimate the spatial relationship between PGDM1400 and PGT121, since PGDM1400 approaches the gp120 apex with an angle similar to VRC26.25. According to the EM data, both VRC26.25 and PGDM1400 are quaternary-dependent antibodies targeting the V1/V2 epitope at the trimer apex (Doria-Rose et al., J Virol 90, 76-91 (2015); Sok et al., Proc Natl Acad Sci USA 111, 17624-17629 (2014)) (FIG. 1), with VH/VL termini close to one another. Based on the above stated analysis, a linker of sufficient length (e.g. a linker with five G4S units) is utilized to connect VRC26.25 or PGDM1400 with PGT121.

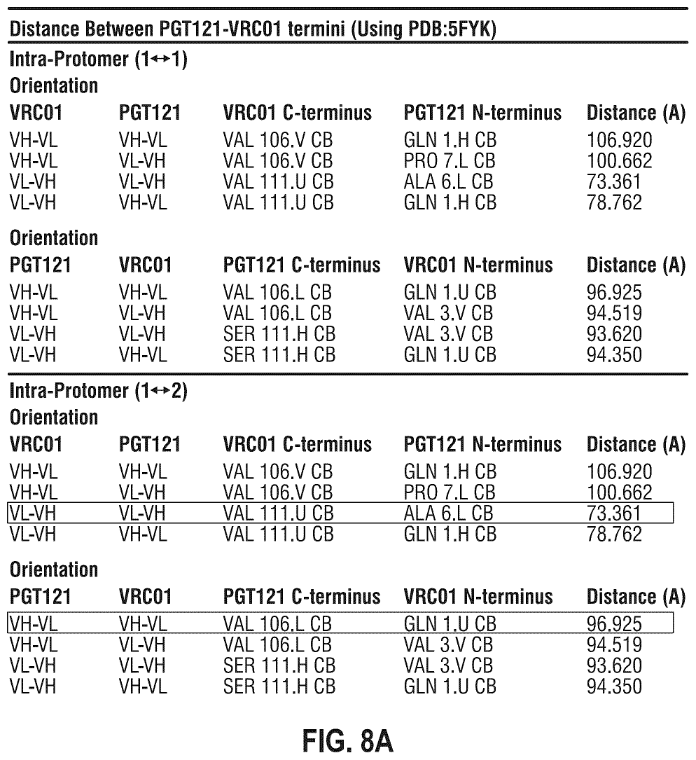

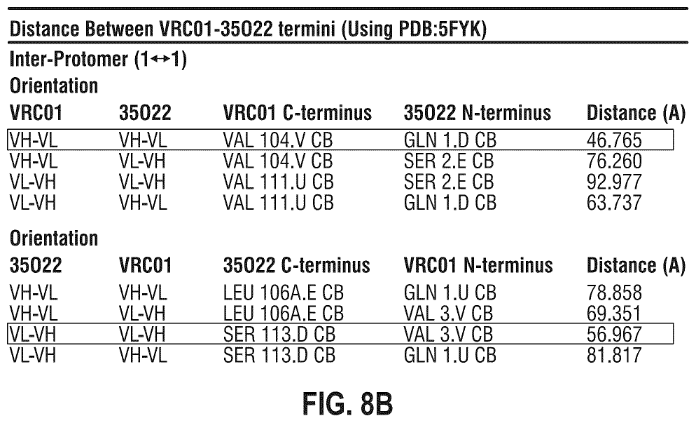

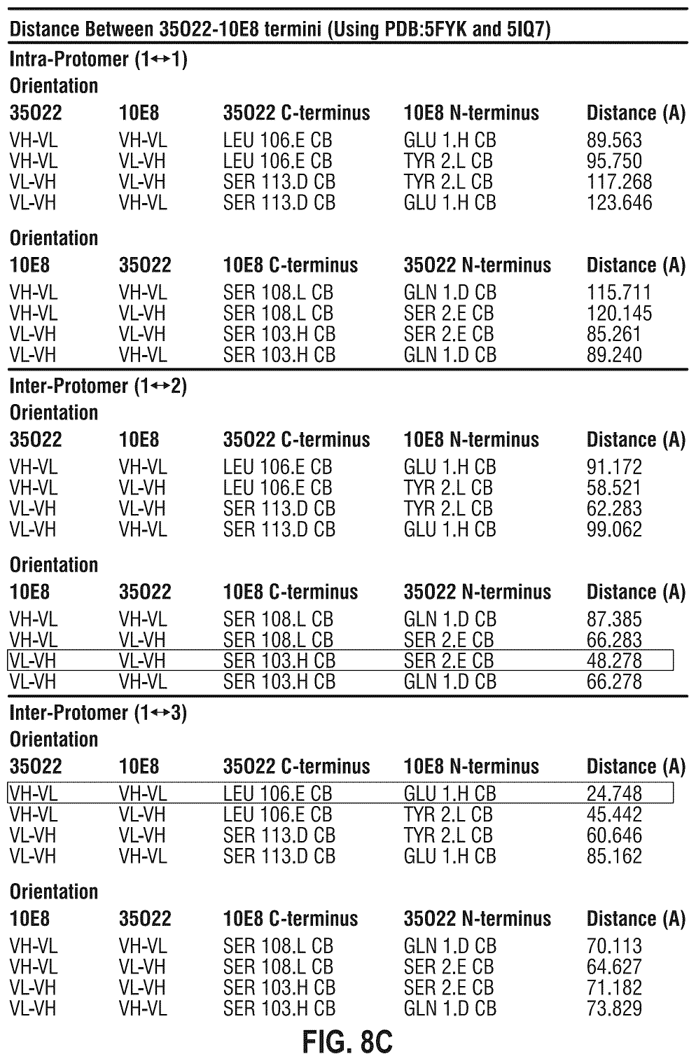

Five G4S linkers were used to connect the V3-glycan targeting PGT121 and the CD4bs targeting N6 ScFv entities. Previous studies determined that the shortest distance between PGT122 (a surrogate for PGT121) and VRC01 (a surrogate for N6) is between adjacent protomers within an Env trimer (inter-protomer). Using the PDB model 5FYK (Stewart-Jones et al., Cell 165, 813-826 (2016)), determined the distances between the VRC01 and PGT121 functional variable domains as the following: 37 Å between the C-terminus of VRC01 VH domain and the N-terminus of PGT121 VL domain with VRC01 (VL-VH)-PGT121 (VL-VH) topology, and 53 Å between the C-terminus of PGT121 VL and the N-terminus of VRC01 VH with PGT121 (VH-VL)-VRC01 (VH-VL) topology (FIG. 8A). Furthermore, negative stain electron microscopy data demonstrated that one of the Bi-ScFvs previously generated, dVRC01 (VL-VH)-5X-PGT121 (VL-VH), was able to simultaneously engage both the PGT121 and VRC01 epitopes in an inter-protomer manner as we had predicted (Steinhardt et al., Nat Commun 9, 877 (2018)), which supports rationale for this linker design.

The shortest distance between the CD4bs targeting bNAb moiety, N6 (with VRC01 as surrogate), and the gp120/gp41 interface targeting 35O22 entity was determined to be an intra-protomer distance. Using the model in PDB 5FYK (Stewart-Jones et al., Cell 165, 813-826 (2016)), the distance between the 35O22 and N6 functional variable domains were determined to be 57 Å between the C-terminus of 35O22 VH domain and the N-terminus of N6 VL domain with the topology of 35O22 (VL-VH)-N6 (VL-VH), and 47 Å between the C-terminus of N6 VL and the N-terminus of 35O22 VH with the topology of N6 (VH-VL)-35O22 (VH-VL) (FIG. 8B), respectively. Thus, five G4S linkers were used to connect N6 and 35022.

Finally, the Shortest Distances Between the Gp120/Gp41 Interface Targeting 35O22 entity and the MPER targeting 10E8v4 entity was determined to be inter-protomer manner. Using PDB 5IQ7 superimposed with PDB 5FYK (Stewart-Jones et al., Cell 165, 813-826 (2016); Kwon et al., J Virol 90, 5899-5914 (2016)), a model for Env engaged with both 35O22 and 10E8 was generated. In this model, the distance between the 10E8v4 and 35O22 functional variable domains were determined to be 48 Å between the C-terminus of 10E8v4 VH domain and the N-terminus of 35O22 VL domain with the topology of 10E8v4 (VL-VH)-35O22 (VL-VH), and 25 Å between the C-terminus of 35O22 VL and the N-terminus of 10E8v4 VH with the topology of 35O22 (VH-VL)-10E8v4 (VH-VL) (FIG. 8C), respectively. Therefore, five G4S linkers were used to connect 35O22 and 10E8v4.

### PentaNAbs Display Exceptional Neutralization Potency and Breadth

Based on the analysis of spatial relationship between the five major bNAb epitopes on HIV-1 Env surface, and the design of connecting linker length stated above, PentaNAb2.0 (FIG. 2, Table 5) was composed consisting of bNAb moieties of VRC26.25, PGT121, N6, 35022, and 10E8v4_S100cF from the V1/V2, V3-glycan, CD4bs, gp120/gp41 interface, and MPER epitope cluster, respectively.

TABLE 5
Summary of PentaNAb names, order of individual antibody entities
and orientation of variable light and variable heavy chains.

| Name | Order | Orientation |
|---|---|---|
| PentaNAb_{1.0} | 10E8v4, 35O22, N6, PGT121, | 10E8v4-5X-35O22- |
| | VRC26.25 | 5X-N6-5X-PGT121- |
| | | 5X-VRC26.25 |
| PentaNAb_{2.0} | 10E8v4 S100cF, 35O22, N6, | 10E8v4_S100cF-5X- |
| | PGT121, VRC26.25 | 35O22-5X-N6-5X- |
| | | PGT121-5X- |
| | | VRC26.25 |
| PentaNAb_{2.0} | VRC26.25, PGT121, N6, | VRC26.25-5X- |
| Reverse | 35O22, 10E8v4_S100cF | PGT121-5X-N6- |
| | | 35O22- |
| | | 10E8v4_S100cF |
| PentaNAb_{3.0} | 10E8v4_S100cF, 35O22, N6, | 10E8v4_S100cF-5X- |
| | PGT121, PGDM1400 | 35O22-5X-N6-5X- |
| | | PGT121-5X- |
| | | PGDM1400 |
| PentaNAb_{3.0} | PGDM1400, PGT121, N6, | PGDM1400-5X- |
| Reverse | 35O22, 10E8v4_S100cF | PGT121-5X-N6-5X- |
| | | 35O22-5X- |
| | | 10E8v4_S100cF |
| PentaNAb_{4.0} | 10E8v4 V5R_S100cF, | 10E8v4 |
| | 35O22, N6, PGT121, | V5R_S100cF-5X- |
| | PGDM1400 | 35O22-5X-N6-5X- |
| | | PGT121-5X- |
| | | PGDM1400 |
| PentaNAb_{4.0} | PGDM1400, PGT121, N6, | PGDM1400-5X- |
| Reverse | 35O22, 10E8v4_V5R_S100cF | PGT121-5X-N6-5X- |
| | | 35O22-5X- |
| | | 10E8v4_V5R_S100cF |

The PentaNAb2.0-ScFvs and full length IgGs were expressed in mammalian 293FreeStyle cells and purified by Ni+ and protein A columns, respectively. All molecules were characterized by SDS-PAGE gels, size exclusion chromatography (SEC), and dynamic light scattering (DLS) (data not shown). Furthermore, the binding specificities of these constructs were validated by ELISA binding assay (not shown), and Bio-Layer 10 Interferometry (BLI), which demonstrated binding to numerous Env ligands including the CD4bs specific RSC3 probe, an MPER peptide, and full length BG505 SOSIP.664 and JR-FL SOSIP.664 Env trimers (data not shown).

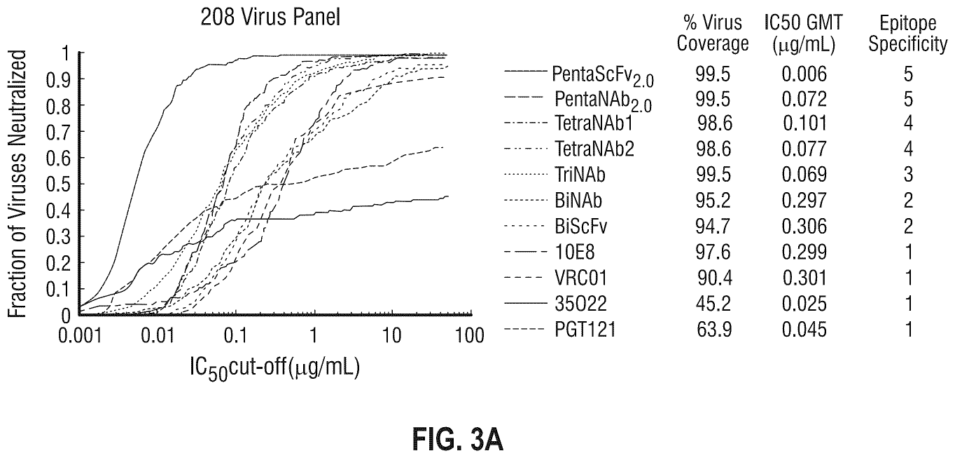

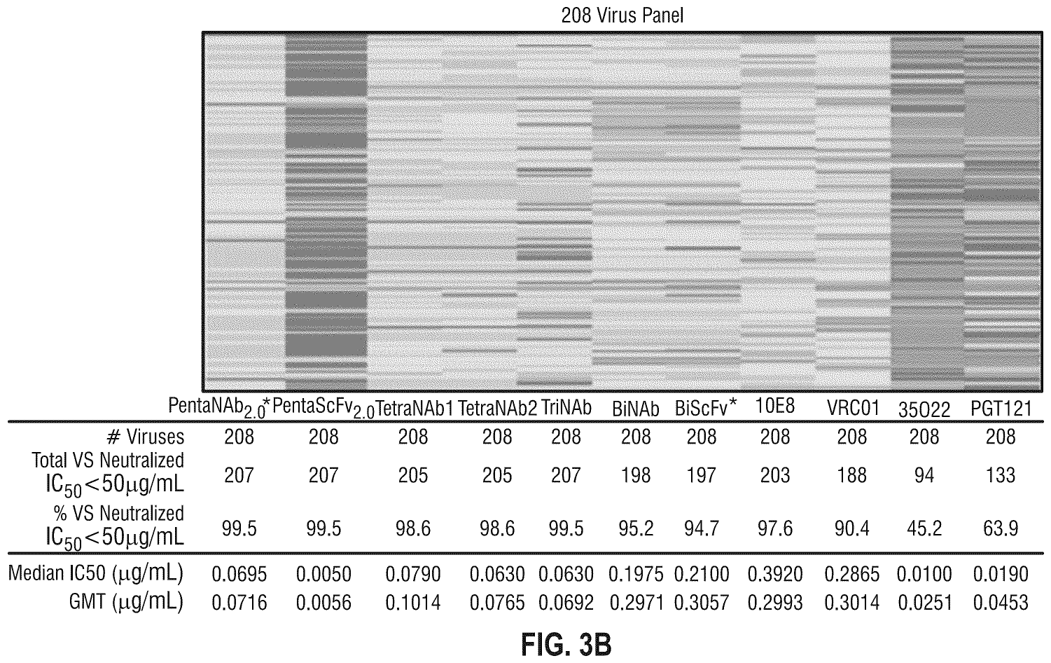

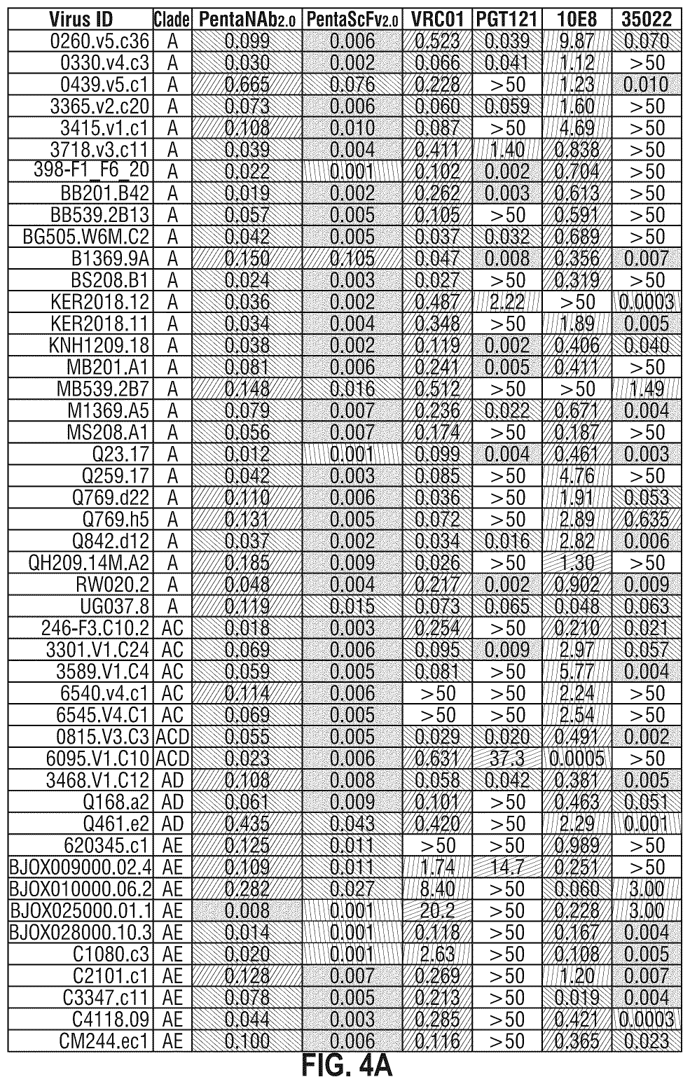

A comprehensive virus panel consisting of 208 pseudotype virus strains representing the worldwide diversified circulating HIV-1 virus isolates was used to analyze and compare the PentaNAb2.0 ScFv and IgG with TetraNAb, TriNAb and BiNAb designs in a previous invention disclosure (PCT/US2017/057053), as well as prototypic bNAbs including 10E8, VRC01 and PGT121. Both PentaNAb2.0 ScFv and IgG displayed substantially greater breadth and higher potency than the prototypical bNAbs and BiNAbs, while PentaNAb2.0 ScFv displays potency higher than TetraNAb and TriNAb (FIGS. 3A, 3B & 4) by approximately 10-fold. The PentaNAb2.0, in both ScFv and IgG form, displayed improved virus isolate coverage of 99.6%, as compared to bNAbs 10E8, VRC01 and PGT121, which had 97.6%, 90.4% and 63.9%, respectively. The PentaNAb2.0, in both ScFv and IgG form, displayed significantly improved IC50 geomeans of 0.006 and 0.072 μg/mL, respectively (FIGS. 3 & 4). Most significantly, with an IC50 mean of 0.006 μg/mL, the PentaNAb2.0 ScFv is the most potent HIV-1 multiNAb tested to date by the comprehensive 208-virus panel.

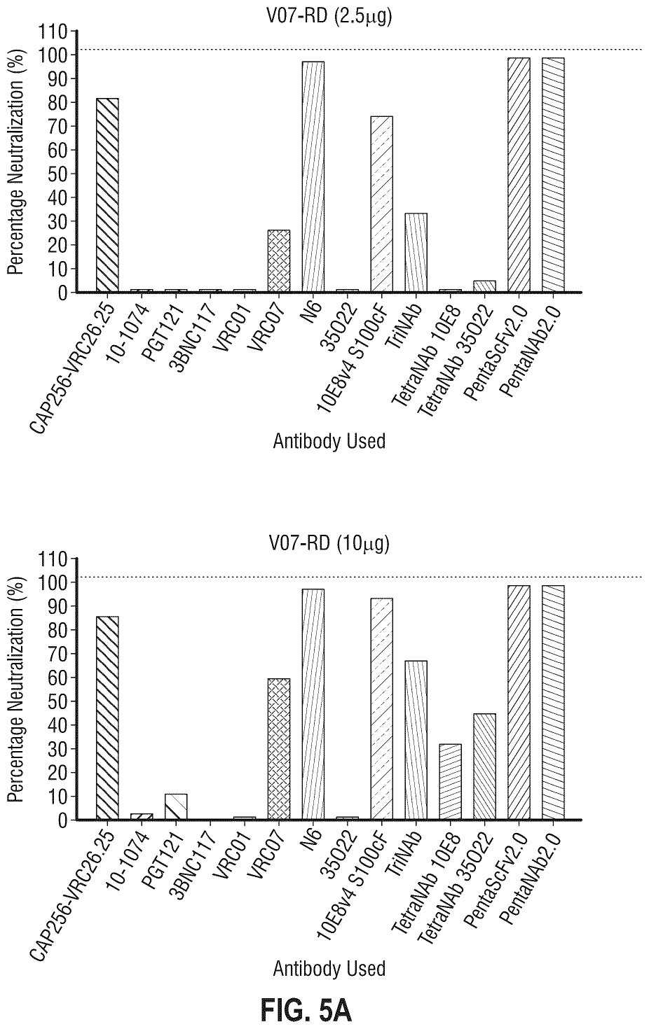

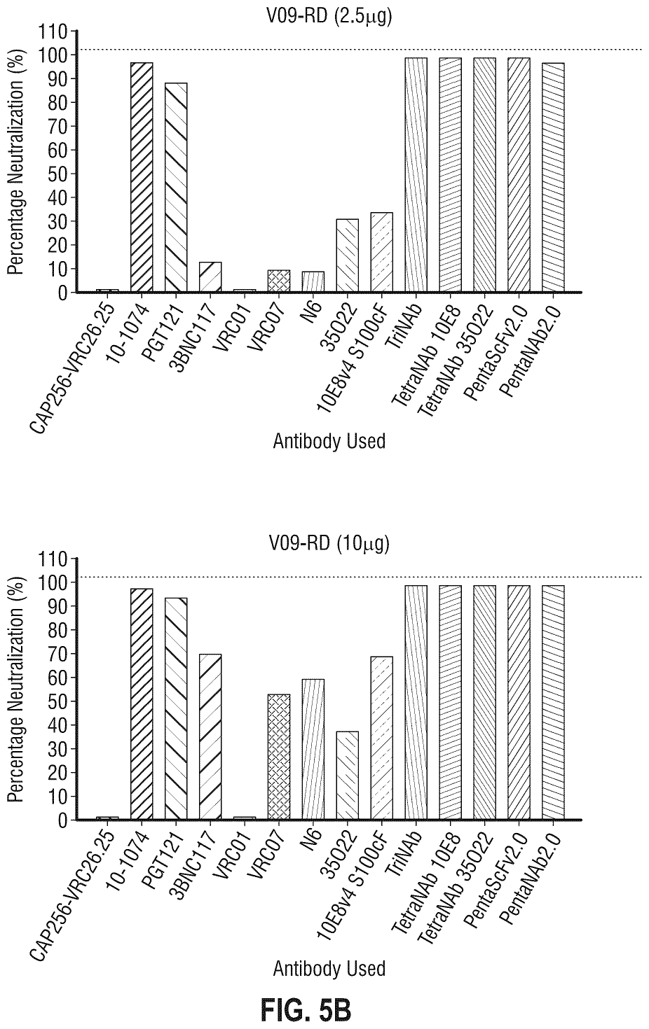

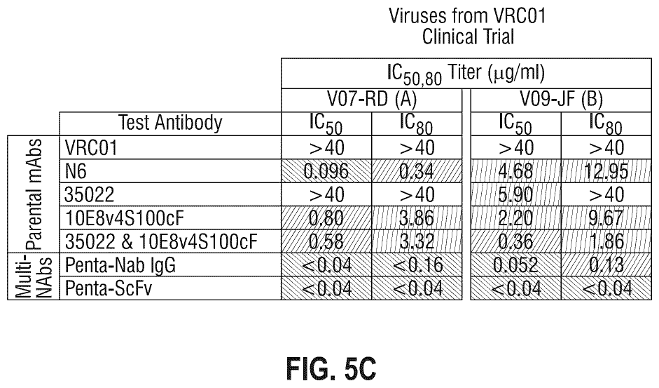

Finally, when tested against VRC01-resistant replication-competent viral isolates from patients participating in the clinical trial involving passive transfer of VRC01, the PentaNAb2.0 ScFv and IgG displayed neutralization capacity superior to previous multiNAb designs as well as the prototypical bNAbs (FIG. 5A-5C). Taken together, the data suggest that the PentaNAb could serve as a multifunctional therapeutic agent template for the prevention and treatment of HIV-1 infection, outperforming the conventional bNAbs and multi-specific bNAbs engineered previously.

Optimized bNAb Orientations Enhance the Potency of PentaNAbs

PentaNAb2.0 was initially designed and purified in the “Forward” orientation (FIG. 2). While PentaNAb2.0 ScFv shows superior potency to Tri- and Tetra-NAb designs, PentaNAb2.0 IgG with Fc fragment fused to the V1V2 glycan antibody moieties at the C terminus of the PentaNAb2.0 ScFv (FIG. 2) shows potency virtually identical to Tri- and Tetra-NAb designs. We hypothesized that the steric hindrance imposed by the Fc fragment to the potent V1V2 glycan antibody moieties such as VRC26.25 may result in deleterious effect on neutralization potency.

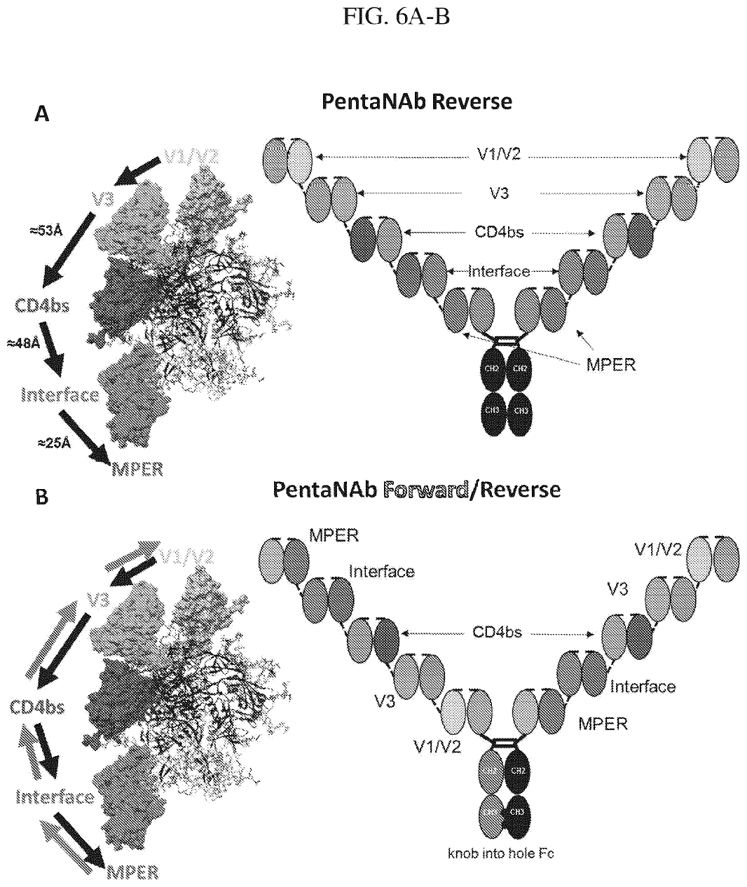

To test this hypothesis, a series of Penta-ScFvs and full length IgGs (FIG. 2, FIG. 6) were generated, with combinations and permutations of VH-VL orientations and linker length for optimization (Table 4). Particularly, these designs include either i) the “Forward” orientation: 10E8v4 (VL-VH)-35O22 (VL-VH)-VRC01 (VL-VH)-5X-PGT121 (VL-VH)-VRC26.25/PGDM1400 (VL-VH)-Fc whereby the Fc fragment connects with VRC26.25 or PGDM1400 (V1V2 glycan bNAb) and protrudes from the apex of the trimer (FIG. 2), or ii) the “Reverse” orientation: VRC26.25/PGDM1400 (VH-VL)-PGT121 (VH-VL)-VRC01 (VH-VL)-35O22 (VH-VL)-10E8v4 (VH-VL)-Fc whereby the Fc fragment connects with 10E8v4 (MPER bNAb) and protrudes from the trimer MPER region (FIG. 6A). Additionally, “knob into hole” technology (Ridgway et al., Protein Eng 9, 617-621 (1996)) was utilized to generate a heterodimer of the “Forward” and “Reverse” PentaNAb arms for subsequent evaluation, with LS mutations (M428L/N434S) in the Fc fragment for elongated antibody in vivo half-lives as well as improved biodistribution into the mucosal compartment (Ko et al., Nature 514, 642-645 (2014)) (FIG. 6B).

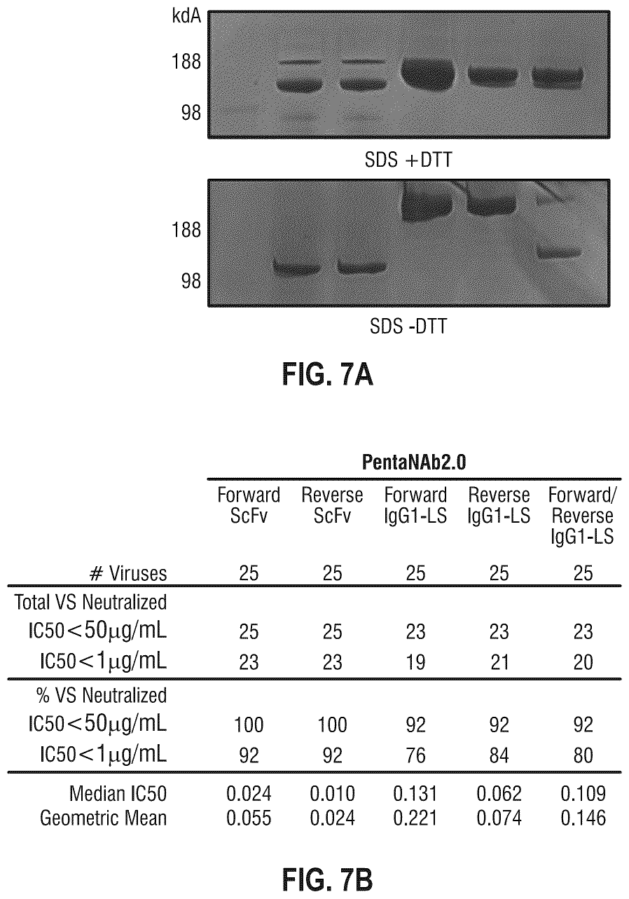

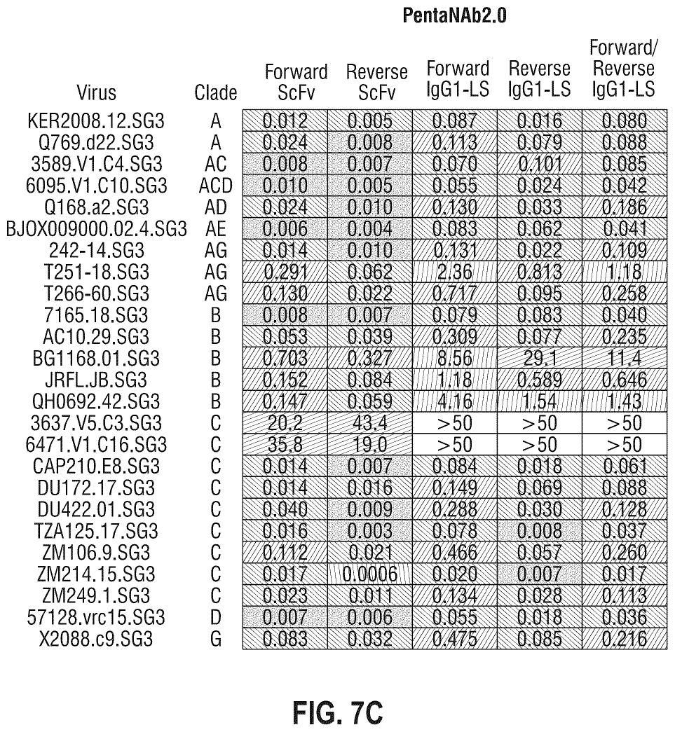

Three selected Penta-ScFvs and full length IgGs were expressed in mammalian 293FreeStyle cells and purified by Ni+ and protein A columns (FIG. 7), respectively. All the proteins have expected molecular weight, as shown in representative SDS-PAGE gels (FIG. 7A), size exclusion chromatography (SEC), and dynamic light scattering (DLS) analysis (data not shown). Furthermore, the binding specificities of these antibodies were validated by ELISA binding assay (not shown), and Bio-Layer Interferometry (BLI) (data not shown).

To compare the neutralization potency and breadth of the Penta-ScFvs and IgGs in the “Forward”, “Reverse” and “Forward/Reverse” orientations, a virus panel of high stringency consisting of 26 tier-2 virus strains (FIGS. 7B & 7C) was used. Of the multiple iterations tested, it was found that the Penta-ScFv and Penta-IgG in the “Reverse” orientation displayed improved potency while maintaining the same breadth (virus coverage) as those in the “Forward” orientation (FIGS. 7B & 7C). Somewhat expectedly, the “Forward/Reverse” IgG orientation displayed a potency in between that of the “Forward” and “Reverse” IgG (FIGS. 7B & 7C). Thus, the “Reverse” orientation of Penta-ScFv and Penta-IgG could be applied to generate antiviral agent in the future.

### Example 2. Design of Pentaspecific HIV Antibodies

Advances in the treatment of human immunodeficiency virus (HIV) infection have led to dramatic improvements in health outcomes of infected individuals receiving antiretroviral therapy (ART) over the course of the last several decades. By suppressing plasma viremia with ART, life expectancy has approached that of uninfected individuals, turning what was once an almost uniformly fatal illness into a manageable chronic disease. Nonetheless, the vast majority of HIV-infected individuals must remain on continuous therapy given that virus inevitably rebounds following discontinuation of ART. Furthermore, current ART requires life-long adherence to daily medication regimens and is associated with significant costs, cumulative toxicities, and the potential for emergence of drug-resistant virus. Consequently, intense efforts have been made in recent years to develop therapeutic strategies aimed at achieving ART-free virologic remission in infected individuals. Among those, recent discoveries of a number of highly potent broadly neutralizing HIV-specific antibodies (bNAbs) isolated from B cells of viremic HIV-infected individuals have materialized as a basis for realistic and feasible approaches for HIV prevention and immunotherapy.

Recent clinical trials conducted to assess the safety, tolerability, and virologic efficacy of multiple infusions of several bNAbs have shown to be safe, well-tolerated, and exhibit antiretroviral activities against HIV. However, these human trials have concomitantly shown that therapies involving single bNAbs are unlikely to suppress plasma viremia for prolonged periods of time due to both pre-existing and emergent HIV mutations that are resistant to the antibody-mediated neutralization. Therefore, it is of great interest to develop a combination approach to increase the specificity and potency of bNAbs (or bNAb-like) molecules against a broad spectrum of infectious HIV in patients. In order to achieve this goal, we have developed synergistically combined functional epitope-binding moieties from five bNAbs that target the CD4 binding site, V2 and V3 conserved glycans, gp120/gp41 interface, as well as the membrane exterior proximal region into a ‘single’ penta-specific antibody (penta-NAb).

Thus far, our penta-Nabs exhibited superior degrees of HIV inhibition (neutralization of 99.5% of a 208 pseudo-typed HIV panel), exceptional neutralization capacity (IC50 at 0.006 μg/ml) compared to individual bNAbs, and the capacity to neutralize replication-competent HIV isolates from infected individuals whose virus became resistant to various single bNAbs inferring our penta-Nabs the potential to overcome immune-evasion and eliminate persistent HIV reservoirs.

In this example, it was proposed to:
1) generate multiple penta-Nabs by incorporating five candidate bNAbs exhibiting the highest levels of neutralization against >300 individual infectious viral isolates obtained from the persistent viral reservoir of infected individuals receiving ART.
2) engineer/modify the Fc region to extend the in vivo half-life of each penta-NAb.
3) generate several penta-bNAb/anti-CD3 antibody-based molecules that are capable of binding cell-surface Env and inducing CD8+ T cell-mediated killing of HIV reservoirs in vitro and in vivo.
4) measure the efficacy of penta-NAbs in NSG mice following administration of peripheral blood mononuclear cells (PBMCs) from ART-naïve, HIV-infected individuals whose viral reservoirs carry sensitive and/or resistant virus to individual bNAbs.
5) evaluate the capacity of penta-bNAbs and DART molecules to prevent plasma viral rebound in NSG mice injected with from HIV-infected individuals following discontinuation of ART
   The overall outcome of this study will provide the first proof of concept that a “5-in-1” antibody can achieve long-lasting broad-spectrum HIV inhibition in vivo, which will substantially advance our basic understanding of protective immunity against persistent virus infection and contribute to the development of safe and effective strategies toward ART-free virologic remission.

### Results

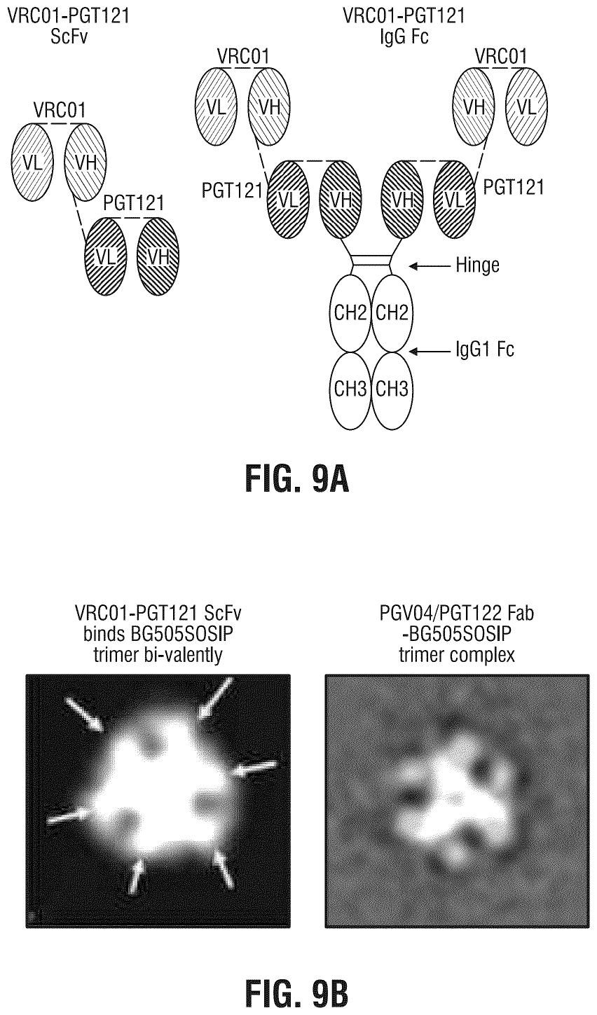

A clinical trial was previously conducted to investigate whether a bNAb VRC01 could prevent plasma viral rebound in infected individuals upon discontinuation of ART. While multiple infusions of VRC01 were safe and well-tolerated, the majority of patients experienced plasma viral rebound due to pre-existing and emergent antibody resistant HIV, suggesting therapeutic strategies involving passive transfer of bNAbs may require a combination(s) of bNAbs that targets more than one region on HIV Env protein in order to achieve sustained virologic control in HIV-infected individuals upon withdrawal of ART. Thus, building upon the outcome of the data, it was proposed to test HIV Env-specific antibodies possessing multi-functional moieties with improved antigen binding and/or virus neutralization capacities, which may translate into superior protective efficacy in subsequent studies. The preliminary data, derived from five of the constructs, demonstrated the feasibility of the methodology and linkage to the studies proposed here. Taking the first step toward generating multi-epitope targeting bNAbs, it was sought to test if one could engineer bi-specific single-chain variable fragment (bi-ScFv) consisting of bNAbs VRC01 and PGT121. As shown in FIG. 9A, a series of VRC01-PGT121 bi-ScFvs and full length IgGs were generated with combinations and permutations of VH-VL orientations and linker length. It was shown using single molecule electron microscopy (EM) analysis that the VRC01-PGT121 bi-ScFv binds Env trimer in a bi-specific manner (FIG. 9B). The virus neutralization capacity of the bispecific molecules (Bi-BNAb) against a virus panel consisting of 208 primary virus isolates was evaluated and demonstrated ˜95% virus neutralization breadth, which is substantially expanded compared to that of the parent mAbs (VRC01 90% and PGT121 64%).

Using the knob-into-hole technology, the VRC01-PGT121 Bi-NAb was combined with 1) the moiety of MPER mAb 10E8 to form Tri-NAb; and 2) another Bi-NAb consisting of gp120/gp41 interface mAb and 10E8 in two different N-term to C-term orientations to form Tet-NAb1 and 2. Finally, a scFV was created connecting variable regions of five bNAbs, namely penta-NAb, which engages all of the five conserved epitopes of HIV Env trimer (FIG. 2).

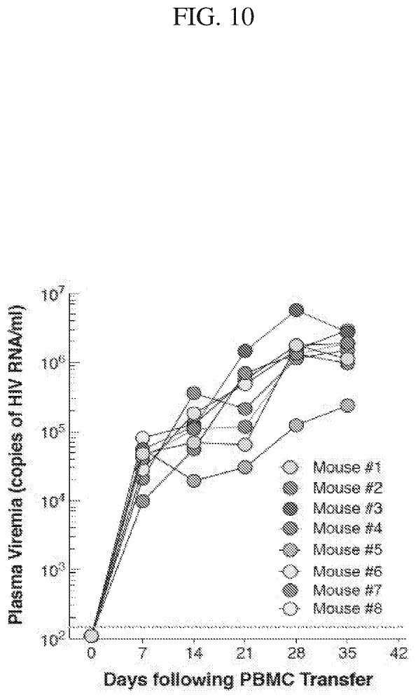

Interestingly, Tri-NAb, Tet-NAbs and Penta-NAb demonstrate remarkably improved neutralization breadth and potency compared to the parental mAbs (FIG. 3A). The superior neutralization breadth and potency possessed by the multi-epitope targeting NAbs are likely caused by cooperative and sometimes synergistic effect. Thus, the engineered multi-epitope targeting NAbs, which target three to five out of the five major conserved HIV Env neutralizing epitopes, could achieve nearly perfect virus coverage with IC50 geometric mean below 0.1 μg/ml. Furthermore, the neutralization capacity of the Penta-scFV and Penta-Nab was tested, which display neutralization breadth and potency outperforming the parental monoclonal antibodies and combinations (FIG. 5C). These multi-NAbs possessing remarkably improved breadth and potency represent excellent functional modules for next generation candidate therapeutic molecules. In order to generate in vivo efficacy data, NSG mice will be utilized that are injected with PBMCs from HIV-infected viremic and aviremic individuals. As shown in FIG. 10, all 8 mice exhibited robust HIV replication following transfer of 8×106 PBMCs from an viremic individual who was ART-naïve. These data demonstrate that the above animal system could be used to measure the efficacy of penta-NAbs and DART molecules against HIV derived from viral reservoirs from infected individuals who may carry sensitive and resistant HIV to certain bNAb.

### Example 3

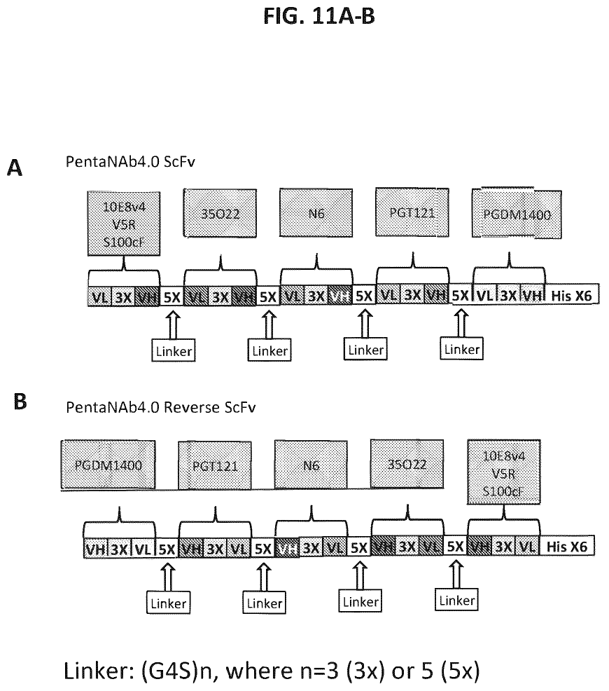

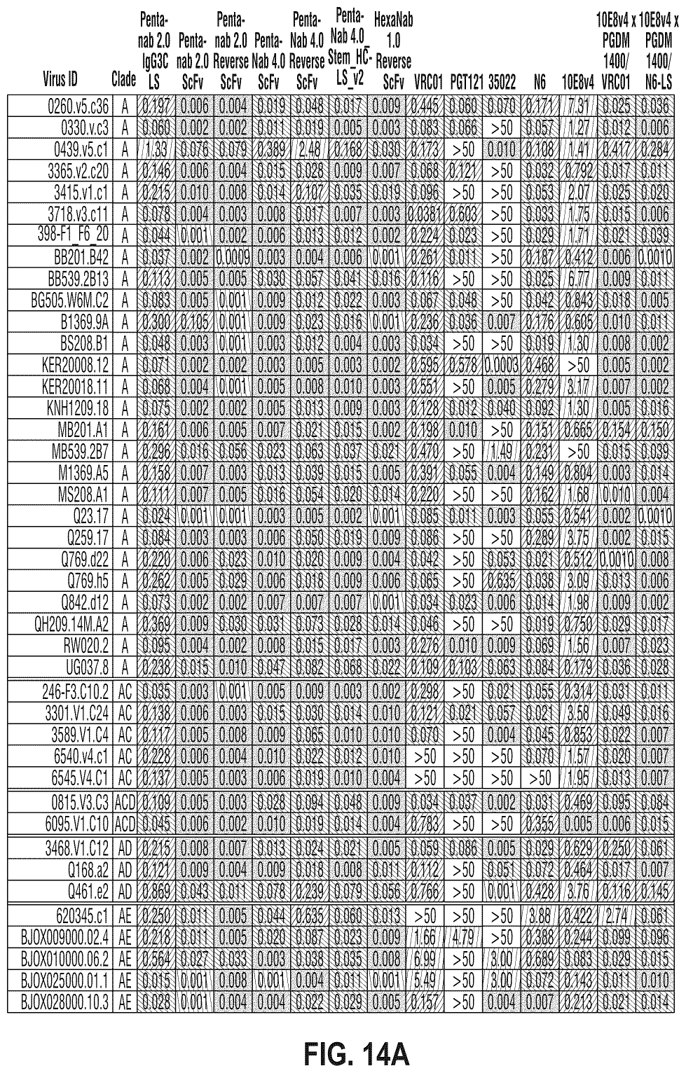

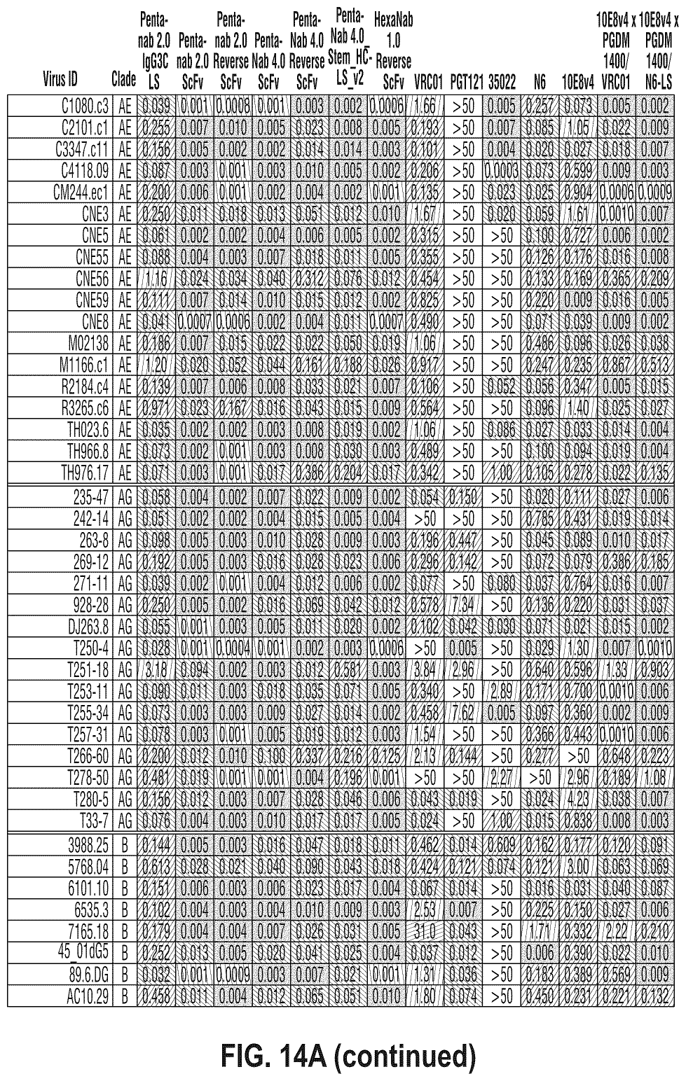

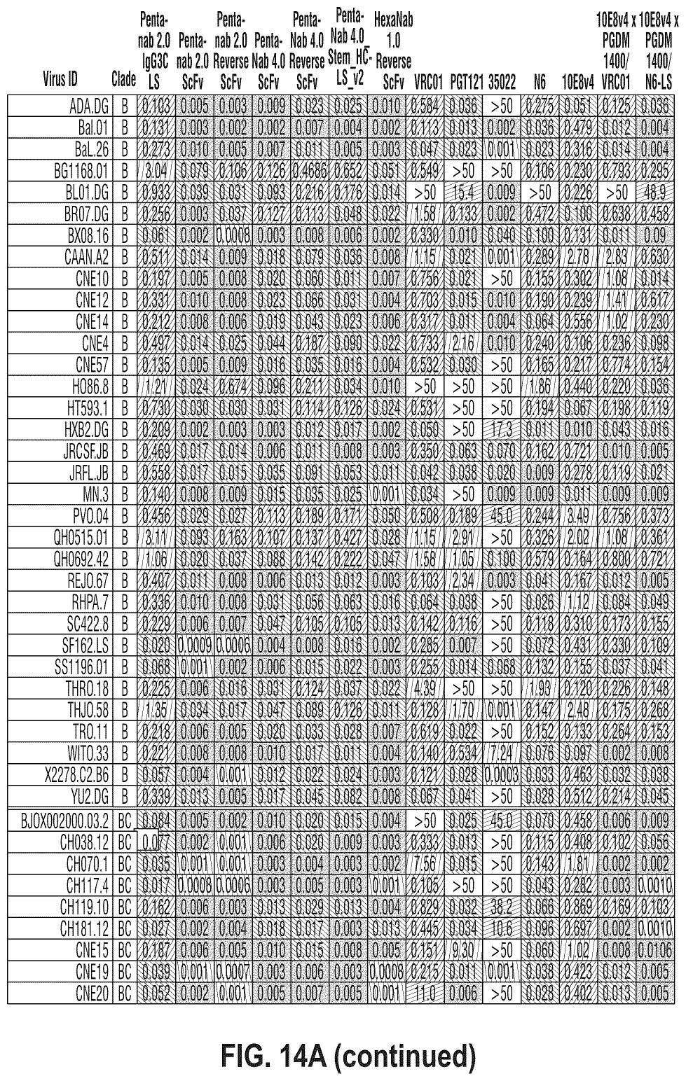

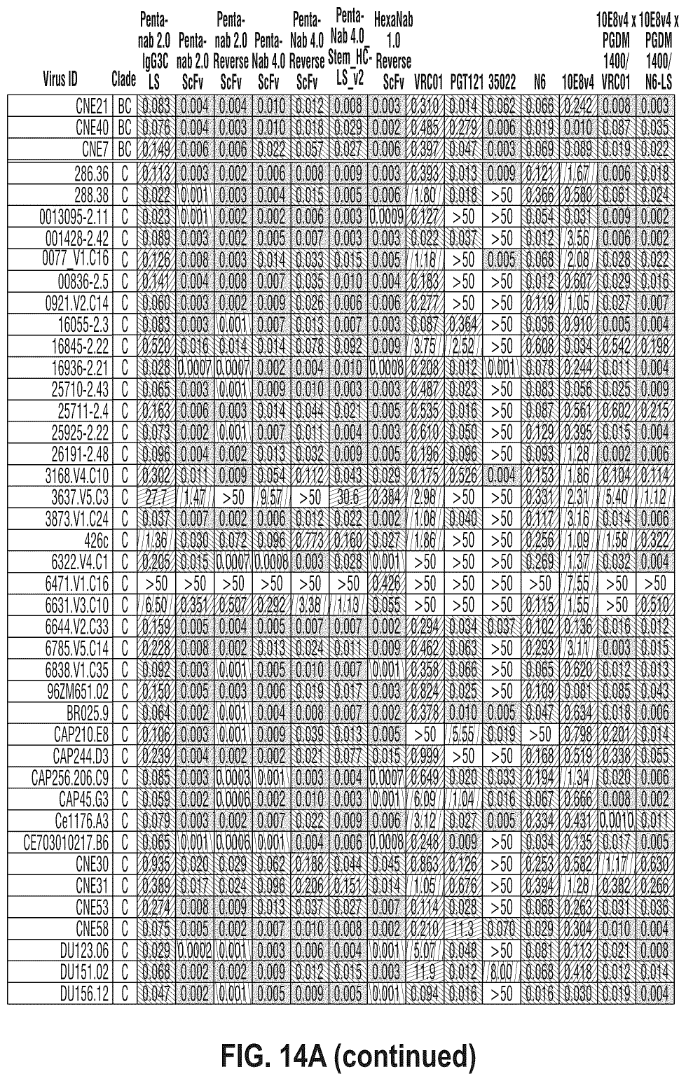

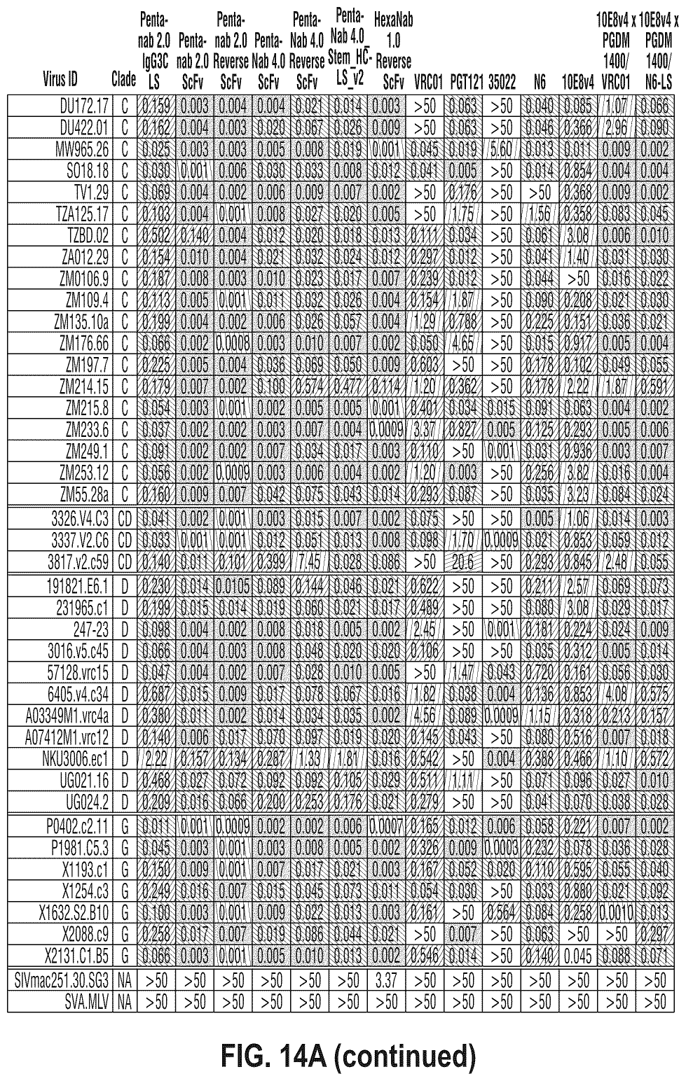

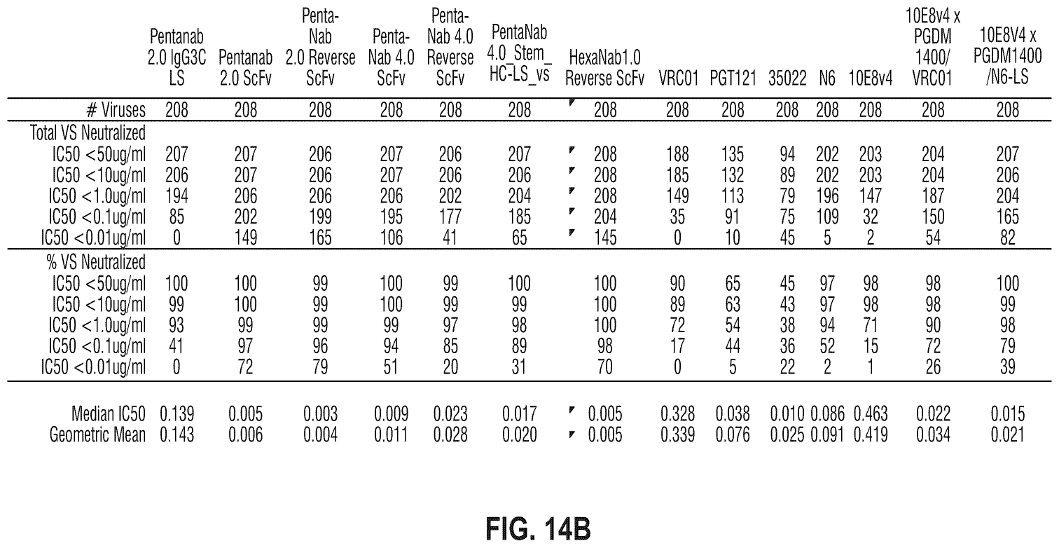

The neutralization profiles of PentaNAb2.0 Reverse ScFv, PentaNAb4.0 Forward ScFv (depicted in FIG. 11A), and PentaNAb4.0 Reverse ScFv (depicted in FIG. 11B) were tested with 208-virus panel (FIGS. 14A & B). It was confirmed that PentaNAb2.0 Reverse ScFv has incremental improvement in potency (IC 50 geometric mean) compared to the initial design, PentaNAb2.0 ScFv (FIGS. 14A & B).

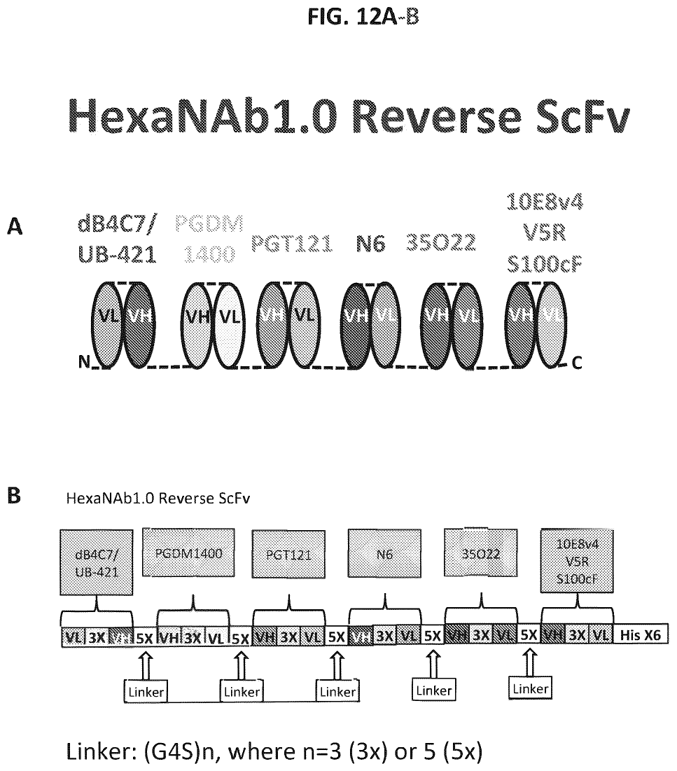

A new multi-specific antibody was constructed, namely HexaNAb 1.0 ScFv (FIG. 12), which contains PentaNAb4.0 Reverse ScFv and variable domains of dB4C7/UB-421, an anti-CD4 antibody (N Engl J Med. 2019 Apr. 18; 380 (16): 1535-1545. PMID: 30995373; patent U.S. Pat. No. 7,501,494B2) to 1) enhance the antiviral breadth, and 2) potentially inhibit the emergence of resistant virus variants as demonstrated by a recent clinical trial described in this cited reference. The HexaNAb 1.0 ScFv shows 100% virus neutralization breadth and promising potency (IC50 geometric mean ˜0.005 ug/mL) (FIGS. 14A & B).

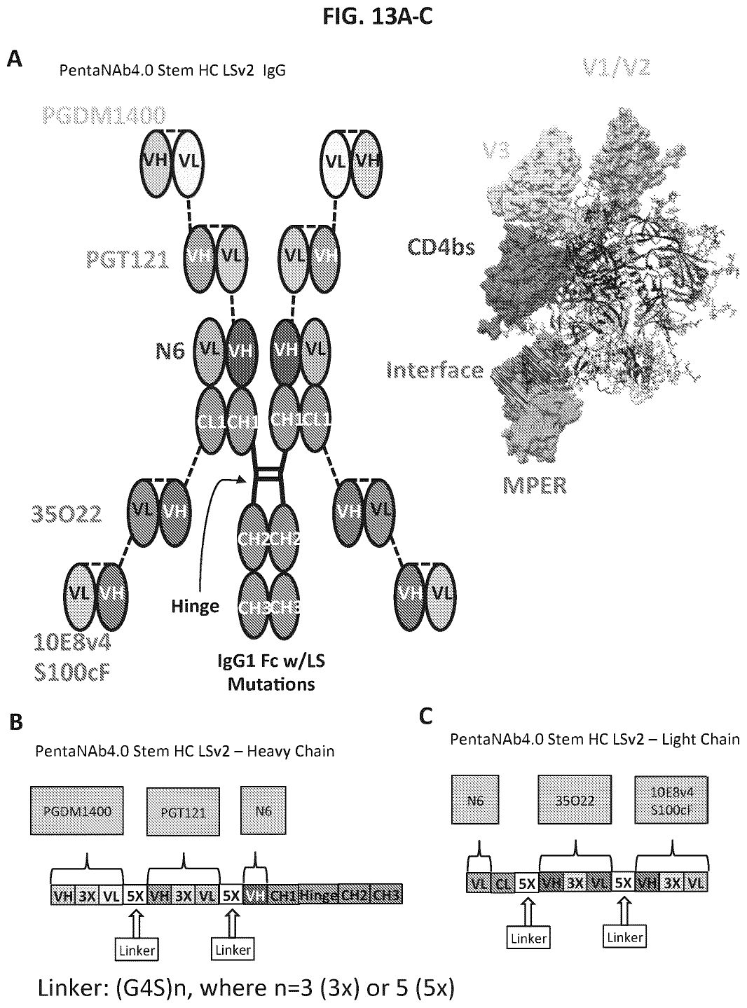

A new version of Penta-specific antibody in full-length IgG form was constructed (FIG. 13), namely PentaNAb4.0 Stem HC LS v2 IgG, expressed as an IgG assembled from a heavy chain and a light chain. With theM428L/N434S (LS) mutation in the Fc domain to elongate antibody in vivo half-life, the heavy chain consists of V2 bNAb PGDM1400, V3 bNAb PGT121, and the VH domain of CD4bs bNAb N6. The light chain contains the VL domain of N6 and 35O22 and 10E8v4 S100cF.

The PentaNAb4.0 Stem HC LS v2 IgG demonstrates 99.5% virus coverage (207/208 viruses tested) and high potency (IC50 geometric mean ˜0.02 ug/mL) (FIGS. 14A & B), which is comparable to the Sanofi Tri-specific antibodies, 10E8v4xPGDM1400/VRC01 and 10E8v4xPGDM1400/N6-LS (FIG. 14B). The 5-specificity feature of the PentaNAb4.0 Stem HC LS v2 IgG may inhibit the emergence of neutralization resistant virus better than the other antibodies possessing less specificities (e.g. Tri-specific antibodies).

PentaNAb4.0 Reverse ScFv
[PGDM1400(VH-3X-VL)]-5X-[PGT121(VH-3X-VL)]-5X-[N6(VH-3X-
VL)]-5X-[35O22(VH-3X-VL)]-5X-[10E8v4 V5R S100cF(VH-3X-
VL)]-His
Amino acid sequence
(SEQ ID NO: 90)
MGWSCIILFLVATATGVHSQAQLVQSGPEVRKPGTSVKVSCKAPGNTLKTYDLHWVRSVPGQGLQWMGWISH
EGDKKVIVERFKAKVTIDWDRSTNTAYLQLSGLTSGDTAVYYCAKGSKHRLRDYALYDDDGALNWAVDVDYL
SNLEFWGQGTAVTVSSGGGGSGGGGSGGGGSDFVLTQSPHSLSVTPGESASISCKSSHSLIHGDRNNYLAWY
VQKPGRSPQLLIYLASSRASGVPDRESGSGSDKDFTLKISRVETEDVGTYYCMQGRESPWTFGQGTKVDIKG
GGGSGGGGSGGGGSGGGGSGGGGSQMQLQESGPGLVKPSETLSLTCSVSGASISDSYWSWIRRSPGKGLEWI
GYVHKSGDTNYSPSLKSRVNLSLDTSKNQVSLSLVAATAADSGKYYCARTLHGRRIYGIVAFNEWFTYFYMD
VWGNGTQVTVSSGGGGSGGGGSGGGGSSDISVAPGETARISCGEKSLGSRAVQWYQHRAGQAPSLIIYNNQD
RPSGIPERFSGSPDSPFGTTATLTITSVEAGDEADYYCHIWDSRVPTKWVFGGGTTLTVLGGGGSGGGGSGG
GGSGGGGSGGGGSRAHLVQSGTAMKKPGASVRVSCQTSGYTFTAHILFWFRQAPGRGLEWVGWIKPQYGAVN
FGGGFRDRVTLTRDVYREIAYMDIRGLKPDDTAVYYCARDRSYGDSSWALDAWGQGTTVVVSAGGGGSGGGG
SGGGGSYIHVTQSPSSLSVSIGDRVTINCQTSQGVGSDLHWYQHKPGRAPKLLIHHTSSVEDGVPSRESGSG
FHTSFNLTISDLQADDIATYYCQVLQFFGRGSRLHIKGGGGSGGGGSGGGGSGGGGSGGGGSQGQLVQSGAE
LKKPGASVKISCKTSGYRFNFYHINWIRQTAGRGPEWMGWISPYSGDKNLAPAFQDRVIMTTDTEVPVTSFT
STGAAYMEIRNLKFDDTGTYFCAKGLLRDGSSTWLPYLWGQGTLLTVSSGGGGSGGGGSGGGGSQSVLTQSA
SVSGSLGQSVTISCTGPNSVCCSHKSISWYQWPPGRAPTLIIYEDNERAPGISPRESGYKSYWSAYLTISDL
RPEDETTYYCCSYTHNSGCVFGTGTKVSVLGGGGSGGGGSGGGGSGGGGSGGGGSEVRLRESGGGLVKPGGS
LRLSCSASGFDFDNAWMTWVRQPPGKGLEWVGRITGPGEGWSVDYAESVKGRFTISRDNTKNTLYLEMNNVR
TEDTGYYFCARTGKYYDFWFGYPPGEEYFQDWGQGTLVIVSSGGGGSGGGGSGGGGSSELTQDPAVSVALKQ
TVTITCRGDSLRSHYASWYQKKPGQAPVLLFYGKNNRPSGIPDRESGSASGNRASLTITGAQAEDEADYYCS
SRDKSGSRLSVFGGGTKLTVLGSGHHHHHH
DNA sequence
(SEQ ID NO. 91)
ATGGGCTGGAGCTGCATCATCCTGTTCCTGGTGGCAACCGCAACAGGAGTGCACAGCCAGGCACAGCTGGTG
CAGAGCGGACCCGAAGTGAGAAAACCTGGGACTAGCGTCAAAGTGTCATGTAAAGCCCCTGGAAATACCCTG
AAGACCTACGATCTGCACTGGGTGCGGTCCGTGCCTGGACAGGGCCTGCAGTGGATGGGATGGATCTCTCAC
GAGGGCGACAAGAAAGTGATCGTGGAGCGGTTCAAGGCCAAGGTGACAATCGATTGGGACAGATCCACCAAC
ACAGCCTACCTGCAGCTGTCTGGCCTGACCAGCGGCGATACAGCCGTGTACTACTGTGCCAAGGGCTCTAAG
CACCGGCTGAGAGACTACGCCCTGTATGACGATGACGGCGCCCTGAACTGGGCCGTGGATGTGGACTATCTG
TCCAATCTGGAGTTCTGGGGACAGGGAACCGCAGTGACAGTGAGCTCCGGAGGAGGAGGCTCCGGCGGCGGA
GGCTCTGGGGGAGGCGGCAGCGATTTTGTGCTGACCCAGTCTCCACACAGCCTGTCCGTGACACCCGGCGAG
TCTGCCAGCATCTCCTGCAAGTCTAGCCACAGCCTGATCCACGGCGACAGGAACAATTACCTGGCCTGGTAC
GTGCAGAAGCCAGGCCGCAGCCCTCAGCTGCTGATCTATCTGGCATCCTCTAGGGCCTCCGGAGTGCCAGAT
CGCTTCTCTGGCAGCGGCTCCGATAAGGACTTTACCCTGAAGATCAGCCGGGTGGAGACAGAGGACGTGGGC
ACATACTATTGTATGCAGGGCCGAGAATCACCTTGGACATTTGGGCAGGGAACTAAAGTTGACATCAAAGGG
GGGGGGGGCTCCGGCGGCGGGGGCTCTGGCGGCGGCGGCAGCGGAGGAGGCGGCTCCGGAGGAGGCGGCTCT
CAGATGCAGCTGCAGGAGAGCGGACCAGGACTGGTGAAGCCTTCCGAGACCCTGTCTCTGACATGTTCTGTG
AGCGGCGCCTCCATCTCTGATAGCTACTGGAGCTGGATCAGACGGAGCCCTGGCAAGGGCCTGGAGTGGATC
GGCTACGTGCACAAGTCTGGCGATACAAACTATTCCCCATCTCTGAAGAGCCGGGTGAACCTGAGCCTGGAC
ACCTCCAAGAATCAGGTGAGCCTGTCCCTGGTGGCAGCAACCGCAGCAGACAGCGGCAAGTACTATTGCGCC
AGAACACTGCACGGCAGGCGCATCTACGGCATCGTGGCCTTTAACGAGTGGTTCACCTACTTTTATATGGAC
GTGTGGGGCAATGGCACCCAGGTGACAGTGTCCTCTGGCGGCGGCGGCTCTGGCGGAGGAGGCAGCGGAGGA
GGAGGCAGCTCCGACATCTCTGTGGCACCTGGAGAGACCGCAAGGATCAGCTGTGGAGAGAAGTCTCTGGGC
AGCAGGGCCGTGCAGTGGTACCAGCACAGGGCAGGACAGGCACCATCCCTGATCATCTATAACAATCAGGAC
CGGCCATCTGGCATCCCCGAGAGATTCTCTGGCAGCCCCGATAGCCCTTTTGGCACCACAGCCACCCTGACA
ATCACCTCCGTGGAGGCCGGCGACGAAGCAGATTACTATTGCCACATCTGGGACTCCAGAGTGCCAACCAAG
TGGGTGTTCGGAGGAGGAACCACACTGACAGTGCTGGGCGGCGGAGGCTCCGGCGGGGGCGGCTCTGGAGGC
GGCGGCAGCGGAGGGGGCGGCTCCGGCGGCGGCGGCTCTAGGGCACACCTGGTGCAGAGCGGAACCGCAATG
AAGAAGCCTGGCGCCTCTGTGCGCGTGAGCTGTCAGACATCCGGCTACACCTTCACCGCCCACATCCTGTTC
TGGTTTAGGCAGGCACCAGGAAGAGGACTGGAGTGGGTGGGCTGGATCAAGCCCCAGTATGGAGCAGTGAAC
TTCGGAGGAGGCTTTCGGGACAGAGTGACACTGACCCGGGACGTGTACAGAGAGATCGCCTATATGGATATC
AGGGGCCTGAAGCCCGACGATACCGCCGTGTACTATTGCGCCAGGGACCGCTCCTACGGCGATTCTAGCTGG
GCACTGGACGCATGGGGACAGGGAACCACAGTGGTGGTGAGCGCCGGAGGCGGGGGCAGCGGCGGCGGGGGC
TCCGGAGGCGGAGGCTCTTACATCCACGTGACCCAGTCCCCTTCCTCTCTGTCCGTGTCTATCGGCGATCGC
GTGACCATCAACTGTCAGACAAGCCAGGGAGTGGGCTCCGACCTGCACTGGTATCAGCACAAGCCTGGCAGG
GCCCCAAAGCTGCTGATCCACCACACAAGCTCCGTGGAGGATGGAGTGCCAAGCCGCTTCAGCGGCTCCGGA
TTCCACACCAGCTTTAATCTGACAATCTCCGACCTGCAGGCCGACGATATCGCCACCTACTATTGCCAGGTG
CTGCAGTTCTTTGGCAGGGGCTCCCGCCTGCACATCAAGGGCGGCGGCGGCTCTGGGGGCGGGGGCAGCGGC
GGGGGGGGCTCCGGGGGAGGAGGCTCTGGCGGAGGGGGCAGCCAGGGCCAGCTGGTGCAGAGCGGAGCAGAG
CTGAAGAAGCCAGGAGCCTCTGTGAAGATCAGCTGTAAGACATCCGGCTATCGGTTCAACTTTTATCACATC
AATTGGATCAGGCAGACCGCAGGAAGGGGACCAGAGTGGATGGGCTGGATCTCCCCCTACTCTGGCGATAAG
AACCTGGCCCCAGCCTTCCAGGACAGAGTGATCATGACCACAGATACCGAGGTGCCAGTGACCAGCTTCACC
TCCACCGGAGCCGCCTACATGGAGATCCGGAATCTGAAGTTCGACGATACAGGCACCTATTTTTGCGCCAAG
GGCCTGCTGAGAGACGGCTCTAGCACATGGCTGCCATACCTGTGGGGACAGGGCACCCTGCTGACAGTGTCC
TCTGGAGGAGGAGGCTCCGGGGGCGGCGGCTCTGGAGGAGGAGGCTCTCAAAGCGTGCTGACCCAGTCCGCC
TCTGTGAGCGGCTCCCTGGGCCAGTCTGTGACCATCAGCTGTACAGGCCCCAACTCCGTGTGCTGTTCTCAC
AAGTCTATCAGCTGGTACCAGTGGCCACCAGGAAGGGCACCTACCCTGATCATCTATGAGGACAATGAGAGG
GCACCAGGAATCAGCCCTCGCTTCTCCGGCTACAAGTCTTATTGGAGCGCCTACCTGACCATTTCCGACCTG
CGCCCCGAGGATGAGACCACATACTATTGCTGTAGCTATACCCACAACTCCGGCTGCGTGTTTGGCACAGGC
ACCAAGGTGAGCGTGCTGGGAGGAGGGGGCTCTGGCGGCGGGGGCAGCGGCGGAGGCGGCTCCGGAGGGGGC
GGCTCTGGCGGAGGCGGCAGCGAGGTGCGGCTGCGGGAGAGCGGCGGCGGCCTGGTGAAGCCAGGCGGCTCT
CTGAGACTGTCCTGTTCTGCCAGCGGCTTCGACTTTGATAATGCCTGGATGACATGGGTGCGGCAGCCTCCT
GGCAAGGGGCTGGAGTGGGTGGGAAGAATCACCGGACCAGGAGAGGGATGGTCTGTGGACTACGCCGAGAGC
GTGAAGGGCCGGTTCACCATCAGCAGAGATAACACTAAAAATACACTGTATCTGGAGATGAACAATGTGCGG
ACCGAGGACACAGGCTACTATTTCTGCGCCAGAACCGGCAAGTACTATGATTTCTGGTTTGGCTACCCCCCT
GGCGAGGAGTATTTTCAGGACTGGGGCCAGGGCACCCTGGTCATCGTGAGCAGCGGCGGGGGAGGCTCCGGC
GGGGGGGGCTCTGGAGGAGGGGGCTCTAGCGAGCTGACCCAGGACCCCGCCGTGTCCGTGGCCCTGAAGCAG
ACAGTGACCATCACATGCAGGGGCGACTCCCTGCGCTCTCACTACGCCAGCTGGTATCAGAAGAAGCCAGGA
CAGGCACCCGTGCTGCTGTTCTACGGCAAGAACAATCGGCCTTCCGGCATCCCAGATAGATTTTCCGGCTCT
GCCAGCGGAAACAGGGCCAGCCTGACCATCACAGGAGCACAGGCAGAGGATGAAGCAGATTACTATTGTTCC
TCTCGGGACAAGTCCGGCTCTAGACTGAGCGTGTTCGGCGGCGGAACCAAGCTGACAGTGCTGGGATCCGGT
CACCACCATCACCACCACTAG
HexaNAb1.0 Reverse ScFv
[dB4C7-UB-421(VL-3X-VH)]-5X-[PGDM1400(VH-3X-VL)]-5X-
[PGT121(VH-3X-VL)]-5X-[N6(VH-3X-VL)]-5X-[35O22(VH-3X-
VL)]-5X-[10E8v4 V5R S100cF(VH-3X-VL)]-His
Amino acid sequence
(SEQ ID NO: 92)
MGWSCIILFLVATATGVHSDIVLTQSPASLAVSLGQRATITCKAGQSVDYDGDSYMNWYQQKPGQPPKLLIY
VASNLESGIPARFSGSGSGTDFTLNIHPVEENDAATYYCQQSYKDPLTFGQGTKLEIKGGGGSGGGGSGGGG
SQVQLVQSGPELKKPGASVKVSCKASGYTFTDYVIHWVKQATGQGLEWIGEIYPGSGSAYSNAKFKDRVTMT
ADKSSNTAYMELSSLTSDDTAVYFCARRGNGTGFAYWGQGTLVTVSSGGGGSGGGGSGGGGSGGGGSGGGGS
QAQLVQSGPEVRKPGTSVKVSCKAPGNTLKTYDLHWVRSVPGQGLQWMGWISHEGDKKVIVERFKAKVTIDW
DRSTNTAYLQLSGLTSGDTAVYYCAKGSKHRLRDYALYDDDGALNWAVDVDYLSNLEFWGQGTAVTVSSGGG
GSGGGGSGGGGSDFVLTQSPHSLSVTPGESASISCKSSHSLIHGDRNNYLAWYVQKPGRSPQLLIYLASSRA
SGVPDRFSGSGSDKDFTLKISRVETEDVGTYYCMQGRESPWTFGQGTKVDIKGGGGSGGGGSGGGGSGGGGS
GGGGSQMQLQESGPGLVKPSETLSLTCSVSGASISDSYWSWIRRSPGKGLEWIGYVHKSGDTNYSPSLKSRV
NLSLDTSKNQVSLSLVAATAADSGKYYCARTLHGRRIYGIVAFNEWFTYFYMDVWGNGTQVTVSSGGGGSGG
GGSGGGGSSDISVAPGETARISCGEKSLGSRAVQWYQHRAGQAPSLIIYNNQDRPSGIPERFSGSPDSPFGT
TATLTITSVEAGDEADYYCHIWDSRVPTKWVFGGGTTLTVLGGGGGSGGGSGGGGSGGGGSGGGGSRAHLVQ
SGTAMKKPGASVRVSCQTSGYTFTAHILFWFRQAPGRGLEWVGWIKPQYGAVNFGGGFRDRVTLTRDVYREI
AYMDIRGLKPDDTAVYYCARDRSYGDSSWALDAWGQGTTVVVSAGGGGSGGGGSGGGGSYIHVTQSPSSLSV
SIGDRVTINCQTSQGVGSDLHWYQHKPGRAPKLLIHHTSSVEDGVPSRFSGSGFHTSFNLTISDLQADDIAT
YYCQVLQFFGRGSRLHIKGGGGSGGGGSGGGGSGGGGSGGGGSQGQLVQSGAELKKPGASVKISCKTSGYRF
NFYHINWIRQTAGRGPEWMGWISPYSGDKNLAPAFQDRVIMTTDTEVPVTSFTSTGAAYMEIRNLKEDDTGT
YFCAKGLLRDGSSTWLPYLWGQGTLLTVSSGGGGSGGGGSGGGGSQSVLTQSASVSGSLGQSVTISCTGPNS
VCCSHKSISWYQWPPGRAPTLIIYEDNERAPGISPRFSGYKSYWSAYLTISDLRPEDETTYYCCSYTHNSGC
VFGTGTKVSVLGGGGSGGGGSGGGGSGGGGSGGGGSEVRLRESGGGLVKPGGSLRLSCSASGEDEDNAWMTW
VRQPPGKGLEWVGRITGPGEGWSVDYAESVKGRFTISRDNTKNTLYLEMNNVRTEDTGYYFCARTGKYYDFW
FGYPPGEEYFQDWGQGTLVIVSSGGGGSGGGGSGGGGSSELTQDPAVSVALKQTVTITCRGDSLRSHYASWY
QKKPGQAPVLLFYGKNNRPSGIPDRESGSASGNRASLTITGAQAEDEADYYCSSRDKSGSRLSVFGGGTKLT
VLGSGHHHHHH
DNA sequence
(SEQ ID NO: 93)
ATGGGCTGGAGCTGCATCATCCTGTTCCTGGTGGCAACCGCAACAGGAGTGCACAGCGACATCGTGCTGACC
CAGTCTCCTGCCAGCCTGGCCGTGAGCCTGGGACAGAGGGCCACCATCACATGCAAGGCCGGCCAGTCTGTG
GACTACGATGGCGACAGCTACATGAACTGGTATCAGCAGAAGCCAGGCCAGCCCCCTAAGCTGCTGATCTAT
GTGGCCTCCAATCTGGAGTCTGGCATCCCTGCACGCTTCAGCGGCTCCGGCTCTGGAACCGACTTCACCCTG
AACATCCACCCAGTGGAGGAGAATGACGCCGCCACCTACTATTGCCAGCAGAGCTACAAGGACCCCCTGACC
TTCGGCCAGGGCACAAAGCTGGAGATCAAGGGAGGAGGAGGCAGCGGGGGAGGAGGCTCCGGAGGCGGCGGC
TCTCAGGTGCAGCTGGTGCAGTCCGGACCAGAGCTGAAGAAGCCAGGAGCCAGCGTGAAGGTGTCCTGTAAG
GCCTCTGGCTACACCTTCACAGATTATGTGATCCACTGGGTGAAGCAGGCAACAGGACAGGGCCTGGAGTGG
ATCGGAGAGATCTACCCAGGCAGCGGCTCCGCCTATTCCAACGCCAAGTTTAAGGATCGGGTGACCATGACA
GCCGACAAGAGCTCCAATACCGCCTATATGGAGCTGTCTAGCCTGACCTCTGACGATACAGCCGTGTACTTC
TGTGCCCGGAGAGGCAACGGCACAGGCTTTGCCTATTGGGGCCAGGGCACCCTGGTGACAGTGTCCTCTGGA
GGAGGAGGCAGCGGCGGCGGCGGCTCCGGAGGAGGCGGCTCTGGCGGCGGCGGCAGCGGAGGAGGCGGCTCC
CAGGCACAGCTGGTGCAGAGCGGACCCGAAGTGAGAAAACCTGGGACTAGCGTCAAAGTGTCATGTAAAGCC
CCTGGAAATACCCTGAAGACCTACGATCTGCACTGGGTGCGGTCCGTGCCTGGACAGGGCCTGCAGTGGATG
GGATGGATCTCTCACGAGGGCGACAAGAAAGTGATCGTGGAGCGGTTCAAGGCCAAGGTGACAATCGATTGG
GACAGATCCACCAACACAGCCTACCTGCAGCTGTCTGGCCTGACCAGCGGCGATACAGCCGTGTACTACTGT
GCCAAGGGCTCTAAGCACCGGCTGAGAGACTACGCCCTGTATGACGATGACGGCGCCCTGAACTGGGCCGTG
GATGTGGACTATCTGTCCAATCTGGAGTTCTGGGGACAGGGAACCGCAGTGACAGTGAGCTCCGGAGGAGGA
GGCTCCGGCGGCGGAGGCTCTGGGGGAGGCGGCAGCGATTTTGTGCTGACCCAGTCTCCACACAGCCTGTCC
GTGACACCCGGCGAGTCTGCCAGCATCTCCTGCAAGTCTAGCCACAGCCTGATCCACGGCGACAGGAACAAT
TACCTGGCCTGGTACGTGCAGAAGCCAGGCCGCAGCCCTCAGCTGCTGATCTATCTGGCATCCTCTAGGGCC
TCCGGAGTGCCAGATCGCTTCTCTGGCAGCGGCTCCGATAAGGACTTTACCCTGAAGATCAGCCGGGTGGAG
ACAGAGGACGTGGGCACATACTATTGTATGCAGGGCCGAGAATCACCTTGGACATTTGGGCAGGGAACTAAA
GTTGACATCAAAGGGGGGGGGGGCTCCGGCGGCGGGGGCTCTGGCGGCGGCGGCAGCGGAGGAGGCGGCTCC
GGAGGAGGCGGCTCTCAGATGCAGCTGCAGGAGAGCGGACCAGGACTGGTGAAGCCTTCCGAGACCCTGTCT
CTGACATGTTCTGTGAGCGGCGCCTCCATCTCTGATAGCTACTGGAGCTGGATCAGACGGAGCCCTGGCAAG
GGCCTGGAGTGGATCGGCTACGTGCACAAGTCTGGCGATACAAACTATTCCCCATCTCTGAAGAGCCGGGTG
AACCTGAGCCTGGACACCTCCAAGAATCAGGTGAGCCTGTCCCTGGTGGCAGCAACCGCAGCAGACAGCGGC
AAGTACTATTGCGCCAGAACACTGCACGGCAGGCGCATCTACGGCATCGTGGCCTTTAACGAGTGGTTCACC
TACTTTTATATGGACGTGTGGGGCAATGGCACCCAGGTGACAGTGTCCTCTGGCGGCGGCGGCTCTGGCGGA
GGAGGCAGCGGAGGAGGAGGCAGCTCCGACATCTCTGTGGCACCTGGAGAGACCGCAAGGATCAGCTGTGGA
GAGAAGTCTCTGGGCAGCAGGGCCGTGCAGTGGTACCAGCACAGGGCAGGACAGGCACCATCCCTGATCATC
TATAACAATCAGGACCGGCCATCTGGCATCCCCGAGAGATTCTCTGGCAGCCCCGATAGCCCTTTTGGCACC
ACAGCCACCCTGACAATCACCTCCGTGGAGGCCGGCGACGAAGCAGATTACTATTGCCACATCTGGGACTCC
AGAGTGCCAACCAAGTGGGTGTTCGGAGGAGGAACCACACTGACAGTGCTGGGCGGCGGAGGCTCCGGCGGG
GGCGGCTCTGGAGGCGGCGGCAGCGGAGGGGGCGGCTCCGGCGGCGGCGGCTCTAGGGCACACCTGGTGCAG
AGCGGAACCGCAATGAAGAAGCCTGGCGCCTCTGTGCGCGTGAGCTGTCAGACATCCGGCTACACCTTCACC
GCCCACATCCTGTTCTGGTTTAGGCAGGCACCAGGAAGAGGACTGGAGTGGGTGGGCTGGATCAAGCCCCAG
TATGGAGCAGTGAACTTCGGAGGAGGCTTTCGGGACAGAGTGACACTGACCCGGGACGTGTACAGAGAGATC
GCCTATATGGATATCAGGGGCCTGAAGCCCGACGATACCGCCGTGTACTATTGCGCCAGGGACCGCTCCTAC
GGCGATTCTAGCTGGGCACTGGACGCATGGGGACAGGGAACCACAGTGGTGGTGAGCGCCGGAGGCGGGGGC
AGCGGCGGCGGGGGCTCCGGAGGCGGAGGCTCTTACATCCACGTGACCCAGTCCCCTTCCTCTCTGTCCGTG
TCTATCGGCGATCGCGTGACCATCAACTGTCAGACAAGCCAGGGAGTGGGCTCCGACCTGCACTGGTATCAG
CACAAGCCTGGCAGGGCCCCAAAGCTGCTGATCCACCACACAAGCTCCGTGGAGGATGGAGTGCCAAGCCGC
TTCAGCGGCTCCGGATTCCACACCAGCTTTAATCTGACAATCTCCGACCTGCAGGCCGACGATATCGCCACC
TACTATTGCCAGGTGCTGCAGTTCTTTGGCAGGGGCTCCCGCCTGCACATCAAGGGCGGCGGCGGCTCTGGG
GGCGGGGGCAGCGGCGGGGGGGGCTCCGGGGGAGGAGGCTCTGGCGGAGGGGGCAGCCAGGGCCAGCTGGTG
CAGAGCGGAGCAGAGCTGAAGAAGCCAGGAGCCTCTGTGAAGATCAGCTGTAAGACATCCGGCTATCGGTTC
AACTTTTATCACATCAATTGGATCAGGCAGACCGCAGGAAGGGGACCAGAGTGGATGGGCTGGATCTCCCCC
TACTCTGGCGATAAGAACCTGGCCCCAGCCTTCCAGGACAGAGTGATCATGACCACAGATACCGAGGTGCCA
GTGACCAGCTTCACCTCCACCGGAGCCGCCTACATGGAGATCCGGAATCTGAAGTTCGACGATACAGGCACC
TATTTTTGCGCCAAGGGCCTGCTGAGAGACGGCTCTAGCACATGGCTGCCATACCTGTGGGGACAGGGCACC
CTGCTGACAGTGTCCTCTGGAGGAGGAGGCTCCGGGGGCGGCGGCTCTGGAGGAGGAGGCTCTCAAAGCGTG
CTGACCCAGTCCGCCTCTGTGAGCGGCTCCCTGGGCCAGTCTGTGACCATCAGCTGTACAGGCCCCAACTCC
GTGTGCTGTTCTCACAAGTCTATCAGCTGGTACCAGTGGCCACCAGGAAGGGCACCTACCCTGATCATCTAT
GAGGACAATGAGAGGGCACCAGGAATCAGCCCTCGCTTCTCCGGCTACAAGTCTTATTGGAGCGCCTACCTG
ACCATTTCCGACCTGCGCCCCGAGGATGAGACCACATACTATTGCTGTAGCTATACCCACAACTCCGGCTGC
GTGTTTGGCACAGGCACCAAGGTGAGCGTGCTGGGAGGAGGGGGCTCTGGCGGCGGGGGCAGCGGCGGAGGC
GGCTCCGGAGGGGGCGGCTCTGGCGGAGGCGGCAGCGAGGTGCGGCTGCGGGAGAGCGGCGGCGGCCTGGTG
AAGCCAGGCGGCTCTCTGAGACTGTCCTGTTCTGCCAGCGGCTTCGACTTTGATAATGCCTGGATGACATGG
GTGCGGCAGCCTCCTGGCAAGGGGCTGGAGTGGGTGGGAAGAATCACCGGACCAGGAGAGGGATGGTCTGTG
GACTACGCCGAGAGCGTGAAGGGCCGGTTCACCATCAGCAGAGATAACACTAAAAATACACTGTATCTGGAG
ATGAACAATGTGCGGACCGAGGACACAGGCTACTATTTCTGCGCCAGAACCGGCAAGTACTATGATTTCTGG
TTTGGCTACCCCCCTGGCGAGGAGTATTTTCAGGACTGGGGCCAGGGCACCCTGGTCATCGTGAGCAGCGGC
GGGGGAGGCTCCGGCGGGGGGGGCTCTGGAGGAGGGGGCTCTAGCGAGCTGACCCAGGACCCCGCCGTGTCC
GTGGCCCTGAAGCAGACAGTGACCATCACATGCAGGGGCGACTCCCTGCGCTCTCACTACGCCAGCTGGTAT
CAGAAGAAGCCAGGACAGGCACCCGTGCTGCTGTTCTACGGCAAGAACAATCGGCCTTCCGGCATCCCAGAT
AGATTTTCCGGCTCTGCCAGCGGAAACAGGGCCAGCCTGACCATCACAGGAGCACAGGCAGAGGATGAAGCA
GATTACTATTGTTCCTCTCGGGACAAGTCCGGCTCTAGACTGAGCGTGTTCGGCGGCGGAACCAAGCTGACA
GTGCTGGGATCCGGTCACCACCATCACCACCACTAG
PentaNAb4.0 Stem HC LS v2
HC: PGDM1400-5X-PGT121-5X-N6 HC
[PGDM1400(VH-3X-VL)]-5X-[PGT121(VH-3X-VL)]-
5X-[N6(VH)]-CH1-CH2-CH3
Amino acid sequence
(SEQ ID NO: 94)
MGWSCIILFLVATATGVHSQAQLVQSGPEVRKPGTSVKVSCKAPGNTLKTYDLHWVRSVPGQGLQWMGWISH
EGDKKVIVERFKAKVTIDWDRSTNTAYLQLSGLTSGDTAVYYCAKGSKHRLRDYALYDDDGALNWAVDVDYL
SNLEFWGQGTAVTVSSGGGGSGGGGSGGGGSDFVLTQSPHSLSVTPGESASISCKSSHSLIHGDRNNYLAWY
VQKPGRSPQLLIYLASSRASGVPDRFSGSGSDKDFTLKISRVETEDVGTYYCMQGRESPWTFGQGTKVDIKG
GGGSGGGGSGGGGSGGGGSGGGGSQMQLQESGPGLVKPSETLSLTCSVSGASISDSYWSWIRRSPGKGLEWI
GYVHKSGDTNYSPSLKSRVNLSLDTSKNQVSLSLVAATAADSGKYYCARTLHGRRIYGIVAFNEWFTYFYMD
VWGNGTQVTVSSGGGGSGGGGSGGGGSSDISVAPGETARISCGEKSLGSRAVQWYQHRAGQAPSLIIYNNQD
RPSGIPERFSGSPDSPFGTTATLTITSVEAGDEADYYCHIWDSRVPTKWVFGGGTTLTVLGGGGSGGGGSGG
GGSGGGGSGGGGSRAHLVQSGTAMKKPGASVRVSCQTSGYTFTAHILFWFRQAPGRGLEWVGWIKPQYGAVN
FGGGFRDRVTLTRDVYREIAYMDIRGLKPDDTAVYYCARDRSYGDSSWALDAWGQGTTVVVSAASTKGPSVF
PLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVDK
SRWQQGNVFSCSVLHEALHSHYTQKSLSLSPGK
DNA sequence
(SEQ ID NO: 95)
ATGGGCTGGAGCTGCATCATCCTGTTCCTGGTGGCAACCGCAACCGGTGTGCACAGCCAGGCACAGCTGGTG
CAGAGCGGACCCGAAGTGAGAAAACCTGGGACTAGCGTCAAAGTGTCATGTAAAGCCCCTGGAAATACCCTG
AAGACCTACGATCTGCACTGGGTGCGGTCCGTGCCTGGACAGGGCCTGCAGTGGATGGGATGGATCTCTCAC
GAGGGCGACAAGAAAGTGATCGTGGAGCGGTTCAAGGCCAAGGTGACAATCGATTGGGACAGATCCACCAAC
ACAGCCTACCTGCAGCTGTCTGGCCTGACCAGCGGCGATACAGCCGTGTACTACTGTGCCAAGGGCTCTAAG
CACCGGCTGAGAGACTACGCCCTGTATGACGATGACGGCGCCCTGAACTGGGCCGTGGATGTGGACTATCTG
TCCAATCTGGAGTTCTGGGGACAGGGAACCGCAGTGACAGTGAGCTCCGGAGGAGGAGGCTCCGGCGGCGGA
GGCTCTGGGGGAGGCGGCAGCGATTTTGTGCTGACCCAGTCTCCACACAGCCTGTCCGTGACACCCGGCGAG
TCTGCCAGCATCTCCTGCAAGTCTAGCCACAGCCTGATCCACGGCGACAGGAACAATTACCTGGCCTGGTAC
GTGCAGAAGCCAGGCCGCAGCCCTCAGCTGCTGATCTATCTGGCATCCTCTAGGGCCTCCGGAGTGCCAGAT
CGCTTCTCTGGCAGCGGCTCCGATAAGGACTTTACCCTGAAGATCAGCCGGGTGGAGACAGAGGACGTGGGC
ACATACTATTGTATGCAGGGCCGAGAATCACCTTGGACATTTGGGCAGGGAACTAAAGTTGACATCAAAGGG
GGGGGGGGCTCCGGCGGCGGGGGCTCTGGCGGCGGCGGCAGCGGAGGAGGCGGCTCCGGAGGAGGCGGCTCT
CAGATGCAGCTGCAGGAGAGCGGACCAGGACTGGTGAAGCCTTCCGAGACCCTGTCTCTGACATGTTCTGTG
AGCGGCGCCTCCATCTCTGATAGCTACTGGAGCTGGATCAGACGGAGCCCTGGCAAGGGCCTGGAGTGGATC
GGCTACGTGCACAAGTCTGGCGATACAAACTATTCCCCATCTCTGAAGAGCCGGGTGAACCTGAGCCTGGAC
ACCTCCAAGAATCAGGTGAGCCTGTCCCTGGTGGCAGCAACCGCAGCAGACAGCGGCAAGTACTATTGCGCC
AGAACACTGCACGGCAGGCGCATCTACGGCATCGTGGCCTTTAACGAGTGGTTCACCTACTTTTATATGGAC
GTGTGGGGCAATGGCACCCAGGTGACAGTGTCCTCTGGCGGCGGCGGCTCTGGCGGAGGAGGCAGCGGAGGA
GGAGGCAGCTCCGACATCTCTGTGGCACCTGGAGAGACCGCAAGGATCAGCTGTGGAGAGAAGTCTCTGGGC
AGCAGGGCCGTGCAGTGGTACCAGCACAGGGCAGGACAGGCACCATCCCTGATCATCTATAACAATCAGGAC
CGGCCATCTGGCATCCCCGAGAGATTCTCTGGCAGCCCCGATAGCCCTTTTGGCACCACAGCCACCCTGACA
ATCACCTCCGTGGAGGCCGGCGACGAAGCAGATTACTATTGCCACATCTGGGACTCCAGAGTGCCAACCAAG
TGGGTGTTCGGAGGAGGAACCACACTGACAGTGCTGGGCGGCGGAGGCTCCGGCGGGGGCGGCTCTGGAGGC
GGCGGCAGCGGAGGGGGCGGCTCCGGCGGCGGCGGCTCTAGGGCACACCTGGTGCAGAGCGGAACCGCAATG
AAGAAGCCTGGCGCCTCTGTGCGCGTGAGCTGTCAGACATCCGGCTACACCTTCACCGCCCACATCCTGTTC
TGGTTTAGGCAGGCACCAGGAAGAGGACTGGAGTGGGTGGGCTGGATCAAGCCCCAGTATGGAGCAGTGAAC
TTCGGAGGAGGCTTTCGGGACAGAGTGACACTGACCCGGGACGTGTACAGAGAGATCGCCTATATGGATATC
AGGGGCCTGAAGCCCGACGATACCGCCGTGTACTATTGCGCCAGGGACCGCTCCTACGGCGATTCTAGCTGG
GCACTGGACGCATGGGGACAGGGAACCACAGTGGTGGTGAGCGCCGCGTCGACCAAGGGCCCATCGGTCTTC
CCCCTGGCACCCTCCTCCAAGAGCACCTCTGGGGGCACAGCGGCCCTGGGCTGCCTGGTCAAGGACTACTTC
CCCGAACCTGTGACGGTGTCGTGGAACTCAGGCGCCCTGACCAGCGGCGTGCACACCTTCCCGGCTGTCCTA
CAGTCCTCAGGACTCTACTCCCTCAGCAGCGTGGTGACCGTGCCCTCCAGCAGCTTGGGCACCCAGACCTAC
ATCTGCAACGTGAATCACAAGCCCAGCAACACCAAGGTGGACAAGAAAGTTGAGCCCAAATCTTGTGACAAA
ACTCACACATGCCCACCGTGCCCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTGGTGGACGTGAGCCACGAAGAC
CCTGAGGTCAAGTTCAACTGGTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATCTCCAAAGCCAAAGGGCAG
CCCCGAGAACCACAGGTGTACACCCTGCCCCCATCCCGGGAGGAGATGACCAAGAACCAGGTCAGCCTGACC
TGCCTGGTCAAAGGCTTCTATCCCAGCGACATCGCCGTGGAGTGGGAGAGCAATGGGCAGCCGGAGAACAAC
TACAAGACCACGCCTCCCGTGCTGGACTCCGACGGCTCCTTCTTCCTCTACAGCAAGCTCACCGTGGACAAG
AGCAGGTGGCAGCAGGGGAACGTCTTCTCATGCTCCGTGCTGCATGAGGCTCTGCACAGCCACTACACGCAG
AAGAGCCTCTCCCTGTCTCCGGGTAAATGA
LC: N6 KC-CK-35O22-5X-10E8v4 V5R S100cF
[N6(VL)]-CL-5X-[35O22(VH-3X-VL)]-5X-[10E8v4
S100cF (VH-3X-VL)]
Amino acid sequence
(SEQ ID NO: 96)
MGWSCIILFLVATATGVHSYIHVTQSPSSLSVSIGDRVTINCQTSQGVGSDLHWYQHKPGRAPKLLIHHTSS
VEDGVPSRFSGSGFHTSFNLTISDLQADDIATYYCQVLQFFGRGSRLHIKRTVAAPSVFIFPPSDEQLKSGT
ASVVCLLNNFYPREAKVQWKVDNALQSGNSQESVTEQDSKDSTYSLSSTLTLSKADYEKHKVYACEVTHQGL
SSPVTKSFNRGECGGGGSGGGGSGGGGSGGGGSGGGGSQGQLVQSGAELKKPGASVKISCKTSGYRFNFYHI
NWIRQTAGRGPEWMGWISPYSGDKNLAPAFQDRVIMTTDTEVPVTSFTSTGAAYMEIRNLKFDDTGTYFCAK
GLLRDGSSTWLPYLWGQGTLLTVSSGGGGSGGGGSGGGGSQSVLTQSASVSGSLGQSVTISCTGPNSVCCSH
KSISWYQWPPGRAPTLIIYEDNERAPGISPRESGYKSYWSAYLTISDLRPEDETTYYCCSYTHNSGCVFGTG
TKVSVLGGGGSGGGGSGGGGSGGGGSGGGGSEVRLVESGGGLVKPGGSLRLSCSASGFDEDNAWMTWVRQPP
GKGLEWVGRITGPGEGWSVDYAESVKGRFTISRDNTKNTLYLEMNNVRTEDTGYYFCARTGKYYDFWFGYPP
GEEYFQDWGQGTLVIVSSGGGGSGGGGSGGGGSSELTQDPAVSVALKQTVTITCRGDSLRSHYASWYQKKPG
QAPVLLFYGKNNRPSGIPDRFSGSASGNRASLTITGAQAEDEADYYCSSRDKSGSRLSVFGGGTKLTVL
DNA sequence
(SEQ ID NO: 97)
ATGGGCTGGAGCTGCATCATCCTGTTCCTGGTGGCAACCGCAACAGGAGTGCACAGCTACATCCACGTGACC
CAGTCCCCTTCCTCTCTGTCCGTGTCTATCGGCGATCGCGTGACCATCAACTGTCAGACAAGCCAGGGAGTG
GGCTCCGACCTGCACTGGTATCAGCACAAGCCTGGCAGGGCCCCAAAGCTGCTGATCCACCACACAAGCTCC
GTGGAGGATGGAGTGCCAAGCCGCTTCAGCGGCTCCGGATTCCACACCAGCTTTAATCTGACAATCTCCGAC
CTGCAGGCCGACGATATCGCCACCTACTATTGCCAGGTGCTGCAGTTCTTTGGCAGGGGCTCCCGCCTGCAC
ATCAAGCGTACGGTGGCTGCACCATCTGTCTTCATCTTCCCGCCATCTGATGAGCAGTTGAAATCTGGAACT
GCCTCTGTTGTGTGCCTGCTGAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACAGCACCTACAGCCTCAGCAGC
ACCCTGACGCTGAGCAAAGCAGACTACGAGAAACACAAAGTCTACGCCTGCGAAGTCACCCATCAGGGCCTG
AGCTCGCCCGTCACAAAGAGCTTCAACAGGGGAGAGTGTGGAGGAGGGGGCTCTGGCGGCGGGGGCAGCGGC
GGAGGCGGCTCCGGAGGGGGCGGCTCTGGCGGAGGCGGCAGCCAGGGCCAGCTGGTGCAGAGCGGAGCAGAG
CTGAAGAAGCCAGGAGCCTCTGTGAAGATCAGCTGTAAGACATCCGGCTACCGGTTCAACTTTTATCACATC
AATTGGATCAGGCAGACCGCAGGAAGGGGACCAGAGTGGATGGGCTGGATCTCCCCCTACTCTGGCGATAAG
AACCTGGCCCCAGCCTTCCAGGACAGAGTGATCATGACCACAGATACCGAGGTGCCAGTGACCAGCTTCACC
TCCACCGGAGCCGCCTACATGGAGATCCGGAATCTGAAGTTCGACGATACAGGCACCTATTTTTGCGCCAAG
GGCCTGCTGAGAGACGGCTCTAGCACATGGCTGCCATACCTGTGGGGACAGGGCACCCTGCTGACAGTGTCC
TCTGGAGGAGGAGGCTCCGGGGGCGGCGGCTCTGGAGGAGGAGGCTCTCAAAGCGTGCTGACCCAGTCCGCC
TCTGTGAGCGGCTCCCTGGGCCAGTCTGTGACCATCAGCTGTACAGGCCCCAACTCCGTGTGCTGTTCTCAC
AAGTCTATCAGCTGGTACCAGTGGCCACCAGGAAGGGCACCTACCCTGATCATCTATGAGGACAATGAGAGG
GCACCAGGAATCAGCCCTCGCTTCTCCGGCTACAAGTCTTATTGGAGCGCCTACCTGACCATTTCCGACCTG
CGCCCCGAGGATGAGACCACATACTATTGCTGTAGCTATACCCACAACTCCGGCTGCGTGTTTGGCACAGGC
ACCAAGGTGAGCGTGCTGGGAGGAGGGGGCTCTGGCGGCGGGGGCAGCGGCGGAGGCGGCTCCGGAGGGGGC
GGCTCTGGCGGAGGCGGCAGCGAGGTGCGGCTGGTGGAGAGCGGCGGCGGCCTGGTGAAGCCAGGCGGCTCT
CTGAGACTGTCCTGTTCTGCCAGCGGCTTCGACTTTGATAATGCCTGGATGACATGGGTGCGGCAGCCTCCT
GGCAAGGGGCTGGAGTGGGTGGGAAGAATCACCGGACCAGGAGAGGGATGGTCTGTGGACTACGCCGAGAGC
GTGAAGGGCCGGTTCACCATCAGCAGAGATAACACTAAAAATACACTGTATCTGGAGATGAACAATGTGCGG
ACCGAGGACACAGGCTACTATTTCTGCGCCAGAACCGGCAAGTACTATGATTTCTGGTTTGGCTACCCCCCT
GGCGAGGAGTATTTTCAGGACTGGGGCCAGGGCACCCTGGTCATCGTGAGCAGCGGCGGGGGAGGCTCCGGC
GGGGGGGGCTCTGGAGGAGGGGGCTCTAGCGAGCTGACCCAGGACCCCGCCGTGTCCGTGGCCCTGAAGCAG
ACAGTGACCATCACATGCAGGGGCGACTCCCTGCGCTCTCACTACGCCAGCTGGTATCAGAAGAAGCCAGGA
CAGGCACCCGTGCTGCTGTTCTACGGCAAGAACAATCGGCCTTCCGGCATCCCAGATAGATTTTCCGGCTCT
GCCAGCGGAAACAGGGCCAGCCTGACCATCACAGGAGCACAGGCAGAGGATGAAGCAGATTACTATTGTTCC
TCTCGGGACAAGTCCGGCTCTAGACTGAGC
mAb dB4C7/UB-421's perfect heavy chain Fv region
(SEQ ID NO 98)
QVQLVQSGPELKKPGASVKVSCKASGYTFTDYVIHWVKQATGQGLEWIGEIYPGSGSAYSNAKFKDRVTMTA
DKSSNTAYMELSSLTSDDTAVYFCARRGNGTGFAYWGQGTLVTVSS
mAb dB4C7/UB-421's perfect light chain Fv region
(SEQ ID NO. 99)
DIVLTQSPASLAVSLGQRATITCKAGQSVDYDGDSYMNWYQQKPGQPPKLLIYVASNLESGIPARFSGSGSG
TDFTLNIHPVEENDAATYYCQQSYKDPLTFGQGTKLEIK
CDR1 of Heavy Chain of murine antibody B4
SEQ ID NO. 100
DYVIH
CDR2 of Heavy Chain of murine antibody B4
SEQ ID NO. 101
EIYPGSGSAYSNAKFKD
CDR3 of Heavy Chain of murine antibody B4
SEQ ID NO. 102
RGNGTGFAY
CDR1 of Light Chain of murine antibody B4
SEQ ID NO. 103
KAGQSVDYDGDSYMN
CDR2 of Light Chain of murine antibody B4
SEQ ID NO. 104
VASNLES
CDR3 of Light Chain of murine antibody B4
SEQ ID NO. 105
QQSYKDPLT

| SEQ ID | | | |
|---|---|---|---|
| NO: | Name | Orientation | Amino Acid Sequence |
| 92 | HexaNAb1.0 | (dB4C7/UB- | MGWSCIILFLVATATGVHSDIVLTQSPASLAV |
| | Reverse | 421)-5X- | SLGQRATITCKAGQSVDYDGDSYMNWYQQKPG |
| | ScFv | PGDM1400-5X- | QPPKLLIYVASNLESGIPARFSGSGSGTDFTL |
| | | PGT121-5X-N6- | NIHPVEENDAATYYCQQSYKDPLTFGQGTKLE |
| | | 5X-35O22-5X- | IKGGGGSGGGGSGGGGSQVQLVQSGPELKKPG |
| | | 10E8v4 V5R | ASVKVSCKASGYTFTDYVIHWVKQATGQGLEW |
| | | S100cF-His | IGEIYPGSGSAYSNAKFKDRVTMTADKSSNTA |
| | | | YMELSSLTSDDTAVYFCARRGNGTGFAYWGQG |
| | | | TLVTVSSGGGGSGGGGSGGGGSGGGGSGGGGS |
| | | | QAQLVQSGPEVRKPGTSVKVSCKAPGNTLKTY |
| | | | DLHWVRSVPGQGLQWMGWISHEGDKKVIVERF |
| | | | KAKVTIDWDRSTNTAYLQLSGLTSGDTAVYYC |
| | | | AKGSKHRLRDYALYDDDGALNWAVDVDYLSNL |
| | | | EFWGQGTAVTVSSGGGGSGGGGSGGGGSDFVL |
| | | | TQSPHSLSVTPGESASISCKSSHSLIHGDRNN |
| | | | YLAWYVQKPGRSPQLLIYLASSRASGVPDRES |
| | | | GSGSDKDFTLKISRVETEDVGTYYCMQGRESP |
| | | | WTFGQGTKVDIKGGGGSGGGGSGGGGSGGGGS |
| | | | GGGGSQMQLQESGPGLVKPSETLSLTCSVSGA |
| | | | SISDSYWSWIRRSPGKGLEWIGYVHKSGDTNY |
| | | | SPSLKSRVNLSLDTSKNQVSLSLVAATAADSG |
| | | | KYYCARTLHGRRIYGIVAFNEWFTYFYMDVWG |
| | | | NGTQVTVSSGGGGSGGGGSGGGGSSDISVAPG |
| | | | ETARISCGEKSLGSRAVQWYQHRAGQAPSLII |
| | | | YNNQDRPSGIPERFSGSPDSPFGTTATLTITS |
| | | | VEAGDEADYYCHIWDSRVPTKWVFGGGTTLTV |
| | | | LGGGGSGGGGSGGGGSGGGGSGGGGSRAHLVQ |
| | | | SGTAMKKPGASVRVSCQTSGYTFTAHILFWER |
| | | | QAPGRGLEWVGWIKPQYGAVNFGGGFRDRVTL |
| | | | TRDVYREIAYMDIRGLKPDDTAVYYCARDRSY |
| | | | GDSSWALDAWGQGTTVVVSAGGGGSGGGGSGG |
| | | | GGSYIHVTQSPSSLSVSIGDRVTINCQTSQGV |
| | | | GSDLHWYQHKPGRAPKLLIHHTSSVEDGVPSR |
| | | | FSGSGFHTSFNLTISDLQADDIATYYCQVLQF |
| | | | FGRGSRLHIKGGGGSGGGGSGGGGSGGGGSGG |
| | | | GGSQGQLVQSGAELKKPGASVKISCKTSGYRF |
| | | | NFYHINWIRQTAGRGPEWMGWISPYSGDKNLA |
| | | | PAFQDRVIMTTDTEVPVTSFTSTGAAYMEIRN |
| | | | LKFDDTGTYFCAKGLLRDGSSTWLPYLWGQGT |
| | | | LLTVSSGGGGSGGGGSGGGGSQSVLTQSASVS |
| | | | GSLGQSVTISCTGPNSVCCSHKSISWYQWPPG |
| | | | RAPTLIIYEDNERAPGISPRESGYKSYWSAYL |
| | | | TISDLRPEDETTYYCCSYTHNSGCVFGTGTKV |
| | | | SVLGGGGSGGGGSGGGGSGGGGSGGGGSEVRL |
| | | | RESGGGLVKPGGSLRLSCSASGFDEDNAWMTW |
| | | | VRQPPGKGLEWVGRITGPGEGWSVDYAESVKG |
| | | | RFTISRDNTKNTLYLEMNNVRTEDTGYYFCAR |
| | | | TGKYYDFWFGYPPGEEYFQDWGQGTLVIVSSG |
| | | | GGGSGGGGSGGGGSSELTQDPAVSVALKQTVT |
| | | | ITCRGDSLRSHYASWYQKKPGQAPVLLFYGKN |
| | | | NRPSGIPDRFSGSASGNRASLTITGAQAEDEA |
| | | | DYYCSSRDKSGSRLSVFGGGTKLTVLGSGHHH |
| | | | HHH |
| 94 | PentaNAb4.0 | PGDM1400-5X- | MGWSCIILFLVATATGVHSQAQLVQSGPEVRK |
| | Stem HC | PGT121-5X-N6 | PGTSVKVSCKAPGNTLKTYDLHWVRSVPGQGL |
| | LS v2 | HC | QWMGWISHEGDKKVIVERFKAKVTIDWDRSTN |
| | (heavy | | TAYLQLSGLTSGDTAVYYCAKGSKHRLRDYAL |
| | chain) | | YDDDGALNWAVDVDYLSNLEFWGQGTAVTVSS |
| | | | GGGGSGGGGSGGGGSDFVLTQSPHSLSVTPGE |
| | | | SASISCKSSHSLIHGDRNNYLAWYVQKPGRSP |
| | | | QLLIYLASSRASGVPDRFSGSGSDKDFTLKIS |
| | | | RVETEDVGTYYCMQGRESPWTFGQGTKVDIKG |
| | | | GGGSGGGGSGGGGSGGGGSGGGGSQMQLQESG |
| | | | PGLVKPSETLSLTCSVSGASISDSYWSWIRRS |
| | | | PGKGLEWIGYVHKSGDTNYSPSLKSRVNLSLD |
| | | | TSKNQVSLSLVAATAADSGKYYCARTLHGRRI |
| | | | YGIVAFNEWFTYFYMDVWGNGTQVTVSSGGGG |
| | | | SGGGGSGGGGSSDISVAPGETARISCGEKSLG |
| | | | SRAVQWYQHRAGQAPSLIIYNNQDRPSGIPER |
| | | | FSGSPDSPFGTTATLTITSVEAGDEADYYCHI |
| | | | WDSRVPTKWVFGGGTTLTVLGGGGSGGGGSGG |
| | | | GGSGGGGSGGGGSRAHLVQSGTAMKKPGASVR |
| | | | VSCQTSGYTFTAHILFWFRQAPGRGLEWVGWI |
| | | | KPQYGAVNFGGGFRDRVTLTRDVYREIAYMDI |
| | | | RGLKPDDTAVYYCARDRSYGDSSWALDAWGQG |
| | | | TTVVVSAASTKGPSVFPLAPSSKSTSGGTAAL |
| | | | GCLVKDYFPEPVTVSWNSGALTSGVHTFPAVL |
| | | | QSSGLYSLSSVVTVPSSSLGTQTYICNVNHKP |
| | | | SNTKVDKKVEPKSCDKTHTCPPCPAPELLGGP |
| | | | SVFLFPPKPKDTLMISRTPEVTCVVVDVSHED |
| | | | PEVKFNWYVDGVEVHNAKTKPREEQYNSTYRV |
| | | | VSVLTVLHQDWLNGKEYKCKVSNKALPAPIEK |
| | | | TISKAKGQPREPQVYTLPPSREEMTKNQVSLT |
| | | | CLVKGFYPSDIAVEWESNGQPENNYKTTPPVL |
| | | | DSDGSFFLYSKLTVDKSRWQQGNVFSCSVLHE |
| | | | ALHSHYTQKSLSLSPGK |
| 96 | PentaNAb4.0 | N6 KC-CK- | MGWSCIILFLVATATGVHSYIHVTQSPSSLSV |
| | Stem HC | 35O22-5X- | SIGDRVTINCQTSQGVGSDLHWYQHKPGRAPK |
| | LS v2 | 10E8v4 S100cF | LLIHHTSSVEDGVPSRFSGSGFHTSFNLTISD |
| | (light | | LQADDIATYYCQVLQFFGRGSRLHIKRTVAAP |
| | chain) | | SVFIFPPSDEQLKSGTASVVCLLNNFYPREAK |
| | | | VQWKVDNALQSGNSQESVTEQDSKDSTYSLSS |
| | | | TLTLSKADYEKHKVYACEVTHQGLSSPVTKSF |
| | | | NRGECGGGGSGGGGSGGGGSGGGGSGGGGSQG |
| | | | QLVQSGAELKKPGASVKISCKTSGYRFNFYHI |
| | | | NWIRQTAGRGPEWMGWISPYSGDKNLAPAFQD |
| | | | RVIMTTDTEVPVTSFTSTGAAYMEIRNLKEDD |
| | | | TGTYFCAKGLLRDGSSTWLPYLWGQGTLLTVS |
| | | | SGGGGSGGGGSGGGGSQSVLTQSASVSGSLGQ |
| | | | SVTISCTGPNSVCCSHKSISWYQWPPGRAPTL |
| | | | IIYEDNERAPGISPRFSGYKSYWSAYLTISDL |
| | | | RPEDETTYYCCSYTHNSGCVFGTGTKVSVLGG |
| | | | GGSGGGGSGGGGSGGGGSGGGGSEVRLVESGG |
| | | | GLVKPGGSLRLSCSASGFDFDNAWMTWVRQPP |
| | | | GKGLEWVGRITGPGEGWSVDYAESVKGRFTIS |
| | | | RDNTKNTLYLEMNNVRTEDTGYYFCARTGKYY |
| | | | DFWFGYPPGEEYFQDWGQGTLVIVSSGGGGSG |
| | | | GGGSGGGGSSELTQDPAVSVALKQTVTITCRG |
| | | | DSLRSHYASWYQKKPGQAPVLLFYGKNNRPSG |
| | | | IPDRFSGSASGNRASLTITGAQAEDEADYYCS |
| | | | SRDKSGSRLSVFGGGTKLTVL |

PentaNAb2.0:10E8v4 S100cF-5X-35O22-5X-N6-5X-
PGT121-5X-VRC26.25-IgG3C hinge-IgG1 LS
(SEQ ID NO. 106)
ATGGGATGGAGCTGTATTATTCTGTTTCTGGTCGCTACCGCTACCGGAGTGCATTCTTCTGAACTGACCCAG
GACCCCGCCGTGAGCGTGGCCCTGAAGCAGACCGTGACAATCACCTGCAGGGGCGACAGCCTGCGCTCCCAC
TACGCCAGCTGGTATCAGAAGAAGCCTGGCCAGGCCCCAGTGCTGCTGTTCTACGGCAAGAACAATAGGCCC
TCCGGCATCCCTGATCGCTTTTCCGGCTCTGCCAGCGGAAACAGGGCCAGCCTGACAATCACCGGAGCACAG
GCAGAGGACGAGGCAGATTACTATTGCAGCTCCCGGGACAAGTCCGGCTCTAGACTGAGCGTGTTCGGCGGC
GGCACCAAGCTGACAGTGCTGGGAGGAGGAGGCAGCGGCGGAGGAGGCTCCGGAGGCGGCGGCTCTGAGGTG
CGGCTGGTGGAGTCTGGAGGAGGCCTGGTGAAGCCAGGAGGCAGCCTGAGACTGAGCTGTTCCGCCTCTGGC
TTCGACTTTGATAATGCCTGGATGACATGGGTGCGGCAGCCACCTGGCAAGGGCCTGGAGTGGGTGGGAAGA
ATCACCGGACCAGGAGAGGGATGGTCTGTGGACTACGCCGAGAGCGTGAAGGGCCGGTTCACCATCTCCAGA
GATAACACCAAGAATACACTGTATCTGGAGATGAACAATGTGCGGACCGAGGACACAGGCTACTATTTCTGC
GCCAGAACCGGCAAGTACTATGATTTTTGGTTTGGCTACCCACCCGGCGAGGAGTATTTTCAGGACTGGGGC
CAGGGCACCCTGGTCATCGTGAGCAGCGGCGGCGGCGGCAGCGGCGGCGGCGGCTCCGGAGGAGGCGGCTCT
GGAGGAGGAGGCAGCGGGGGAGGGGGCAGCCAGTCCGTGCTGACCCAGTCTGCCAGCGTGTCCGGCTCTCTG
GGACAGAGCGTGACCATCTCCTGTACAGGCCCCAACAGCGTGTGCTGTAGCCACAAGAGCATCTCCTGGTAC
CAGTGGCCTCCAGGAAGGGCACCTACCCTGATCATCTATGAGGACAATGAGCGGGCCCCAGGCATCTCCCCC
AGATTCTCTGGCTACAAGTCTTATTGGAGCGCCTACCTGACAATCAGCGACCTGCGCCCCGAGGATGAGACA
ACATACTATTGCTGTTCCTATACCCACAACTCTGGCTGCGTGTTTGGCACAGGCACCAAGGTGTCCGTGCTG
GGCGGCGGCGGCAGCGGGGGCGGGGGCTCCGGAGGGGGCGGCTCTCAGGGCCAGCTGGTGCAGAGCGGAGCA
GAGCTGAAGAAGCCTGGAGCCAGCGTGAAGATCTCCTGTAAGACATCTGGCTACCGGTTCAACTTTTATCAC
ATCAATTGGATCAGGCAGACCGCAGGAAGGGGACCAGAGTGGATGGGCTGGATCTCCCCCTACTCTGGCGAT
AAGAACCTGGCCCCAGCCTTCCAGGACAGAGTGATCATGACCACAGATACCGAGGTGCCAGTGACCAGCTTC
ACCTCCACCGGAGCCGCCTACATGGAGATCAGGAATCTGAAGTTCGACGATACAGGCACCTATTTTTGCGCA
AAGGGCCTGCTGAGGGACGGCTCCTCTACCTGGCTGCCTTACCTGTGGGGACAGGGCACCCTGCTGACAGTG
AGCTCCGGCGGCGGGGGCAGCGGCGGCGGGGGCTCCGGAGGAGGAGGCTCTGGAGGAGGGGGCAGCGGAGGA
GGCGGCTCCTACATCCACGTGACCCAGTCCCCATCTAGCCTGTCTGTGAGCATCGGCGATCGGGTGACCATC
AACTGTCAGACATCTCAGGGCGTGGGCAGCGACCTGCACTGGTATCAGCACAAGCCTGGCAGGGCCCCAAAG
CTGCTGATCCACCACACATCCTCTGTGGAGGATGGAGTGCCAAGCCGCTTCTCCGGCTCTGGATTCCACACC
TCCTTTAATCTGACAATCTCTGACCTGCAGGCCGACGATATCGCCACCTACTATTGCCAGGTGCTGCAGTTC
TTTGGCCGGGGCTCCAGACTGCACATCAAGGGAGGAGGAGGCTCCGGGGGCGGAGGCTCTGGCGGCGGCGGC
AGCCGGGCCCACCTGGTGCAGAGCGGCACCGCCATGAAGAAGCCTGGCGCCAGCGTGAGAGTGTCCTGTCAG
ACATCTGGCTACACCTTCACCGCCCACATCCTGTTCTGGTTTAGGCAGGCACCAGGAAGAGGCCTGGAGTGG
GTGGGCTGGATCAAGCCCCAGTATGGAGCAGTGAACTTCGGAGGAGGCTTTCGGGACAGAGTGACACTGACC
CGGGACGTGTACAGAGAGATCGCCTATATGGATATCAGGGGCCTGAAGCCAGACGATACCGCCGTGTACTAT
TGCGCCAGGGACCGCTCCTACGGCGATAGCTCCTGGGCACTGGACGCATGGGGACAGGGCACCACAGTGGTG
GTGAGCGCCGGCGGCGGAGGCTCCGGCGGCGGGGGCTCTGGAGGAGGCGGCAGCGGAGGGGGAGGCTCCGGA
GGGGGAGGCTCTAGCGACATCTCCGTGGCCCCTGGCGAGACAGCCAGAATCTCTTGTGGCGAGAAGTCTCTG
GGCAGCAGGGCCGTGCAGTGGTACCAGCACAGGGCAGGACAGGCACCATCTCTGATCATCTATAACAATCAG
GATAGGCCAAGCGGCATCCCTGAGCGGTTCAGCGGCTCCCCCGACAGCCCTTTTGGCACCACAGCCACACTG
ACCATCACATCCGTGGAGGCAGGCGACGAAGCCGATTACTATTGCCACATCTGGGATTCCAGAGTGCCAACC
AAGTGGGTGTTCGGAGGAGGAACCACACTGACAGTGCTGGGAGGGGGGGGCTCTGGCGGCGGGGGCAGCGGG
GGAGGAGGCTCCCAGATGCAGCTGCAGGAGAGCGGACCAGGCCTGGTGAAGCCTAGCGAGACACTGAGCCTG
ACATGTTCTGTGAGCGGCGCCTCCATCTCTGACAGCTACTGGTCTTGGATCAGACGGAGCCCCGGCAAGGGC
CTGGAATGGATCGGCTACGTGCACAAGTCCGGCGATACAAACTATTCCCCATCTCTGAAGTCTCGGGTGAAC
CTGTCTCTGGACACCAGCAAGAATCAGGTGAGCCTGTCCCTGGTGGCAGCAACCGCAGCAGATAGCGGCAAG
TACTATTGCGCCAGAACACTGCACGGCAGGCGCATCTACGGCATCGTGGCCTTTAACGAGTGGTTCACCTAC
TTTTATATGGACGTGTGGGGCAATGGCACCCAGGTGACAGTGTCCTCTGGCGGGGGCGGCTCCGGAGGCGGA
GGCTCTGGCGGGGGCGGCAGCGGCGGGGGCGGCTCCGGGGGAGGCGGCTCTCAGAGCGTGCTGACCCAGCCA
CCTTCCGTGTCTGCCGCACCAGGACAGAAGGTGACCATCAGCTGTTCCGGCAACACATCCAATATCGGCAAC
AATTTCGTGTCTTGGTACCAGCAGAGGCCAGGAAGGGCACCACAGCTGCTGATCTATGAGACAGACAAGCGG
CCTTCCGGCATCCCAGATAGATTTTCTGCCAGCAAGTCCGGCACCAGCGGCACACTGGCAATCACCGGCCTG
CAGACAGGCGACGAAGCTGATTACTATTGCGCAACCTGGGCAGCCTCCCTGAGCTCCGCCAGGGTGTTCGGA
ACCGGAACAAAAGTGATCGTGCTGGTGGGCGGCGGAGGCTCTGGCGGAGGCGGCAGCGGCGGGGGGGGCTCC
CAGGTGCAGCTGGTGGAGAGCGGCGGCGGCGTGGTGCAGCCAGGCACCTCCCTGAGGCTGTCTTGTGCAGCA
AGCCAGTTCAGATTTGATGGCTACGGCATGCACTGGGTGCGCCAGGCACCAGGCAAGGGCCTGGAGTGGGTA
GCCTCTATCAGCCACGACGGCATCAAGAAGTACCACGCCGAGAAAGTGTGGGGCAGGTTCACCATCTCCCGC
GATAACTCTAAAAACACACTGTATCTGCAGATGAACTCCCTGAGGCCCGAGGACACCGCCCTGTACTATTGC
GCCAAGGACCTGCGCGAGGATGAGTGTGAGGAGTGGTGGTCCGACTACTATGATTTTGGCAAGCAGCTGCCT
TGCGCAAAGAGCAGGGGAGGCCTGGTGGGAATCGCCGATAATTGGGGCCAGGGCACCATGGTGACAGTGTCT
AGCGGATCCGGAGAGCTGAAGACCCCTCTGGGCGATACCACACACACATCCCCACGGAGCCCCGAGCCAAAG
TCCTCTGACACCCCACCCCCTAGCCCTAGATCCCCTGAGCCAAAGAGCTCCGATACACCACCCCCTTCTCCA
AGGAGCCCCGAGCCTAAGTCTAGCGACACCCCACCCCCTTGCCCCCGCTGTCCAGCACCAGAGCTGCTGGGA
GGACCAAGCGTGTTCCTGTTTCCACCCAAGCCTAAGGATACACTGATGATCTCTCGCACCCCCGAGGTGACA
TGCGTGGTGGTGGACGTGAGCCACGAGGACCCCGAGGTGAAGTTCAACTGGTACGTGGACGGCGTGGAGGTG
CACAATGCCAAGACCAAGCCCAGGGAGGAGCAGTACAACAGCACCTATCGCGTGGTGTCCGTGCTGACAGTG
CTGCACCAGGACTGGCTGAACGGCAAGGAGTACAAGTGCAAGGTGTCCAATAAGGCCCTGCCTGCCCCAATC
GAGAAGACAATCAGCAAGGCAAAGGGACAGCCAAGGGAGCCACAGGTGTACACCCTGCCTCCAAGCCGCGAG
GAGATGACCAAGAACCAGGTGTCCCTGACATGTCTGGTGAAGGGCTTCTATCCTAGCGATATCGCCGTGGAG
TGGGAGTCCAATGGCCAGCCAGAGAACAATTACAAGACCACACCCCCTGTGCTGGACTCCGATGGCTCTTTC
TTTCTGTATTCTAAGCTGACCGTGGACAAGAGCCGGTGGCAGCAGGGCAACGTGTTCAGCTGCTCTGTGCTG
CACGAAGCCCTGCACAGCCATTACACCCAGAAGAGCCTGAGCCTGAGCCCCGGAAAA
Amino acid:
(SEQ ID NO. 107)
MGWSCIILFLVATATGVHSSELTQDPAVSVALKQTVTITCRGDSLRSHYASWYQKKPGQAPVLLFYGKNNRP
SGIPDRFSGSASGNRASLTITGAQAEDEADYYCSSRDKSGSRLSVFGGGTKLTVLGGGGSGGGGSGGGGSEV
RLVESGGGLVKPGGSLRLSCSASGFDFDNAWMTWVRQPPGKGLEWVGRITGPGEGWSVDYAESVKGRFTISR
DNTKNTLYLEMNNVRTEDTGYYFCARTGKYYDFWFGYPPGEEYFQDWGQGTLVIVSSGGGGSGGGGSGGGGS
GGGGSGGGGSQSVLTQSASVSGSLGQSVTISCTGPNSVCCSHKSISWYQWPPGRAPTLIIYEDNERAPGISP
RFSGYKSYWSAYLTISDLRPEDETTYYCCSYTHNSGCVFGTGTKVSVLGGGGSGGGGSGGGGSQGQLVQSGA
ELKKPGASVKISCKTSGYRFNFYHINWIRQTAGRGPEWMGWISPYSGDKNLAPAFQDRVIMTTDTEVPVTSF
TSTGAAYMEIRNLKFDDTGTYFCAKGLLRDGSSTWLPYLWGQGTLLTVSSGGGGSGGGGSGGGGSGGGGSGG
GGSYIHVTQSPSSLSVSIGDRVTINCQTSQGVGSDLHWYQHKPGRAPKLLIHHTSSVEDGVPSRFSGSGFHT
SFNLTISDLQADDIATYYCQVLQFFGRGSRLHIKGGGGSGGGGSGGGGSRAHLVQSGTAMKKPGASVRVSCQ
TSGYTFTAHILFWFRQAPGRGLEWVGWIKPQYGAVNFGGGFRDRVTLTRDVYREIAYMDIRGLKPDDTAVYY
CARDRSYGDSSWALDAWGQGTTVVVSAGGGGSGGGGSGGGGSGGGGSGGGGSSDISVAPGETARISCGEKSL
GSRAVQWYQHRAGQAPSLIIYNNQDRPSGIPERFSGSPDSPFGTTATLTITSVEAGDEADYYCHIWDSRVPT
KWVFGGGTTLTVLGGGGSGGGGSGGGGSQMQLQESGPGLVKPSETLSLTCSVSGASISDSYWSWIRRSPGKG
LEWIGYVHKSGDTNYSPSLKSRVNLSLDTSKNQVSLSLVAATAADSGKYYCARTLHGRRIYGIVAFNEWFTY
FYMDVWGNGTQVTVSSGGGGSGGGGSGGGGSGGGGSGGGGSQSVLTQPPSVSAAPGQKVTISCSGNTSNIGN
NFVSWYQQRPGRAPQLLIYETDKRPSGIPDRFSASKSGTSGTLAITGLQTGDEADYYCATWAASLSSARVFG
TGTKVIVLVGGGGSGGGGSGGGGSQVQLVESGGGVVQPGTSLRLSCAASQFREDGYGMHWVRQAPGKGLEWV
ASISHDGIKKYHAEKVWGRFTISRDNSKNTLYLQMNSLRPEDTALYYCAKDLREDECEEWWSDYYDFGKQLP
CAKSRGGLVGIADNWGQGTMVTVSSGSGELKTPLGDTTHTSPRSPEPKSSDTPPPSPRSPEPKSSDTPPPSP
RSPEPKSSDTPPPCPRCPAPELLGGPSVFLFPPKPKDTLMISRTPEVTCVVVDVSHEDPEVKENWYVDGVEV
HNAKTKPREEQYNSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSRWQQGNVFSCSVL
HEALHSHYTQKSLSLSPGK

The studies presented above, using prototypic HIV bNAbs to create multispecific Env binding functional moieties with enhanced breadth and/or potency through structure-based rational design and iterative optimization, as well as combinations of the optimized bispecific Abs to form multi-specific Abs will serve as a methodological foundation for the eventual expansion of this line of investigation in regard to the more imaginative anti-viral agents described in this disclosure.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

What is claimed is:

1. One or more vectors comprising a nucleic acid encoding a multispecific anti-HIV antibody that binds to multiple epitopes on HIV envelope protein, wherein the antibody comprises:
i. an amino acid sequence that binds to a V1/V2 apex glycan epitope;
ii. an amino acid sequence that binds to a V3-base glycan region epitope;
iii. an amino acid sequence that binds to a CD4 binding site (CD4bs) epitope;
iv. an amino acid sequence that binds to a gp120/gp41 interface epitope; and
v. an amino acid sequence that binds to a membrane proximal external region (MPER) epitope.

2. The one or more vectors of claim 1, wherein the multispecific anti-HIV antibody that binds to multiple epitopes on HIV envelope protein, comprises
i. amino acid sequences that bind to a V1/V2 apex glycan epitope selected from the group consisting of:
a) an amino acid sequence comprising a CDR H1, CDR H2 and CDR H3, wherein CDR H1 comprises QFRFDGYG (SEQ ID NO: 2), CDR H2 comprises ISHDGIKK (SEQ ID NO: 3) and CDR H3 comprises AKDLREDECEEWWSDDFGKQLPCAKSRGGLVGIADN (SEQ ID NO: 4); and an amino acid sequence comprising a CDR L1, CDR L2 and CDR L3, wherein CDR L1 comprises TSNIGNNF (SEQ ID NO: 6), CDR L2 comprises ETD (SEQ ID NO:7) and CDR L3 comprises ATWAASLSSARV (SEQ ID NO: 8); and
b) an amino acid sequence comprising a CDR H1, CDR H2 and CDR H3, wherein CDR H1 comprises GNTLKTYD SEQ ID NO: 10), CDR H2 comprises ISHEGDKK (SEQ ID NO: 11) and CDR H3 comprises AKGSKHRLRDYALDDDGALNWAVDVDYLSNLEF (SEQ ID NO: 12); and an amino acid sequence comprising a CDR L1, CDR L2 and CDR L3, wherein CDR L1 comprises HSLIHGDRNNY (SEQ ID NO: 14), CDR L2 comprises LAS (SEQ ID NO: 15) and CDR L3 comprises MQGRESPWT (SEQ ID NO: 16);
ii. an amino acid sequence that binds to a V3-base glycan region epitope, wherein the amino acid comprises a CDR H1, CDR H2 and CDR H3, wherein CDR H1 comprises GASISDSY (SEQ ID NO: 18), CDR H2 comprises VHKSGDT (SEQ ID NO:19) and CDR H3 comprises ARTLHGRRIYGIVAFNEWFTYFYMDV (SEQ ID NO: 20); and an amino acid sequence comprising a CDR L1, CDR L2 and CDR L3, wherein CDR L1 comprises SLGSRA (SEQ ID NO: 22), CDR L2 comprises NNQ (SEQ ID NO: 23) and CDR L3 comprises HIWDSRVPTKWV (SEQ ID NO: 24);
iii. an amino acid sequence that binds to a CD4 binding site (CD4bs) epitope wherein the amino acid comprises a CDR H1, CDR H2 and CDR H3, wherein CDR H1 comprises GYTFTAHI (SEQ ID NO: 26), CDR H2 comprises IKPQYGAV (SEQ ID NO: 27) and CDR H3 comprises AR (SEQ ID NO: 28); and an amino acid sequence comprising a CDR L1, CDR L2 and CDR L3, wherein CDR L1 comprises QGVGSD (SEQ ID NO: 30, CDR L2 comprises HTS (SEQ ID NO: 31) and CDR L3 comprises QVLQF (SEQ ID NO: 32);
iv. an amino acid sequence that binds to a gp120/gp41 interface epitope wherein the amino acid sequence comprises a CDR H1, CDR H2 and CDR H3, wherein CDR H1 comprises GYRFNFYH (SEQ ID NO: 34), CDR H2 comprises ISPYSGDK (SEQ ID NO: 35) and CDR H3 comprises DDTGTYFCAKGLLRDGSSTWLPYL (SEQ ID NO: 36); and an amino acid sequence comprising a CDR L1, CDR L2 and CDR L3, wherein CDR L1 comprises NSVCCSHKS (SEQ ID NO: 38), CDR L2 comprises EDN (SEQ ID NO: 39) and CDR L3 comprises CSYTHNSGCV (SEQ ID NO: 40); and
v. an amino acid sequence that binds to a membrane proximal external region (MPER) epitope wherein the amino acid sequence comprises a CDR H1, CDR H2 and CDR H3, wherein CDR H1 comprises GFDFDNAW (SEQ ID NO: 42, CDR H2 comprises ITGPGEGWSV (SEQ ID NO: 43) and CDR H3 comprises TGYYFCARTGKYYDFWSGYPPGEEYFQD (SEQ ID NO: 44); and an amino acid sequence comprising a CDR L1, CDR L2 and CDR L3, wherein CDR L1 comprises RGDSLRSHYAS (SEQ ID NO: 46), CDR L2 comprises GKNNRPS (SEQ ID NO: 47) and CDR L3 comprises SSRDKSGSRLSV (SEQ ID NO: 48).

3. The one or more vectors of claim 1, wherein the antibody simultaneously binds the multiple epitopes.

4. The one or more vectors of claim 1, wherein the amino acid sequences of i-v) are present on a single polypeptide chain.

5. The one or more vectors of claim 1, wherein the antibody is capable of neutralizing at least 99% of the HIV viruses or HIV pseudoviruses listed in Table 1 with an IC50 value of less than 50 μg/mL.

6. The one or more vectors of claim 2, wherein the antibody simultaneously binds the multiple epitopes.

7. The one or more vectors of claim 2, wherein the amino acid sequences of i-v) are present on a single polypeptide chain.

8. The one or more vectors of claim 2, wherein the antibody is capable of neutralizing at least 99% of the HIV viruses or HIV pseudoviruses listed in Table 1 with an IC50 value of less than 50 μg/mL.

9. The one or more vectors of claim 1, wherein the vector is a viral vector.

10. The one or more vector of claim 2, wherein the vector is a viral vector.

11. A cell, or engineered cell, comprising the one or more vectors of claim 1.

12. A cell, or engineered cell, comprising the one or more vectors of claim 2.

13. The cell, or engineered cell of claim 11, wherein the cell is an immune cell.

14. The cell, or engineered cell, of claim 13, wherein the immune cell is a B cell.

15. The cell, or engineered cell of claim 12, wherein the cell is an immune cell.

16. The cell of claim 15, wherein the immune cell is a B cell.

17. A pharmaceutical composition comprising the cell of claim 11, and a pharmaceutically acceptable carrier.

18. A pharmaceutical composition comprising the cell of claim 12, and a pharmaceutically acceptable carrier.

19. A method for treating or preventing HIV infection in a subject, comprising administering to the subject an effective amount of the composition of claim 16.

20. The method of claim 19, wherein the composition is administered in combination with another therapy, and optionally, wherein the therapy is an anti-retroviral therapy.


SEQUENCE LISTING (108 sequences)

SEQ ID NO: 1 - protein, 143 aa, synthetic construct
  REGION 1..143: note = Synthetic: VRC26.25 VH
QVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDDFGKQLPCAKSRGGLVGIADNWGQGTMVTVSS

SEQ ID NO: 2 - protein, 8 aa, synthetic construct
  REGION 1..8: note = Synthetic: VRC26.25 VH CDR1
QFRFDGYG

SEQ ID NO: 3 - protein, 8 aa, synthetic construct
  REGION 1..8: note = Synthetic: VRC26.25 VH CDR2
ISHDGIKK

SEQ ID NO: 4 - protein, 36 aa, synthetic construct
  REGION 1..36: note = Synthetic: VRC26.25 VH CDR3
AKDLREDECEEWWSDDFGKQLPCAKSRGGLVGIADN

SEQ ID NO: 5 - protein, 112 aa, synthetic construct
  REGION 1..112: note = Synthetic: VRC26.25 VL
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLV

SEQ ID NO: 6 - protein, 8 aa, synthetic construct
  REGION 1..8: note = Synthetic: VRC26.25 VL CDR1
TSNIGNNF

SEQ ID NO: 7 - skipped

SEQ ID NO: 8 - protein, 12 aa, synthetic construct
  REGION 1..12: note = Synthetic: VRC26.25 VL CDR3
ATWAASLSSARV

SEQ ID NO: 9 - protein, 140 aa, synthetic construct
  REGION 1..140: note = Synthetic: PGDM1400 VH
QAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALDDDGALNWAVDVDYLSNLEFWGQGTAVTVSS

SEQ ID NO: 10 - protein, 8 aa, synthetic construct
  REGION 1..8: note = Synthetic: PGDM1400 VH CDR1
GNTLKTYD

SEQ ID NO: 11 - protein, 8 aa, synthetic construct
  REGION 1..8: note = Synthetic: PGDM1400 VH CDR2
ISHEGDKK

SEQ ID NO: 12 - protein, 33 aa, synthetic construct
  REGION 1..33: note = Synthetic: PGDM1400 VH CDR3
AKGSKHRLRDYALDDDGALNWAVDVDYLSNLEF

SEQ ID NO: 13 - protein, 112 aa, synthetic construct
  REGION 1..112: note = Synthetic: PGDM1400 VL
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIK

SEQ ID NO: 14 - protein, 11 aa, synthetic construct
  REGION 1..11: note = Synthetic: PGDM1400 VL CDR1
HSLIHGDRNNY

SEQ ID NO: 15 - skipped

SEQ ID NO: 16 - protein, 9 aa, synthetic construct
  REGION 1..9: note = Synthetic: PGDM1400 VL CDR3
MQGRESPWT

SEQ ID NO: 17 - protein, 132 aa, synthetic construct
  REGION 1..132: note = Synthetic: PGT121 VH
QMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 18 - protein, 8 aa, synthetic construct
  REGION 1..8: note = Synthetic: PGT121 VH CDR1
GASISDSY

SEQ ID NO: 19 - protein, 7 aa, synthetic construct
  REGION 1..7: note = Synthetic: PGT121 VH CDR2
VHKSGDT

SEQ ID NO: 20 - protein, 26 aa, synthetic construct
  REGION 1..26: note = Synthetic: PGT121 VH CDR3
ARTLHGRRIYGIVAFNEWFTYFYMDV

SEQ ID NO: 21 - protein, 105 aa, synthetic construct
  REGION 1..105: note = Synthetic: PGT121 VL
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 22 - protein, 6 aa, synthetic construct
  REGION 1..6: note = Synthetic: PGT121 VL CDR1
SLGSRA

SEQ ID NO: 23 - skipped

SEQ ID NO: 24 - protein, 12 aa, synthetic construct
  REGION 1..12: note = Synthetic: PGT121 VL CDR3
HIWDSRVPTKWV

SEQ ID NO: 25 - protein, 122 aa, synthetic construct [sequence omitted]

SEQ ID NO: 26 - protein, 8 aa, synthetic construct
  REGION 1..8: note = Synthetic: N6 VH CDR1
GYTFTAHI

SEQ ID NO: 27 - protein, 8 aa, synthetic construct
  REGION 1..8: note = Synthetic: N6 VH CDR2
IKPQYGAV

SEQ ID NO: 28 - skipped

SEQ ID NO: 29 - protein, 103 aa, synthetic construct [sequence omitted]

SEQ ID NO: 30 - protein, 6 aa, synthetic construct
  REGION 1..6: note = Synthetic: N6 VL CDR1
QGVGSD

SEQ ID NO: 31 - skipped

SEQ ID NO: 32 - protein, 5 aa, synthetic construct
  REGION 1..5: note = Synthetic: N6 VL CDR3
QVLQF

SEQ ID NO: 33 - protein, 131 aa, synthetic construct
  REGION 1..131: note = Synthetic: 35O22 VH
QGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSS

SEQ ID NO: 34 - protein, 8 aa, synthetic construct
  REGION 1..8: note = Synthetic: 35O22 VH CDR1
GYRFNFYH

SEQ ID NO: 35 - protein, 8 aa, synthetic construct
  REGION 1..8: note = Synthetic: 35O22 VH CDR2
ISPYSGDK

SEQ ID NO: 36 - protein, 24 aa, synthetic construct
  REGION 1..24: note = Synthetic: 35O22 VH CDR3
DDTGTYFCAKGLLRDGSSTWLPYL

SEQ ID NO: 37 - protein, 110 aa, synthetic construct
  REGION 1..110: note = Synthetic: 35O22 VL
QSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVL

SEQ ID NO: 38 - protein, 9 aa, synthetic construct
  REGION 1..9: note = Synthetic: 35O22 VL CDR1
NSVCCSHKS

SEQ ID NO: 39 - skipped

SEQ ID NO: 40 - protein, 10 aa, synthetic construct
  REGION 1..10: note = Synthetic: 35O22 VL CDR3
CSYTHNSGCV

SEQ ID NO: 41 - protein, 131 aa, synthetic construct
  REGION 1..131: note = Synthetic: 10E8v4 VH
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSS

SEQ ID NO: 42 - protein, 8 aa, synthetic construct
  REGION 1..8: note = Synthetic: 10E8v4 VH CDR1
GFDFDNAW

SEQ ID NO: 43 - protein, 10 aa, synthetic construct
  REGION 1..10: note = Synthetic: 10E8v4 VH CDR2
ITGPGEGWSV

SEQ ID NO: 44 - protein, 28 aa, synthetic construct
  REGION 1..28: note = Synthetic: 10E8v4 VH CDR3
TGYYFCARTGKYYDFWSGYPPGEEYFQD

SEQ ID NO: 45 - protein, 108 aa, synthetic construct
  REGION 1..108: note = Synthetic: 10E8v4 VL
SELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 46 - protein, 11 aa, synthetic construct
  REGION 1..11: note = Synthetic: 10E8v4 VL CDR1
RGDSLRSHYAS

SEQ ID NO: 47 - protein, 7 aa, synthetic construct
  REGION 1..7: note = Synthetic: 10E8v4 VL CDR2
GKNNRPS

SEQ ID NO: 48 - protein, 12 aa, synthetic construct
  REGION 1..12: note = Synthetic: 10E8v4 VL CDR3
SSRDKSGSRLSV

SEQ ID NO: 49 - protein, 131 aa, synthetic construct
  REGION 1..131: note = Synthetic: 10E8v4_S100cF VH
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSS

SEQ ID NO: 50 - protein, 28 aa, synthetic construct
  REGION 1..28: note = Synthetic: 10E8v4_S100cF VH CDR3
TGYYFCARTGKYYDFWFGYPPGEEYFQD

SEQ ID NO: 51 - protein, 131 aa, synthetic construct
  REGION 1..131: note = Synthetic: 10E8v4_V5R_S100cF VH
EVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSS

SEQ ID NO: 52 - DNA, 429 bp, synthetic construct
  misc_feature 1..429: note = Synthetic: VRC26.25 VH (Original Seq)
  source 1..429: mol_type = other DNA
caggtgcagttggtggagtctgggggaggcgtggtccagcctgggacgtccctgagactctcctgtgcagcctctcaattcaggtttgatggttatggcatgcactgggtccgccaggccccaggcaaggggctggagtgggtggcatctatatcacatgatggaattaaaaagtatcacgcagaaaaagtgtggggccgcttcaccatctccagagacaattccaagaacacactgtatctacaaatgaacagcctgcgacctgaggacacggctctctactactgtgcgaaagatttgcgagaagacgaatgtgaagagtggtggtcggatgattttgggaaacaactcccttgcgcaaagtcacgcggcggcttggttggaattgctgataactggggccaagggacaatggtcaccgtctcttca

SEQ ID NO: 53 - DNA, 333 bp, synthetic construct
  misc_feature 1..333: note = Synthetic: VRC26.25 VL (Original Seq)
  source 1..333: mol_type = other DNA
cagtctgtgctgacgcagccgccctcagtgtctgcggccccaggacagaaggtcaccatctcctgctctggaaacacctccaacattggcaataattttgtgtcctggtaccaacagcgccccggcagagccccccaactcctcatttatgaaactgacaagcgaccctcagggattcctgaccgattctctgcttccaagtctggtacgtcaggcaccctggccatcaccgggctgcagactggggacgaggccgattattactgcgccacatgggctgccagcctgagttccgcgcgtgtcttcggaactgggaccaaggtcatcgtccta

SEQ ID NO: 54 - DNA, 393 bp, synthetic construct
  misc_feature 1..393: note = Synthetic: 35O22 VH (Original Seq)
  source 1..393: mol_type = other DNA
cagggtcaactagtccagtctggagctgaattgaaaaagcctggcgcctcggtgaagatttcctgtaagacttcgggttataggtttaatttctatcatattaattggattcgacaaactgcaggacgtggacctgagtggatgggatggatcagcccttacagtggtgacaaaaacctcgcacctgcctttcaagacagagtcattatgacgacagacacagaagtccctgtgacctcattcacgtccacgggcgcagcctacatggaaataaggaacctgaaatttgacgacacaggcacctatttctgtgcaaaaggcctcctgcgtgacggttcgtcgacgtggcttccttatttgtggggccagggtaccctactcaccgtctcgtca

SEQ ID NO: 55 - DNA, 330 bp, synthetic construct
  misc_feature 1..330: note = Synthetic: 35O22 VL (Original Seq)
  source 1..330: mol_type = other DNA
cagtctgtgctgacgcagtctgcctccgtgtctgggtctcttggacagtcggtcaccatctcctgcactggacccaatagtgtttgttgcagtcacaaatctatctcctggtatcagtggcccccaggcagagcccccacactcatcatttatgaggacaatgaaagggccccgggaatctctcctcgcttctctggctacaagtcgtattggtcggcctacttgacaatctctgatctccggcctgaagacgagaccacttactactgttgctcatacactcacaatagcggctgtgtcttcgggactgggaccaaggtctccgtcttg

SEQ ID NO: 56 - DNA, 393 bp, synthetic construct
  misc_feature 1..393: note = Synthetic: 10E8v4 VH (Original Seq)
  source 1..393: mol_type = other DNA
gaggttagactggtggagtcaggaggggggcttgtgaagcccggtgggtctctccgcctgagctgttctgcctccggctttgatttcgataacgcctggatgacctgggtcaggcagcctccaggtaagggactggagtgggtgggaagaatcacaggtccaggcgagggctggtccgtggactacgcggaatctgttaaagggcggtttacaatctcaagggacaataccaagaataccttgtatttggagatgaacaacgtgagaactgaagacaccggatattacttctgtgccagaacaggcaaatactacgacttctggtccggctatccccctggcgaggaatattttcaagactggggtcagggaacccttgttatcgtgtcctcc

SEQ ID NO: 57 - DNA, 324 bp, synthetic construct
  misc_feature 1..324: note = Synthetic: 10E8v4 VL (Original Seq)
  source 1..324: mol_type = other DNA
tccgaactgactcaggaccctgccgtctctgtggcactgaagcagactgtgactattacttgccgaggcgactcactgcggagccactacgcttcctggtatcagaagaaacccggccaggcacctgtgctgctgttctacggaaagaacaataggccatctggcatccccgaccgcttttctggcagtgcatcagggaaccgagccagtctgaccattaccggcgcccaggctgaggacgaagccgattactattgcagctcccgggataagagcggctccagactgagcgtgttcggaggaggaactaaactgaccgtcctc

SEQ ID NO: 58 - DNA, 429 bp, synthetic construct
  misc_feature 1..429: note = Synthetic: VRC26.25 VH (Optimized Seq 1)
  source 1..429: mol_type = other DNA
caggtgcagctggtggagagcggcggcggcgtggtgcagccaggcacctccctgaggctgtcttgtgcagcaagccagttcagatttgatggctacggcatgcactgggtgcgccaggcaccaggcaagggcctggagtgggtagcctctatcagccacgacggcatcaagaagtaccacgccgagaaagtgtggggcaggttcaccatctcccgcgataactctaaaaacacactgtatctgcagatgaactccctgaggcccgaggacaccgccctgtactattgcgccaaggacctgcgcgaggatgagtgtgaggagtggtggtccgacgattttggcaagcagctgccttgcgcaaagagcaggggaggcctggtgggaatcgccgataattggggccagggcaccatggtgacagtgtctagc

SEQ ID NO: 59 - DNA, 429 bp, synthetic construct
  misc_feature 1..429: note = Synthetic: VRC26.25 VH (Optimized Seq 2)
  source 1..429: mol_type = other DNA
caggtgcagctggtggagtccggaggaggagtggtgcagccaggcacctctctgaggctgagctgtgcagcatcccagttcagatttgatggctacggaatgcactgggtgaggcaggcaccaggcaagggactggagtgggtggccagcatctcccacgacggcatcaagaagtaccacgccgagaaagtgtggggcaggttcaccatctctcgcgataacagcaagaatacactgtatctgcagatgaacagcctgaggcccgaggacaccgccctgtactattgcgccaaggacctgcgcgaggatgagtgtgaggagtggtggtccgacgattttggcaagcagctgccttgcgcaaagagcaggggaggactggtgggaatcgccgacaattggggccagggcaccatggtgacagtgagcagc

SEQ ID NO: 60 - DNA, 336 bp, synthetic construct
  misc_feature 1..336: note = Synthetic: VRC26.25 VL (Optimized Seq 1)
  source 1..336: mol_type = other DNA
cagagcgtgctgacccagccaccttccgtgtctgccgcaccaggacagaaggtgaccatcagctgttccggcaacacatccaatatcggcaacaatttcgtgtcttggtaccagcagaggccaggaagggcaccacagctgctgatctatgagacagacaagcggccttccggcatcccagatagattttctgccagcaagtccggcaccagcggcacactggcaatcaccggcctgcagacaggcgacgaagctgattactattgcgcaacctgggcagcctccctgagctccgccagggtgttcggaaccggaacaaaagtgatcgtgctggtg

SEQ ID NO: 61 - DNA, 336 bp, synthetic construct
  misc_feature 1..336: note = Synthetic: VRC26.25 VL (Optimized Seq 2)
  source 1..336: mol_type = other DNA
cagagcgtgctgacccagccaccttccgtgtctgccgcaccaggacagaaggtgaccatcagctgttccggcaacacatccaatatcggcaacaatttcgtgtcttggtaccagcagaggcctggaagagcaccacagctgctgatctatgagacagacaagaggccctccggcatccctgatcgcttttctgccagcaagtccggcaccagcggcacactggcaatcaccggactgcagacaggcgacgaggcagattactattgcgcaacctgggcagcctccctgtctagcgccagggtgttcggcaccggcacaaaagtgatcgtgctggtg

SEQ ID NO: 62 - DNA, 420 bp, synthetic construct
  misc_feature 1..420: note = Synthetic: PGDM1400 VH (Optimized Seq 1)
  source 1..420: mol_type = other DNA
caggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctggacgatgacggcgccctgaactgggccgtggatgtggactatctgtccaatctggagttctggggacagggaaccgcagtgacagtgagctcc

SEQ ID NO: 63 - DNA, 420 bp, synthetic construct
  misc_feature 1..420: note = Synthetic: PGDM1400 VH (Optimized Seq 2)
  source 1..420: mol_type = other DNA
caggcacagctggtgcagagcggaccagaggtgaggaagccaggcacctctgtgaaggtgagctgtaaggcccctggcaacaccctgaagacatacgatctgcactgggtgcggtctgtgccaggacagggcctgcagtggatgggatggatcagccacgagggcgacaagaaagtgatcgtggagcggtttaaggccaaggtgacaatcgattgggacagaagcaccaatacagcctatctgcagctgtccggcctgacctctggcgatacagccgtgtactattgcgccaagggctccaagcaccggctgagagactacgccctggacgatgacggcgccctgaattgggcagtggacgtggactatctgagtaatctggagttttgggggcagggcaccgcagtgacagtgtctagc

SEQ ID NO: 64 - DNA, 336 bp, synthetic construct
  misc_feature 1..336: note = Synthetic: PGDM1400 VL (Optimized Seq 1)
  source 1..336: mol_type = other DNA
gattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagtcgacatcaaa

SEQ ID NO: 65 - DNA, 336 bp, synthetic construct
  misc_feature 1..336: note = Synthetic: PGDM1400 VL (Optimized Seq 2)
  source 1..336: mol_type = other DNA
gatttcgtgctgacccagtctccacatagtctgagcgtgacacccggcgaaagcgcatcaatttcttgtaaatcatctcatagtctgatccacggcgataggaacaattacctggcctggtacgtgcagaagccaggccgcagccctcagctgctgatctacctggcaagctccagggcatccggagtgccagatcgcttctctggcagcggctccgataaggactttaccctgaagatctcccgggtggagacagaggacgtgggcacatactattgcatgcagggcagagagtctccttggaccttcggccagggcacaaaggtggacatcaag

SEQ ID NO: 66 - DNA, 393 bp, synthetic construct
  misc_feature 1..393: note = Synthetic: 35O22 VH (Optimized Seq 1)
  source 1..393: mol_type = other DNA
cagggccagctggtgcagagcggagcagagctgaagaagcctggagccagcgtgaagatctcctgtaagacatctggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctcc

SEQ ID NO: 67 - DNA, 393 bp, synthetic construct
  misc_feature 1..393: note = Synthetic: 35O22 VH (Optimized Seq 2)
  source 1..393: mol_type = other DNA
cagggccagctggtgcagagcggagcagagctgaagaagccaggagcctctgtgaagatcagctgtaagacatccggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctct

SEQ ID NO: 68 - DNA, 330 bp, synthetic construct
  misc_feature 1..330: note = Synthetic: 35O22 VL (Optimized Seq 1)
  source 1..330: mol_type = other DNA
cagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctg

SEQ ID NO: 69 - DNA, 330 bp, synthetic construct
  misc_feature 1..330: note = Synthetic: 35O22 VL (Optimized Seq 2)
  source 1..330: mol_type = other DNA
caaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctg

SEQ ID NO: 70 - DNA, 393 bp, synthetic construct
  misc_feature 1..393: note = Synthetic: 10E8v4_V5R_S100cF VH (Optimized Seq)
  source 1..393: mol_type = other DNA
gaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagc

SEQ ID NO: 71 - DNA, 324 bp, synthetic construct
  misc_feature 1..324: note = Synthetic: 10E8v4 VL
  source 1..324: mol_type = other DNA
agcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctg

SEQ ID NO: 72 - protein, 1402 aa, synthetic construct
  REGION 1..1402: note = Synthetic: PentaNAb1.0 10E8v4-5X-35O22-5X-N6-5X-PGT121-5X-VRC26.25-His
MGWSCIILFLVATATGVHSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSGGGGSGGGGSQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLVGGGGSGGGGSGGGGSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSGSGHHHHHH

SEQ ID NO: 73 - protein, 1402 aa, synthetic construct
  REGION 1..1402: note = Synthetic: PentaNAb2.0 10E8v4_S100cF-5X-35O22-5X-N6-5X-PGT121-5X-VRC26.25-His
MGWSCIILFLVATATGVHSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSGGGGSGGGGSQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLVGGGGSGGGGSGGGGSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSGSGHHHHHH

SEQ ID NO: 74 - protein, 1402 aa, synthetic construct
  REGION 1..1402: note = Synthetic: PentaNAb2.0 Reverse VRC26.25-5X-PGT121-5X-N6-5X-35O22-5X-10E8v4_S100cF-His
MGWSCIILFLVATATGVHSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSGGGGSGGGGSGGGGSQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLVGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGSGHHHHHH

SEQ ID NO: 75 - protein, 1398 aa, synthetic construct
  REGION 1..1398: note = Synthetic: PentaNAb3.0 10E8v4_S100cF-5X-35O22-5X-N6-5X-PGT121-5X-PGDM1400-His
MGWSCIILFLVATATGVHSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGSGHHHHHH

SEQ ID NO: 76 - protein, 1398 aa, synthetic construct
  REGION 1..1398: note = Synthetic: PentaNAb3.0 Reverse PGDM1400-5X-PGT121-5X-N6-5X-35O22-5X-10E8v4S100cF-His
MGWSCIILFLVATATGVHSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGSGHHHHHH

SEQ ID NO: 77 - protein, 1398 aa, synthetic construct
  REGION 1..1398: note = Synthetic: PentaNAb4.0 10E8v4 V5R_S100cF-5X-35O22-5X-N6-5X-PGT121-5X-PGDM1400-His
MGWSCIILFLVATATGVHSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGGGSGGGGSEVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGSGHHHHHH

SEQ ID NO: 78 - protein, 1398 aa, synthetic construct
  REGION 1..1398: note = Synthetic: PentaNAb4.0 Reverse PGDM1400-5X-PGT121-5X-N6-5X-35O22-5X-10E8v4 V5R S100cF-His
MGWSCIILFLVATATGVHSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGSGHHHHHH

SEQ ID NO: 79 - protein, 1605 aa, synthetic construct
  REGION 1..1605: note = Synthetic: PentaNAb4.0 Reverse PGDM1400-5X-PGT121-5X-N6-5X-35O22-5X-10E8v4 V5R S100cF VH fused to IgG1 LS HC with His Tag
MGWSCIILFLVATATGVHSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGSGHHHHHH

SEQ ID NO: 80 - protein, 1632 aa, synthetic construct
  REGION 1..1632: note = Synthetic: PentaNAb4.0 Reverse PGDM1400-5X-PGT121-5X-N6-5X-35O22-5X-10E8v4 V5R S100cF VH-VL-fused to IgG1 LS FC with His Tag
MGWSCIILFLVATATGVHSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGSGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGKGSGHHHHHH

SEQ ID NO: 81 - DNA, 4233 bp, synthetic construct
  misc_feature 1..4233: note = Synthetic: PentaNAb1.0 10E8v4-5X-35O22-5X-N6-5X-PGT121-5X-VRC26.25-His
  source 1..4233: mol_type = other DNA
gaattcgccgccaccatgggatggagctgtattattctgtttctggtcgctaccgctaccggagtgcattcttctgaactgacccaggaccccgccgtgagcgtggccctgaagcagaccgtgacaatcacctgcaggggcgacagcctgcgctcccactacgccagctggtatcagaagaagcctggccaggccccagtgctgctgttctacggcaagaacaataggccctccggcatccctgatcgcttttccggctctgccagcggaaacagggccagcctgacaatcaccggagcacaggcagaggacgaggcagattactattgcagctcccgggacaagtccggctctagactgagcgtgttcggcggcggcaccaagctgacagtgctgggaggaggaggcagcggcggaggaggctccggaggcggcggctctgaggtgcggctggtggagtctggaggaggcctggtgaagccaggaggcagcctgagactgagctgttccgcctctggcttcgactttgataatgcctggatgacatgggtgcggcagccacctggcaagggcctggagtgggtgggaagaatcaccggaccaggagagggatggtctgtggactacgccgagagcgtgaagggccggttcaccatctccagagataacaccaagaatacactgtatctggagatgaacaatgtgcggaccgaggacacaggctactatttctgcgccagaaccggcaagtactatgatttttggagcggctacccacccggcgaggagtattttcaggactggggccagggcaccctggtcatcgtgagcagcggcggcggcggcagcggcggcggcggctccggaggaggcggctctggaggaggaggcagcgggggagggggcagccagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctgggcggcggcggcagcgggggcgggggctccggagggggcggctctcagggccagctggtgcagagcggagcagagctgaagaagcctggagccagcgtgaagatctcctgtaagacatctggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctccggcggcgggggcagcggcggcgggggctccggaggaggaggctctggaggagggggcagcggaggaggcggctcctacatccacgtgacccagtccccatctagcctgtctgtgagcatcggcgatcgggtgaccatcaactgtcagacatctcagggcgtgggcagcgacctgcactggtatcagcacaagcctggcagggccccaaagctgctgatccaccacacatcctctgtggaggatggagtgccaagccgcttctccggctctggattccacacctcctttaatctgacaatctctgacctgcaggccgacgatatcgccacctactattgccaggtgctgcagttctttggccggggctccagactgcacatcaagggaggaggaggctccgggggcggaggctctggcggcggcggcagccgggcccacctggtgcagagcggcaccgccatgaagaagcctggcgccagcgtgagagtgtcctgtcagacatctggctacaccttcaccgcccacatcctgttctggtttaggcaggcaccaggaagaggcctggagtgggtgggctggatcaagccccagtatggagcagtgaacttcggaggaggctttcgggacagagtgacactgacccgggacgtgtacagagagatcgcctatatggatatcaggggcctgaagccagacgataccgccgtgtactattgcgccagggaccgctcctacggcgatagctcctgggcactggacgcatggggacagggcaccacagtggtggtgagcgccggcggcggaggctccggcggcgggggctctggaggaggcggcagcggagggggaggctccggagggggaggctctagcgacatctccgtggcccctggcgagacagccagaatctcttgtggcgagaagtctctgggcagcagggccgtgcagtggtaccagcacagggcaggacaggcaccatctctgatcatctataacaatcaggataggccaagcggcatccctgagcggttcagcggctcccccgacagcccttttggcaccacagccacactgaccatcacatccgtggaggcaggcgacgaagccgattactattgccacatctgggattccagagtgccaaccaagtgggtgttcggaggaggaaccacactgacagtgctgggaggggggggctctggcggcgggggcagcgggggaggaggctcccagatgcagctgcaggagagcggaccaggcctggtgaagcctagcgagacactgagcctgacatgttctgtgagcggcgcctccatctctgacagctactggtcttggatcagacggagccccggcaagggcctggaatggatcggctacgtgcacaagtccggcgatacaaactattccccatctctgaagtctcgggtgaacctgtctctggacaccagcaagaatcaggtgagcctgtccctggtggcagcaaccgcagcagatagcggcaagtactattgcgccagaacactgcacggcaggcgcatctacggcatcgtggcctttaacgagtggttcacctacttttatatggacgtgtggggcaatggcacccaggtgacagtgtcctctggcgggggcggctccggaggcggaggctctggcgggggcggcagcggcgggggcggctccgggggaggcggctctcagagcgtgctgacccagccaccttccgtgtctgccgcaccaggacagaaggtgaccatcagctgttccggcaacacatccaatatcggcaacaatttcgtgtcttggtaccagcagaggccaggaagggcaccacagctgctgatctatgagacagacaagcggccttccggcatcccagatagattttctgccagcaagtccggcaccagcggcacactggcaatcaccggcctgcagacaggcgacgaagctgattactattgcgcaacctgggcagcctccctgagctccgccagggtgttcggaaccggaacaaaagtgatcgtgctggtgggcggcggaggctctggcggaggcggcagcggcggggggggctcccaggtgcagctggtggagagcggcggcggcgtggtgcagccaggcacctccctgaggctgtcttgtgcagcaagccagttcagatttgatggctacggcatgcactgggtgcgccaggcaccaggcaagggcctggagtgggtagcctctatcagccacgacggcatcaagaagtaccacgccgagaaagtgtggggcaggttcaccatctcccgcgataactctaaaaacacactgtatctgcagatgaactccctgaggcccgaggacaccgccctgtactattgcgccaaggacctgcgcgaggatgagtgtgaggagtggtggtccgactactatgattttggcaagcagctgccttgcgcaaagagcaggggaggcctggtgggaatcgccgataattggggccagggcaccatggtgacagtgtctagcggatccggacaccaccatcaccatcattagtgaaagctt

SEQ ID NO: 82 - DNA, 5052 bp, synthetic construct
  misc_feature 1..5052: note = Synthetic: PentaNAb2.0 10E8v4_S100cF-5X-35O22-5X-N6-5X-PGT121-5X-VRC26.25-His
  source 1..5052: mol_type = other DNA
gaattcgccgccaccatgggatggagctgtattattctgtttctggtcgctaccgctaccggagtgcattcttctgaactgacccaggaccccgccgtgagcgtggccctgaagcagaccgtgacaatcacctgcaggggcgacagcctgcgctcccactacgccagctggtatcagaagaagcctggccaggccccagtgctgctgttctacggcaagaacaataggccctccggcatccctgatcgcttttccggctctgccagcggaaacagggccagcctgacaatcaccggagcacaggcagaggacgaggcagattactattgcagctcccgggacaagtccggctctagactgagcgtgttcggcggcggcaccaagctgacagtgctgggaggaggaggcagcggcggaggaggctccggaggcggcggctctgaggtgcggctggtggagtctggaggaggcctggtgaagccaggaggcagcctgagactgagctgttccgcctctggcttcgactttgataatgcctggatgacatgggtgcggcagccacctggcaagggcctggagtgggtgggaagaatcaccggaccaggagagggatggtctgtggactacgccgagagcgtgaagggccggttcaccatctccagagataacaccaagaatacactgtatctggagatgaacaatgtgcggaccgaggacacaggctactatttctgcgccagaaccggcaagtactatgatttttggtttggctacccacccggcgaggagtattttcaggactggggccagggcaccctggtcatcgtgagcagcggcggcggcggcagcggcggcggcggctccggaggaggcggctctggaggaggaggcagcgggggagggggcagccagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctgggcggcggcggcagcgggggcgggggctccggagggggcggctctcagggccagctggtgcagagcggagcagagctgaagaagcctggagccagcgtgaagatctcctgtaagacatctggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctccggcggcgggggcagcggcggcgggggctccggaggaggaggctctggaggagggggcagcggaggaggcggctcctacatccacgtgacccagtccccatctagcctgtctgtgagcatcggcgatcgggtgaccatcaactgtcagacatctcagggcgtgggcagcgacctgcactggtatcagcacaagcctggcagggccccaaagctgctgatccaccacacatcctctgtggaggatggagtgccaagccgcttctccggctctggattccacacctcctttaatctgacaatctctgacctgcaggccgacgatatcgccacctactattgccaggtgctgcagttctttggccggggctccagactgcacatcaagggaggaggaggctccgggggcggaggctctggcggcggcggcagccgggcccacctggtgcagagcggcaccgccatgaagaagcctggcgccagcgtgagagtgtcctgtcagacatctggctacaccttcaccgcccacatcctgttctggtttaggcaggcaccaggaagaggcctggagtgggtgggctggatcaagccccagtatggagcagtgaacttcggaggaggctttcgggacagagtgacactgacccgggacgtgtacagagagatcgcctatatggatatcaggggcctgaagccagacgataccgccgtgtactattgcgccagggaccgctcctacggcgatagctcctgggcactggacgcatggggacagggcaccacagtggtggtgagcgccggcggcggaggctccggcggcgggggctctggaggaggcggcagcggagggggaggctccggagggggaggctctagcgacatctccgtggcccctggcgagacagccagaatctcttgtggcgagaagtctctgggcagcagggccgtgcagtggtaccagcacagggcaggacaggcaccatctctgatcatctataacaatcaggataggccaagcggcatccctgagcggttcagcggctcccccgacagcccttttggcaccacagccacactgaccatcacatccgtggaggcaggcgacgaagccgattactattgccacatctgggattccagagtgccaaccaagtgggtgttcggaggaggaaccacactgacagtgctgggaggggggggctctggcggcgggggcagcgggggaggaggctcccagatgcagctgcaggagagcggaccaggcctggtgaagcctagcgagacactgagcctgacatgttctgtgagcggcgcctccatctctgacagctactggtcttggatcagacggagccccggcaagggcctggaatggatcggctacgtgcacaagtccggcgatacaaactattccccatctctgaagtctcgggtgaacctgtctctggacaccagcaagaatcaggtgagcctgtccctggtggcagcaaccgcagcagatagcggcaagtactattgcgccagaacactgcacggcaggcgcatctacggcatcgtggcctttaacgagtggttcacctacttttatatggacgtgtggggcaatggcacccaggtgacagtgtcctctggcgggggcggctccggaggcggaggctctggcgggggcggcagcggcgggggcggctccgggggaggcggctctcagagcgtgctgacccagccaccttccgtgtctgccgcaccaggacagaaggtgaccatcagctgttccggcaacacatccaatatcggcaacaatttcgtgtcttggtaccagcagaggccaggaagggcaccacagctgctgatctatgagacagacaagcggccttccggcatcccagatagattttctgccagcaagtccggcaccagcggcacactggcaatcaccggcctgcagacaggcgacgaagctgattactattgcgcaacctgggcagcctccctgagctccgccagggtgttcggaaccggaacaaaagtgatcgtgctggtgggcggcggaggctctggcggaggcggcagcggcggggggggctcccaggtgcagctggtggagagcggcggcggcgtggtgcagccaggcacctccctgaggctgtcttgtgcagcaagccagttcagatttgatggctacggcatgcactgggtgcgccaggcaccaggcaagggcctggagtgggtagcctctatcagccacgacggcatcaagaagtaccacgccgagaaagtgtggggcaggttcaccatctcccgcgataactctaaaaacacactgtatctgcagatgaactccctgaggcccgaggacaccgccctgtactattgcgccaaggacctgcgcgaggatgagtgtgaggagtggtggtccgactactatgattttggcaagcagctgccttgcgcaaagagcaggggaggcctggtgggaatcgccgataattggggccagggcaccatggtgacagtgtctagcggatccggagagctgaagacccctctgggcgataccacacacacatccccacggagccccgagccaaagtcctctgacaccccaccccctagccctagatcccctgagccaaagagctccgatacaccacccccttctccaaggagccccgagcctaagtctagcgacaccccacccccttgcccccgctgtccagcaccagagctgctgggaggaccaagcgtgttcctgtttccacccaagcctaaggatacactgatgatctctcgcacccccgaggtgacatgcgtggtggtggacgtgagccacgaggaccccgaggtgaagttcaactggtacgtggacggcgtggaggtgcacaatgccaagaccaagcccagggaggagcagtacaacagcacctatcgcgtggtgtccgtgctgacagtgctgcaccaggactggctgaacggcaaggagtacaagtgcaaggtgtccaataaggccctgcctgccccaatcgagaagacaatcagcaaggcaaagggacagccaagggagccacaggtgtacaccctgcctccaagccgcgaggagatgaccaagaaccaggtgtccctgacatgtctggtgaagggcttctatcctagcgatatcgccgtggagtgggagtccaatggccagccagagaacaattacaagaccacaccccctgtgctggactccgatggctctttctttctgtattctaagctgaccgtggacaagagccggtggcagcagggcaacgtgttcagctgctctgtgctgcacgaagccctgcacagccattacacccagaagagcctgagcctgagccccggaaaatagtgaaagctt

SEQ ID NO: 83 - DNA, 4232 bp, synthetic construct
  misc_feature 1..4232: note = Synthetic: PentaNAb2.0 Reverse VRC26.25-5X-PGT121-5X-N6-5X-35O22-5X-10E8v4_S100cF-His
  source 1..4232: mol_type = other DNA
gaattcgccgccaccatgggctggagctgcatcatcctgttcctggtggcaaccgcaacaggagtgcacagccaggtgcagctggtggagtccggaggaggagtggtgcagccaggcacctctctgaggctgagctgtgcagcatcccagttcagatttgatggctacggaatgcactgggtgaggcaggcaccaggcaagggactggagtgggtggccagcatctcccacgacggcatcaagaagtaccacgccgagaaagtgtggggcaggttcaccatctctcgcgataacagcaagaatacactgtatctgcagatgaacagcctgaggcccgaggacaccgccctgtactattgcgccaaggacctgcgcgaggatgagtgtgaggagtggtggtccgactactatgattttggcaagcagctgccttgcgcaaagagcaggggaggactggtgggaatcgccgacaattggggccagggcaccatggtgacagtgagcagcggaggaggaggctctggaggaggaggcagcggaggcggcggctctcagagcgtgctgacccagccaccttccgtgtctgccgcaccaggacagaaggtgaccatcagctgttccggcaacacatccaatatcggcaacaatttcgtgtcttggtaccagcagaggcctggaagagcaccacagctgctgatctatgagacagacaagaggccctccggcatccctgatcgcttttctgccagcaagtccggcaccagcggcacactggcaatcaccggactgcagacaggcgacgaggcagattactattgcgcaacctgggcagcctccctgtctagcgccagggtgttcggcaccggcacaaaagtgatcgtgctggtgggaggaggaggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctggtggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatccggccaccaccatcaccatcattagtgaagctt

SEQ ID NO: 84 - DNA, 4221 bp, synthetic construct
  misc_feature 1..4221: note = Synthetic: PentaNAb3.0 10E8v4_S100cF-5X-35O22-5X-N6-5X-PGT121-5X-PGDM1400-His
  source 1..4221: mol_type = other DNA
gaattcgccgccaccatgggatggagctgtattattctgtttctggtcgctaccgctaccggagtgcattcttctgaactgacccaggaccccgccgtgagcgtggccctgaagcagaccgtgacaatcacctgcaggggcgacagcctgcgctcccactacgccagctggtatcagaagaagcctggccaggccccagtgctgctgttctacggcaagaacaataggccctccggcatccctgatcgcttttccggctctgccagcggaaacagggccagcctgacaatcaccggagcacaggcagaggacgaggcagattactattgcagctcccgggacaagtccggctctagactgagcgtgttcggcggcggcaccaagctgacagtgctgggaggaggaggcagcggcggaggaggctccggaggcggcggctctgaggtgcggctggtggagtctggaggaggcctggtgaagccaggaggcagcctgagactgagctgttccgcctctggcttcgactttgataatgcctggatgacatgggtgcggcagccacctggcaagggcctggagtgggtgggaagaatcaccggaccaggagagggatggtctgtggactacgccgagagcgtgaagggccggttcaccatctccagagataacaccaagaatacactgtatctggagatgaacaatgtgcggaccgaggacacaggctactatttctgcgccagaaccggcaagtactatgatttttggtttggctacccacccggcgaggagtattttcaggactggggccagggcaccctggtcatcgtgagcagcggcggcggcggcagcggcggcggcggctccggaggaggcggctctggaggaggaggcagcgggggagggggcagccagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctgggcggcggcggcagcgggggcgggggctccggagggggcggctctcagggccagctggtgcagagcggagcagagctgaagaagcctggagccagcgtgaagatctcctgtaagacatctggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctccggcggcgggggcagcggcggcgggggctccggaggaggaggctctggaggagggggcagcggaggaggcggctcctacatccacgtgacccagtccccatctagcctgtctgtgagcatcggcgatcgggtgaccatcaactgtcagacatctcagggcgtgggcagcgacctgcactggtatcagcacaagcctggcagggccccaaagctgctgatccaccacacatcctctgtggaggatggagtgccaagccgcttctccggctctggattccacacctcctttaatctgacaatctctgacctgcaggccgacgatatcgccacctactattgccaggtgctgcagttctttggccggggctccagactgcacatcaagggaggaggaggctccgggggcggaggctctggcggcggcggcagccgggcccacctggtgcagagcggcaccgccatgaagaagcctggcgccagcgtgagagtgtcctgtcagacatctggctacaccttcaccgcccacatcctgttctggtttaggcaggcaccaggaagaggcctggagtgggtgggctggatcaagccccagtatggagcagtgaacttcggaggaggctttcgggacagagtgacactgacccgggacgtgtacagagagatcgcctatatggatatcaggggcctgaagccagacgataccgccgtgtactattgcgccagggaccgctcctacggcgatagctcctgggcactggacgcatggggacagggcaccacagtggtggtgagcgccggcggcggaggctccggcggcgggggctctggaggaggcggcagcggagggggaggctccggagggggaggctctagcgacatctccgtggcccctggcgagacagccagaatctcttgtggcgagaagtctctgggcagcagggccgtgcagtggtaccagcacagggcaggacaggcaccatctctgatcatctataacaatcaggataggccaagcggcatccctgagcggttcagcggctcccccgacagcccttttggcaccacagccacactgaccatcacatccgtggaggcaggcgacgaagccgattactattgccacatctgggattccagagtgccaaccaagtgggtgttcggaggaggaaccacactgacagtgctgggaggggggggctctggcggcgggggcagcgggggaggaggctcccagatgcagctgcaggagagcggaccaggcctggtgaagcctagcgagacactgagcctgacatgttctgtgagcggcgcctccatctctgacagctactggtcttggatcagacggagccccggcaagggcctggaatggatcggctacgtgcacaagtccggcgatacaaactattccccatctctgaagtctcgggtgaacctgtctctggacaccagcaagaatcaggtgagcctgtccctggtggcagcaaccgcagcagatagcggcaagtactattgcgccagaacactgcacggcaggcgcatctacggcatcgtggcctttaacgagtggttcacctacttttatatggacgtgtggggcaatggcacccaggtgacagtgtcctctggcgggggcggctccggaggcggaggctctggcgggggcggcagcggcgggggcggctccgggggaggcggctctgatttcgtgctgacccagtctccacatagtctgagcgtgacacccggcgaaagcgcatcaatttcttgtaaatcatctcatagtctgatccacggcgataggaacaattacctggcctggtacgtgcagaagccaggccgcagccctcagctgctgatctacctggcaagctccagggcatccggagtgccagatcgcttctctggcagcggctccgataaggactttaccctgaagatctcccgggtggagacagaggacgtgggcacatactattgcatgcagggcagagagtctccttggaccttcggccagggcacaaaggtggacatcaagggaggaggaggcagcggcggaggaggctccggcggcggcggctctcaggcacagctggtgcagagcggaccagaggtgaggaagccaggcacctctgtgaaggtgagctgtaaggcccctggcaacaccctgaagacatacgatctgcactgggtgcggtctgtgccaggacagggcctgcagtggatgggatggatcagccacgagggcgacaagaaagtgatcgtggagcggtttaaggccaaggtgacaatcgattgggacagaagcaccaatacagcctatctgcagctgtccggcctgacctctggcgatacagccgtgtactattgcgccaagggctccaagcaccggctgagagactacgccctgtatgacgatgacggcgccctgaattgggcagtggacgtggactatctgagtaatctggagttttgggggcagggcaccgcagtgacagtgtctagcggatccggacaccaccatcaccatcattagtgaaagctt

SEQ ID NO: 85 - DNA, 4221 bp, synthetic construct
  misc_feature 1..4221: note = Synthetic: PentaNAb3.0 Reverse PGDM1400-5X-PGT121-5X-N6-5X-35O22-5X-10E8v4S100cF-His
  source 1..4221: mol_type = other DNA
gaattcgccgccaccatgggctggagctgcatcatcctgttcctggtggcaaccgcaacaggagtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagtcgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctggtggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatccggacaccaccatcaccatcattagtgaaagctt

SEQ ID NO: 86 - DNA, 4221 bp, synthetic construct
  misc_feature 1..4221: note = Synthetic: PentaNAb4.0 10E8v4 V5R_S100cF-5X-35O22-5X-N6-5X-PGT121-5X-PGDM1400-His
  source 1..4221: mol_type = other DNA
gaattcgccgccaccatgggatggagctgtattattctgtttctggtcgctaccgctaccggagtgcattcttctgaactgacccaggaccccgccgtgagcgtggccctgaagcagaccgtgacaatcacctgcaggggcgacagcctgcgctcccactacgccagctggtatcagaagaagcctggccaggccccagtgctgctgttctacggcaagaacaataggccctccggcatccctgatcgcttttccggctctgccagcggaaacagggccagcctgacaatcaccggagcacaggcagaggacgaggcagattactattgcagctcccgggacaagtccggctctagactgagcgtgttcggcggcggcaccaagctgacagtgctgggaggaggaggcagcggcggaggaggctccggaggcggcggctctgaggtgcggctgcgggagtctggaggaggcctggtgaagccaggaggcagcctgagactgagctgttccgcctctggcttcgactttgataatgcctggatgacatgggtgcggcagccacctggcaagggcctggagtgggtgggaagaatcaccggaccaggagagggatggtctgtggactacgccgagagcgtgaagggccggttcaccatctccagagataacaccaagaatacactgtatctggagatgaacaatgtgcggaccgaggacacaggctactatttctgcgccagaaccggcaagtactatgatttttggtttggctacccacccggcgaggagtattttcaggactggggccagggcaccctggtcatcgtgagcagcggcggcggcggcagcggcggcggcggctccggaggaggcggctctggaggaggaggcagcgggggagggggcagccagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctgggcggcggcggcagcgggggcgggggctccggagggggcggctctcagggccagctggtgcagagcggagcagagctgaagaagcctggagccagcgtgaagatctcctgtaagacatctggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctccggcggcgggggcagcggcggcgggggctccggaggaggaggctctggaggagggggcagcggaggaggcggctcctacatccacgtgacccagtccccatctagcctgtctgtgagcatcggcgatcgggtgaccatcaactgtcagacatctcagggcgtgggcagcgacctgcactggtatcagcacaagcctggcagggccccaaagctgctgatccaccacacatcctctgtggaggatggagtgccaagccgcttctccggctctggattccacacctcctttaatctgacaatctctgacctgcaggccgacgatatcgccacctactattgccaggtgctgcagttctttggccggggctccagactgcacatcaagggaggaggaggctccgggggcggaggctctggcggcggcggcagccgggcccacctggtgcagagcggcaccgccatgaagaagcctggcgccagcgtgagagtgtcctgtcagacatctggctacaccttcaccgcccacatcctgttctggtttaggcaggcaccaggaagaggcctggagtgggtgggctggatcaagccccagtatggagcagtgaacttcggaggaggctttcgggacagagtgacactgacccgggacgtgtacagagagatcgcctatatggatatcaggggcctgaagccagacgataccgccgtgtactattgcgccagggaccgctcctacggcgatagctcctgggcactggacgcatggggacagggcaccacagtggtggtgagcgccggcggcggaggctccggcggcgggggctctggaggaggcggcagcggagggggaggctccggagggggaggctctagcgacatctccgtggcccctggcgagacagccagaatctcttgtggcgagaagtctctgggcagcagggccgtgcagtggtaccagcacagggcaggacaggcaccatctctgatcatctataacaatcaggataggccaagcggcatccctgagcggttcagcggctcccccgacagcccttttggcaccacagccacactgaccatcacatccgtggaggcaggcgacgaagccgattactattgccacatctgggattccagagtgccaaccaagtgggtgttcggaggaggaaccacactgacagtgctgggaggggggggctctggcggcgggggcagcgggggaggaggctcccagatgcagctgcaggagagcggaccaggcctggtgaagcctagcgagacactgagcctgacatgttctgtgagcggcgcctccatctctgacagctactggtcttggatcagacggagccccggcaagggcctggaatggatcggctacgtgcacaagtccggcgatacaaactattccccatctctgaagtctcgggtgaacctgtctctggacaccagcaagaatcaggtgagcctgtccctggtggcagcaaccgcagcagatagcggcaagtactattgcgccagaacactgcacggcaggcgcatctacggcatcgtggcctttaacgagtggttcacctacttttatatggacgtgtggggcaatggcacccaggtgacagtgtcctctggcgggggcggctccggaggcggaggctctggcgggggcggcagcggcgggggcggctccgggggaggcggctctgatttcgtgctgacccagtctccacatagtctgagcgtgacacccggcgaaagcgcatcaatttcttgtaaatcatctcatagtctgatccacggcgataggaacaattacctggcctggtacgtgcagaagccaggccgcagccctcagctgctgatctacctggcaagctccagggcatccggagtgccagatcgcttctctggcagcggctccgataaggactttaccctgaagatctcccgggtggagacagaggacgtgggcacatactattgcatgcagggcagagagtctccttggaccttcggccagggcacaaaggtggacatcaagggaggaggaggcagcggcggaggaggctccggcggcggcggctctcaggcacagctggtgcagagcggaccagaggtgaggaagccaggcacctctgtgaaggtgagctgtaaggcccctggcaacaccctgaagacatacgatctgcactgggtgcggtctgtgccaggacagggcctgcagtggatgggatggatcagccacgagggcgacaagaaagtgatcgtggagcggtttaaggccaaggtgacaatcgattgggacagaagcaccaatacagcctatctgcagctgtccggcctgacctctggcgatacagccgtgtactattgcgccaagggctccaagcaccggctgagagactacgccctgtatgacgatgacggcgccctgaattgggcagtggacgtggactatctgagtaatctggagttttgggggcagggcaccgcagtgacagtgtctagcggatccggacaccaccatcaccatcattagtgaaagctt

SEQ ID NO: 87 - DNA, 4221 bp, synthetic construct
  misc_feature 1..4221: note = Synthetic: PentaNAb4.0 Reverse PGDM1400-5X-PGT121-5X-N6-5X-35O22-5X-10E8v4 V5R S100cF-His
  source 1..4221: mol_type = other DNA
gaattcgccgccaccatgggctggagctgcatcatcctgttcctggtggcaaccgcaacaggagtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatccggacaccaccatcaccatcattagtgaaagctt

SEQ ID NO: 88 - DNA, 4791 bp, synthetic construct
  misc_feature 1..4791: note = Synthetic: PentaNAb4.0 Reverse PGDM1400-5X-PGT121-5X-N6-5X-35O22-5X-10E8v4 V5R S100cFvVH-fused to IgG1 LS HC with His Tag
  source 1..4791: mol_type = other DNA
atgggctggagctgcatcatcctgttcctggtggcaaccgcaacaggagtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcgcgtcgaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaatga

SEQ ID NO: 89 - DNA, 4884 bp, synthetic construct
  misc_feature 1..4884: note = Synthetic: PentaNAb4.0 Reverse PGDM1400-5X-PGT121-5X-N6-5X-35O22-5X-10E8v4 V5R S100cF VH-VL-fused to IgG1 LS FC with His Tag
  source 1..4884: mol_type = other DNA
gaattcgccgccaccatgggctggagctgcatcatcctgttcctggtggcaaccgcaacaggagtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatcccccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaatga

SEQ ID NO: 90 - protein, 1398 aa, synthetic construct; identical to SEQ ID NO: 78
  REGION 1..1398: note = Synthetic: PentaNAb4.0 Reverse ScFv Amino acid sequence

SEQ ID NO: 91 - DNA, 4197 bp, synthetic construct
  misc_feature 1..4197: note = Synthetic: PentaNAb4.0 Reverse ScFv DNA sequence
  source 1..4197: mol_type = other DNA
atgggctggagctgcatcatcctgttcctggtggcaaccgcaacaggagtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatccggtcaccaccatcaccaccactag

SEQ ID NO: 92 - protein, 1667 aa, synthetic construct
  REGION 1..1667: note = Synthetic: HexaNAb1.0 Reverse ScFv (dB4C7/UB-421)-5 X-PGDM1400-5X-PGT121-5X-N6-5X-35O22-5X-10E8v4 V5R S100cF-His
MGWSCIILFLVATATGVHSDIVLTQSPASLAVSLGQRATITCKAGQSVDYDGDSYMNWYQQKPGQPPKLLIYVASNLESGIPARFSGSGSGTDFTLNIHPVEENDAATYYCQQSYKDPLTFGQGTKLEIKGGGGSGGGGSGGGGSQVQLVQSGPELKKPGASVKVSCKASGYTFTDYVIHWVKQATGQGLEWIGEIYPGSGSAYSNAKFKDRVTMTADKSSNTAYMELSSLTSDDTAVYFCARRGNGTGFAYWGQGTLVTVSSGGGGSGGGGSGGGGSGGGGSGGGGSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLRESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGSGHHHHHH

SEQ ID NO: 93 - DNA, 5004 bp, synthetic construct
  misc_feature 1..5004: note = Synthetic: HexaNAb1.0 Reverse ScFv DNA sequence
  source 1..5004: mol_type = other DNA
atgggctggagctgcatcatcctgttcctggtggcaaccgcaacaggagtgcacagcgacatcgtgctgacccagtctcctgccagcctggccgtgagcctgggacagagggccaccatcacatgcaaggccggccagtctgtggactacgatggcgacagctacatgaactggtatcagcagaagccaggccagccccctaagctgctgatctatgtggcctccaatctggagtctggcatccctgcacgcttcagcggctccggctctggaaccgacttcaccctgaacatccacccagtggaggagaatgacgccgccacctactattgccagcagagctacaaggaccccctgaccttcggccagggcacaaagctggagatcaagggaggaggaggcagcgggggaggaggctccggaggcggcggctctcaggtgcagctggtgcagtccggaccagagctgaagaagccaggagccagcgtgaaggtgtcctgtaaggcctctggctacaccttcacagattatgtgatccactgggtgaagcaggcaacaggacagggcctggagtggatcggagagatctacccaggcagcggctccgcctattccaacgccaagtttaaggatcgggtgaccatgacagccgacaagagctccaataccgcctatatggagctgtctagcctgacctctgacgatacagccgtgtacttctgtgcccggagaggcaacggcacaggctttgcctattggggccagggcaccctggtgacagtgtcctctggaggaggaggcagcggcggcggcggctccggaggaggcggctctggcggcggcggcagcggaggaggcggctcccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccggaggcgggggcagcggcggcgggggctccggaggcggaggctcttacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagggcggcggcggctctgggggcgggggcagcggcggggggggctccgggggaggaggctctggcggagggggcagccagggccagctggtgcagagcggagcagagctgaagaagccaggagcctctgtgaagatcagctgtaagacatccggctatcggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctgcgggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagcgtgttcggcggcggaaccaagctgacagtgctgggatccggtcaccaccatcaccaccactag

SEQ ID NO: 94 - protein, 1041 aa, synthetic construct
  REGION 1..1041: note = Synthetic: PentaNAb4.0 Stem HC LS_v2 Amino acid sequence
MGWSCIILFLVATATGVHSQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSSGGGGSGGGGSGGGGSDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKGGGGSGGGGSGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 95 - DNA, 3126 bp, synthetic construct
  misc_feature 1..3126: note = Synthetic: PentaNAb4.0 Stem HC LS_v2 DNA sequence
  source 1..3126: mol_type = other DNA
atgggctggagctgcatcatcctgttcctggtggcaaccgcaaccggtgtgcacagccaggcacagctggtgcagagcggacccgaagtgagaaaacctgggactagcgtcaaagtgtcatgtaaagcccctggaaataccctgaagacctacgatctgcactgggtgcggtccgtgcctggacagggcctgcagtggatgggatggatctctcacgagggcgacaagaaagtgatcgtggagcggttcaaggccaaggtgacaatcgattgggacagatccaccaacacagcctacctgcagctgtctggcctgaccagcggcgatacagccgtgtactactgtgccaagggctctaagcaccggctgagagactacgccctgtatgacgatgacggcgccctgaactgggccgtggatgtggactatctgtccaatctggagttctggggacagggaaccgcagtgacagtgagctccggaggaggaggctccggcggcggaggctctgggggaggcggcagcgattttgtgctgacccagtctccacacagcctgtccgtgacacccggcgagtctgccagcatctcctgcaagtctagccacagcctgatccacggcgacaggaacaattacctggcctggtacgtgcagaagccaggccgcagccctcagctgctgatctatctggcatcctctagggcctccggagtgccagatcgcttctctggcagcggctccgataaggactttaccctgaagatcagccgggtggagacagaggacgtgggcacatactattgtatgcagggccgagaatcaccttggacatttgggcagggaactaaagttgacatcaaagggggggggggctccggcggcgggggctctggcggcggcggcagcggaggaggcggctccggaggaggcggctctcagatgcagctgcaggagagcggaccaggactggtgaagccttccgagaccctgtctctgacatgttctgtgagcggcgcctccatctctgatagctactggagctggatcagacggagccctggcaagggcctggagtggatcggctacgtgcacaagtctggcgatacaaactattccccatctctgaagagccgggtgaacctgagcctggacacctccaagaatcaggtgagcctgtccctggtggcagcaaccgcagcagacagcggcaagtactattgcgccagaacactgcacggcaggcgcatctacggcatcgtggcctttaacgagtggttcacctacttttatatggacgtgtggggcaatggcacccaggtgacagtgtcctctggcggcggcggctctggcggaggaggcagcggaggaggaggcagctccgacatctctgtggcacctggagagaccgcaaggatcagctgtggagagaagtctctgggcagcagggccgtgcagtggtaccagcacagggcaggacaggcaccatccctgatcatctataacaatcaggaccggccatctggcatccccgagagattctctggcagccccgatagcccttttggcaccacagccaccctgacaatcacctccgtggaggccggcgacgaagcagattactattgccacatctgggactccagagtgccaaccaagtgggtgttcggaggaggaaccacactgacagtgctgggcggcggaggctccggcgggggcggctctggaggcggcggcagcggagggggcggctccggcggcggcggctctagggcacacctggtgcagagcggaaccgcaatgaagaagcctggcgcctctgtgcgcgtgagctgtcagacatccggctacaccttcaccgcccacatcctgttctggtttaggcaggcaccaggaagaggactggagtgggtgggctggatcaagccccagtatggagcagtgaacttcggaggaggctttcgggacagagtgacactgacccgggacgtgtacagagagatcgcctatatggatatcaggggcctgaagcccgacgataccgccgtgtactattgcgccagggaccgctcctacggcgattctagctgggcactggacgcatggggacagggaaccacagtggtggtgagcgccgcgtcgaccaagggcccatcggtcttccccctggcaccctcctccaagagcacctctgggggcacagcggccctgggctgcctggtcaaggactacttccccgaacctgtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaatga

SEQ ID NO: 96 - protein, 789 aa, synthetic construct
  REGION 1..789: note = Synthetic: PentaNAb4.0 Stem HC LS_v2 (light chain) N6 KC-CK-35O22-5X-10E8v4 S100cF
MGWSCIILFLVATATGVHSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVL

SEQ ID NO: 97 - DNA, 2334 bp, synthetic construct
  misc_feature 1..2334: note = Synthetic: LC: N6 KC-CK-35O22-5X-10E8v4 V5R S100cF DNA sequence
  source 1..2334: mol_type = other DNA
atgggctggagctgcatcatcctgttcctggtggcaaccgcaacaggagtgcacagctacatccacgtgacccagtccccttcctctctgtccgtgtctatcggcgatcgcgtgaccatcaactgtcagacaagccagggagtgggctccgacctgcactggtatcagcacaagcctggcagggccccaaagctgctgatccaccacacaagctccgtggaggatggagtgccaagccgcttcagcggctccggattccacaccagctttaatctgacaatctccgacctgcaggccgacgatatcgccacctactattgccaggtgctgcagttctttggcaggggctcccgcctgcacatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtggaggagggggctctggcggcgggggcagcggcggaggcggctccggagggggcggctctggcggaggcggcagccagggccagctggtgcagagcggagcagagctgaagaagccaggagcctctgtgaagatcagctgtaagacatccggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatccggaatctgaagttcgacgatacaggcacctatttttgcgccaagggcctgctgagagacggctctagcacatggctgccatacctgtggggacagggcaccctgctgacagtgtcctctggaggaggaggctccgggggcggcggctctggaggaggaggctctcaaagcgtgctgacccagtccgcctctgtgagcggctccctgggccagtctgtgaccatcagctgtacaggccccaactccgtgtgctgttctcacaagtctatcagctggtaccagtggccaccaggaagggcacctaccctgatcatctatgaggacaatgagagggcaccaggaatcagccctcgcttctccggctacaagtcttattggagcgcctacctgaccatttccgacctgcgccccgaggatgagaccacatactattgctgtagctatacccacaactccggctgcgtgtttggcacaggcaccaaggtgagcgtgctgggaggagggggctctggcggcgggggcagcggcggaggcggctccggagggggcggctctggcggaggcggcagcgaggtgcggctggtggagagcggcggcggcctggtgaagccaggcggctctctgagactgtcctgttctgccagcggcttcgactttgataatgcctggatgacatgggtgcggcagcctcctggcaaggggctggagtgggtgggaagaatcaccggaccaggagagggatggtctgtggactacgccgagagcgtgaagggccggttcaccatcagcagagataacactaaaaatacactgtatctggagatgaacaatgtgcggaccgaggacacaggctactatttctgcgccagaaccggcaagtactatgatttctggtttggctacccccctggcgaggagtattttcaggactggggccagggcaccctggtcatcgtgagcagcggcgggggaggctccggcggggggggctctggaggagggggctctagcgagctgacccaggaccccgccgtgtccgtggccctgaagcagacagtgaccatcacatgcaggggcgactccctgcgctctcactacgccagctggtatcagaagaagccaggacaggcacccgtgctgctgttctacggcaagaacaatcggccttccggcatcccagatagattttccggctctgccagcggaaacagggccagcctgaccatcacaggagcacaggcagaggatgaagcagattactattgttcctctcgggacaagtccggctctagactgagc

SEQ ID NO: 98 - protein, 118 aa, synthetic construct
  REGION 1..118: note = Synthetic: mAb dB4C7/UB-421s perfect heavy chain Fv region
QVQLVQSGPELKKPGASVKVSCKASGYTFTDYVIHWVKQATGQGLEWIGEIYPGSGSAYSNAKFKDRVTMTADKSSNTAYMELSSLTSDDTAVYFCARRGNGTGFAYWGQGTLVTVSS

SEQ ID NO: 99 - protein, 111 aa, synthetic construct
  REGION 1..111: note = Synthetic: mAb dB4C7/UB-421s perfect light chain Fv region
DIVLTQSPASLAVSLGQRATITCKAGQSVDYDGDSYMNWYQQKPGQPPKLLIYVASNLESGIPARFSGSGSGTDFTLNIHPVEENDAATYYCQQSYKDPLTFGQGTKLEIK

SEQ ID NO: 100 - protein, 5 aa, synthetic construct
  REGION 1..5: note = Synthetic: CDR1 of Heavy Chain of murine
DYVIH

SEQ ID NO: 101 - protein, 17 aa, synthetic construct
  REGION 1..17: note = Synthetic: CDR2 of Heavy Chain of murine
EIYPGSGSAYSNAKFKD

SEQ ID NO: 102 - protein, 9 aa, synthetic construct
  REGION 1..9: note = Synthetic: CDR3 of Heavy Chain of murine
RGNGTGFAY

SEQ ID NO: 103 - protein, 15 aa, synthetic construct
  REGION 1..15: note = Synthetic: CDR1 of Light Chain of murine
KAGQSVDYDGDSYMN

SEQ ID NO: 104 - protein, 7 aa, synthetic construct
  REGION 1..7: note = Synthetic: CDR2 of Light Chain of murine
VASNLES

SEQ ID NO: 105 - protein, 9 aa, synthetic construct
  REGION 1..9: note = Synthetic: CDR3 of Light Chain of murine
QQSYKDPLT

SEQ ID NO: 106 - DNA, 5025 bp, synthetic construct
  misc_feature 1..5025: note = Synthetic: PentaNAb2.0
  source 1..5025: mol_type = other DNA
atgggatggagctgtattattctgtttctggtcgctaccgctaccggagtgcattcttctgaactgacccaggaccccgccgtgagcgtggccctgaagcagaccgtgacaatcacctgcaggggcgacagcctgcgctcccactacgccagctggtatcagaagaagcctggccaggccccagtgctgctgttctacggcaagaacaataggccctccggcatccctgatcgcttttccggctctgccagcggaaacagggccagcctgacaatcaccggagcacaggcagaggacgaggcagattactattgcagctcccgggacaagtccggctctagactgagcgtgttcggcggcggcaccaagctgacagtgctgggaggaggaggcagcggcggaggaggctccggaggcggcggctctgaggtgcggctggtggagtctggaggaggcctggtgaagccaggaggcagcctgagactgagctgttccgcctctggcttcgactttgataatgcctggatgacatgggtgcggcagccacctggcaagggcctggagtgggtgggaagaatcaccggaccaggagagggatggtctgtggactacgccgagagcgtgaagggccggttcaccatctccagagataacaccaagaatacactgtatctggagatgaacaatgtgcggaccgaggacacaggctactatttctgcgccagaaccggcaagtactatgatttttggtttggctacccacccggcgaggagtattttcaggactggggccagggcaccctggtcatcgtgagcagcggcggcggcggcagcggcggcggcggctccggaggaggcggctctggaggaggaggcagcgggggagggggcagccagtccgtgctgacccagtctgccagcgtgtccggctctctgggacagagcgtgaccatctcctgtacaggccccaacagcgtgtgctgtagccacaagagcatctcctggtaccagtggcctccaggaagggcacctaccctgatcatctatgaggacaatgagcgggccccaggcatctcccccagattctctggctacaagtcttattggagcgcctacctgacaatcagcgacctgcgccccgaggatgagacaacatactattgctgttcctatacccacaactctggctgcgtgtttggcacaggcaccaaggtgtccgtgctgggcggcggcggcagcgggggcgggggctccggagggggcggctctcagggccagctggtgcagagcggagcagagctgaagaagcctggagccagcgtgaagatctcctgtaagacatctggctaccggttcaacttttatcacatcaattggatcaggcagaccgcaggaaggggaccagagtggatgggctggatctccccctactctggcgataagaacctggccccagccttccaggacagagtgatcatgaccacagataccgaggtgccagtgaccagcttcacctccaccggagccgcctacatggagatcaggaatctgaagttcgacgatacaggcacctatttttgcgcaaagggcctgctgagggacggctcctctacctggctgccttacctgtggggacagggcaccctgctgacagtgagctccggcggcgggggcagcggcggcgggggctccggaggaggaggctctggaggagggggcagcggaggaggcggctcctacatccacgtgacccagtccccatctagcctgtctgtgagcatcggcgatcgggtgaccatcaactgtcagacatctcagggcgtgggcagcgacctgcactggtatcagcacaagcctggcagggccccaaagctgctgatccaccacacatcctctgtggaggatggagtgccaagccgcttctccggctctggattccacacctcctttaatctgacaatctctgacctgcaggccgacgatatcgccacctactattgccaggtgctgcagttctttggccggggctccagactgcacatcaagggaggaggaggctccgggggcggaggctctggcggcggcggcagccgggcccacctggtgcagagcggcaccgccatgaagaagcctggcgccagcgtgagagtgtcctgtcagacatctggctacaccttcaccgcccacatcctgttctggtttaggcaggcaccaggaagaggcctggagtgggtgggctggatcaagccccagtatggagcagtgaacttcggaggaggctttcgggacagagtgacactgacccgggacgtgtacagagagatcgcctatatggatatcaggggcctgaagccagacgataccgccgtgtactattgcgccagggaccgctcctacggcgatagctcctgggcactggacgcatggggacagggcaccacagtggtggtgagcgccggcggcggaggctccggcggcgggggctctggaggaggcggcagcggagggggaggctccggagggggaggctctagcgacatctccgtggcccctggcgagacagccagaatctcttgtggcgagaagtctctgggcagcagggccgtgcagtggtaccagcacagggcaggacaggcaccatctctgatcatctataacaatcaggataggccaagcggcatccctgagcggttcagcggctcccccgacagcccttttggcaccacagccacactgaccatcacatccgtggaggcaggcgacgaagccgattactattgccacatctgggattccagagtgccaaccaagtgggtgttcggaggaggaaccacactgacagtgctgggaggggggggctctggcggcgggggcagcgggggaggaggctcccagatgcagctgcaggagagcggaccaggcctggtgaagcctagcgagacactgagcctgacatgttctgtgagcggcgcctccatctctgacagctactggtcttggatcagacggagccccggcaagggcctggaatggatcggctacgtgcacaagtccggcgatacaaactattccccatctctgaagtctcgggtgaacctgtctctggacaccagcaagaatcaggtgagcctgtccctggtggcagcaaccgcagcagatagcggcaagtactattgcgccagaacactgcacggcaggcgcatctacggcatcgtggcctttaacgagtggttcacctacttttatatggacgtgtggggcaatggcacccaggtgacagtgtcctctggcgggggcggctccggaggcggaggctctggcgggggcggcagcggcgggggcggctccgggggaggcggctctcagagcgtgctgacccagccaccttccgtgtctgccgcaccaggacagaaggtgaccatcagctgttccggcaacacatccaatatcggcaacaatttcgtgtcttggtaccagcagaggccaggaagggcaccacagctgctgatctatgagacagacaagcggccttccggcatcccagatagattttctgccagcaagtccggcaccagcggcacactggcaatcaccggcctgcagacaggcgacgaagctgattactattgcgcaacctgggcagcctccctgagctccgccagggtgttcggaaccggaacaaaagtgatcgtgctggtgggcggcggaggctctggcggaggcggcagcggcggggggggctcccaggtgcagctggtggagagcggcggcggcgtggtgcagccaggcacctccctgaggctgtcttgtgcagcaagccagttcagatttgatggctacggcatgcactgggtgcgccaggcaccaggcaagggcctggagtgggtagcctctatcagccacgacggcatcaagaagtaccacgccgagaaagtgtggggcaggttcaccatctcccgcgataactctaaaaacacactgtatctgcagatgaactccctgaggcccgaggacaccgccctgtactattgcgccaaggacctgcgcgaggatgagtgtgaggagtggtggtccgactactatgattttggcaagcagctgccttgcgcaaagagcaggggaggcctggtgggaatcgccgataattggggccagggcaccatggtgacagtgtctagcggatccggagagctgaagacccctctgggcgataccacacacacatccccacggagccccgagccaaagtcctctgacaccccaccccctagccctagatcccctgagccaaagagctccgatacaccacccccttctccaaggagccccgagcctaagtctagcgacaccccacccccttgcccccgctgtccagcaccagagctgctgggaggaccaagcgtgttcctgtttccacccaagcctaaggatacactgatgatctctcgcacccccgaggtgacatgcgtggtggtggacgtgagccacgaggaccccgaggtgaagttcaactggtacgtggacggcgtggaggtgcacaatgccaagaccaagcccagggaggagcagtacaacagcacctatcgcgtggtgtccgtgctgacagtgctgcaccaggactggctgaacggcaaggagtacaagtgcaaggtgtccaataaggccctgcctgccccaatcgagaagacaatcagcaaggcaaagggacagccaagggagccacaggtgtacaccctgcctccaagccgcgaggagatgaccaagaaccaggtgtccctgacatgtctggtgaagggcttctatcctagcgatatcgccgtggagtgggagtccaatggccagccagagaacaattacaagaccacaccccctgtgctggactccgatggctctttctttctgtattctaagctgaccgtggacaagagccggtggcagcagggcaacgtgttcagctgctctgtgctgcacgaagccctgcacagccattacacccagaagagcctgagcctgagccccggaaaa

SEQ ID NO: 107 - protein, 1675 aa, synthetic construct
  REGION 1..1675: note = Synthetic: PentaNAb2.0
MGWSCIILFLVATATGVHSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVLGGGGSGGGGSGGGGSEVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWFGYPPGEEYFQDWGQGTLVIVSSGGGGSGGGGSGGGGSGGGGSGGGGSQSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSVLGGGGSGGGGSGGGGSQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSSGGGGSGGGGSGGGGSGGGGSGGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKGGGGSGGGGSGGGGSRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGGSGGGGSGGGGSGGGGSGGGGSSDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGGGGSGGGGSGGGGSQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSGGGGSGGGGSGGGGSGGGGSGGGGSQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTKVIVLVGGGGSGGGGSGGGGSQVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGKQLPCAKSRGGLVGIADNWGQGTMVTVSSGSGELKTPLGDTTHTSPRSPEPKSSDTPPPSPRSPEPKSSDTPPPSPRSPEPKSSDTPPPCPRCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 108 - protein, 24 aa, synthetic construct
  REGION 1..24: note = Synthetic: membrane-proximal external region or MPER
LLELDKWASLWNWFBITNWLWYIK